(19) United States
(12) Patent Application Publication
(10) Pub. No.: US 2026/0035334 A1
(43) Pub. Date: Feb. 5, 2026

(54) METHOD OF SYNTHESIS

(71) Applicant: Beckley Psytech Limited, Oxford (GB)

(72) Inventor: Jason GRAY, Oxford (GB)

(21) Appl. No.: 19/295,404

(22) Filed: Aug. 8, 2025

Related U.S. Application Data
(63) Continuation-in-part of application No. 18/718,630, now abandoned.

(30) Foreign Application Priority Data
Dec. 13, 2021 (GB) 2118005.4
Dec. 14, 2021 (GB) 2118099.7
Dec. 15, 2021 (GB) 2118156.5
Aug. 19, 2022 (GB) 2212113.1
Aug. 19, 2022 (GB) 2212117.2

(51) Int. Cl. C07C 67/54

(52) U.S. Cl. CPC C07C 67/54

(57) ABSTRACT

A method of synthesizing the benzoate salt of 5-MeO-DMT comprising the step of treating the hydrochloride salt of 5-MeO-DMT with a base, prior to the addition of benzoic acid.

### FIELD OF THE INVENTION

This invention relates to methods of synthesis of the benzoate salt of 5-methoxy-N,N-dimethyltryptamine.

### BACKGROUND OF THE INVENTION

5-methoxy-N,N-dimethyltryptamine benzoate (hereafter 5-MeO-DMT benzoate) is the benzoate salt of the pharmacologically active compound of the tryptamine class, 5-MeO-DMT, and has the following structure:

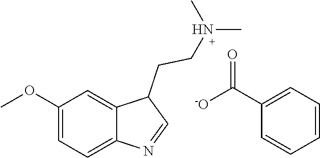

5-MeO-DMT is a psychoactive/psychedelic substance found in nature and is believed to act mainly through serotonin receptors. It is also believed to have a high affinity for the 5-HT₂ and 5-HT_{1A} subtypes, and/or inhibits monoamine reuptake.

There remains a need in the art for improved synthesis routes of 5-MeO-DMT benzoate.

### SUMMARY OF THE INVENTION

Herein disclosed, there is provided a method for the synthesis of 5-MeO-DMT benzoate.

In a first aspect of the invention there is provided a method of synthesizing the benzoate salt of 5-MeO-DMT comprising the step of treating the hydrochloride salt of 5-MeO-DMT with a base, prior to the addition of benzoic acid.

In an embodiment, the benzoate salt is crystalline.

In an embodiment, the benzoate salt is crystalline, as described subsequently.

In an embodiment, the benzoate salt is crystalline and conforms to Pattern A, B, C, D, E, F, G or H.

In an embodiment, the benzoate salt is crystalline Pattern A.

In an embodiment, the method comprises the step of suspending the hydrochloride salt in a suspending organic solvent; wherein optionally the suspending organic solvent is an alcohol, ester, an acetate and/or an acetate ester.

In an embodiment, the benzoic acid is in solution in an organic solvent; wherein optionally the organic solvent is an alcohol, ester, an acetate and/or an acetate ester.

In an embodiment, the benzoic acid and the hydrochloride salt are present in substantially equal molar amounts.

In an embodiment, the reaction with the benzoic acid takes place at an elevated temperature, optionally at a temperature between 40-65, 45-60, 50-55° C., or at/near the boiling point of the resultant reaction mixture. In an embodiment, the reaction of the hydrochloride salt with the benzoic acid takes place at an elevated temperature, optionally at a temperature between 40-65, 45-60, 50-55° C., or at/near the boiling point of the resultant reaction mixture. In an embodiment, the reaction of the hydrochloride salt with the benzoic acid takes place at an elevated temperature, and the resultant reaction mixture is allowed to cool to room temperature or lower, is allowed to cool to below 10° C., is allowed to cool to below 5° C., or is allowed to cool to between 5 and 0° C.

In an embodiment, the reaction with the benzoic acid takes place at an elevated temperature, and the resultant reaction mixture is allowed to cool to room temperature or lower, is allowed to cool to below 10° C., is allowed to cool to below 5° C., or is allowed to cool to between 5 and 0° C.

In an embodiment, the benzoate salt is filtered from the resultant reaction mixture.

In an embodiment, the filtered benzoate salt is washed with a washing organic solvent; wherein optionally the washing organic solvent is an alcohol, ester, an acetate and/or an acetate ester.

In an embodiment, the benzoate salt is washed with cooled washing organic solvent, optionally the washing organic solvent is cooled to below room temperature, is cooled to below 10° C., is cooled to below 5° C., or cooled to between 5 and 0° C.

In an embodiment, the filtered benzoate salt is dried under vacuum.

In an embodiment, the hydrochloride salt is base-treated with an aqueous basic solution, prior to the addition of benzoic acid.

In an embodiment, the hydrochloride salt is base-treated prior to the reaction with benzoic acid, optionally the hydrochloride salt is base-treated with an aqueous basic solution.

In an embodiment, the basic solution comprises an alkoxide, optionally the basic solution comprises sodium hydroxyl, further optionally the basic solution is 5% aqueous sodium hydroxyl.

In an embodiment, the hydrochloride salt is suspended in the suspending organic solvent prior to being base-treated, wherein optionally the suspending organic solvent is an alcohol, ester, an acetate and/or an acetate ester.

In an embodiment, the resultant based-treated reaction is partitioned with an extracting organic solvent to give an extract comprising the base-conditioned hydrochloride salt; wherein optionally the extracting organic solvent is an alcohol, ester, an acetate and/or an acetate ester.

In an embodiment, the suspending organic solvent is isopropyl acetate (IPAc).

In an embodiment, the organic solvent is IPAc.

In an embodiment, the washing organic solvent is IPAc.

In an embodiment, the extracting organic solvent is IPAc.

In an embodiment, the organic phase is washed with water.

In an embodiment, the extract is reduced under vacuum to give a concentrate, optionally the extract is concentrated to approximately 8 volumes.

In an embodiment, the extract is azeotropically dried with one or more batches of fresh extracting organic solvent, optionally the extracting organic solvent is IPAc.

In an embodiment, the method comprises the steps of:
combining 5-MeO-DMT hydrochloride salt and an organic solvent; optionally the organic solvent is IPAc
adding a basic solution to the combined 5-MeO-DMT hydrochloride salt and organic solvent;
optionally the basic solution is aqueous 5% NaOH;
Partitioning;
washing the resulting organic phase with water;
drying the solvent; optionally azeotropically with IPAc
concentrating under vacuum;
adjusting the solvent temperature to between about 50-55° C.;
adding a solution of benzoic acid in further organic solvent; optionally the further organic solvent is IPAc
adjusting the temperature to between about 0-5° C.;
filtering and washing with cold solvent; optionally the cold solvent is IPAc
drying under vacuum to obtain the 5-MeO-DMT benzoate salt as a crystalline solid.

In an embodiment, the crystalline 5-MeO-DMT benzoate produced is characterised by one or more of:
peaks in an XRPD diffractogram at 17.5, 17.7 and 21.0°2θ±0.1°2θ; and/or
endothermic event in a DSC thermograph having an onset temperature of between 12° and 130° C.;
and/or
enthalpy in a DSC thermograph of between −130 and −140 J/g; and/or
onset of decomposition in a TGA thermograph of between 128 and 135° C., between 129 and 134° C., between 13° and 133° C. or between 13° and 132° C.

Herein disclosed, there is provided a method for obtaining a purified mass of 5-MeO-DMT benzoate. In an embodiment, there is provided 5-MeO-DMT benzoate produced by the previously or subsequently described methods. In an embodiment, the source of 5-MeO-DMT hydrochloride is obtained as described in the Examples.

Herein disclosed, there is provided a purified mass of 5-MeO-DMT benzoate obtained from a source of 5-MeO-DMT hydrochloride.

In an embodiment, there is provided a purified mass of 5-MeO-DMT benzoate.

In an embodiment, there is provided a pure form of 5-MeO-DMT benzoate. In an embodiment, the purified mass contains no more than 0.1, 0.2, 0.25, 0.5, 1, 2, 3, 4 or 5% of any impurity.

In an embodiment, the purified mass is substantially free of the hydroxyl impurity shown below:

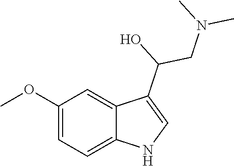

In an embodiment, the purified mass contains no more than 0.1, 0.2, 0.25, 0.5, 1, 2, 3, 4 or 5% of the hydroxyl impurity.

In an embodiment, the purified mass has a chemical purity of greater than 95, 96, 97, 98, 99 or 99.5%.

In an embodiment, the purified mass has a chemical purity of greater than 95, 96, 97, 98, 99 or 99.5% by HPLC or RP-HPLC. In an embodiment, the 5-MeO-DMT has chemical purity of greater than 95, 96, 97, 98, 99 or 99.5% by HPLC. In an embodiment, there is no single impurity of greater than 1%.

In an embodiment, the purified mass of 5-MeO-DMT benzoate is crystalline.

In an embodiment, there is provided >100 g of purified 5-MeO-DMT benzoate.

In an embodiment, there is provided >200 g of purified 5-MeO-DMT benzoate.

In an embodiment, there is provided >300 g of purified 5-MeO-DMT benzoate.

In an embodiment, there is provided >400 g of purified 5-MeO-DMT benzoate.

In an embodiment, there is provided >500 g of purified 5-MeO-DMT benzoate.

In an embodiment, there is provided a method of synthesizing >100 g, >200 g, >300 g, >400 g or >500 g of purified 5-MeO-DMT benzoate.

Herein disclosed, there is provided a method of synthesising a purified mass of 5-MeO-DMT benzoate. Herein disclosed, there is provided a method for the synthesis of 5-MeO-DMT benzoate.

In an embodiment, the method of synthesis comprises the step of treating the hydrochloride salt of 5-MeO-DMT with benzoic acid.

In an embodiment, the method of synthesis is a method of large scale synthesis.

In an embodiment, the method of synthesis is a method of synthesis of >100 g of 5-MeO-DMT benzoate.

In an embodiment, the method of synthesis is a method of synthesis of >200 g of 5-MeO-DMT benzoate.

In an embodiment, the method of synthesis is a method of synthesis of >300 g of 5-MeO-DMT benzoate.

In an embodiment, the method of synthesis is a method of synthesis of >400 g of 5-MeO-DMT benzoate.

In an embodiment, the method of synthesis is a method of synthesis of >500 g of 5-MeO-DMT benzoate.

In an embodiment, the method of synthesis is a method of recrystallization of >100 g of 5-MeO-DMT benzoate.

In an embodiment, the method of synthesis is a method of recrystallization of >200 g of 5-MeO-DMT benzoate.

In an embodiment, the method of synthesis is a method of recrystallization of >300 g of 5-MeO-DMT benzoate.

In an embodiment, the method of synthesis is a method of recrystallization of >400 g of 5-MeO-DMT benzoate.

In an embodiment, the method of synthesis is a method of recrystallization of >500 g of 5-MeO-DMT benzoate.

In an embodiment, the method of synthesis is a method of synthesis of an amorphous dry powder of 5-MeO-DMT benzoate.

In an embodiment, there is provided an amorphous dry powder of 5-MeO-DMT benzoate.

In an embodiment, there is provided a method of synthesis of 5-MeO-DMT benzoate wherein the 5-MeO-DMT benzoate is synthesised by reacting 5-MeO-DMT hydrochloride with a suitable solvent and benzoic acid.

Use of 5-MeO-DMT benzoate salt, produced by any of the methods described herein, in a method of medical treatment.

A method of synthesizing an organic acid salt of 5-MeO-DMT comprising the step of treating the hydrochloride salt of 5-MeO-DMT with the corresponding organic acid.

A method of synthesizing an organic acid salt of 5-MeO-DMT comprising the step of treating the hydrochloride salt of 5-MeO-DMT with the corresponding organic acid in an organic solvent.

A method of synthesizing an organic acid salt of 5-MeO-DMT comprising the step of treating the hydrochloride salt of 5-MeO-DMT with the corresponding organic acid in an organic solvent, wherein the resultant organic acid salt of 5-MeO-DMT is less soluble than the HCl salt of 5-MeO-DMT in the organic solvent.

A method of synthesizing an organic acid salt of 5-MeO-DMT comprising the step of treating the hydrochloride salt of 5-MeO-DMT with the corresponding organic acid in an organic solvent, wherein the HCl salt is placed in the organic solvent and the resultant organic acid salt of 5-MeO-DMT is less soluble in the organic solvent than the HCl salt of 5-MeO-DMT, and wherein the organic acid salt of 5-MeO-DMT remains in solution when the organic solvent is at elevated temperature, but falls out of solution when the reaction mixture is cooled.

A method of synthesizing an organic acid salt of 5-MeO-DMT, wherein the HCl salt is base-treated prior to the reaction with the organic acid, optionally the hydrochloride salt is base-treated with an aqueous basic solution, further optionally base-treated with aqueous NaOH (e.g. 5% NaOH).

In an embodiment the organic acid is selected from any of the known organic acids.

In an embodiment the organic acid is selected from: lactic acid, acetic acid, formic acid, citric acid, oxalic acid, uric acid, malic acid, benzoic acid or tartaric acid.

In an embodiment the organic acid is benzoic acid.

A method of recrystallizing the benzoate salt of 5-MeO-DMT from an organic solvent, wherein the solvent is selected from one or more of an alcohol, ester, an acetate and/or an acetate alcohol, ester, and optionally IPAc or IPA.

A method of purifying the HCl salt of 5-MeO-DMT, comprising the step of base-treating the HCl salt, optionally the hydrochloride salt is base-treated with an aqueous basic solution, further optionally base-treated with aqueous NaOH (e.g. 5% NaOH).

In an embodiment, the 5-MeO-DMT salt contains no more than 1% of the hydroxyl impurity, shown below:

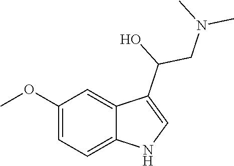

In an embodiment, the 5-MeO-DMT salt contains no more than 2% of the hydroxyl impurity. In an embodiment, the 5-MeO-DMT salt contains no more than 3% of the hydroxyl impurity. In an embodiment, the 5-MeO-DMT salt contains no more than 4% of the hydroxyl impurity. In an embodiment, the 5-MeO-DMT salt contains no more than 5% of the hydroxyl impurity.

In an embodiment, the 5-MeO-DMT HCl salt contains no more than 1% of the hydroxyl impurity. In an embodiment, the 5-MeO-DMT HCl salt contains no more than 2% of the hydroxyl impurity. In an embodiment, the 5-MeO-DMT HCl salt contains no more than 3% of the hydroxyl impurity. In an embodiment, the 5-MeO-DMT HCl salt contains no more than 4% of the hydroxyl impurity. In an embodiment, the 5-MeO-DMT HCl salt contains no more than 5% of the hydroxyl impurity.

In an embodiment, the 5-MeO-DMT benzoate salt contains no more than 1% of the hydroxyl impurity. In an embodiment, the 5-MeO-DMT benzoate salt contains no more than 2% of the hydroxyl impurity. In an embodiment, the 5-MeO-DMT benzoate salt contains no more than 3% of the hydroxyl impurity. In an embodiment, the 5-MeO-DMT benzoate salt contains no more than 4% of the hydroxyl impurity. In an embodiment, the 5-MeO-DMT benzoate salt contains no more than 5% of the hydroxyl impurity.

In an embodiment, the 5-MeO-DMT benzoate salt contains no more than 1% of any impurity. In an embodiment, the 5-MeO-DMT benzoate salt contains no more than 2% of any impurity. In an embodiment, the 5-MeO-DMT benzoate salt contains no more than 3% of any impurity. In an embodiment, the 5-MeO-DMT benzoate salt contains no more than 4% of any impurity. In an embodiment, the 5-MeO-DMT benzoate salt contains no more than 5% of any impurity.

In an embodiment, the 5-MeO-DMT benzoate synthesised by the methods of the invention is substantially free of the hydroxyl impurity.

In an embodiment, the 5-MeO-DMT benzoate synthesised by the methods of the invention contain no more than 1%, no more than 2%, no more than 3%, no more than 4% and/or no more than 5% of the hydroxyl impurity.

In an embodiment, purity of the 5-MeO-DMT is determined by HPLC or RP-HPLC.

In an embodiment, the 5-MeO-DMT has chemical purity of greater than 95, 96, 97, 98, 99 or 99.5% by HPLC.

In an embodiment, the 5-MeO-DMT has chemical purity of greater than 95, 96, 97, 98, 99 or 99.5% by RP-HPLC.

In an embodiment, there is no single impurity of greater than 1% by HPLC or RP-HPLC.

A method of synthesizing the benzoate salt of 5-MeO-DMT according to any one of the aspects or embodiments herein disclosed, wherein the hydrochloride salt is instead any non-benzoate salt, optionally the non-benzoate salt is a hydro-halide salt (e.g. the resultant counter ion is fluoride, chloride, bromide, iodide, fumarate, succinate, oxalate, acetate, citrate, triflate, phosphate, tartrate, benzenesulfonate, tosylate, adipate, glycolate, ketoglutarate, malate, saccharinate).

Herein disclosed, there is provided a composition comprising a pharmaceutically effective amount of a pharmaceutically acceptable salt of 5-methoxy-N,N-dimethyltryptamine (5-MeO-DMT).

In an embodiment, the salt anion is an aryl carboxylate. In an embodiment, the aryl carboxylate is substituted with one to three R groups.

In an embodiment the one or more R groups are independently selected from: alkynyl, carbonyl, aldehyde, haloformyl, alkyl, halide, hydroxy, alkoxy, carbonate ester, carboxylate, carboxyl, carboalkoxy, methoxy, hydroperoxy, peroxy, ether, hemiacetal, hemiketal, acetal, ketal, orthoester, methylenedioxy, orthocarbonate ester, carboxylic anhydride, carboxamide, secondary, tertiary or quaternary amine, primary or secondary ketimine, primary or secondary aldimine, imide, azide, azo, cyanate, isocyanate, nitrate, nitrile, isonitrile, nitrosooxy, nitro, nitroso, oxime, pyridyl, carbamate, sulfhydryl, sulfide, disulfide, sulfinyl, sulfonyl, sulfino, sulfo, thiocyanate, isothiocyanate, carbonothioyl, carbothioic S-acid, carbothioic O-acid, thiolester, thionoester, carbodithioic acid, carbodithio, phosphino, phosphono, phosphate, borono, boronate, borino or borinate.

In an embodiment the one or more R groups are independently selected from: C₁-C₆ alkyl, C₁-C₆ alkoxy, C₁-C₆ alkenyl or C₁-C₆ alkynyl, and where each of these may be optionally substituted with one to three R groups as previously described.

In a first aspect of the invention, there is provided a composition comprising a pharmaceutically effective amount of a pharmaceutically acceptable benzoate salt of 5-methoxy-N,N-dimethyltryptamine (5-MeO-DMT).

The invention provides for improved formulations and uses of 5-MeO-DMT salts.

In an embodiment the composition comprises a dosage amount of 5-MeO-DMT in the range of 0.05 mg to 100 mg.

In an embodiment the composition comprises a dosage amount of 5-MeO-DMT in the range of 0.1 mg to 50 mg.

In an embodiment the composition comprises a dosage amount of 5-MeO-DMT in the range of 0.5 mg to 25 mg.

In an embodiment the composition comprises a dosage amount of 5-MeO-DMT in the range of 0.5 mg to 10 mg.

In an embodiment the composition comprises a dosage amount of 5-MeO-DMT in the range of 1 mg to 10 mg.

In an embodiment the composition comprises a dosage amount of 5-MeO-DMT in the range of 1 mg to 8 mg.

In an embodiment the composition comprises a dosage amount of 5-MeO-DMT in the range of 3 mg to 15 mg.

In an embodiment the composition comprises a dosage amount of 5-MeO-DMT in the range of 0.005 mg to 100 mg.

In an embodiment the composition comprises a dosage amount of 5-MeO-DMT in the range of 0.001 mg to 100 mg.

In an embodiment the composition comprises a dosage amount of 5-MeO-DMT in the range of 0.0005 mg to 100 mg.

The level of the active agent can be adjusted as required by need for example to suit a certain patient group (e.g. the elderly) or the conditions being treated.

In an embodiment the composition is formulated in a dosage form selected from: oral, transdermal, inhalable, intravenous, or rectal dosage form.

It is advantageous to be able to deliver the active agent in different forms, for example to suit a certain patient group (e.g. the elderly) or the conditions being treated.

In an embodiment the composition is formulated in a dosage form selected from: tablet, capsule, granules, powder, free-flowing powder, inhalable powder, aerosol, nebulised, vaping, buccal, sublingual, sublabial, injectable, or suppository dosage form.

In an embodiment the powder is suitable for administration by inhalation via a medicament dispenser selected from a reservoir dry powder inhaler, a unit-dose dry powder inhaler, a pre-metered multi-dose dry powder inhaler, a nasal inhaler or a pressurized metered dose inhaler.

In an embodiment the powder comprises particles, the particles having a median diameter of less than 2000 μm, 1000 μm, 500 μm, 250 μm, 100 μm, 50 μm, or 1 μm.

In an embodiment the powder comprises particles, the particles having a median diameter of less than 15, 14, 13, 12, 11, or 10 μm.

In an embodiment the powder comprises particles, the particles having a median diameter of less than 9 μm.

In an embodiment the powder comprises particles, the particles having a median diameter of greater than 500 μm, 250 μm, 100 μm, 50 μm, 1 μm or 0.5 μm.

In an embodiment the powder comprises particles, and wherein the powder has a particle size distribution of d10=20-60 μm, and/or d50=80-120 μm, and/or d90=130-300 μm.

The nature of the powder can be adjusted to suit need. For example, if being made for nasal inhalation, then the particles may be adjusted to be much finer than if the powder is going to be formulated into a gelatine capsule, or differently again if it is going to be compacted into a tablet.

In an embodiment the 5-MeO-DMT salt is amorphous or crystalline.

In an embodiment the 5-MeO-DMT salt is in a polymorphic crystalline form, optionally 5-MeO-DMT salt is Polymorph A.

In an embodiment the 5-MeO-DMT salt is a benzoate, fumarate, citrate, acetate, succinate, halide, phosphate, tartrate, benzenesulfonate, tosylate, adipate, glycolate, ketoglutarate, malate, saccharinate, fluoride, chloride, bromide, iodide, oxalate, or triflate salt, optionally the salt is the chloride, benzoate or fumarate salt.

In an embodiment the 5-MeO-DMT salt is formulated into a composition for mucosal delivery. In an embodiment, the 5-MeO-DMT salt is a benzoate salt.

In an embodiment, the 5-MeO-DMT benzoate conforms to Pattern A as characterised by an XRPD diffractogram.

In an embodiment, the 5-MeO-DMT benzoate is characterised by peaks in an XRPD diffractogram at 17.5, 17.7 and 21.0°2θ±0.1°2θ as measured by x-ray powder diffraction using an x-ray wavelength of 1.5406 Å.

In an embodiment, 5-MeO-DMT benzoate is characterised by peaks in an XRPD diffractogram as substantially illustrated in FIG. 6 or FIG. 7.

In an embodiment, the 5-MeO-DMT benzoate is characterised by bands at ca. 3130, 1540, 1460, 1160 and 690 cm−1 in a Fourier-transform infrared spectroscopy (FTIR) spectra.

In an embodiment, the 5-MeO-DMT benzoate is characterised by a FTIR spectra for lot FP2 as substantially illustrated in FIG. 93.

In an embodiment, the 5-MeO-DMT benzoate conforms to Pattern B by XRPD.

In an embodiment, the 5-MeO-DMT benzoate conforms to Pattern B as characterised by peaks in an XRPD diffractogram between 18.5 and 20°2θ±0.1°2θ.

In an embodiment, the 5-MeO-DMT benzoate conforms to Pattern B as substantially illustrated by the XRPD diffractogram for lots P1, R1 and Q1 as substantially illustrated in FIG. 24.

In an embodiment, the 5-MeO-DMT benzoate conforms to Pattern B as substantially illustrated by the XRPD diffractogram for lot R2 as substantially illustrated in FIG. 28.

In an embodiment, the 5-MeO-DMT benzoate conforms to Pattern B as substantially illustrated by the XRPD diffractogram for lots A1 and B1 as substantially illustrated in FIG. 38 or 39.

In an embodiment, the 5-MeO-DMT benzoate corresponds to the Pattern B form as characterised by FTIR spectra for lot C2 as substantially illustrated in FIG. 93.

In an embodiment, the 5-MeO-DMT benzoate corresponds to the Pattern C form as characterised by a minor broad endotherm with a peak temperature of 108° C. in a DSC thermograph.

In an embodiment, the 5-MeO-DMT benzoate corresponds to Pattern C as characterised by a DSC thermograph as substantially illustrated in FIG. 65.

In an embodiment, the 5-MeO-DMT benzoate corresponds to the Pattern C form as characterised by a DSC thermograph as substantially illustrated in FIG. 66.

In an embodiment, the 5-MeO-DMT benzoate conforms to Pattern C by XRPD.

In an embodiment, the 5-MeO-DMT benzoate conforms to Pattern C as characterised by a peak in an XRPD diffractogram at 10.3°2θ±0.1°2θ.

In an embodiment, the 5-MeO-DMT benzoate conforms to Pattern C as substantially illustrated by the XRPD diffractogram for lot A1 as substantially illustrated in FIG. 68.

In an embodiment, the 5-MeO-DMT benzoate corresponds to the Pattern C form as characterised by FTIR spectra for lot C1 as substantially illustrated in FIG. 93.

In an embodiment, the 5-MeO-DMT benzoate conforms to Pattern D by XRPD.

In an embodiment, the 5-MeO-DMT benzoate conforms to Pattern D as substantially illustrated by the XRPD diffractogram in FIG. 73 or FIG. 74.

In an embodiment, the 5-MeO-DMT benzoate corresponds to Pattern D as characterised by an endothermic event in a DSC thermograph at 118° C.

In an embodiment, the 5-MeO-DMT benzoate corresponds to the Pattern D form as characterised by an endothermic event in a DSC thermograph at 118.58° C.

In an embodiment, the 5-MeO-DMT benzoate conforms to Pattern E by XRPD.

In an embodiment, the 5-MeO-DMT benzoate corresponds to Pattern E as substantially illustrated by the XRPD diffractogram for lot D in FIG. 77 or FIG. 78.

In an embodiment, the 5-MeO-DMT corresponds to the Pattern E form as characterised by a major bimodal endothermic event with peak temperatures of 110.31° C. and 113.13° C. in a DSC thermograph.

In an embodiment, the 5-MeO-DMT corresponds to Pattern E as characterised by a minor endothermic event with a peak temperature of 119.09° C. in a DSC thermograph.

In an embodiment, the 5-MeO-DMT corresponds to the Pattern E form as characterised by a DSC thermograph as substantially illustrated in FIG. 79.

In an embodiment, the 5-MeO-DMT benzoate corresponds to Pattern E as substantially illustrated by the XRPD diffractogram in FIG. 80.

In an embodiment, the 5-MeO-DMT benzoate corresponds to Pattern F by XRPD.

In an embodiment, the 5-MeO-DMT benzoate conforms to Pattern F as characterised by an XRPD diffractogram for lot F (rerun) as substantially illustrated in FIG. 84.

In an embodiment, the 5-MeO-DMT benzoate conforms to Pattern F as characterised by an XRPD diffractogram for lot F (rerun) as substantially illustrated in FIG. 85.

In an embodiment, the 5-MeO-DMT benzoate conforms to Pattern F as characterised by an XRPD diffractogram for lot F (rerun) as substantially illustrated in FIG. 89.

In an embodiment, the 5-MeO-DMT benzoate corresponds to the Pattern F form as characterised by endothermic events at 90° C., 106° C. and 180° C. in a DSC thermograph.

In an embodiment, the 5-MeO-DMT benzoate corresponds to the Pattern F form as characterised by endothermic events at 90.50° C., 106.65° C. and 180.35° C. in a DSC thermograph.

In an embodiment, the 5-MeO-DMT benzoate conforms to Pattern G by XRPD.

In an embodiment, the 5-MeO-DMT benzoate conforms to Pattern G, as characterised by an XRPD diffractogram for lot K as substantially illustrated in FIG. 87.

In an embodiment, the 5-MeO-DMT benzoate corresponds to the Pattern G form, as characterised by an endothermic event in a DSC thermograph of 119.61° C.

In an embodiment, the composition comprises 5-MeO-DMT benzoate which conforms to a mixture of two or more of Patterns A to G by XRPD.

In another aspect, there is provided a purified mass of 5-MeO-DMT hydrochloride obtained from a source of 5-MeO-DMT hydrochloride.

In some embodiments, the mass is greater than 0.5, 1, 2, 5, 10, 20, 50, 100, 250, 500 or 1000 grams. In some embodiments, the purified mass contains no more than 0.1, 0.2, 0.25, 0.5, 1, 2, 3, 4 or 5% of any impurity. In some embodiments, the purified mass is substantially free of the hydroxyl impurity shown below:

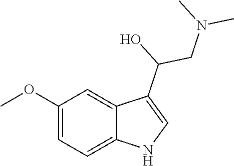

In some embodiments, wherein the purified mass contains no more than 0.1, 0.2, 0.25, 0.5, 1, 2, 3, 4 or 5% of the hydroxyl impurity. In some embodiments, the purified mass has a chemical purity of greater than 95, 96, 97, 98, 99 or 99.5% by HPLC or RP-HPLC.

In some embodiments, the purified mass of 5-MeO-DMT hydrochloride is crystalline. In some embodiments, wherein the purified mass is characterised by one or more of:
peaks in an XRPD diffractogram at 9.2, 12.2, 14.1, 15.0, 18.5, 19.5, 23.9, 24.5, 25.1, 26.0, 26.9 and 28.3°2θ±0.1°2θ as measured by x-ray powder diffraction using an x-ray wavelength of 1.5406 Å;
endothermic event in a DSC thermograph having an onset temperature of between 14° and 150° C. and/or a peak of between 142 and 148° C.;
enthalpy in a DSC thermograph of between 113 J/g and −123 J/g;
onset of decomposition in a TGA thermograph of between 12° and 165° C.

In some embodiments, a source of 5-MeO-DMT hydrochloride is base-treated, optionally the source of 5-MeO-DMT hydrochloride is base-treated with an aqueous basic solution. In some embodiments, the basic solution comprises an alkoxide, optionally the basic solution comprises sodium hydroxide, further optionally the basic solution is 5% aqueous sodium hydroxide. In some embodiments, the source of 5-MeO-DMT hydrochloride is suspended in a suspending organic solvent prior to being base-treated, wherein optionally the suspending organic solvent is an alcohol, ester, an acetate and/or an acetate ester.

In some embodiments, the resultant base-treated reaction is partitioned with an extracting organic solvent to give an extract comprising the base-conditioned hydrochloride salt; wherein optionally the extracting organic solvent is an alcohol, ester, an acetate and/or an acetate ester. In some embodiments, the extract is reduced under vacuum to give a concentrate of the purified mass of 5-MeO-DMT hydrochloride, optionally the extract is concentrated to approximately 8 volumes, or further optionally the solvent is removed to give the purified mass of 5-MeO-DMT hydrochloride in a solid form. In some embodiments, the extract or solid form of the purified mass of 5-MeO-DMT hydrochloride is azeotropically dried with one or more batches of fresh extracting organic solvent wherein optionally the extracting organic solvent is an alcohol, ester, an acetate and/or an acetate ester. In some embodiments, the purified mass of 5-MeO-DMT hydrochloride is obtained by filtration.

In some embodiments, the filtered purified mass of 5-MeO-DMT hydrochloride is washed with a washing organic solvent. In some embodiments, the purified mass of 5-MeO-DMT hydrochloride is washed with cooled washing organic solvent, optionally the washing organic solvent is cooled to below room temperature, is cooled to below 10° C., is cooled to below 5° C., or cooled to between 5 and 0° C. In some embodiments, the purified mass of 5-MeO-DMT hydrochloride is dried under vacuum. In some embodiments, the suspending organic solvent, the washing organic solvent, and/or the extracting organic solvent is IPAc.

In some embodiments, the purified mass of 5-MeO-DMT hydrochloride is isolated and subjected to a recrystallisation process.

In some embodiments, the method comprises the steps of:
combining the source of 5-MeO-DMT hydrochloride and an organic solvent; optionally the organic solvent is IPAc
adding a basic solution to the combined 5-MeO-DMT hydrochloride salt and organic solvent;
optionally the basic solution is aqueous 5% NaOH;
partitioning;
washing the resulting organic phase with water;
drying the solvent; optionally azeotropically with IPAc; and
concentrating under vacuum.

In some embodiments, the method further comprises the steps of concentrating under vacuum to dryness. In some embodiments, the method further comprises the steps of:
adjusting the solvent temperature to between about 50-55° C.;
adding one or more counter solvents in which the 5-MeO-DMT hydrochloride is substantially insoluble in; and/or adjusting the temperature to between about 0-5° C.;
filtering and washing with cold solvent; optionally the cold solvent is IPAc; and
drying under vacuum to obtain the purified mass of 5-MeO-DMT hydrochloride in a solid form, optionally as a crystalline solid.

In some embodiments, the purified mass of 5-MeO-DMT hydrochloride produced is characterised by one or more of:
peaks in an XRPD diffractogram at 9.2, 12.2, 14.1, 15.0, 18.5, 19.5, 23.9, 24.5, 25.1, 26.0, 26.9 and 28.3°2θ±0.1°2θ as measured by x-ray powder diffraction using an x-ray wavelength of 1.5406 Å;
endothermic event in a DSC thermograph having an onset temperature of between 14° and 150° C. and/or a peak of between 142 and 148° C.;
enthalpy in a DSC thermograph of between 113 J/g and −123 J/g;
onset of decomposition in a TGA thermograph of between 12° and 165° C.

In some embodiments, the purified mass of 5-MeO-DMT hydrochloride is obtained by any of the above methods.

In an aspect, there is an inorganic or organic acid salt of 5-MeO-DMT obtained by the step of treating the purified mass of 5-MeO-DMT hydrochloride as defined in any one of the above methods, with a base, prior to the addition of an inorganic acid or organic acid; wherein the resultant counter anion is the deprotonated form of the acid used, wherein optionally the resultant counter anion is a fluoride, bromide, iodide, fumarate, acetate, succinate, oxalate, acetate, citrate, phosphate, tartrate, benzenesulfonate, tosylate, adipate, glycolate, ketoglutarate, malate, saccharinate, triflate or benzoate anion; further optionally the anion is the benzoate.

For the salt, the dosage amount is the equivalent amount of the free base delivered when the salt is taken. So 100 mg dosage amount of 5-MeO-DMT corresponds to 117 mg of the hydrochloride salt (i.e. both providing the same molar amount of the active substance). The greater mass of the salt needed is due to the larger formula weight of the hydrogen chloride salt (i.e. 218.3 g/mol for the free base as compared to 254.8 g/mol for the salt). Similarly, for the deuterated or triturated version of 5-MeO-DMT (also considered within the scope of the invention), a slight increase in mass can be expected due to the increased formula weight of these isotopic compounds.

Amorphous and crystalline substances often show different chemical/physical properties, e.g. improved rate of dissolution in a solvent, or improved thermal stability. Similarly, different polymorphs may also show different and useful chemical/physical properties.

In an embodiment the composition comprises one or more pharmaceutically acceptable carriers or excipients.

In an embodiment the composition comprises one or more of: mucoadhesive enhancer, penetrating enhancer, cationic polymers, cyclodextrins, Tight Junction Modulators, enzyme inhibitors, surfactants, chelators, and polysaccharides.

In an embodiment the composition comprises one or more of: chitosan, chitosan derivatives (such as N,N,N-trimethyl chitosan (TMC), n-propyl-(QuatPropyl), n-butyl-(QuatButyl) and n-hexyl (QuatHexyl)-N,N-dimethyl chitosan, chitosan chloride), β-cyclodextrin, Clostridium perfringens enterotoxin, zonula occludens toxin (ZOT), human neutrophil elastase inhibitor (ER143), sodium taurocholate, sodium deoxycholate sodium, sodium lauryl sulphate, glycodeoxycholat, palmitic acid, palmitoleic acid, stearic acid, oleyl acid, oleyl alcohol, capric acid sodium salt, DHA, EPA, dipalmitoyl phophatidyl choline, soybean lecithin, lysophosphatidylcholine, dodecyl maltoside, tetradecyl maltoside, EDTA, lactose, cellulose, and citric acid.

In an embodiment the composition disclosed herein is for use as a medicament. In an embodiment the composition disclosed herein is for use in a method of treatment of a human or animal subject by therapy.

In an embodiment the method of treatment is a method of treatment of:
conditions caused by dysfunctions of the central nervous system,
conditions caused by dysfunctions of the peripheral nervous system,
conditions benefiting from sleep regulation (such as insomnia),
conditions benefiting from analgesics (such as chronic pain), migraines,
trigeminal autonomic cephalgias (such as short-lasting unilateral neuralgiform headache with conjunctival injection and tearing (SUNCT), and short-lasting neuralgiform headaches with cranial autonomic symptoms (SUNA)),
conditions benefiting from neurogenesis (such as stroke, traumatic brain injury, Parkinson's dementia),
conditions benefiting from anti-inflammatory treatment,
depression,
treatment resistant depression
anxiety,
substance use disorder,
addictive disorder,
gambling disorder,
eating disorders,
obsessive-compulsive disorders, or
body dysmorphic disorders,
optionally the condition is SUNCT and/or SUNA.

Treatment of the above conditions may be beneficially improved by taking the invention.

In an embodiment, the method of treatment is a method of treatment of alcohol-related diseases and disorders, eating disorders, impulse control disorders, nicotine-related disorders, tobacco-related disorders, methamphetamine-related disorders, amphetamine-related disorders, cannabis-related disorders, cocaine-related disorders, hallucinogen use disorders, inhalant-related disorders, benzodiazepine abuse or dependence related disorders, and/or opioid-related disorders.

In an embodiment, the method of treatment is a method of treatment of tobacco addiction. In an embodiment, the method is a method of reducing tobacco use. In an embodiment, the method of treatment is a method of treatment of nicotine addiction. In an embodiment, the method is a method of reducing nicotine use.

In an embodiment, the method of treatment is a method of treating alcohol abuse and/or addiction. In an embodiment, the method of treatment is a method of reducing alcohol use.

In an embodiment, the method of treatment is a method of treating or preventing heavy drug use.

In an embodiment, the method of treatment is a method of treating or preventing heavy drug use, including, but not limited to, alcohol, tobacco, nicotine, cocaine, methamphetamine, other stimulants, phencyclidine, other hallucinogens, marijuana, sedatives, tranquilizers, hypnotics, and opiates. It will be appreciated by one of ordinary skill in the art that heavy use or abuse of a substance does not necessarily mean the subject is dependent on the substance.

In an embodiment the method of treatment is a method of treatment of more than one of the above conditions, for example, the method of treatment may be a method of treatment of depression and anxiety.

In an embodiment the composition is administered one or more times a year.

In an embodiment the composition is administered one or more times a month.

In an embodiment the composition is administered one or more times a week.

In an embodiment the composition is administered one or more times a day.

In an embodiment the composition is administered at such a frequency as to avoid tachyphylaxis.

In an embodiment the composition is administered together with a complementary treatment and/or with a further active agent.

In an embodiment the further active agent is a psychedelic compound, optionally a tryptamine.

In an embodiment the further active agent is lysergic acid diethylamide (LSD), psilocybin, psilocin or a prodrug thereof.

In an embodiment the further active agent is an antidepressant compound.

In an embodiment the further active agent is selected from an SSRI, SNRI, TCA or other antidepressant compounds.

In an embodiment the further active agent is selected from Citalopram (Celexa, Cipramil), Escitalopram (Lexapro, Cipralex), Fluoxetine (Prozac, Sarafem), Fluvoxamine (Luvox, Faverin), Paroxetine (Paxil, Seroxat), Sertraline (Zoloft, Lustral), Desvenlafaxine (Pristiq), Duloxetine (Cymbalta), Levomilnacipran (Fetzima), Milnacipran (Ixel, Savella), Venlafaxine (Effexor), Vilazodone (Viibryd), Vortioxetine (Trintellix), Nefazodone (Dutonin, Nefadar, Serzone), Trazodone (Desyrel), Reboxetine (Edronax), Teniloxazine (Lucelan, Metatone), Viloxazine (Vivalan), Bupropion (Wellbutrin), Amitriptyline (Elavil, Endep), Amitriptylinoxide (Amioxid, Ambivalon, Equilibrin), Clomipramine (Anafranil), Desipramine (Norpramin, Pertofrane), Dibenzepin (Noveril, Victoril), Dimetacrine (Istonil), Dosulepin (Prothiaden), Doxepin (Adapin, Sinequan), Imipramine (Tofranil), Lofepramine (Lomont, Gamanil), Melitracen (Dixeran, Melixeran, Trausabun), Nitroxazepine (Sintamil), Nortriptyline (Pamelor, Aventyl), Noxiptiline (Agedal, Elronon, Nogedal), Opipramol (Insidon), Pipofezine (Azafen/Azaphen), Protriptyline (Vivactil), Trimipramine (Surmontil), Amoxapine (Asendin), Maprotiline (Ludiomil), Mianserin (Tolvon), Mirtazapine (Remeron), Setiptiline (Tecipul), Isocarboxazid (Marplan), Phenelzine (Nardil), Tranylcypromine (Parnate), Selegiline (Eldepryl, Zelapar, Emsam), Caroxazone (Surodil, Timostenil), Metralindole (Inkazan), Moclobemide (Aurorix, Manerix), Pirlindole (Pirazidol), Toloxatone (Humoryl), Agomelatine (Valdoxan), Esketamine (Spravato), Ketamine (Ketalar), Tandospirone (Sediel), Tianeptine (Stablon, Coaxil), Amisulpride (Solian), Aripiprazole (Abilify), Brexpiprazole (Rexulti), Lurasidone (Latuda), Olanzapine (Zyprexa), Quetiapine (Seroquel), Risperidone (Risperdal), Trifluoperazine (Stelazine), Buspirone (Buspar), Lithium (Eskalith, Lithobid), Modafinil (Provigil), Thyroxine (T4), Triiodothyronine (T3).

In an embodiment the further active agent is selected from Celexa (citalopram), Cymbalta (duloxetine), Effexor (venlafaxine), Lexapro (escitalopram), Luvox (fluvoxamine), Paxil (paroxetine), Prozac (fluoxetine), Remeron (mirtazapine), Savella (milnacipran), Trintellix (vortioxetine), Vestra (reboxetine), Viibryd (vilazodone), Wellbutrin (bupropion), Zoloft (sertraline).

In an embodiment the complementary treatment is psychotherapy.

In an embodiment, there is provided a composition comprising a pharmaceutically effective amount of a pharmaceutically acceptable benzoate salt of 5-MeO-DMT for use in a method of treatment of treatment resistant depression.

In an embodiment, there is provided a composition comprising a pharmaceutically effective amount of a pharmaceutically acceptable benzoate salt of 5-MeO-DMT for use in a method of treatment of depression.

In an embodiment, there is provided a composition comprising a pharmaceutically effective amount of a pharmaceutically acceptable benzoate salt of 5-MeO-DMT for use in a method of treatment of PTSD.

In an embodiment, there is provided a composition comprising a pharmaceutically effective amount of a pharmaceutically acceptable benzoate salt of 5-MeO-DMT for use in a method of treatment of addiction/substance misuse disorders.

In an embodiment, there is provided a nasal inhalation composition comprising a pharmaceutically effective amount of a pharmaceutically acceptable benzoate salt of 5-MeO-DMT for use in a method of treatment of treatment resistant depression.

Treatment of the above conditions may be beneficially improved by taking the invention together with some complementary treatments; also these treatments may occur much less regularly than some other treatments that require daily treatments or even multiple treatments a day.

For the sake of brevity only, various forms of the 5-MeO-DMT benzoate salt may be referred to herein below as ‘Pattern #’, wherein the # refers to the corresponding XRPD pattern obtained for that form. For example ‘Pattern A’ may be used as an abbreviation to refer to the form of 5-MeO-DMT benzoate salt giving rise to the Pattern A by XRPD. Likewise, ‘Pattern B’ may be used as an abbreviation to refer to the form of 5-MeO-DMT benzoate salt giving rise to the Pattern B by XRPD, and so on.

The present invention will now be further described with reference to the following, and the accompanying drawings, of which:

### BRIEF DESCRIPTION OF THE DRAWINGS

FIG. 1 is a schematic route for the synthesis of 5-MeO-DMT.

FIG. 2 is a further schematic route for the synthesis of 5-MeO-DMT.

FIG. 3 is a schematic route for the preparation of a powder form of 5-MeO-DMT.

FIG. 4 is an overview of the slug mucosal irritation (SMI) test. (A) First 15 minute contact period between slug and test item. (B) Slug is transferred onto a wet paper towel in a new Petri dish for 1 hour. (C) Second 15 minute contact period between slug and test item. (D) Slug is transferred onto a wet paper towel in a new Petri dish for 1 hour. (E) Third 15 minute contact period between slug and test item.

FIG. 5 is a graph showing that the benzoate salt of 5-MeO-DMT has higher permeation compared with the hydrochloride salt, as per the experiment detailed in Example 9.

FIG. 6 shows an XRPD diffractogram of 5-MeO-DMT benzoate prior to particle size reduction.

FIG. 7 shows an XRPD diffractogram of 5-MeO-DMT benzoate following particle size reduction.

FIG. 8 shows the XRPD diffractograms of FIGS. 6 and 7 overlaid on one another.

FIG. 9 shows a DSC thermograph of 5-MeO-DMT benzoate.

FIG. 10 shows a TGA thermograph of 5-MeO-DMT benzoate.

FIG. 11 shows a combined TGA/DSC thermograph of 5-MeO-DMT benzoate.

FIG. 12 shows a Dynamic Vapour Sorption (DVS) isotherm for 5-MeO-DMT benzoate.

FIG. 13 shows an optical micrograph of 5-MeO-DMT benzoate salt (A) and dark field (B) at ×4 magnification.

FIG. 14 shows two further optical micrographs of 5-MeO-DMT benzoate salt (A) and (B) at ×4 magnification.

FIG. 15 shows optical micrographs of 5-MeO-DMT benzoate salt (A) and (B) at ×10 magnification.

FIG. 16 shows further optical micrographs of 5-MeO-DMT benzoate salt (A) and (B) at 10× magnification.

FIG. 17 shows a DVS isotherm of 5-MeO-DMT hydrochloride (lot 20/20/126-FP).

FIG. 18 shows a DVS isotherm of 5-MeO-DMT hydrochloride (lot 20/45/006-FP).

FIG. 19 shows XRPD pattern comparison of two different lots of 5-MeO-DMT benzoate.

FIG. 20 shows a DSC thermograph of another lot of 5-MeO-DMT benzoate.

FIG. 21 shows additional XRPD characterisation of multiple lots of 5-MeO-DMT benzoate.

FIG. 22 shows DSC thermograph results for 5-MeO-DMT benzoate lots C1, D1 and E1.

FIG. 23 shows TGA thermograph results for 5-MeO-DMT benzoate lots C1, D1 and E1 at 10° C.min⁻¹.

FIG. 24 shows XRPD pattern comparison of 5-MeO-DMT benzoate P1 (Toluene), Q1 (Chlorobenzene), and R1 (Anisole) against the XRPD pattern of Pattern A.

FIG. 25 shows DSC thermographs of 5-MeO-DMT lots P1, Q1 and R1 at 10° C.min⁻¹.

FIG. 26 shows DSC thermograph expansions of 5-MeO-DMT lots P1, Q1 and R1 at 10° C.min⁻¹.

FIG. 27 shows TGA thermographs of 5-MeO-DMT lots P1, Q1 and R1 at 10° C.min⁻¹.

FIG. 28 shows XRPD pattern comparison of 5-MeO-DMT benzoate lots R1 and R2 (thermally cycled suspensions) compared with a reference Pattern A XRPD diffractogram.

FIG. 29 shows DSC thermographs of 5-MeO-DMT benzoate lots P2, Q2 and R2 at 10° C.min⁻¹.

FIG. 30 shows DSC thermograph expansions of 5-MeO-DMT benzoate lots P2, Q2 and R2 at 10° C.min⁻¹.

FIG. 31 shows TGA thermographs of 5-MeO-DMT benzoate lots P2, Q2 and R2 at 10° C.min⁻¹.

FIG. 32 shows XRPD pattern overlay of samples isolated via anti-solvent mediated crystallisation 5-MeO-DMT benzoate.

FIG. 33 shows XRPD pattern overlay of 5-MeO-DMT benzoate lot F1 and a reference Pattern A form/material.

FIG. 34 shows XRPD pattern overlay of 5-MeO-DMT benzoate samples isolated from cooling and a Pattern A reference.

FIG. 35 shows XRPD pattern overlay of 5-MeO-DMT benzoate samples isolated from cooling post-particle size reduction and Pattern A reference.

FIG. 36 shows XRPD pattern comparison for all samples from the reverse addition anti-solvent driven crystallisation of 5-MeO-DMT benzoate except for A1 and B1.

FIG. 37 shows XRPD pattern comparison for 5-MeO-DMT benzoate F3 with a known Pattern A reference.

FIG. 38 shows XRPD pattern comparison of 5-MeO-DMT benzoate A1 and B1.

FIG. 39 shows XRPD patterns for 5-MeO-DMT benzoate A1, Q1 and a reference Pattern A pattern.

FIG. 40 shows XRPD patterns for 5-MeO-DMT benzoate B1, Q1 and a reference Pattern A pattern.

FIG. 41 shows a DSC thermograph of 5-MeO-DMT benzoate sample A1 at 10° C.min⁻¹ isolated from methanol and toluene.

FIG. 42 shows a DSC thermograph of 5-MeO-DMT benzoate B1 at 10° C.min⁻¹ isolated from isopropanol and toluene.

FIG. 43 shows a DSC thermograph expansion of 5-MeO-DMT benzoate. B1 at 10° C.min⁻¹ isolated from isopropanol and toluene.

FIG. 44 shows XRPD comparison of 5-MeO-DMT benzoate lot 21-01-051 A, E; E Particle size reduced and Pattern A reference.

FIG. 45 shows XRPD of 5-MeO-DMT benzoate lot 21-01-051 B, obtained from quenching the melt.

FIG. 46 shows XRPD of 5-MeO-DMT benzoate lot 21-01-051 C, obtained by lyophilisation.

FIG. 47 shows XRPD comparison of 5-MeO-DMT benzoate lot 21-01-051 B after 20 hours, C after 20 hours, and Pattern A reference.

FIG. 48 shows XRPD comparison of 5-MeO-DMT benzoate lot 21-01-051 A, E; E particle size reduced, and Pattern A reference.

FIG. 49 shows DSC thermograph comparison of 5-MeO-DMT benzoate lot 21-01-051 A, C, and D at 10° C.min⁻¹, isolated from acetone concentrate, 051 A, and lyophilisation, 051 C and 051 D.

FIG. 50 shows DSC thermograph comparison of 5-MeO-DMT benzoate lot 21-01-051 C and C post 20 hours at 10° C.min⁻¹.

FIG. 51 shows DSC thermograph of 5-MeO-DMT benzoate lot 21-01-051 D, large scale lyophilised material, with temperature stamps corresponding to hot-stage microscopy images.

FIG. 52 shows Micrograph image of 5-MeO-DMT benzoate lot 21-01-051 D at 30.02° C.

FIG. 53 shows Micrograph image of 5-MeO-DMT benzoate lot 21-01-051 D at 54.21° C.

FIG. 54 shows Micrograph image of 5-MeO-DMT benzoate lot 21-01-051 D at 74.21° C.

FIG. 55 shows Micrograph image of 5-MeO-DMT benzoate lot 21-01-051 D at 114.23° C.

FIG. 56 shows Micrograph image of 5-MeO-DMT benzoate lot 21-01-051 D at 120.14° C.

FIG. 57 shows XRPD pattern comparison of 5-MeO-DMT benzoate lot 21-01-054 solids isolated from the equilibration of amorphous 5-MeO-DMT benzoate with thermal modulation.

FIG. 58 shows XRPD pattern comparison of 5-MeO-DMT benzoate lot 21-01-054 M isolated from the equilibration of amorphous 5-MeO-DMT benzoate in α,α,α-trifluorotoluene with thermal modulation with lot 20-37-64 (Pattern A).

FIG. 59 shows DSC thermograph comparison of a selection of 5-MeO-DMT benzoate lot 21-01-054 solids isolated from the equilibration of amorphous 5-MeO-DMT benzoate with thermal modulation classified as Pattern A.

FIG. 60 shows DSC thermograph expansion comparison of 5-MeO-DMT benzoate lot 21-01-054 solids isolated from the equilibration of amorphous 5-MeO-DMT benzoate with thermal modulation classified as Pattern A, highlighting an event in lot 21-01-054 Q, solid isolated from anisole.

FIG. 61 shows Expanded DSC thermograph expansion highlighting an event in lot 21-01-054 Q, isolated from anisole.

FIG. 62 shows XRPD pattern comparison of 5-MeO-DMT benzoate lot 21-01-060 A1 air dried 2 minutes, lot 21-01−049 B1, Pattern B, and lot 20-37-64, Pattern A.

FIG. 63 shows XRPD pattern comparison of 5-MeO-DMT benzoate lot 21-01-060 A1-air dried 1 hour and lot 21-01−060 A1-air dried 2 minutes.

FIG. 64 shows XRPD pattern comparison of 5-MeO-DMT benzoate lot 21-01-060 A1-air dried 2 minutes, lot 21-01−060 A1-air dried 1 hour, and lot 21-01-049 B1, Pattern B.

FIG. 65 shows DSC thermograph of 5-MeO-DMT benzoate lot 21-01-060 A1, isolated immediately from IPA/toluene and air dried for 1 hour.

FIG. 66 shows DSC thermograph expansion of 5-MeO-DMT benzoate lot 21-01-060 A1, isolated immediately from IPA/toluene and air dried for 1 hour.

FIG. 67 shows XRPD pattern comparison of 5-MeO-DMT benzoate lot 21-01-060 A1 air dried 20 hours, lot 21-01−060 A1 air dried 2 minutes, and lot 21-01-049 B1, Pattern B reference.

FIG. 68 shows XRPD pattern comparison of 5-MeO-DMT benzoate lot 21-01-060 B1, isolated after 3 hours equilibration then air dried for 2 mins and A1 isolated immediately then air dried for 2 minutes.

FIG. 69 shows XRPD pattern comparison of 5-MeO-DMT benzoate lot 21-01-060 B1, isolated after 3 hours equilibration then air dried for 20 hours and B1 isolated after 3 hours equilibration then air dried for 2 minutes, and lot 21-01-049 B1, Pattern B.

FIG. 70 shows XRPD pattern comparison of 5-MeO-DMT benzoate lot 21-01-058 solids isolated from amorphous 5-MeO-DMT benzoate exposed to solvent vapour.

FIG. 71 shows XRPD pattern comparison of 5-MeO-DMT benzoate lot 21-01-058 K, isolated from amorphous 5-MeO-DMT benzoate exposed to solvent vapour, with lot 20-37-64, Pattern A.

FIG. 72 shows DSC thermograph comparison of 5-MeO-DMT benzoate lot 21-01-058 B, lot 21-01-058 F, lot 21-01−058 K, and lot 21-01-062 G.

FIG. 73 shows XRPD pattern comparison of 5-MeO-DMT benzoate lot 21-01-058 D, lot 20-37-64, Pattern A, lot 21−01-049 B1, Pattern B, and lot 21-01-060 B1, Pattern C (air dried 20 hours).

FIG. 74 shows XRPD pattern comparison of 5-MeO-DMT benzoate lot 21-01-058 D, lot 21-01-049 B1, Pattern B, and lot 21-01-060 B1, Pattern C (air dried 20 hours).

FIG. 75 shows XRPD pattern expansion comparison of 5-MeO-DMT benzoate lot 21-01-058 D, lot 21-01-049 B1, Pattern B, and lot 21-01-060 B1, Pattern C (air dried 20 hours).

FIG. 76 shows DSC thermograph of 5-MeO-DMT benzoate lot 21-01-058 D, isolated from exposure of anisole vapour to amorphous form.

FIG. 77 shows XRPD pattern comparison of 5-MeO-DMT benzoate lot 21-01-064 D, and 21-01-060 B1 (air dried 2 minutes).

FIG. 78 shows XRPD pattern expansion comparison of 5-MeO-DMT benzoate lot 21-01-064 D, and 21-01-060 B1 (air dried 2 minutes).

FIG. 79 shows DSC thermograph of 5-MeO-DMT benzoate lot 21-01-064 D at 10° C.min⁻¹.

FIG. 80 shows XRPD pattern comparison of 5-MeO-DMT benzoate lot 21-01-064 C, and 21-01-064 D.

FIG. 81 shows XRPD pattern expansion comparison of 5-MeO-DMT benzoate lot 21-01-064 C, and 21-01-064 D.

FIG. 82 shows DSC thermograph of 5-MeO-DMT benzoate lot 21-01-064 C at 10° C.min⁻¹.

FIG. 83 shows XRPD pattern comparison of 5-MeO-DMT benzoate lot 21-01-073 A, 21-01-049 B1, Pattern B, and 20-37-64, Pattern A.

FIG. 84 shows XRPD pattern comparison of 5-MeO-DMT benzoate lot 21-01-073 F and 21-01-073 F rerun.

FIG. 85 shows XRPD pattern comparison of 5-MeO-DMT benzoate lot 21-01-073 F rerun, 21-01-049 B1, Pattern B, and 20-37-64, Pattern A.

FIG. 86 shows XRPD pattern expansion comparison of 5-MeO-DMT benzoate lot 21-01-073 F rerun, 21-01-049 B1, Pattern B, and 20-37-64, Pattern A.

FIG. 87 shows XRPD pattern comparison of 5-MeO-DMT benzoate lot 21-01-073 K, 21-01-049 B1, Pattern B, and 20-37-64.

FIG. 88 shows XRPD of 5-MeO-DMT benzoate lot 21-01-078.

FIG. 89 shows DVS isothermal plot of 5-MeO-DMT benzoate lot 21-01-078.

FIG. 90 shows XRPD pattern comparison of 5-MeO-DMT benzoate lot 21-01-078 (post-DVS) and 20-37-64.

FIG. 91 shows FTIR overlay of 5-MeO-DMT benzoate Pattern A form (20-20-150FP2), Pattern B form (21-01-071 C2) and Pattern C form (21-010071 C1).

FIG. 92 shows FTIR overlay of 5-MeO-DMT benzoate Pattern A form (20-20-150FP2), Pattern B form (21-01-071 C2) and Pattern C form (21-010071 C1) at 450 to 2000 cm−1.

FIG. 93 shows FTIR overlay of 5-MeO-DMT benzoate Pattern A form (20-20-150FP2), Pattern B form (21-01-071 C2) and Pattern C form (21-010071 C1) at 450 to 2000 cm−1; spectra separated.

FIG. 94 shows Forced Swim Test results, Time Immobile, for 5-MeO-DMT benzoate, vehicle and imipramine.

FIG. 95 shows Forced Swim Test results, Latency to Immobility, for 5-MeO-DMT benzoate, vehicle and imipramine.

FIG. 96 shows 5-MeO-DMT Group Mean Plasma Concentration (ng/mL) in Male Beagle Dogs—Group 2 (HCl salt) and Group 4 (benzoate salt)—Dose Level (0.4 mg/kg); wherein the Mean Plasma Concentration of Groups 2 and 4 are substantially the same with dose time.

FIG. 97 shows an XRPD of Pattern H.

FIG. 98 shows a DSC thermograph of Pattern H.

FIG. 99 shows a DSC thermograph of Pattern H.

FIG. 100 shows a DSC thermograph of Pattern H.

FIG. 101 shows a FTIR spectra of Pattern H compared with Pattern A.

FIG. 102 shows a FTIR spectra of Pattern H compared with Pattern A.

FIG. 103 shows a FTIR spectra of Pattern H.

FIG. 104 shows a FTIR spectra of Pattern A.

FIG. 105 shows an XRPD diffractogram for 5-MeO-DMT hydrochloride lot 20/20/126-FP.

FIG. 106 shows an XRPD diffractogram for 5-MeO-DMT hydrochloride lot 20/45/006-FP.

FIG. 107 shows the XRPD diffractogram of FIGS. 105 and 106 overlaid on top of one another.

FIG. 108 shows a DSC and TGA thermograph of 5-MeO-DMT Hydrochloride, lot 20/20/126-FP at 10° C./Min heating rate.

FIG. 109 shows DSC thermographs of 5-MeO-DMT hydrochloride, lot 20/20/126-FP at 5° C./Min (Black), 10° C./Min (Red), 20° C./Min (Blue) and 40° C./Min (Green) heating rates.

FIG. 110 shows a DSC and TGA thermograph of 5-MeO-DMT Hydrochloride, lot 20/45/006-FP at 10° C./Min heating rate.

FIG. 111 shows DSC thermographs of 5-MeO-DMT hydrochloride, lot 20/45/06-FP at 5° C./Min (Black), 10° C./Min (Red), 20° C./Min (Blue) and 40° C./Min (Green) heating rates.

FIG. 112 shows the DVS isotherm of 5-MeO-DMT Hydrochloride, lot 20/20/126-FP.

FIG. 113 shows the DVS isotherm of 5-MeO-DMT Hydrochloride, lot 20/45/006-FP.

FIG. 114 shows an optical micrograph of lot 20/20/126-FP of 5-MeO-DMT Hydrochloride at ×10 magnification (A) and polarised (B).

FIG. 115 shows optical micrographs of lot 20/20/126-FP of 5-MeO-DMT Hydrochloride at ×50 magnification (A) and (B).

FIG. 116 shows an optical micrograph of lot 20/45/006-FP of 5-MeO-DMT Hydrochloride at ×10 magnification (A) and polarised (B).

FIG. 117 shows an optical micrograph of lot 20/45/006-FP of 5-MeO-DMT Hydrochloride at ×50 magnification (A) and (B).

### DETAILED DESCRIPTION OF THE INVENTION

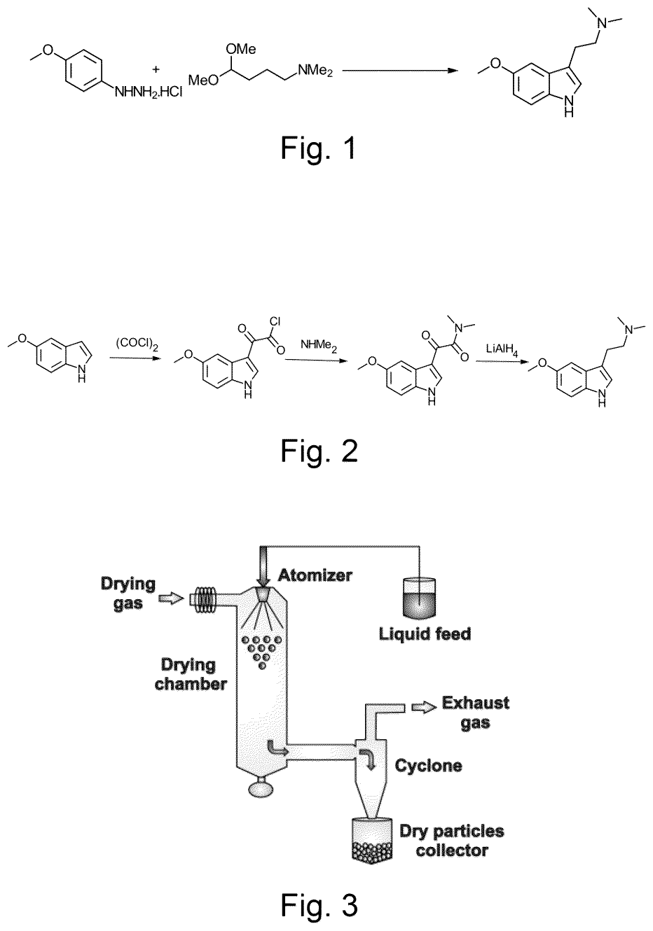

FIG. 1 shows a one-step synthesis of 5-MeO-DMT from the reaction of 4-methoxyphenylhydrazine hydrochloride with (N,N)-dimethylamino)butanal dimethyl acetal.

FIG. 2 shows a three step synthesis of 5-MeO-DMT. The first step involves the reaction of 5-methoxyindole with oxalyl chloride. The resultant product is aminated with dimethylamine and then is reduced with lithium aluminium hydride.

FIG. 3 shows the schematic route for the formation of a powder form of 5-MeO-DMT using a spray drying process.

### Examples

### Example 1: Synthesis of 5-MeO-DMT (the Free Base) in on Step (the Free Base)

A schematic representation of this reaction is shown in FIG. 1.

Hydrazine (1.0 eq), diethyl acetal (1.2 eq), and aqueous sulfuric acid (0.1 eq) where heated together at 65-75° C. for 18 hours. MTBE (10 vol) was added, followed by adjustment to about pH10 using 12% caustic (about 1.1 eq.). The layers were separated and the aqueous fraction back extracted with MTBE (10 vol). The combined organic fractions were washed with water (10 vol) twice, then evaporated to dryness under vacuum. Yield 100%.

### Example 2: Synthesis of 5-MeO-DMT (the Free Base) in Three Steps

A schematic representation of this reaction is shown in FIG. 2.

Step 1—Add methyl tert-butyl ether (MTBE) (15 vol) into the reaction vessel and cool to-20 to −30° C., before adding oxalyl chloride (1.5 eq), maintaining the temperature at no more than −20° C. Add a solution of 5-methoxyindole (1.0 eq) in THF (1 vol) to the reaction vessel, maintaining the temperature at no more than −20° C. Allow the reaction to warm to 0-5° C. and stir for at least 1 hour, ensuring that no more than 2% of the starting material indole remains.

Cool the reaction to between −20 to −30° C. and add a solution of methanol (1 vol) and MTBE (1 vol), maintaining the temperature at no more than −20° C. Allow the reaction to warm to 0-5° C. over no less than 30 minutes and stir for at least 1 hour.

Filter and wash the solids with MTBE cooled to 0-5° C. Add the washed filtered solids and methanol (20 vol) to a reaction vessel. Heat to 60-65° C. and stir for no more than 30 minutes. Cool to 0-5° C. over no less than 2 hours and stir for no less than 2 hours. Filter and wash the solids with MTBE cooled to 0-5° C. Dry the solids obtained at no more than 40° C. for no less than 12 hours. Yield 95%.

Step 2—Add the compound obtained in step 1 (1.0 eq) to a reaction vessel together with dimethylamine hydrochloride (3.0 eq) and methanol (2 vol). Add 25% NaOMe in methanol (3.5 eq), to the reaction maintaining the temperature at no more than 30° C. Warm to and stir for no less than 5 hours, ensuring that no more than 0.5% of the starting material from step 1 remains. Adjust the temperature to 0-5° C. over no less than 2 hours, then add water (5 vol) over no less than 1 hour with stirring at 0-5° C. for no less than 1 hour.

Filter and wash the solids with water cooled to 0-5° C., and dry the solids obtained at no more than 40° C. for no less than 12 hours. Yield 85%.

Step 3—Add the compound obtained in step 2 (1.0 eq) to a reaction vessel. Add 1M LiAlH4 in THF (1.5 eq) in THF (8 vol) to the reaction maintaining no more than 40° C. Heat at reflux for no less than 4 hours ensuring that no more than 2% of the starting material from step 2 remains.

Adjust to 0-5° C. and add water (0.25 vol) in THF (0.75 vol) over no less than 30 minutes, maintaining no more than 10° C. Then add 15% caustic (0.25 vol) maintaining the temperature at no more than 10° C. Add water (0.65 vol) maintaining the temperature at no more than 10° C. Add THF (0.25 vol) as a vessel rinse and stir the contents at 0−5° C. for no less than 30 minutes. Add sodium sulfate (100 wt %) and stir contents at 0-5° C. for no less than 30 minutes.

Filter and wash the solids with toluene (2×10 vol) and keep liquors separate. Recharge THF liquors to a clean vessel and distil under vacuum to minimum stir. Charge toluene liquors and distil under vacuum to about 10 vol. Then add water (5 vol) and stir for no less than 15 minutes. Stop, settle and remove aqueous layer to waste. Charge with 4% HCl to a pH of between 1-2 (about 4 vol) and stir for no less than 15 minutes. Stop, settle and remove organic layer to waste. Charge MTBE (15 vol). Charge with 15% caustic to a pH between 11-13 (about 0.9 vol). Stir for no less than 15 minutes. Stop, settle and remove aqueous layer to waste. Charge with water (5 vol). Stir for no less than 15 minutes. Stop, settle and remove the aqueous layer to waste.

### Example 3: Synthesis of 5-MeO-DMT Hydrochloride Salt

5-MeO-DMT (the free base) is dissolved in toluene (1.0 to 2.5 vol). Isopropyl alcohol (IPA) was then added (2.5 vol) followed by 1.25M HCl in IPA (1.0 eq) and the temperature adjusted to 0-5° C. over 1 hour.

If no precipitation/crystallization occurs, toluene (6.25 vol) is added over 30 minutes. The mixture was then stirred at 0-5° C. for 2 hours. The resultant solid is filtered, washed with toluene (3.8 vol). The solid was dried under vacuum at ambient temperature. Yield 58%.

### Example 3a: Synthesis of 5-MeO-DMT HCl

Herein disclosed, there is provided a method for the synthesis of 5-MeO-DMT hydrochloride salt (also referred to herein as 5-MeO-DMT hydrochloride or the hydrochloride salt).

Herein disclosed, there is provided a purified form of 5-MeO-DMT hydrochloride obtained from a source of 5-MeO-DMT hydrochloride.

In a first aspect there is provided a purified mass of 5-MeO-DMT hydrochloride obtained from a source of 5-MeO-DMT hydrochloride.

The skilled person would understand that a mass of 5-MeO-DMT hydrochloride is a commercially useful amount, for example not just a few crumbs of 5-MeO-DMT hydrochloride, or a few crystals, or a single crystal of 5-MeO-DMT hydrochloride.

In an embodiment, the purified mass is greater than 0.5, 1, 2, 5, 10, 20, 50, 100, 250, 500 or 1000 grams.

In an embodiment, the purified mass is a commercially useful amount of 5-MeO-DMT hydrochloride. In an embodiment, a useful amount of 5-MeO-DMT hydrochloride is sufficient to provide more than 20, 50, 100, 250, 500, 1,000, 5,000, 10,000, 25,000, 50,000 or 100,000 pharmaceutically effective treatment doses for human subjects in need thereof.

In an embodiment, the purified mass is not a single crystal. In an embodiment, the purified mass is not a few crystals 5-MeO-DMT hydrochloride. In an embodiment, the purified mass is not a few crumbs of 5-MeO-DMT hydrochloride.

In an embodiment, the purified mass is not sufficient to provide less than 1, 2, 3, 4, 5, 6, 7, 8, 9, 10, or 15 treatment doses for human subjects in need thereof.

In an embodiment, the source of 5-MeO-DMT hydrochloride contains impurities. In an embodiment, the source of 5-MeO-DMT hydrochloride is less pure than the purified mass of 5-MeO-DMT hydrochloride. In an embodiment, the purified mass of 5-MeO-DMT hydrochloride contains less/fewer impurities than the source of 5-MeO-DMT hydrochloride. In an embodiment, the source of 5-MeO-DMT hydrochloride contains more impurities than the purified mass of 5-MeO-DMT hydrochloride.

In an embodiment, there is provided a purified mass of 5-MeO-DMT hydrochloride.

In an embodiment, the purified mass contains no more than 0.1, 0.2, 0.25, 0.5, 1, 2, 3, 4 or 5% of any impurity.

In an embodiment, the phrase ‘any impurity’ can be understood to mean ‘any one impurity’.

In an embodiment, the term ‘purified’ is may be understood to be equivalent with the term ‘pure’.

In an embodiment, the purified mass is substantially free of the hydroxyl impurity shown below:

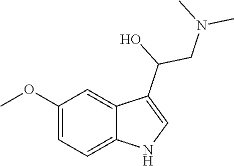

In an embodiment, the purified mass contains no more than 0.1, 0.2, 0.25, 0.5, 1, 2, 3, 4 or 5% of the hydroxyl impurity.

In an embodiment, the source of 5-MeO-DMT hydrochloride contains more hydroxyl impurity than the purified mass of 5-MeO-DMT hydrochloride. In an embodiment, the purified mass of 5-MeO-DMT hydrochloride contains less hydroxyl impurity than the source of 5-MeO-DMT hydrochloride.

In an embodiment, the purified mass has a chemical purity of greater than 95, 96, 97, 98, 99 or 99.5% by HPLC or RP-HPLC.

In an embodiment, the purified mass of 5-MeO-DMT hydrochloride is crystalline.

In an embodiment, the purified mass is characterised by one or more of:
peaks in an XRPD diffractogram at 9.2, 12.2, 14.1, 15.0, 18.5, 19.5, 23.9, 24.5, 25.1, 26.0, 26.9 and 28.3°2θ±0.1°2θ as measured by x-ray powder diffraction using an x-ray wavelength of 1.5406 A;
endothermic event in a DSC thermograph having an onset temperature of between 14° and 150° C. and/or a peak of between 142 and 148° C.;
enthalpy in a DSC thermograph of between 113 J/g and −123 J/g;
onset of decomposition in a TGA thermograph of between 12° and 165° C.

In an embodiment, the purified mass is characterised as described elsewhere in this document, such as in the Examples.

In an embodiment, there is provided of obtaining a purified mass of 5-MeO-DMT hydrochloride, wherein a source of 5-MeO-DMT hydrochloride is base-treated, optionally the source of 5-MeO-DMT hydrochloride is base-treated with an aqueous basic solution.

In an embodiment, of obtaining a purified mass of 5-MeO-DMT hydrochloride, wherein 5-MeO-DMT hydrochloride is base-treated, optionally the source of 5-MeO-DMT hydrochloride is base-treated with an aqueous basic solution.

In an embodiment, the basic solution comprises an alkoxide, optionally the basic solution comprises sodium hydroxide, further optionally the basic solution is 5% aqueous sodium hydroxide.

In an embodiment, the source of 5-MeO-DMT hydrochloride is suspended in a suspending organic solvent prior to being base-treated, wherein optionally the suspending organic solvent is an alcohol, ester, an acetate and/or an acetate ester.

In an embodiment, the resultant base-treated reaction is partitioned with an extracting organic solvent to give an extract comprising the base-conditioned hydrochloride salt; wherein optionally the extracting organic solvent is an alcohol, ester, an acetate and/or an acetate ester.

In an embodiment, the extract is reduced under vacuum to give a concentrate of the purified mass of 5-MeO-DMT hydrochloride, optionally the extract is concentrated to approximately 8 volumes, or further optionally the solvent is removed to give the purified mass of 5-MeO-DMT hydrochloride in a solid form.

In an embodiment, the extract or solid form of the purified mass of 5-MeO-DMT hydrochloride is azeotropically dried with one or more batches of fresh extracting organic solvent wherein optionally the extracting organic solvent is an alcohol, ester, an acetate and/or an acetate ester.

In an embodiment, the purified mass of 5-MeO-DMT hydrochloride is obtained by filtration.

In an embodiment, the filtered purified mass of 5-MeO-DMT hydrochloride is washed with a washing organic solvent.

In an embodiment, the purified mass of 5-MeO-DMT hydrochloride is washed with cooled washing organic solvent, optionally the washing organic solvent is cooled to below room temperature, is cooled to below 10° C., is cooled to below 5° C., or cooled to between 5 and 0° C.

In an embodiment, the purified mass of 5-MeO-DMT hydrochloride is dried under vacuum.

In an embodiment, the suspending organic solvent, the washing organic solvent, and/or the extracting organic solvent is IPAc.

In an embodiment, the purified mass of 5-MeO-DMT hydrochloride is isolated and subjected to a recrystallisation process.

In an embodiment, the method comprises the steps of:
combining the source of 5-MeO-DMT hydrochloride and an organic solvent; optionally the organic solvent is IPAc
adding a basic solution to the combined 5-MeO-DMT hydrochloride salt and organic solvent; optionally the basic solution is aqueous 5% NaOH;
partitioning;
washing the resulting organic phase with water;
drying the solvent; optionally azeotropically with IPAc; and
concentrating under vacuum.

In an embodiment, the method further comprises the steps of concentrating under vacuum to dryness.

In an embodiment, the method further comprises the steps of:
adjusting the solvent temperature to between about 50-55° C.;
adding one or more counter solvents in which the 5-MeO-DMT hydrochloride is substantially insoluble in; and/or adjusting the temperature to between about 0-5° C.;
filtering and washing with cold solvent; optionally the cold solvent is IPAc; and
drying under vacuum to obtain the purified mass of 5-MeO-DMT hydrochloride in a solid form, optionally as a crystalline solid.

In an embodiment, the purified mass of 5-MeO-DMT hydrochloride produced is characterised by one or more of:
peaks in an XRPD diffractogram at 9.2, 12.2, 14.1, 15.0, 18.5, 19.5, 23.9, 24.5, 25.1, 26.0, 26.9 and 28.3°2θ±0.1°2θ as measured by x-ray powder diffraction using an x-ray wavelength of 1.5406 A;
endothermic event in a DSC thermograph having an onset temperature of between 14° and 150° C. and/or a peak of between 142 and 148° C.;
enthalpy in a DSC thermograph of between 113 J/g and −123 J/g;
onset of decomposition in a TGA thermograph of between 12° and 165° C.

In an embodiment, there is provided a purified mass of 5-MeO-DMT hydrochloride obtained by the method described previously or subsequently.

In an embodiment, there is provided an inorganic or organic acid salt of 5-MeO-DMT obtained by the step of treating the purified mass of 5-MeO-DMT hydrochloride as defined previously or subsequently, or obtained by the method previously or subsequently, with an inorganic acid or organic acid; wherein the resultant counter anion is the deprotonated form of the acid used, wherein optionally the resultant counter anion is a fluoride, bromide, iodide, fumarate, acetate, succinate, oxalate, acetate, citrate, phosphate, tartrate, benzenesulfonate, tosylate, adipate, glycolate, ketoglutarate, malate, saccharinate, triflate or benzoate anion; further optionally the anion is the benzoate.

In an embodiment, there is provided >100 g of purified 5-MeO-DMT hydrochloride.

In an embodiment, there is provided >200 g of purified 5-MeO-DMT hydrochloride.

In an embodiment, there is provided >300 g of purified 5-MeO-DMT hydrochloride.

In an embodiment, there is provided >400 g of purified 5-MeO-DMT hydrochloride.

In an embodiment, there is provided >500 g of purified 5-MeO-DMT hydrochloride.

In an embodiment, there is provided a method of synthesizing >100 g, >200 g, >300 g, >400 g or >500 g of purified 5-MeO-DMT hydrochloride.

Herein disclosed, there is provided 5-MeO-DMT hydrochloride which contains no more than 0.1, 0.2, 0.25, 0.5, 1, 2, 3, 4 or 5% of any impurity.

In an embodiment, the 5-MeO-DMT hydrochloride is substantially free of the hydroxyl impurity shown below:

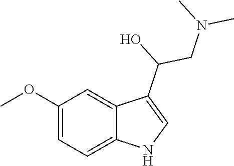

In an embodiment, the 5-MeO-DMT hydrochloride contains no more than 0.1, 0.2, 0.25, 0.5, 1, 2, 3, 4 or 5% of the hydroxyl impurity.

In an embodiment, the 5-MeO-DMT hydrochloride has a chemical purity of greater than 95, 96, 97, 98, 99 or 99.5%.

In an embodiment, the 5-MeO-DMT hydrochloride has a chemical purity of greater than 95, 96, 97, 98, 99 or 99.5% by HPLC or RP-HPLC.

In an embodiment, the 5-MeO-DMT hydrochloride is crystalline.

In an embodiment, the crystalline 5-MeO-DMT hydrochloride is characterised as described elsewhere in this document, such as in the Examples.

In an embodiment, there is provided pure 5-MeO-DMT hydrochloride. In an embodiment, this may be crystalline.

In an embodiment, the source of 5-MeO-DMT hydrochloride is isolated as a solid or a solution or dispersed in a carrier medium e.g. a solvent.

In an embodiment, the source of 5-MeO-DMT hydrochloride is obtained from the reaction of the free base of 5-MeO-DMT with hydrochloride.

In an embodiment, the source of 5-MeO-DMT hydrochloride is obtained from the reaction of the free base of 5-MeO-DMT with hydrochloride wherein the reaction takes place in a solvent wherein the solvent may be one or more of toluene, IPA or IPAc.

### Example 4: Synthesis of 5-MeO-DMT Benzoate Salt

5-MeO-DMT (the free base) is dissolved in toluene (1 eq) and benzoic acid (1 eq) in toluene (10 vol) is added over a period of 20 minutes and stirred at room temperature for 2 hours. The resultant precipitation/crystallization was filtered and washed with toluene (2.5 vol) and dried under vacuum at room temperature.

Isopropyl acetate (IPAc) (15.8 vol) was added to the solids obtained above and the temperature was raised to about 73° C. until the solid dissolved. The solution was allowed to cool to 0-5° C. over 2 hours and this temperature was maintained for 1 hour with stirring. The resultant benzoate salt was filtered and vacuum dried at room temperature.

Yield 68%.

The benzoate salt of 5-MeO-DMT has improved characteristics over the common hydrochloride salt, including reduced mucosal irritation, increased epithelial permeability and increased stability. 5-MeO-DMT benzoate is a white to off white solid powder, soluble in water at >50 mg/ml with a pH of 7-8 at 50 mg/ml and a pKa of 9.71.

### Example 5: Synthesis of 5-MeO-DMT Fumarate Salt

5-MeO-DMT (the free base) is added to a solution of fumaric acid (0.5 eq) in IPA over 15 minutes at 40-45° C. The resultant solution was cooled at room temperature and stirred for 16 hours. The solution was then cooled to 0-5° C. with stirring for 2 hours. The resulting precipitation/crystallization was filtered and was rinsed with toluene (2.5 vol).

Yield 68%.

### Example 6: 5-MeO-DMT Powder

A schematic route for the preparation of a powder form of 5-MeO-DMT (or the salt thereof) is shown in FIG. 3.

The three main steps in the process are:
1. Spray drying a solution containing the substance(s) of interest (e.g. 5-MeO-DMT, or the salt, thereof inclusive of any excipients). This can be done via an atomizing nozzle such as with rotary atomizers, pressure atomizers, twin fluid nozzles, ultrasonic atomizers, four-fluid nozzles. This is done so as to form droplets capable of generating co-formed particles in the desired particle size range.
2. Drying of the atomized droplets (e.g. with nitrogen gas, optionally at an elevated temperature).
3. Separating and collecting the dried particles from the gas stream (e.g. using a cyclone separator to capture the required size fraction).

In an embodiment, a ProCepT spray dryer is used. In an embodiment, a ProCepT spray dryer with an ultrasonic nozzle is used.

In an embodiment, there is dissolution of 5-MeO-DMT benzoate and HPMC in water to make input solution at a 50:50 ratio.

### Example 7: Slug Mucosal Irritation Assay

The Slug Mucosal Irritation (SMI) assay was initially developed at the Laboratory of Pharmaceutical Technology (UGent) to predict the mucosal irritation potency of pharmaceutical formulations and ingredients. The test utilizes the terrestrial slug Arion lusitanicus. The body wall of the slugs is a mucosal surface composed of different layers. The outer single-layered columnar epithelium that contains cells with cilia, cells with micro-villi and mucus secreting cells covers the subepithelial connective tissue. Slugs that are placed on an irritating substance will produce mucus. Additionally tissue damage can be induced which results in the release of proteins and enzymes from the mucosal surface. Several studies have shown that the SMI assay is a useful tool for evaluating the local tolerance of pharmaceutical formulations and ingredients. A classification prediction model that distinguishes between irritation (mucus production) and tissue damage (release of proteins and enzymes) has been developed. Furthermore, several studies with ophthalmic preparations have shown that an increased mucus production is related to increased incidence of stinging, itching and burning sensations. In 2010 a clinical trial was set up to evaluate the stinging and burning sensations of several diluted shampoos. A 5% shampoo dilution or artificial tears were instilled in the eye and the discomfort was scored by the participants on a 5 point scale during several time points up to 30 min after instillation. The same shampoos were tested in the SMI assay using the Stinging, Itching and Burning (SIB) protocol. This study showed that an increased mucus production was related with an increased incidence of stinging and burning sensations in the human eye irritation test. The relevance of the assay to reliably predict nasal irritation and stinging and burning sensations was demonstrated using several OTC nasal formulations, isotonic, and hypertonic saline.

Furthermore, the test was validated using reference chemicals for eye irritation (ECETOC eye reference data bank). These studies have shown that the SMI assay can be used as an alternative to the in vivo eye irritation tests. Moreover, a multi-center prevalidation study with four participating laboratories showed that the SMI assay is a relevant, easily transferable and reproducible alternative to predict the eye irritation potency of chemicals.

The purpose of this assay was to assess the stinging, itching or burning potential of the test item(s) defined below. Using the objective values obtained for the mucus production the stinging, itching or burning potential of the test item(s) can be estimated by means of the prediction model that is composed of four categories (no, mild, moderate and severe).

### Control Items:

Negative control—Name: Phosphate buffered saline (PBS)
Positive control—Name: 1% (w/v) Benzalkonium chloride in PBS

### Test Items:

### Compound 1

Name: 10% (w/v) Disodium fumarate in PBS
CASRN: 17013-01-3
Batch: KBSJ-P0
Description: colourless solution
Storage condition: room temperature (compounded on the day of the experiment)

### Compound 2

Name: 10% (w/v) Sodium phosphate monobasic in PBS
CASRN: 7558-80-7
Batch: 2A/220991
Description: colourless solution
Storage condition: room temperature (compounded on the day of the experiment)

### Compound 3

Name: 10% (w/v) Sodium acetate in PBS
CASRN: 127-09-3
Batch: 5A/233258
Description: colourless solution
Storage condition: room temperature (compounded on the day of the experiment)

### Compound 4

Name: 10% (w/v) Sodium citrate in PBS
CASRN: 68-04-2
Batch of vial: 5A/241516
Description: colourless solution
Storage condition: room temperature (compounded on the day of the experiment)

Test System: Slugs (Arion lusitanicus); 3 slugs per treatment group. The parental slugs of Arion lusitanicus collected in local gardens along Gent and Aalter (Belgium) are bred in the laboratory in an acclimatized room (18-20° C.). The slugs are housed in plastic containers and fed with lettuce, cucumber, carrots and commercial dog food.

Test Design: A single study was performed. Treatment time was 15 minutes three times on the same day.

### Preparation of Slugs:

Slugs weighing between 3 and 6 g were isolated from the cultures two days before the start of an experiment. The body wall was inspected carefully for evidence of macroscopic injuries. Only slugs with clear tubercles and with a foot surface that shows no evidence of injuries were used for testing purposes. The slugs were placed in a plastic box lined with paper towel moistened with PBS and were kept at 18-20° C. Daily the body wall of the slugs was wetted with 300 μl PBS using a micropipette.

### Test Procedure:

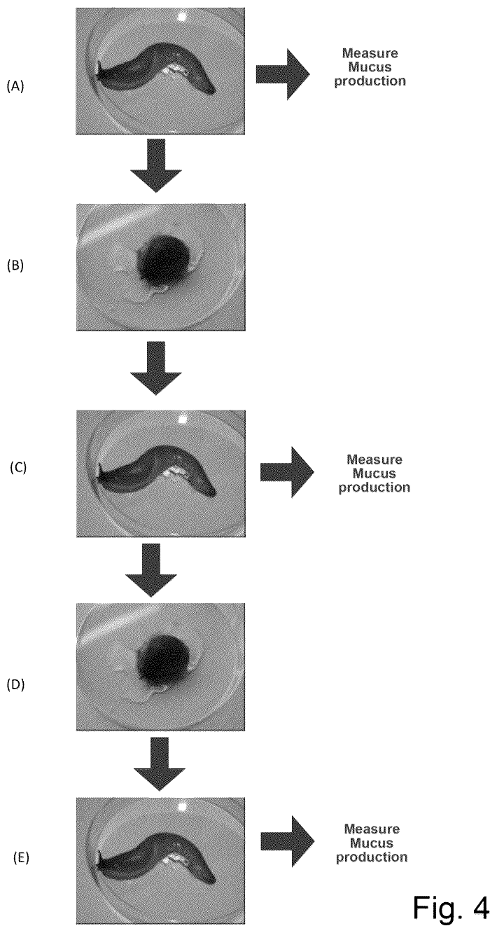

The stinging, itching or burning potency of the test item(s), was evaluated by placing 3 slugs per treatment group 3 times a day on 100 μL of test item in a Petri dish for 15±1 min. After each 15-min contact period the slugs were transferred for 60 min into a fresh Petri dish on paper towel moistened with 1 mL PBS to prevent desiccation. An overview of this can be seen in FIG. 4.

### Mucus Production:

The amount of mucus produced during each contact period was measured by weighing the Petri dishes with the test item before and after each 15-min contact period. The mucus production was expressed as % of the body weight.

The slugs were weighed before and after each 15-min contact.

### Classification Prediction Model

Based on the endpoint of the SMI assay the stinging, itching or burning potency of the test item(s) was estimated using a classification prediction model.

The evaluation of the test results was based upon the total amount of mucus production during 3 repeated contact periods with the test item.

For each slug, the mucus production was expressed in % of the body weight by dividing the weight of the mucus produced during each contact period by the body weight of the slug before the start of that contact period. The total mucus was calculated for each slug and then the mean per treatment group was calculated. The classification prediction model shown in Table 1 was used to classify the compounds.

TABLE 1
Cut-off values for classification -
potency for nasal mucosal discomfort

| Total Mucus production in % | Stinging, Itching and |
|---|---|
| (mean of n = 3) | Burning (SIB) |
| <5.5% | No |
| ≥5.5 and <10% | Mild |
| ≥10 and <17.5% | Moderate |
| ≥17.5% | Severe |

### Acceptance Criteria

Before a test was considered valid, the following criteria must be met:
the negative control should be classified as causing no stinging, itching and burning (Total mucus production <5.5%)
the positive control item should be classified as causing severe stinging, itching and burning (Total mucus production 17.5%) Irritation Potential

TABLE 2
Amount of mucus produced (MP) during each 15-min contact
period (CP) and total amount of mucus produced

| | MP CP1¹ | MP CP2¹ | MP CP3¹ | Total MP¹ | SIB |
|---|---|---|---|---|---|
| Formulation | (%) | (%) | (%) | (%) | Category² |
| NC - PBS | −0.2 ± 0.3 | −0.6 ± 0.1 | 0.3 ± 0.6 | −0.5 ± 0.7 | No |
| PC - 1% BAC | 9.2 ± 1.5 | 8.4 ± 1.2 | 5.9 ± 3.1 | 23.4 ± 3.6 | Severe |
| Disodium fumarate, 10% | 5.0 ± 2.5 | 4.7 ± 1.7 | 3.6 ± 0.8 | 13.3 ± 1.8 | Moderate |
| Sodium phosphate, 10% | 3.3 ± 0.9 | 5.6 ± 0.3 | 6.2 ± 1.3 | 15.2 ± 1.8 | Moderate |
| Sodium acetate, 10% | 3.3 ± 0.2 | 3.9 ± 0.4 | 3.9 ± 0.2 | 11.0 ± 0.8 | Moderate |
| Sodium citrate, 10% | 4.2 ± 0.5 | 4.2 ± 0.3 | 4.1 ± 1.1 | 12.5 ± 1.4 | Moderate |

NC: negative control;
PC: positive control;
BAC: benzalkonium chloride
¹Mean ± SD, n = 3
²No: total MP < 5.5%; Mild: 5.5% ≤ total MP < 10%; Moderate: 10% ≤ total MP < 17.5%; Severe: total MP ≥ 17.5%

The average amount of mucus produced during each 15-min contact period and total mucus production (total MP) is presented in Table 2. According to the classification prediction model of the SMI test, the negative control (untreated slugs) did not induce reactions in the slugs (mean total MP<5.5%). The positive control on the other hand (DDWM/SLS 80/20) induced a high mucus production during each contact period (mean total MP 17.5%) resulting in a classification as severe stinging, itching, and burning (SIB) reactions. The acceptance criteria were met and the experiment was considered valid.

In total, 4 different solutions were tested. The amount of mucus produced during each 15-min contact period was between 10% and 17.5%, indicating moderate SIB reactions. The test items can be ranked according to increasing total mucus production: sodium acetate (10% w/v)<sodium citrate (10% w/v)<disodium fumarate (10% w/v)<sodium phosphate (10% w/v).

Numerical Data

| Treatment | Replicate | MP CP1 | MP CP2 | MP CP3 | Total MP |
|---|---|---|---|---|---|
| NC | 1 | −0.32 | −0.59 | 0.97 | 0.06 |
| | 2 | −0.44 | −0.57 | −0.32 | −1.33 |
| | 3 | 0.14 | −0.70 | 0.35 | −0.21 |
| PC | 1 | 8.08 | 7.91 | 9.29 | 25.28 |
| | 2 | 10.82 | 9.71 | 5.23 | 25.77 |
| | 3 | 8.59 | 7.49 | 3.17 | 19.25 |
| Disodium fumarate, 10% | 1 | 7.83 | 3.56 | 3.14 | 14.53 |
| | 2 | 4.39 | 6.64 | 3.11 | 14.14 |
| | 3 | 2.87 | 3.84 | 4.47 | 11.17 |
| Sodium phosphate, 10% monobasic | 1 | 4.33 | 5.34 | 7.41 | 17.07 |
| | 2 | 2.93 | 5.69 | 6.40 | 15.02 |
| | 3 | 2.74 | 5.83 | 4.89 | 13.46 |
| Sodium acetate, 10% | 1 | 3.47 | 4.24 | 4.10 | 11.80 |
| | 2 | 3.44 | 3.93 | 3.81 | 11.18 |
| | 3 | 3.06 | 3.43 | 3.69 | 10.17 |
| Sodium citrate, 10% | 1 | 4.16 | 4.01 | 3.78 | 11.95 |
| | 2 | 4.75 | 4.03 | 5.33 | 14.12 |
| | 3 | 3.68 | 4.55 | 3.25 | 11.48 |

TABLE 3
Amount of mucus produced (MP) during each 15-min contact
period (CP) and total amount of mucus produced

| | MP CP1¹ | MP CP2¹ | MP CP3¹ | Total MP¹ | SIB |
|---|---|---|---|---|---|
| Formulation | (%) | (%) | (%) | (%) | Category² |
| NC - PBS | −0.2 ± 0.3 | −0.6 ± 0.1 | 0.3 ± 0.6 | −0.5 ± 0.7 | No |
| PC - 1% BAC | 9.2 ± 1.5 | 8.4 ± 1.2 | 5.9 ± 3.1 | 23.4 ± 3.6 | Severe |
| Disodium fumarate, 10% | 5.0 ± 2.5 | 4.7 ± 1.7 | 3.6 ± 0.8 | 13.3 ± 1.8 | Moderate |
| Sodium phosphate, 10% | 3.3 ± 0.9 | 5.6 ± 0.3 | 6.2 ± 1.3 | 15.2 ± 1.8 | Moderate |
| Sodium acetate, 10% | 3.3 ± 0.2 | 3.9 ± 0.4 | 3.9 ± 0.2 | 11.0 ± 0.8 | Moderate |
| Sodium citrate, 10% | 4.2 ± 0.5 | 4.2 ± 0.3 | 4.1 ± 1.1 | 12.5 ± 1.4 | Moderate |

NC: negative control;
PC: positive control;
BAC: benzalkonium chloride
¹Mean ± SD, n = 3
²No: total MP < 5.5%; Mild: 5.5% ≤ total MP < 10%; Moderate: 10% ≤ total MP < 17.5%; Severe: total MP ≥ 17.5%

TABLE 4
Amount of mucus produced (MP) during each 30-min contact period
(CP) and total amount of mucus produced (Code 00E04)

| Treatment | CP1 30-min | CP2 30-min | Total MP |
|---|---|---|---|
| PBS | −1.0 ± 0.6 | −1.1 ± 0.8 | −2.2 ± 0.6 |
| BAC (1%) | 13.2 ± 4.2 | 18.6 ± 9.8 | 31.8 ± 12.6 |
| Sodium oxalate (1%) | 4.5 ± 1.3 | 6.6 ± 1.0 | 11.1 ± 2.0 |

TABLE 5
Amount of mucus produced (MP) during each 60-min contact
period (CP) and total amount of mucus produced

| | Day 1 | Day 2 | |
|---|---|---|---|
| Treatment | CP1 60-min | CP2 60-min | Total MP |
| PBS | −0.2 ± 0.7 | −0.7 ± 0.5 | −0.9 ± 0.5 |
| BAC (1% CP1 & 3.5% CP2) | 21.9 ± 4.8 | 9.7 ± 3.2 | 31.6 ± 2.5 |
| Sodium oxalate | 11.2 ± 3.9 | 16.0 ± 4.0 | 27.1 ± 2.3 |
| (1% CP1 & 3.5% CP2) | | | |

TABLE 6
Amount of mucus produced (MP) during
a 60-min contact period (CP)

| Treatment | CP1 60-min |
|---|---|
| PBS | −0.2 ± 1.0 |
| BAC (1%) | 15.0 ± 1.9 |
| Sodium benzoate (1%) | 2.6 ± 0.3 |
| Sodium benzoate (10%) | 6.9 ± 1.2 |

### Results

The total MP for a 60-min treatment (historical data) was compared with the total MP of the SIB protocol (3×15-min treatment; current data). In the table below a ranking is proposed from least SIB reactions to highest SIB reactions:

| | | | Total MP (% |
|---|---|---|---|
| Compound | Concentration | Treatment time | body weight) |
| Sodium benzoate | 1% | 60-min | 2.6 |
| Sodium benzoate | 10% | 60-min | 6.9 |
| Sodium acetate | 10% | 45-min (3x 15-min) | 11.0 |
| Sodium citrate | 10% | 45-min (3x 15-min) | 12.5 |
| Disodium fumarate | 10% | 45-min (3x 15-min) | 13.3 |
| Sodium phosphate | 10% | 45-min (3x 15-min) | 15.2 |
| Sodium oxalate | 1% | 60-min | 11.2 |

Sodium oxalate appears to be the most irritating salt since a 1% concentration results in 11.2% total MP after 1 hour of contact. Sodium benzoate is the least irritating salt.

### Example 8: Further Slug Mucosal Irritation (SMI) Testing

5-MeO-DMT as a freebase compound is known to be highly irritating to the mucosal lining; therefore, it is commonly prepared as a salt for insufflation. The hydrochloride (HCl) salt of 5-MeO-DMT is most commonly used due to ease of crystallisation. However, it is known that the HCl salt of 5-MeO-DMT is still quite irritating to the mucosal lining.

Following the results above indicating that sodium benzoate is the least irritating salt of those studied; further SMI testing was performed on 5-MeO-DMT benzoate and the common 5-MeO-DMT HCl salt according to the previously described methods (of Example 7). The results of this are shown below:

| Compound | Concentration (w/v) | Total MP (% body weight) |
|---|---|---|
| 5-MeO-DMT benzoate | 10% | 7.38 |
| 5-MeO-DMT HCl | 10% | 10.27 |
| Benzylkonium (positive control) | 10% | 17.56 |
| PBS (negative control) | 10% | −0.77 |

The 5-MeO-DMT benzoate produced ‘mild’ irritation compared to the 5-MeO-DMT HCl which scored as ‘moderate’ on testing.

### Example 9: Permeation Data

The use of ovine nasal epithelium to study nasal drug absorption is a technique which is well known to the person skilled in the art.

The permeation of 5-MeO-DMT benzoate and 5-MeO-DMT HCl has been studied by the current applicants. Dosing solutions corresponding to 1.25% concentration were prepared in water and applied to ovine nasal epithelium. The average cumulative (μg/cm²) of permeation of the benzoate and hydrochloride salt are shown in the table below (mean±SD, n=5):

| Time (min) | | 0.0 | 10.0 | 20.0 | 30.0 | 40.0 | 50.0 | 60.0 | 75.0 | 90.0 |
|---|---|---|---|---|---|---|---|---|---|---|
| Cumulative | 5-MeO-DMT | 0.00 | 0.20 | 3.46 | 9.30 | 15.46 | 21.51 | 27.30 | 33.34 | 39.77 |
| amount | Benzoate | (0.00) | (0.35) | (3.07) | (6.46) | (10.00) | (11.42) | (13.73) | (14.80) | (14.81) |
| (μg/cm² | 5-MeO-DMT | 0.00 | 0.33 | 3.30 | 8.26 | 13.33 | 18.77 | 23.43 | 29.52 | 35.36 |
| (SD)) | Hydrochloride | (0.00) | (0.52) | (3.51) | (6.70) | (8.58) | (10.75) | (11.38) | (12.77) | (13.29) |

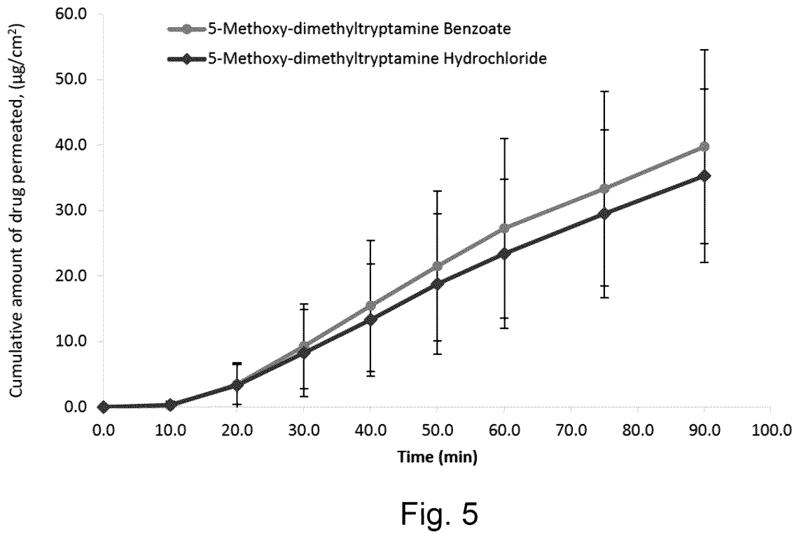

The cumulative amount of 5-MeO-DMT benzoate and 5-MeO-DMT hydrochloride which permeated through ovine nasal epithelium per unit area following application of 1.25% dosing solutions prepared in water (mean±SD, n=5) can be seen in FIG. 5.

As can clearly be seen, the benzoate salt has higher permeation across the epithelium.

The above data obtained in the above test show that the 5-MeO-DMT benzoate salt gives higher permeation with less mucosal irritation than the commonly used HCl salt; and so this combination of properties makes the benzoate salt an ideal candidate for mucosal delivery. For example, less 5-MeO-DMT benzoate salt may be needed by inhalation to provide the same benefit as the HCl salt and the benzoate salt is less irritating, and so provides a synergistic benefit. Smaller amounts of compound also make inhalation easier to accomplish.

### Example 10: Effects on the Central Nervous System Function

In the following examples, BPL-5MEO refers to 5-methoxy-N,N-dimethyltryptamine (5-MeO-DMT).

In the following examples, the hydrochloride salt of 5-MeO-DMT was used.

The following Examples (10-14) summarizes applicant-sponsored safety pharmacology studies to assess the effects of BPL-5MEO on CNS, cardiovascular system, and respiratory system function. The study designs are based on in the International Council for Harmonisation (ICH) S7A/B Guidance and were conducted in compliance with GLP regulations.

The pharmacological effects of BPL-5MEO on CNS function was assessed using a Functional Observational Battery (FOB) in male Sprague-Dawley rats following a single intranasal administration (ITR study 15951).

The test and control/vehicle items were administered by single dose intranasal administration to both nostrils, as shown in Table 7.

TABLE 7
Experimental Design of Study 15951

| | | | Dose | | |
|---|---|---|---|---|---|
| | Group | Dose | Concen- | Dose | No. of |
| Group | Designation | Level | tration | Volume c | Male |
| No. | a | (mg/kg) | (mg/mL) | (μL/kg) | Animals |
| 4 | Control b | 0 | 0 | 75 Right | 6 |
| 3 | Low Dose | 1.5 | 10 | Nostril + | 6 |
| 2 | Mid Dose | 3 | 20 | 75 Left | 6 |
| 1 | High Dose | 10 | 66.67 | Nostril | 6 |

a The observers performing the FOB were not aware of the specific treatment administered to the animals.
b Control animals were administered 0.1% hydroxypropyl methyl cellulose (HPMC) in water.
c Dose volume did not exceed 25 μL/nostril for all animals regardless of their bodyweight.

Parameters monitored included mortality and clinical signs. General behavioral changes were assessed using FOB at 6 timepoints: before dosing, and at 15 minutes, 1, 2, 4, and 24 hours postdosing. On each occasion, the FOB was performed at 4 stages: when the animals were in their home cage, while handling the animals, when the animals were freely moving in an open-field, and when they received diverse stimuli for reactivity evaluation. The body temperature and neuromuscular strength were also measured on each of the occasions detailed above.

The FOB examinations were grouped according to functional domains of the nervous system as shown in Table 8.

TABLE 8
Functional Domains of the Nervous
System and Associated Observations

| Domain | Behavioral Observations Performed |
|---|---|
| Behavioral | Posture and activity in home cage/bin |
| | Ease of removal from the cage/bin |
| | Handling reactivity |
| | Arousal |
| | Rearing |
| | Exploratory activity |
| | Touch response |
| | Abnormal or stereotyped behavior |
| Neurological | Vision test |
| (sensorimotor)/ | Touch response |
| Neuromuscular | Auditory test |
| | Tail pinch response |
| | Eye blink response |
| | Flexor reflex |
| | Extensor thrust reflex |
| | Pinna reflex |
| | Proprioceptive positioning |
| | Righting reaction |
| | Hindlimb foot splay |
| | Involuntary motor movements (such as convulsion |
| | and tremors) |
| | Gait |
| | Forelimb and hindlimb grip strength |
| Autonomic | Lacrimation |
| | Salivation |
| | Pupil response to light |
| | Palpebral closure |
| | Defecation |
| | Urination |
| | Piloerection |
| | Exophthalmos |
| | Body temperature |

There was no treatment-related mortality/morbidity. Transient BPL-5MEO-related clinical signs were noted immediately following dosing and consisted mainly of decreased activity, lying on the cage floor, shallow/increased respiration and dilated pupils at all dose groups. Tremors, salivation, and gasping were observed in some animals at the 3 and 10 mg/kg doses, and twitching was noted in one animal at 10 mg/kg.

In the behavioral domain of the FOB, a single intranasal administration of BPL-5MEO at doses of 1.5, 3, and 10 mg/kg resulted in transient decreased activity, lying on the cage floor, and decreased rearing at 15 minutes postdose. All behavioral parameters were comparable to control animals at 1-hour postdose.

In the neurological (sensorimotor)/neuromuscular domain of the FOB, a single intranasal administration of BPL-5MEO at 1, 5, and 10 mg/kg resulted in transient changes in gait (difficulty in movement) at all dose levels. All neurological (sensorimotor)/neuromuscular parameters were comparable to control animals at 1-hour postdose.

In the autonomic domain, a single intranasal administration of BPL-5MEO of 1, 5, and 10 mg/kg was associated with salivation, piloerection, increased respiration, dilated pupils and changes in body temperature was noted across all dose levels. All autonomic parameters were comparable with control animals at 2 hours postdose.

In conclusion, the single intranasal administration of BPL-5MEO at doses of 1.5, 3, and 10 mg/kg resulted in transient clinical signs, consistent with observable changes in behavior, neurological (sensorimotor)/neuromuscular and autonomic parameters which were fully resolved within 1 or 2 hours following dosing.

### Example 11: Effects on Cardiovascular Function

### In Vitro Study

The in vitro effect of 5-MeO-DMT on the hERG potassium channel current (I_{Kr}), the rapidly activating, delayed rectifier cardiac potassium current, was assessed using the patch clamp technique in stably transfected human embryonic kidney (HEK-293) cells that expressed the hERG gene (CRL study 1020-5458). This assay is employed as a screen to assess potential risks for QT interval prolongation.

The study was conducted in 2 phases: Phase 1 assessed the onset and steady-state inhibition of hERG at a selected concentration of 30 μm 5-MeO-DMT; Phase 2 assessed the concentration response if the results from Phase 1 showed inhibition of 20% or more. The initial concentration of 30 μm was selected based on the results of an exploratory dose-range finding study in dogs, where intranasal administration of 2.5 mg/kg BPL-5MEO resulted in a mean Cₘₐₓ of 803 ng/mL (3.67 μM) 5-MeO-DMT. A solution of 30 μM used in Phase 1 provided an 8-fold margin over this concentration.

In Phase 1, the 30 μM concentration of 5-MeO-DMT in protein free perfusate inhibited hERG potassium ion current by 77.8±7.4% (n=3). Therefore, Phase 2 was undertaken using concentrations of 1, 3, 10, and 35 μm 5-MeO-DMT in protein free perfusate (corresponding to 0.2, 0.6, 2.0, and 7.2 μg/mL of unbound drug substance).

In Phase 2, 5-MeO-DMT inhibited hERG potassium ion channel current in a concentration-dependent manner as presented in Table 9.

TABLE 9
Mean Percent Inhibition of hERG Potassium ion Channel
Current by 5-MeO-DMT (in protein free perfusate)

| | Concentration of 5-MeO-DMT (μM) | | | |
|---|---|---|---|---|
| | 1 | 3 | 10 | 35 |
| Mean ± SD % inhibition | 5.03 ± | 23.77 ± | 52.72 ± | 82.22 ± |
| (n = 3 cells) | 1.95% | 6.10% | 2.61% | 1.91% |

The calculated IC₅₀ of 5-MeO-DMT for hERG potassium channel current was 8.69 μm (95% confidence limits 5.78−13.06 μm) compared to 12.8 nM (95% confidence limits 6.8-24.3 nM) for the positive control, terfenadine.

### In Vivo Study

The pharmacological effects of BPL-5MEO on cardiovascular function (arterial blood pressure and ECG) was monitored by telemetry, in conscious male beagle dogs, following a single intranasal administration.

The highest dose level was selected based on the results from an intranasal maximum tolerated dose (MTD) toxicity study in dogs (Study 62958) where repeated daily dosing 2.5 mg/kg/day of BPL-MEO once daily for 5 consecutive days was marginally tolerable and associated with transient clinical observations of moderate to severe incoordination, vocalization, salivation, shaking, circling, sneezing, decreased activity, and labored respiration that resolved within 60 minutes post dosing. Therefore, the highest dose selected for this study was 1.2 mg/kg/day. The lowest dose of 0.4 mg/kg/day was based on consideration of a maximum clinical dose of 14 mg/day, with the mid-dose of 0.8 mg/kg/day selected to provide a dose-response assessment.

BPL-5MEO and control/vehicle were administered by intranasal instillation to both nostrils per session to a total of 4 dogs. Each dog received 4 administrations (control/vehicle and 3 dose levels of BPL-5MEO) according to a Latin-square design, such that each dog received the various administrations in a unique sequence, as in Table 10. A washout period of at least 2 days was allowed between each successive dose.

TABLE 10
Latin-square design for Dog Cardiovascular Study

| Test | Treatment | | | | |
|---|---|---|---|---|---|
| Session | 1001A | 1002A | 1003A | 1004A^{a} | 1104A |
| 1 | Control/Vehicle | Low Dose | Mid Dose | High Dose | — |
| 2 | High Dose | Control/Vehicle | Low Dose | Mid Dose | — |
| 3 | Mid Dose | High Dose | Control/Vehicle | — | Low Dose |
| 4 | Low Dose | Mid Dose | High Dose | — | Control/Vehicle |

^{a}Animal 1004A was replaced prior to dosing for Test Session 3 with animal 1104A due to low implant battery.

Low Dose, Mid Dose, High Dose were 0.4, 0.8, and 1.2 mg/kg/day, respectively. The nominal dose levels refer to the freebase of 5-MeO-DMT salt form.

The dose volume administered to each animal was 7 μL/kg/nostril. No animal exceeded a dose volume of 100 μL/nostril.

The Control/Vehicle was 0.1% hydroxypropyl methyl cellulose (HPMC) in water.

The telemetry signals for arterial blood pressure and pulse rate, ECGs (heart rate [HR], RR, PR, QT, and QTcV intervals and QRS complex duration), body temperature, and locomotor activity, were recorded continuously over the telemetry recording period of at least 1.5 hours before the start of dosing and for at least 24 hours postdosing. Systolic, diastolic and mean arterial blood pressures and pulse rate were obtained from transmitter catheter inserted into the femoral artery. ECGs were obtained from the biopotential leads, from the telemetry transmitter, in a Lead II configuration.

During the study, all animals were also monitored for mortality and clinical signs. Body weights were recorded for general health status check and for dose calculation purposes only.

There were no deaths and no BPL-5MEO-related clinical signs during the study.

The morphology of the P-QRS-T waveforms remained normal and no rhythm or conduction abnormalities were observed in the ECGs between control and treated groups. There were minor differences in the % change of mean HR averaged between approximately 0 and 150 minutes postdose between all dose levels and the control vehicle. While mean % increases in mean HR increased by 3.7% in the control vehicle during this period, compared to baseline, the observed increases with the low, mid and high dose levels of BPL-5MEO were respectively 7.6%, 10.3%, and 17.2%. However, arterial blood pressure did not seem to show any appreciable differences that were sufficient to have any effect on HR. No other findings were observed. The observed increases in mean HR with all dose levels were non-adverse, reversible and did not show a typical dose relationship.

In conclusion, the single intranasal instillation of BPL-5MEO to both nostrils at doses of 0.4, 0.8, and 1.2 mg/kg/day was well tolerated and did not result in any effects on the cardiovascular system of conscious male Beagle dogs.

### Example 12: Absorption and Pharmacokinetics

In a 14-day intranasal toxicology in male and female rats (ITR report 700041), plasma concentrations of 5-MeO-DMT increased as a function of the dose administered. Peak (Cₘₐₓ) concentrations were reached within 2 to 5 minutes post dosing (Tₘₐₓ) with apparent t_{1/2} ranging from 6.8 to 9.4 minutes. Values trended lower on Day 14 compared to Day 1. There was no apparent sex difference and no evidence of accumulation with repeated dosing.

In a 14-day intranasal toxicology study in male and female dogs (ITR report 62959), plasma concentration of 5-MeO-DMT increased as a function of the dose administered. Peak concentrations were reached within 3 to 14 minutes (Tₘₐₓ), post dosing with apparent elimination half-lives ranging from 19 to 95 minutes. The values were not markedly different on Day 1 and Day 14. There was no apparent sex difference and no evidence of accumulation with repeated dosing.

The data shows that across the dose ranges studied in rats (5, 20, 75 mg/kg), and dogs (0.4, 0.8, 1.5, and 2.5 mg/kg), exposure was generally increased dose-dependently, but not consistently in a dose-proportional manner as some increases were more or less than dose-proportional between different doses. The results do not indicate a saturation of MAOA-mediated metabolism at the doses studied in these species as seen previously in mice.

### Example 13: Toxicology

The toxicology program completed with BPL-5MEO consisted of non-pivotal single/repeat dose intranasal studies to determine the MTD in order to help select the highest doses for the pivotal 14-day GLP intranasal toxicology studies in male and female Sprague Dawley rats and Beagle dogs. The intranasal route of administration was used as this is the clinical route of administration. The species selected were based upon information from the published literature, preliminary PK information, availability of historical control information from the testing laboratory, and their standard use and acceptance as appropriate surrogates for intranasal administration. The experimental design of the pivotal 14-day studies included an assessment of systemic exposures (toxicokinetics) and a 14-day recovery period to assess reversibility of any adverse or delayed responses. The once daily dosing for 14 consecutive days in the pivotal studies was intended to provide sufficient systemic exposure to characterize the toxicity potential for a drug substance with a very short half-life.

### 1. Non-Pivotal Single/Repeat Dose and Tolerance Studies

a. Maximum Tolerated Dose Followed by 7-Day Repeat-Dose Toxicology in Rats (Study 700040)

The objectives of this non-GLP study were to determine the maximum tolerated dose and the toxicity profile of BPL-5MEO following intranasal instillation in the rat. The study consisted of 2 parts. The objective of the first part (Dose Escalation Phase), was to determine the MTD of BPL-5MEO following a single intranasal administration to Sprague-Dawley rats. The doses used in part 1 were 15, 30, 50, 65, and 75 mg/kg. Each subsequent dose was administered following at least 24 hours from the commencement of the previous dose. There were 2 males and 2 females in each dose group. The objective of the second part (Main Study Phase), was to determine the toxicity of BPL-5MEO at the MTD of 75 mg/kg following once daily intranasal administration for 7 consecutive days to Sprague-Dawley rats.

All the dose formulation samples collected and analyzed were between 89.2% and 101.3% of nominal concentration, and as such met the acceptance criteria for accuracy (100±15% of their nominal concentration). Analysis was performed using a non-GLP HPLC-UV assay.

All female groups received their targeted doses in both parts. However, as the maximum feasible loading dose was not to exceed 25 μL/naris, regardless of body weight, mean achieved doses for the males at the 30 were still 99.3%, 90.0%, 88.2%, and 89.6%, respectively and were considered to be acceptable.

During Phase I, assessments of mortality, clinical signs and body weights were performed. All animals were observed for 14 days after dosing, following which they were euthanized on Day 15 and subjected to a gross necropsy examination. The necropsy consisted of an external examination, including reference to all clinically-recorded lesions, as well as a detailed internal examination.

Single intranasal administration of 5-MeO-DMT at the dose levels up to 75 mg/kg was tolerated. There was no mortality and gross pathology findings at any dose. Body weight gain was slightly suppressed females at 75 mg/kg. A range of clinical signs were observed and included incoordination, shallow or increased respiration, sneezing, salivation, decreased activity, piloerection, white pasty material around penis (for males), ptosis, laying on the cage floor, and sensitive to touch and shaking. The incidence and severity of these findings evolved as a function of the administered dose and were transient, with most being resolved within 1-hour post dose. Based on the clinical signs and maximal feasible volume/dose, 75 mg/kg was judged to be the MTD, and this dose was selected for Phase 2.

During Phase 2, assessments of mortality, clinical signs and body weights were performed. Following dosing, all animals were euthanized and subjected to a necropsy examination on Day 8. The necropsy consisted of an external examination, including reference to all clinically-recorded lesions, as well as a detailed internal examination. Study plan specific tissues/organs were collected and retained, then trimmed and preserved promptly once the animal was euthanized but these were not further examined microscopically.

Intranasal administration of 5-MeO-DMT at 75 mg/kg for 7 consecutive days was tolerated. There were no mortalities. Body weight gain was slightly suppressed for both sexes. Transient clinical signs similar to those of the Phase I included incoordination, mydriasis, increased or shallow respiration, gasping, sneezing, salivation, pale in colour, decreased activity, lying on the cage floor, piloerection, white pasty material around penis (for males), erect penis (for males), cold to touch, partially or completely closed eyes, sensitive to touch and shaking. These signs were generally less pronounced in terms of severity and incidence during the last few dosing days of this phase, and were resolved daily following dosing within 1-hour post administration. Macroscopic observations of note were limited to dark/pale area of the lungs in 2/10 animals; however, in the absence of histopathological examination, a possible test item-relationship of these findings could not be excluded.

b. Maximum Tolerated Dose Followed by 7-Day Repeat-Dose Toxicology in Dogs (Study 62958)

The objectives of this study were to determine the maximum tolerated dose and the toxicity of the test item, 5-MeO-DMT (as the hydrochloride salt), following intranasal instillation in the dogs. In support of these objectives, the study consisted of 2 individual phases.

The test item was administered once by intranasal instillation to one male and female dog for up to 5 dose levels until the highest tolerable dose (MTD) was determined as described in Table 11.

TABLE 11
Doses Administered in the Dose Escalation Phase in Study 62958

| Dosing | Group | Total Dose Level ^{b} | Dose Concentration | Dose Volume | Number of Animals | |
|---|---|---|---|---|---|---|
| Day ^{a} | Designation | (mg/kg) | (mg/mL) | (μL/kg) | Males | Females |
| Day 1 | Dose 1 | 2 | 100 | 10 Right Nostril + | 1 | 1 |
| Day 7 | Dose 2 | 4 | 200 | 10 Left Nostril | | |
| Day 10 | Dose 3 | 5 ^{d} | 250 | | | |
| Day 14 | Dose 4 | 3 | 150 | | | |
| Day 17 | Dose 5 | 3.5 | 175 | | | |

^{a} Each subsequent dose was administered following a washout period of minimum 3 days between doses.
^{b} Dose levels refer to the freebase of BPL-5MEO salt form.
c Targeted dose concentrations were calculated based on an estimated body weight of 10 kg.
^{d} These animals were dosed at higher dose level of 5 mg/kg.

There were no BPL-5MEO-related effects on mortality or bodyweights. Slight decreases in food intake were observed following administration for the male on Days 1 (Dose 1) and 9 (Dose 2) and for the female on Days 4 (Dose 1) and 9 (Dose 2). A range of clinical signs were observed and included gnawing cage wire, dilated pupils, changes in respiration, incoordination, decreased activity, vocalization, salivation, erect penis (for males) and shaking. After the last escalating dose at 3.5 mg/kg/day, the male animal presented a convulsion shortly after dosing which lasted for 8 minutes. All clinical signs disappeared within an hour after the dosing except for decreased activity, dilated pupils and lying on the cage floor which were present on few occasions at 1-hour post dose or a few minutes after. The MTD for the test item was considered to be 2.5 mg/kg.

In the phase 2 (dose confirmation), BPL-5MEO was administered at the MTD to one male and female dog once daily by intranasal instillation for 5 consecutive days and then twice daily on Days 6 and 7 (minimum 4 hours apart). During Phase 2, assessments of mortality, clinical signs, body weights and food consumption were performed. A series of blood samples were collected on Days 1 and 7 for determination of plasma concentrations of 5-MeO-DMT using an LC/MS/MS method. Following the last dosing, all animals were euthanized and subjected to a necropsy examination on Day 8. The necropsy consisted of an external examination; including reference to all clinically-recorded lesions, as well as a detailed internal examination. Study plan specific tissues/organs were collected and preserved following necropsy but were not further examined microscopically.

There were no test item-related effects on mortality or bodyweights. Slight decreases in food intake were observed for the male animal on Day 7 and for the female animal on Days 5 and 7. A range of clinical signs were observed and included muscle stiffness, gnawing cage wire, dilated pupils, changes in respiration, decreased activity, incoordination, vocalization, salivation, erect penis (for the male) and shaking. All clinical signs disappeared within an hour after the dosing except for decreased activity, dilated pupils, and lying on the cage floor which were present on few occasions at 1-hour post dose or a few minutes after. All observations were considered transient.

Toxicokinetic assessments were performed on Days 1 and 7; the maximum BPL-5MEO plasma concentration (Cₘₐₓ) ranged from 541 to 803 ng/mL and was reached (Tₘₐₓ) within 2 to 15 minutes post dose in both sexes. Dose normalized AUCs ranged from 2980 to 7320 min*kg*ng/mL/mg in both sexes. After Tₘₐₓ, BPL-5MEO plasma concentrations declined at an estimated t_{1/2} from 19.1 to 34 minutes in both sexes. There were no sex differences in any of the measured toxicokinetic parameters on either occasion. Over the 7-day treatment period, BPL-5MEO did not accumulate when administered daily by intranasal instillation.

### 2. Pivotal Studies

a. A 14-Day Repeat-Dose Intranasal Toxicity Study Followed by a 14-Day Recovery Period in Rats (Study 700041)

The objective of this GLP study was to determine the toxicity and toxicokinetic (TK) profile of BPL-5MEO following intranasal instillation in Sprague Dawley rats for 14 consecutive days and to assess the persistence, delayed onset, or reversibility of any changes following a 14-day recovery period.

BPL-5MEO and control/vehicle were administered to groups of rats once daily by intranasal instillation for 14 consecutive days as described in Table 12.

TABLE 12
Doses Administered in 14-Day Repeat Dose Study in Rats

| | | Total | | | | | | | | |
|---|---|---|---|---|---|---|---|---|---|---|
| | | Dose | | | | | | | | |
| | | Level ^{b} | Dose | Dose | Number of Animals | | | | | |
| Group | Group | (mg/kg/ | Conc. | Volume′ ^{d} | Main | | Recovery | | Toxicokinetic | |
| No. | Designation | day) | (mg/mL) | (μL/kg) | Male | Female | Male | Female | Male | Female |
| 1 | Vehicle | 0 | 0 | 75 | 10 | 10 | 5 | 5 | 3 | 3 |
| | Control a | | | Right | | | | | | |
| 2 | Low Dose | 5 | 33.3 | Nostril + | 10 | 10 | — | — | 6 | 6 |
| | | | | 75 | | | | | | |
| 3 | Mid Dose | 20 | 133.3 | Left | 10 | 10 | — | — | 6 | 6 |
| 4 | High Dose | 75 | 500 | Nostril | 10 | 10 | 5 | 5 | 6 | 6 |

a Vehicle control animals were administered 0.1% Hydroxypropyl methyl cellulose (HPMC) in water.
^{b} Nominal dose levels refer to the freebase of 5-MeO-DMT salt form.
c The dose volume administered to each animal was 75 μL/kg/nostril.
^{d} Dose volume was not to exceed 25 μL/nostril for all animals regardless of their bodyweight.

The animals were monitored for mortality, clinical signs, respiratory measurements, body weights, food consumption, and body temperature. Ophthalmoscopic examinations and respiratory function tests were performed on all animals at scheduled timepoints. Clinical pathology assessments (hematology, coagulation, clinical chemistry, and urinalysis) were evaluated at termination. Blood samples were collected from the jugular vein from the TK animals on Days 1 and 14, for up to 8 hours after treatment for bioanalysis of 5-MeO-DMT concentrations in plasma and the subsequent calculation of toxicokinetic parameters. Following dosing, the Main animals were euthanized and subjected to a complete necropsy examination on Day 15. The Recovery animals were observed for an additional 14 days and then euthanized and subjected to a complete necropsy examination on Day 28. TK animals were euthanized after the last blood collection and discarded without further examination. At terminal euthanasia, selected tissues/organs were weighed, and microscopic evaluations of a standard set of tissues including the nasal turbinates (4 sections) and brain (7 sections) were performed for all Main and Recovery study animals.

Following dosing, animals in the Main group were euthanized and subjected to a necropsy examination on Day 15. The animals in the Recovery group were observed for 14 days and then euthanized and subjected to a necropsy examination on Day 28. For toxicokinetics, a series of 8 blood samples (approximately 0.5 mL each) were collected from all rats in the Toxicokinetic group (3 rats/sex/timepoint) on Days 1 and 14 of the treatment period at 2, 5, 10, 15 and 30 minutes, and 1.0, 3.0 and 8 hours after treatment. For control rats (3 rats/sex) in the Toxicokinetic group only 1 sample was collected at the 15 minutes post dosing timepoint on Days 1 and 14.

Toxicity was based on the following parameters monitored: mortality/morbidity, clinical observations, body weights/gains, food consumption, ophthalmoscopy, clinical pathology (hematology, coagulation, chemistry, and urinalysis), necropsy observations, selected organ weights, and microscopic examination of a complete set of standard tissues including 4 cross levels of the nasal cavity and 7 sections of the brain.

### Results

All the samples met the acceptance criteria for accuracy (100±10% of their nominal concentration).

All animals were dosed without any major incidents and no sneezing was noted. All groups received their targeted doses on Days 1 to 10. As the maximum feasible loading dose was not to exceed 25 μL/naris (due to limited nasal surface area), once the bodyweights exceeded 333 g, male animals in all groups received slightly lower dose levels on Days 11 to 14. This was considered to have no impact on the study data as the differences were negligible.

No mortality occurred over the course of this study.

The observed clinical signs were as follows:

### Group 2 (Low Dose)

Both male and female animals exhibited incoordination, shaking, salivation, decreased activity, lying on cage floor and sensitive to touch. For one female animal on Day 3, increased respiration was also observed.

### Group 3 (Mid Dose)

Both male and female animals exhibited incoordination, shaking (or tremor), increased or shallow respiration, mydriasis, salivation, decreased activity, partially closed eyes, lying on cage floor and sensitive to touch. Male animals also exhibited erect penis.

### Group 4 (High Dose)

Both male and female animals exhibited incoordination, shaking (or tremor), increased or shallow respiration, mydriasis, salivation, decreased activity, partially closed eyes, lying on cage floor and sensitive to touch. Male animals also exhibited erect penis.

Increased respiration was recorded for the mid and high dose group, however, measured respiratory values using plethysmographs proved that there were actually decreases in respiratory rates.

All the above clinical signs were considered to be transient for all groups.

Slight, generally dose-dependent body weight gain suppression was observed for both sexes between Days 1 to 14. There were no changes in food consumption that could be attributed to treatment with at dose levels 75 mg/kg/day for 14 days.

On Day 14, slight body temperature increases were observed at 15 minutes and 30 minutes postdose for all treated male animals, for females on Day 14, the body temperature increases were observed in one or all treated groups for all the timepoints (until 2 hours postdose). These increases in body temperature were more pronounced in the mid (20 mg/kg/day) and high (75 mg/kg/day) dose groups.

When compared to pretreatment or control group, decreases in respiratory rates were observed at 20 minutes postdose timepoint which resulted in decreases in respiratory minute volumes. Tidal volume values were either comparable to pre-dose or to control values. The 20-minute postdose respiratory measurements on Day 1 was not performed for Group 2 female animals inadvertently. This considered to have no impact on the study data as the data could be extrapolated form the male animals in the same group. There were no significant between the sexes.

There was no adverse ocular effect, caused by the administration of BPL-5MEO at dose levels 75 mg/kg/day for 14 days.

All other clinical observations, bodyweight changes, food consumption changes, and body temperature changes were considered to be not BPL-5MEO-related as they were sporadic, comparable to pretreatment signs or control animals, and not dose-related.

When compared to control Group, platelet, neutrophil, monocyte and basophil counts were slightly increased in mid and high dose groups in both sexes, however, these values were still within the historical ranges. On Day 28, all these values were compared to those in control group.

All changes in the hematology parameters, including those that reached statistical significance, were not attributed to the administration of BPL-5MEO as they were minor (within the normal physiological range), comparable to control values, and/or not dose-related.

When compared to control Group, activated partial thromboplastin times (APTT) were increased for both sexes in the mid (20 mg/kg/day) and high (75 mg/kg/day) dose groups. All the coagulation values on Day 28 were comparable to control group. All other changes in the coagulation parameters were not attributed to the administration of BPL-5MEO as they were minor (within the normal physiological range), comparable to control values, and/or not dose-related.

There were no changes in clinical chemistry and urinalysis parameters that could be attributed to the administration of BPL-5MEO at dose levels 75 mg/kg/day for 14 days. All changes in the parameters, including those clinical chemistry parameters that reached statistical significance, were not attributed to the administration of BPL-5MEO as they were minor (within the normal physiological range), comparable to control values, and/or not dose-related.

Compared to control values, there were decreases in thymus weights (absolute and relative to terminal body weight) observed in male animals as shown in Table 13.

TABLE 13
Thymus Weights for Male Animals Compared to Control Group

Thymus

| | Mean Absolute | Mean Relative to |
|---|---|---|
| Group (Males only) | Weight ^{a} | the Body Weight ^{a} |
| Control (Group 1) | 0.6028 | 0.1756 |
| Group 2 | −4 | −6 |
| Group 3 | 18 | −16 |
| Group 4 | −31 | −28 |

^{a} For Control group, the organ weight in grams is reported, for other groups, the percentage compared to the control value is shown.

All changes in the organ weight parameters, including those that reached statistical significance, were not attributed to the administration of BPL-5MeO as they were minor, comparable to control values, and/or not dose related.

There were no macroscopic findings related to treatment with BPL-5MEO in rats in either the Main Recovery groups.

For animals in the Main group, microscopic findings related to treatment with BPL-5MEO, were noted in the nasal cavity sections 1, 2, 3 and 4 of Main rats.

A range of minimal to mild changes were noted in the respiratory, transitional, and/or olfactory epithelium of the nasal cavities, 1, 2, 3, and 4. The incidence and severity of changes were greater in males compared to females and were proportional to the dose of BPL-5MEO.

Microscopic changes observed in rats dosed with 75 mg/kg/day of BPL-5MEO (Group 4) included: respiratory epithelium, minimal to mild degeneration, hyperplasia, and squamous metaplasia, minimal mononuclear infiltrate and/or lumen exudate in nasal cavities 1, 2, 3, and/or 4; transitional epithelium, minimal hyperplasia in nasal cavity 1, and; olfactory epithelium, minimal to mild degeneration and/or minimal mononuclear infiltrate and erosion in nasal cavities 2, 3, and/or 4. Minimal degeneration of the olfactory epithelium of the nasal cavities 2 and 3 was noted in male and/or female rats dosed with 5 and/or 20 mg/kg/day of BPL-5MEO (Group 2 and 3). Minimal degeneration of the respiratory epithelium of the nasal cavities 1 and 2 was noted in male and/or female rats dosed with 20 mg/kg/day of BPL-5MEO (Group 3).

For animals in the Recovery group, microscopic findings related to treatment with BPL-5MEO, were noted in the nasal cavity sections 1, 2, 3, and 4 of Recovery rats. Minimal to mild changes were noted in the respiratory and olfactory epithelium of the nasal cavities, 1, 2, 3, and/or 4. The incidence and severity of changes were greater in males compared to females. Microscopic changes included minimal to mild degeneration of respiratory epithelium in nasal cavities 1 and 2 and minimal degeneration olfactory epithelium in nasal cavities 2, 3, and 4 indicating incomplete but progressive ongoing reversal of epithelial degeneration following a 14-day recovery period. There was complete reversal of all other microscopic changes noted previously in the nasal cavities of Main rats following a 14-day recovery period including reversal of epithelial hyperplasia, squamous metaplasia, mononuclear infiltrate, erosion, and lumen exudate.

Other microscopic findings in both the Main and Recovery groups were considered to be procedure-related or incidental as they were not dose-related, of low incidence or severity, and/or as they were also seen in the control animals.

### Toxicokinetics

Over the dose range, exposure to 5-MeO-DMT (based on the area under the plasma drug concentration-time curve from the time of dosing to the last quantifiable concentration [AUC_{0-Tlast}] values) on Days 1 and 14 generally increased dose-dependently (except for Group 4 as stated below), but not consistently in a dose-proportional manner as some increases were more or less than dose-proportional between different doses. Furthermore, on Day 14, the exposure in Female group 4 (75 mg/kg/day) decreased compared to Female Group 3 (20 mg/kg/day).

The sex ratios ranged between 0.4 and 6.2, but as the sex ratio randomly varied between dose groups and occasions, it was considered there was no sex-related difference.

Accumulation ratios (based on AUC_{0-Tlast}) ranged sporadically from 0.3 to 2.9 (Day 14/Day 1) suggesting that 5-MeO-DMT does not accumulate when administered once daily for 14 consecutive days (2 weeks) by intranasal instillation in the Sprague Dawley rats at doses up to 75 mg/kg/days.

The mean toxicokinetic parameters for Groups 2, 3, and 4 are presented in Table 14.

TABLE 14
Mean Toxicokinetic Parameters From Study 700041

| | Dose | | Day 1 | | Day 14 | |
|---|---|---|---|---|---|---|
| Group | (mg/kg/day) | Parameter | Male | Female | Male | Female |
| 2 | 5 | Tₘₐₓ (h) | 0.0833 | 0.166 | 0.0833 | 0.0333 |
| | | AUC_{0-Tlast} [SE] | 39.9 [7.35] | 53.2 [15.9] | 114 [13.8] | 63.8 [4.55] |
| | | (AUC_{INF}_{_{—}}_{obs}) (h*ng/mL) | (40.1) | (53.7) | (115) | (64.0) |
| | | Cₘₐₓ [SE] (ng/mL) | 191 [45.6] | 186 [98.7] | 627 [102] | 645 [106] |
| | | t_{1/2} (h) | 0.137 | 0.150 | 0.142 | 0.113 |
| 3 | 20 | Tₘₐₓ (h) | 0.0333 | 0.0833 | 0.0333 | 0.0833 |
| | | AUC_{0-Tlast} [SE] | 420 [62.1] | 198 [15.2] | 133 [57.2] | 169 [21.2] |
| | | (AUC_{INF}_{_{—}}_{obs}) (h*ng/mL) | (421) | (198) | (133) | (169) |
| | | Cₘₐₓ [SE] (ng/mL) | 4190 [1040] | 679 [162] | 1200 [857] | 795 [115] |
| | | t_{1/2} (h) | 0.125 | 0.140 | 0.143 | 0.147 |
| 4 | 75 | Tₘₐₓ (h) | 0.0333 | 0.0333 | 0.0333 | 0.0333 |
| | | AUC_{0-Tlast} [SE] | 1030 [114] | 228 [49.7] | 391 [228] | 155 [53.8] |
| | | (AUC_{INF}_{_{—}}_{obs}) (h*ng/mL) | (1040) | (228) | (392) | (156) |
| | | Cₘₐₓ [SE] (ng/mL) | 7010 [1010] | 1310 [802] | 3290 [2510] | 870 [361] |
| | | t_{1/2} (h) | 0.133 | 0.156 | 0.116 | 0.130 |

Abbreviations: AUC_{0-Tlast} = Area under the plasma drug concentration-time curve from the time of dosing to the last quantifiable concentration; AUC_{INF}_{_{—}}_{obs} = Area under the plasma drug concentration-time curve from the time of dosing extrapolated to infinity; Cₘₐₓ = The maximum plasma concentration; h = hours; SE = standard error of mean; t_{1/2} = Terminal elimination half-life; Tₘₐₓ = Time to maximum plasma concentration.

### Conclusion

Intranasal administration of BPL-5MEO at dose levels 75 mg/kg/day for 14 consecutive days was tolerated with no BPL-5MEO-related effects on mortality, ophthalmology, clinical chemistry, macroscopic findings and urinalysis. Slight dose-dependent body weight gain suppression was observed for both sexes. Transient clinical signs included incoordination, shaking (or tremor), increased or shallow respiration, mydriasis, salivation, decreased activity, partially closed eyes, lying on cage floor and sensitive to touch. Male animals also exhibited erect penis. Slight dose dependent body temperature increases were observed for both sexes.

Decreases in respiratory rates were observed at 20 minutes post dose timepoint which resulted in decreases in respiratory minute volumes. Platelet, neutrophil, monocyte and basophil counts were slightly increased in mid and high dose groups in both sexes. APTT were increased for both sexes for main animals in the mid (20 mg/kg/day) and high (75 mg/kg/day) dose groups. There were decreases in thymus weights (absolute and relative to terminal bodyweight) observed in male animals. Microscopic changes were noted in nasal cavities 1, 2, 3, and/or 4 involving the respiratory, olfactory, and transitional epithelium. The incidence and severity of findings were greater in males compared to females and were proportional to the dose of BPL-5MEO with incomplete but progressive on-going reversal following a 14-day recovery period.

The NOAEL was reported as the lowest dose of 5 mg/kg.

b. A 14-Day Repeat-Dose Intranasal Toxicity Study Followed by a 14-Day Recovery Period in Dogs (Study 62959)

The objective of this GLP study (Study 62959) was to determine the toxicity and TK profile of BPL-5MEO following intranasal instillation in Beagle dogs for 14 consecutive days and to assess the persistence, delayed onset, or reversibility of any changes following a 14-day recovery period.

BPL-5MEO and control/vehicle were administered to groups of dogs once daily by intranasal instillation for 14 consecutive days as described in Table 15.

TABLE 15
Doses Administered in 14-Day Repeat Dose Study in Dogs

| | | Total Dose | Dose | Dose | Number of Animals | | | |
|---|---|---|---|---|---|---|---|---|
| Group | Group | Level ^{b} | Conc. | Volume ^{d, e} | Main | | Recovery | |
| Number | Designation | (mg/kg/day) | (mg/mL) | (μL/kg) | Male | Female | Male | Female |
| 1 | Vehicle | 0 | 0 | 10 Right | 3 | 3 | 2 | 2 |
| | Control ^{a} | | | Nostril + 10 | | | | |
| 2 | Low Dose | 0.4 | 20 | Left Nostril | 3 | 3 | — | — |
| 3 | Mid Dose | 0.8 | 40 | | 3 | 3 | — | — |
| 4 | High Dose | 2.5 & 1.5^{c} | 125 & 75^{c} | | 3 | 3 | 2 | 2 |

^{a} Vehicle control animals were administered 0.1% Hydroxypropyl methyl cellulose (HPMC) in water.
^{b} Dose levels refer to the freebase of 5-MeO-DMT salt form.
^{c}Replicate A high dose animals showed severe clinical signs of muscle stiffness (rigidity), tachycardia, tachypnea, hyperthermia and aggressiveness after dosing on Day 1 at the dose level of 2.5 mg/kg. The dose level was subsequently decreased on Day 1 for the Replicates B and C to 1.5 mg/kg. Replicate A received 1.5 mg/kg on Days 2 to 14.
^{d} The dose volume administered to each animal was 10 μL/kg/nostril.
^{e} Dose volume was not to exceed 100 μL/nostril for all animals regardless of their bodyweight.

Assessments of mortality, clinical signs, olfactory reflex, body weights, food consumption, ophthalmology, and electrocardiograms were performed. In addition, clinical pathology assessments (hematology, coagulation, clinical chemistry and urinalysis) were evaluated once pretreatment and at termination. Blood samples were collected from the jugular vein of all animals on Days 1 and 14, at up to 8 time points relative to treatment, for analysis of test item concentration in plasma and the subsequent calculation of toxicokinetic parameters. Following dosing, the Main animals were euthanized and subjected to a complete necropsy examination on Day 15. The Recovery animals were observed for an additional 14 days test article free and then euthanized and subjected to a complete necropsy examination on Day 28. All Main and Recovery study animals underwent complete necropsy examinations, selected tissues/organs were retained, and microscopic evaluations of a standard set of tissues were performed.

For toxicokinetics, a series of 8 blood samples were collected from the jugular vein from all treated animals on each of Days 1 and 14 of the treatment period at 2, 5, 10, 15, 30, and 60 minutes as well as 3 and 8 hours after treatment.

For Group 1, only one sample was taken at 15 minutes post dosing on Days 1 and 14 in order to confirm the absence of BPL-5MEO in animals in the vehicle control group. Blood samples were analysed for the BPL-5MEO concentration in plasma and the subsequent calculation of TK parameters.

### Results

All the dose formulation samples collected and analyzed met the acceptance criteria for accuracy (100±10% of their nominal concentration).

Daily intranasal administration of BPL-5MEO to both nostrils of Beagle dogs once daily for 14 consecutive days at dose levels up to 1.5 mg/kg/day did not cause any mortality. High dose animals initially given to a subset of dogs at 2.5 mg/kg and showed severe clinical signs of muscle stiffness (rigidity), tachycardia, tachypnea, hyperthermia and aggressiveness after dosing on Day 1 and this dose exceeded the MTD. The high dose was subsequently lowered on Day 2 to 1.5 mg/kg/day and this dose was tolerated. Animals in all treated Groups exhibited transient clinical observation of incoordination, vocalization, mydriasis, decreased or increased activity, increased respiration, gnawing cage wire, excessive licking of nose or lips and circling. In addition, eye discharge and shaking were observed in the Mid and High dose groups. Erect penis was also recorded for the high dose male animals. All these clinical signs were considered to be exacerbated pharmacology manifestations, occurred within 10 to 30 minutes of dosing, and were resolved within 90 minutes.

When compared to control Group, the triglyceride level of ⅓ Group 3 female, ⅕ Group 4 male and ⅘ Group 4 females were increased, these data are presented in Table 16. There were no other treatment-related clinical pathology findings.

TABLE 16
Mean ± SD Day 14 Triglyceride
Values Compared to Control Group

| | |
|---|---|
| Dose | Triglyceride (mmol/L) |

| Group | (mg/kg/day) | Males ^{a} | Females ^{a} |
|---|---|---|---|
| Group 1 | Control | 0.38 ± 0.13 | 0.34 ± 0.12 |
| Group 2 | 0.4 | 0.40 ± 0.11 | 0.46 ± 0.61 |
| Group 3 | 0.8 | 0.44 ± 0.07 | 0.47 ± 0.22 |
| Group 4 | 2.5 & 1.5^{b} | 0.42 ± 0.16 | 0.69 ± 0.24 |

Abbreviations: SD = standard deviation
^{a} for Control group, the control value is mentioned, for other groups, the percentage compared to the control value is shown.
^{b}Replicate A high dose animals showed severe clinical signs of muscle stiffness (rigidity), tachycardia, tachypnea, hyperthermia and aggressiveness after dosing on Day 1 at the dose level of 2.5 mg/kg. The dose level was subsequently decreased on Day 1 for the Replicates B and C to 1.5 mg/kg. Replicate A received 1.5 mg/kg on Days 2 to 14.

All other changes in the clinical chemistry parameters, including those that reached statistical significance, were not attributed to the administration of BPL-5MEO as they were minor (within the normal physiological range), comparable to control values, and/or not dose related.

There were no changes in olfactory reflex, food consumption, body weight, ocular effect, or ECG that could be clearly attributed to treatment with BPL-5MEO at a dose level 1.5 mg/kg/day for 14 days. All body weight changes were not attributed to the administration of the test item as they were minor, and not toxicologically relevant. All food consumption changes, including those that were statistically significant, were not attributed to the administration of the test item as they were minor, and not toxicologically relevant.

Animals showed hyperthermia at the dose level of 2.5 mg/kg/day on Day 1. Transient body temperature increases were observed on Day 14 for high dose group in both sexes at 15 and 30 minutes postdose. All other body temperature changes were not attributed to the administration of the test item as they were minor, and not toxicologically relevant.

Histopathological examination results for Main animals included minimal to moderate decreased cellularity of the thymic lymphocytes at dose levels of 0.8 (1 male) and 1.5 mg/kg/day (3 males), which was determined as stress related. Minimal epithelial metaplasia of respiratory epithelium in the nasal cavities found at dose levels of 0.8 (1 female) and 1.5 mg/kg/day (2 males) and minimal to mild mononuclear cell infiltrate of the olfactory epithelium in the nasal cavities seen at a dose level of 1.5 mg/kg/day (1 male/1 female) were considered to be signs of irritation caused by BPL-5MEO but not adverse.

In animals euthanized after a 14-day recovery period, only minimal mononuclear cell infiltrate of the olfactory epithelium in the nasal cavities was still present at a dose level of 1.5 mg/kg/day (1 female) but at a lower severity when compared with animals euthanized terminally, indicative of recovery. Decreased cellularity of thymic lymphocytes was no longer observed.

### Toxicokinetics

BPL-5MEO was not detected in any of the samples collected from the Control (Group 1) animals on Days 1 and 14.

The mean toxicokinetic parameters for Groups 2, 3, and 4 are presented in the table below.

### Mean Toxicokinetic Parameters From Study 62959

| | Dose | | Day 1 | | Day 14 | |
|---|---|---|---|---|---|---|
| Group | (mg/kg/day) | Parameter | Male | Female | Male | Female |
| 2 | 0.4 | Tₘₐₓ (h) | 0.0942 | 0.194 | 0.111 | 0.0942 |

| | | | | | | | |
|---|---|---|---|---|---|---|---|
| AUC_{0-Tlast} (AUC_{INF}_{_{—}}_{obs}) | 77.9 | (80.9) | 104 (106) | 70.6 | (77.7) | 86.4 | (95.9) |

| | | | | | | |
|---|---|---|---|---|---|---|
| | | (h*ng/mL) | | | | |
| | | C_{max (}ng/mL) | 343 | 242 | 285 | 196 |
| | | t_{1/2} (h) | 0.571 | 0.312 | 0.429 | 0.706 |
| 3 | 0.8 | Tₘₐₓ (h) | 0.111 | 0.139 | 0.111 | 0.0833 |

| | | | | | | | |
|---|---|---|---|---|---|---|---|
| AUC_{0-Tlast} (AUC_{INF}_{_{—}}_{obs}) | 152 | (160) | 261 (265) | 298 | (322) | 248 | (279) |

| | | | | | | |
|---|---|---|---|---|---|---|
| | | (h*ng/mL) | | | | |
| | | Cₘₐₓ (ng/mL) | 300 | 328 | 411 | 244 |
| | | t_{1/2} (h) | 0.595 | 0.730 | 1.32 | 1.59 |
| 4 | 2.5 & 1.5^{a} | Tₘₐₓ (h) | 0.146 | 0.111 | 0.223 | 0.0898 |

| | | | | | | | |
|---|---|---|---|---|---|---|---|
| AUC_{0-Tlast} (AUC_{INF}_{_{—}}_{obs}) | 277 | (280) | 263 (271) | 260 | (287) | 165 | (167) |

| | | | | |
|---|---|---|---|---|
| (h*ng/mL) | | | | |
| Cₘₐₓ (ng/mL) | 561 | 348 | 464 | 379 |
| t_{1/2} (h) | 0.718 | 0.848 | 0.816 | 0.725 |

Abbreviations: AUC_{0-Tlast} = Area under the plasma drug concentration-time curve from the time of dosing to the last quantifiable concentration; AUC_{INF}_{_{—}}_{obs} = Area under the plasma drug concentration-time curve from the time of dosing extrapolated to infinity; Cₘₐₓ = The maximum plasma concentration; h = hours; t_{1/2} = Terminal elimination half-life; Tₘₐₓ = Time to maximum plasma concentration.
^{a}Replicate A high dose animals showed severe clinical signs of muscle stiffness (rigidity), tachycardia, tachypnea, hyperthermia and aggressiveness after dosing on Day 1 at the dose level of 2.5 mg/kg. The dose level was subsequently decreased on Day 1 for the Replicates B and C to 1.5 mg/kg. Replicate A received 1.5 mg/kg on Days 2 to 14.

Over the dose range, exposure to BPL-5MEO (based on AUC_{0-Tlast} values) on Days 1 and 14 generally increased dose-dependently (except for Group 4 as stated below), but not consistently in a dose-proportional manner as some increases were more or less than dose-proportional between different doses. Furthermore, on Day 14, the exposure in Group 4 (1.5 mg/kg/day) decreased compared to Group 3 (0.8 mg/kg/day).

There were no marked sex-related differences in any of the measured toxicokinetic parameters, except on Day 14 where Tₘₐₓ occurred slightly later in Group 4 males as compared to Group 4 females. The sex ratios (male/female), with the exception of Group 4 Tₘₐₓ, ranged sporadically from 0.5 to 1.7 on Days 1 and 14.

Accumulation ratios (based on AUC_{0-Tlast}) ranged sporadically from 0.6 to 2.0 (Day 14/Day) suggesting that BPL-5MEO does not accumulate when administered once daily for 14 consecutive days (2 weeks) by intranasal instillation in beagle dogs at doses up to 1.5 mg/kg/day.

### Conclusion

Based on the parameters examined where all the changes noted were considered either non-adverse or related to exaggerated pharmacological effects, the reported NOAEL for BPL-5MEO, when dosed for 14 consecutive days by intranasal administration, followed by a 14-day recovery period was considered to be 1.5 mg/kg/day, corresponding to a Cₘₐₓ of 421 ng/mL, and AUC_{0-Tlast} (AUC_{INF_obs}) of 213 (220) h*ng/mL (combined for both sexes).

### Toxicokinetic Considerations

Based on preliminary data from another ongoing study in dogs, it has been observed that the site of blood sampling in dogs may impact the measured plasma exposure. Samples from the jugular vein may result in higher apparent exposure levels than samples from the cephalic vein, which might be due to the local transmucosal route of administration (also reported in the scientific literature (Illum, 2003; Sohlberg, 2013)). Therefore, dose escalation criteria for the Phase 1 Single Ascending Dose study are based on assessment of clinical criteria, safety factors and exposure. A maximum dose of 14 mg has been designated. The Table below summarizes the clinical observations in the rat and dog toxicity studies performed with BPL-5MEO. These clinical signs are considered to be related to the pharmacological activity of BPL-5MEO and demonstrate a dose-related increase in severity of findings on both species, generally ranging from mild to moderate at 0.4 to 1.5 mg/kg in dogs and 1.5 to 5 mg/kg in rats.

### Summary of Clinical Observations in Applicant-Sponsored Animal Studies

Dog (HED)

| 0.4 mg/kg | 0.8 mg/kg | 1.5 mg/kg^{a} | 2.5 mg/kg | 3.0-5.0 mg/kg |
|---|---|---|---|---|
| (14 mg) | (26 mg) | (50 mg) | (83 mg) | (100-166 mg) |
| Salivation | Mydriasis | Mydriasis | Salivation | Mydriasis |
| Mydriasis | Salivation | Salivation, | Pupil dilated | Salivation |
| Incoordination | Excessive licking | Excessive licking | Circling | Excessive licking |
| Vocalization | Incoordination | Dilated pupil | Muscle stiffness | Dilated pupil |
| Decreased activity | Vocalization | Vocalizing | Activity decreased | Vocalizing |
| Increased activity | Decreased activity | Tachypnea | Increased | Labored respiration |
| Increased | Increased activity | Increased | respiration | Gnawing cage |
| respiration | Increased | respiration | Diarrhea | Tongue outside |
| Gnawing cage wire | respiration | Tachycardia | Hunched | Hunched |
| Excessive licking | Gnawing cage wire | Muscle rigidity | Erect penis | Erect penis |
| Circling | Circling | Erect penis | Excessive grooming | Tremor |
| | Eye discharge | Twitches | Excessive fear | Shaking |
| | Shaking | Tense abdomen | Hypersensitive to | Lying |
| | Head shaking | Splay posture | stimuli | Decreased activity |
| | Slight tremor | Lying on cage floor | Aggressiveness | Uncoordinated |
| | (1.0 mg/kg) ^{b} | Uncoordinated | Tachycardia | Aggressiveness |
| | | Circling | Loss of righting | Circling |
| | | Head shaking | reflex | Not responsive to |
| | | Tremor | Hyperthermia | stimuli |
| | | Myoclonic jerk ^{b} | (single dose) | Hyperthermia |
| | | | Shaking | Convulsion |
| | | | Tremors | |

Rat (HED)

| 1.5 mg/kg | 3.0 mg/kg | 5.0 mg/kg^{a} | 10 mg/kg | 20-75 mg/kg |
|---|---|---|---|---|
| (14 mg) | (29 mg) | (48 mg) | (96 mg) | (194-726 mg) |
| Salivation | Salivation | Salivation | Salivation | Increased |
| Piloerection | Piloerection | Piloerection | Piloerection | respiration |
| Increased | Increased | Increased | Decreased activity | Shallow respiration |
| respiration | respiration | respiration | Increased or | Mydriasis |
| Dilated pupils | Gasping | Dilated pupils | shallow respiration | Salivation |
| Decreased activity | Dilated pupils | Slight hyperthermia | Gasping | Decreased activity |
| Decreased rearing | Decreased activity | (repeated dose) | Lying | Partially closed eyes |
| Lying | Decreased rearing | Uncoordinated | Decreased rearing | Lying on cage floor |
| Hypothermia | Lying | Shaking | Hypothermia | Sensitive to touch |
| (single dose) | Hypothermia | Decreased activity | (single dose) | Erect penis |
| | (single dose) | Lying | Twitching | Hyperthermia |
| | Uncoordinated | Sensitive to touch | Tremor | Uncoordinated |
| | Tremor | | | Shaking (or tremor) |

Abbreviations: HED = Human Equivalent Dose (for a 60 kg human)
^{a}NOAEL determined in the 14-day toxicology studies for both species.
^{b} Preliminary data, ongoing study (Slight tremor was observed at 1.0 mg/kg = 33 mg HED)
Note:
these signs were of short duration, and generally resolved within one to two hours in both species.

### Example 14: Genotoxicity

The genotoxicity potential of 5-MeO-DMT was evaluated in silico (computational analysis) for structural alerts and in vitro in GLP assays to assess mutagenic and clastogenic potential following the ICH S2 (R1) Guidance.

### In Silico

5-MeO-DMT, its primary active metabolite, bufotenine, and an identified drug substance impurity, MW234, were evaluated for quantitative structural activity relationships for potential mutagenicity and/or carcinogenicity using two computation analytical methods, Derek Nexus and the Leadscope Genetox Statistical Models. The evaluation from both analyses did not identify any structural alerts associated with 5-MeO-DMT or bufotenine, and a possible nor an identified drug substance impurity MW234.

### In Vitro Mutagenicity

The mutagenic potential of 5-MeO-DMT was evaluated in a GLP Bacterial Reverse Mutation Test (Ames test) for the ability to induce reverse mutations at selected loci of Salmonella typhimurium tester strains TA98, TA100, TA1535, and TA1537 and the Escherichia coli tester strain WP2uvrA. These strains were treated with 5-MeO-DMT at concentrations of 1.6, 5, 16, 50, 160, 500, 1600 and 5000 μg per plate along with the vehicle/negative and appropriate positive controls. The assay was performed in triplicate using the pre-incubation method in the absence and presence of an exogenous metabolic activation system, phenobarbital/5,6-benzoflavone-induced rat liver S9 microsomal enzyme mix (S9 mix)

A slight cytotoxicity was seen at the concentration of 1600 μg/plate in all S. typhimurium strains. Although higher levels of cytotoxicity were observed at 5000 μg/plate in the absence of S9 mix, it remained slight in the presence of S9 mix in these strains. No cytotoxicity was noted in the E. coli strain in either the absence or presence of S9 mix.

Overall, no increases (>2× of the vehicle/negative values) in the number of revertant colonies per plate was observed with 5-MeO-DMT in S. typhimurium tester strains TA1535, TA100, E. coli WP2uvrA in either the absence and presence of S9 or with TA1537 and TA98 in the presence of S9 mix. Three exceptions were a 2.1-fold increase at 1600 μg/plate without S9 seen in E. coli WP2uvrA, a 2.0-fold increase in S. typhimurium TA1537 at 50 μg/plate with S9, and 2.1-fold increase in S. typhimurium TA1535 at 1600 μg/plate with S9. However, these values were not considered biologically relevant as the values were within laboratory's historical vehicle/negative control range and were not dose-related.

Two of the 5-MeO-DMT-treated S. typhimurium strains, TA1537 and TA98, in the absence of S9 mix, showed a number of revertant colony counts slightly higher than twice of the vehicle/negative values at 160 μg/plate and 500 g/plate with fold-increases at 2.3- and 2.7-fold in TA1537 and 2.2- and 2.4-fold in TA98. The increased colony counts observed in these strains were still within the laboratory's historical vehicle/negative control range and were not overall dose-related; therefore, they did not meet the criteria of positive results. However, as the increases were seen in TA98 and TA1537 in 2 adjacent dose levels and that 2 strains showed a similar trend of increases in revertant colony counts at the same concentration levels, the results were judged equivocal. Therefore, the bacterial reverse mutation test was repeated in the absence of 59 mix for these 2 strains in order to investigate these equivocal results. The repeat test used a narrower concentration range of 15, 30, 60, 120, 250, 500, 1000, and 2000 μg per plate. The results from repeated test showed no increases in the revertant colonies number per plate for both 5-MeO-DMT-treated strains in all concentration levels tested up to the maximal dose of 2000 μg/plate. Therefore, it was concluded that the small increases observed in the first test for S. typhimurium tester stains TA 1537 and TA98 were not biologically relevant.

In conclusion, the results of the bacterial reverse mutation assays indicated that 5-MeO-DMT did not induce any increase in revertant colony numbers with any of the bacteria strains tested either in the absence or presence of the rat liver S9 microsomal metabolic activation system. 5-MeO-DMT has no mutagenic potential in the bacterial reverse mutation test. The expected response of the positive and negative controls affirmed the sensitivity and validity of assay.

### In Vitro Clastogenicity

The clastogenic potential of 5-MeO-DMT was evaluated in a GLP in vitro micronucleus test using Chinese hamster ovary (CHO)-K1 cells using flow cytometry. Exponentially growing cells were treated in duplicate with the 5-MeO-DMT at 9 concentrations up to the recommended upper limit of 1 mM (corresponding to approximately 300 μg/mL): 1.25, 2.5, 5.0, 10, 20, 40, 80, 150 and 300 g/mL. The treatment with the vehicle/negative and positive controls was concurrently performed. There were 3 treatment regimens: a 4-hour-short exposure in either absence or presence of an exogenous metabolic activation system, phenobarbital/5,6 benzoflavone rat liver S9 microsomal enzyme mix (S9 mix), and a 26 hour-extended exposure, considered a confirmatory phase, in the absence of 59 mix.

No cytotoxicity or precipitation was observed in 5-MeO-DMT-treated cells up to the maximal dose level of 300 μg/mL throughout the treatment periods. In all treatment regimens, the results of the in vitro micronucleus test indicate that 5-MeO-DMT did not induce any increases in micronuclei or hypodiploid cells either in the absence or presence of the rat liver S9 microsomal metabolic activation system. In conclusion, 5-MeO-DMT showed no chromosome-damaging potential in the in vitro micronucleus test with CHO-K1 cells. The expected response of the positive and negative controls affirmed the sensitivity and validity of assay.

### Reproductive and Development Toxicity

Reproductive and developmental toxicity studies have not been conducted. In the 14-day pivotal GLP intranasal toxicity studies in rats and dogs, there was no evidence of an adverse effect on reproductive tissues with systemic exposure to BPL-5MEO.

### Example 15: Formulation

BPL-5MEO has been synthesised to Good Manufacturing Practice (GMP) standards and prefilled into the Aptar Unidose Intranasal Liquid Delivery System device. The device allows a single fixed dose of BPL-5MEO to be administered intranasally. The liquid is prefilled into and administered using a standard single unit dose nasal pump device. Excipients used in the formulation are water, 0.1% hydroxypropyl methylcellulose (HPMC) and sodium hydroxyl (NaOH). Two concentrations of the formulation will be used, 70 mg/mL (for dose levels below 7 mg), and 140 mg/mL (for dose levels above 7 mg).

In an embodiment, there is provided a composition comprising 5-MeO-DMT hydrochloride, wherein the composition comprises:
water;
0.1% hydroxypropyl methylcellulose (HPMC);
0.1% sodium hydroxyl (NaOH); and
70 mg/ml 5-MeO-DMT.

In an embodiment, there is provided a composition comprising 5-MeO-DMT benzoate, wherein the composition comprises:
water;
0.1% hydroxypropyl methylcellulose (HPMC);
0.1% sodium hydroxyl (NaOH); and
70 mg/ml 5-MeO-DMT.

In an embodiment, there is provided a composition comprising 5-MeO-DMT hydrochloride, wherein the composition comprises:
water;
0.1% hydroxypropyl methylcellulose (HPMC);
0.1% sodium hydroxyl (NaOH); and
140 mg/ml 5-MeO-DMT.

In an embodiment, there is provided a composition comprising 5-MeO-DMT benzoate, wherein the composition comprises:
water;
0.1% hydroxypropyl methylcellulose (HPMC);
0.1% sodium hydroxyl (NaOH); and
140 mg/ml 5-MeO-DMT.

In an embodiment, there is provided an intranasal composition comprising 5-MeO-DMT hydrochloride, wherein the composition comprises:
water;
0.1% hydroxypropyl methylcellulose (HPMC);
0.1% sodium hydroxyl (NaOH); and
70 mg/ml 5-MeO-DMT.

In an embodiment, there is provided an intranasal composition comprising 5-MeO-DMT benzoate, wherein the composition comprises:
water;
0.1% hydroxypropyl methylcellulose (HPMC);
0.1% sodium hydroxyl (NaOH); and
70 mg/ml 5-MeO-DMT.

In an embodiment, there is provided an intranasal composition comprising 5-MeO-DMT hydrochloride, wherein the composition comprises:
water;
0.1% hydroxypropyl methylcellulose (HPMC);
0.1% sodium hydroxyl (NaOH); and
140 mg/ml 5-MeO-DMT.
In an embodiment, there is provided an intranasal composition comprising 5-MeO-DMT benzoate, wherein the water;
0.1% hydroxypropyl methylcellulose (HPMC);
0.1% sodium hydroxyl (NaOH); and
140 mg/ml 5-MeO-DMT.
composition comprises:

In an embodiment, the composition comprises 25-400 mg/mL; 25-300 mg/mL; 25-200 mg/mL; 25-100 mg/mL; 25−50 mg/mL; 50-400 mg/mL; 50-300 mg/mL; 60-400 mg/mL; 60-300 mg/mL; 150-400 mg/mL; 150-300 mg/mL; 200−300 mg/mL; 200-400 mg/mL; 30-100 mg/mL; 300-400 mg/mL; 300-500 mg/mL; 45-75 mg/mL; 50-70 mg/mL; 55−65 mg/mL; or 50-60 mg/mL 5-MeO-DMT.

In an embodiment, there is provided an intranasal liquid delivery system comprising a composition of 5-MeO-DMT.

In an embodiment, there is provided a single unit dose capsule of a composition of 5-MeO-DMT.

In an embodiment, there is provided an intranasal composition comprising a dosage amount 50-150 mg/ml 5-MeO-DMT in a liquid medium, wherein the 5-MeO-DMT is formulated as the benzoate salt of 5-MeO-DMT (5-MeO-DMT benzoate).

In an embodiment, 5-MeO-DMT benzoate is present as a suspension or emulsion in the liquid medium.

In an embodiment, there is provided an intranasal liquid delivery system comprising:
70 to 140 mg/ml of 5-MeO-DMT benzoate as a suspension or emulsion in a liquid medium.

### Example 16: Administration

BPL-5MEO is administered to subjects by a trained member of the research team using a single unit dose pump spray. The unit contains only 1 spray, so should not be tested before use. While sitting down the subject is asked to blow their nose to clear the nasal passages. Once the tip of the device is placed into the nostril the clinic staff will press the plunger to release the dose.

In an embodiment, there is provided a method for the administration of 5-MeO-DMT comprising administering the 5-MeO-DMT as an intranasal spray to a human subject wherein the human subject has followed patient preparation parameters that include blowing their nose to clear their nasal passages immediately prior to administration.

In an embodiment, the human subject is seated.

In an embodiment, there is provided a method for the delivery of 5-MeO-DMT to the brain of a human subject comprising administering the 5-MeO-DMT as an intranasal spray to a human subject wherein the human subject has followed patient preparation parameters that include blowing their nose to clear their nasal passages immediately prior to administration.

### Example 17: X-Ray Powder Diffraction (XRPD) of 5-MeO-DMT Benzoate

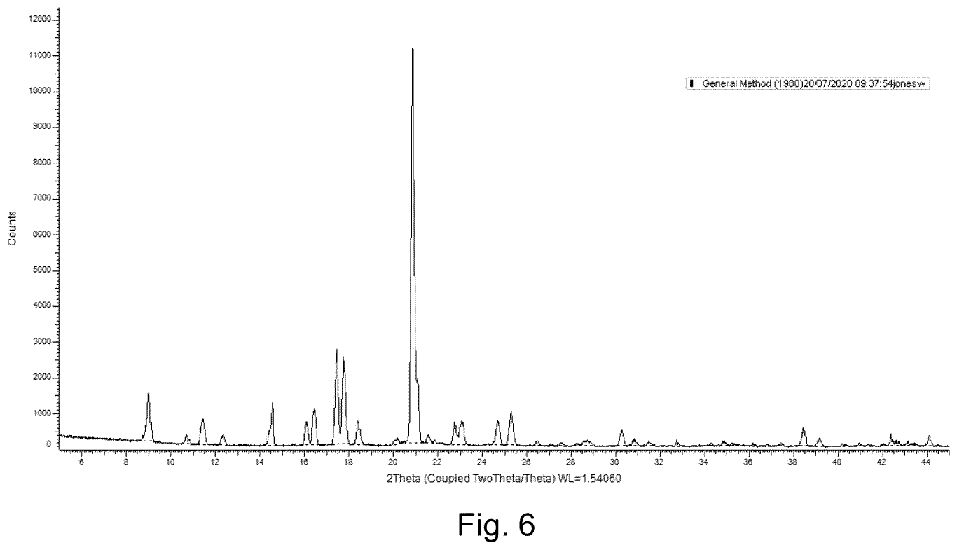

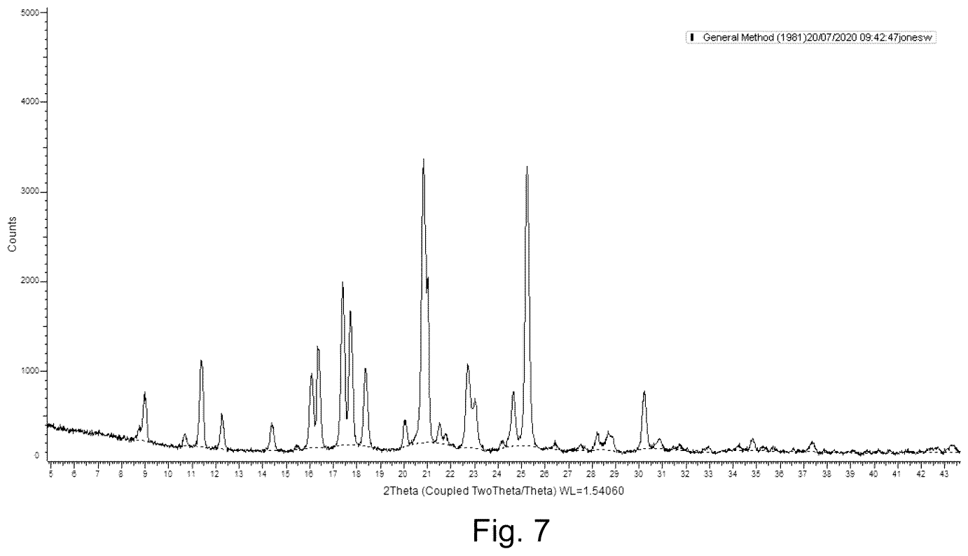

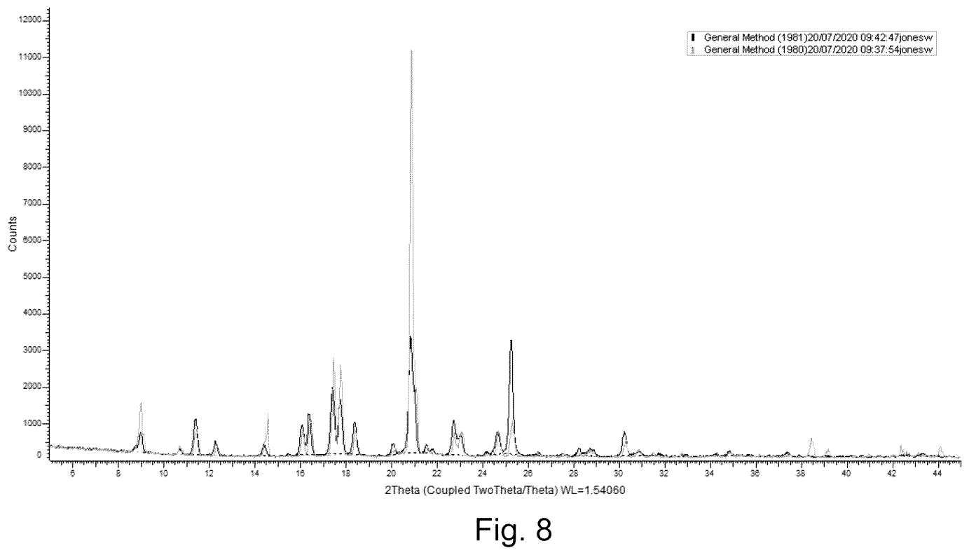

The XRPD pattern of 5-MeO-DMT benzoate salt, was acquired before and following particle size reduction with a mortar and pestle. This reduced the intensity of dominant diffractions and revealed that the XRPD pattern of the benzoate salt was prone to preferred orientation prior to particle size reduction, which is a function of the habit and particle size of the material. XRPD patterns of the benzoate salt prior to and following particle size reduction can be seen in FIGS. 6 and 7 respectively. The XRPD patterns of the benzoate salt prior to and following particle size reduction overlaid on one another can be seen in FIG. 8.

In an embodiment, there is provided crystalline 5-MeO-DMT benzoate, characterised by peaks in an XRPD diffractogram at 17.5, 17.7 and 21.0°2θ±0.1°2θ.

In an embodiment, there is provided crystalline 5-MeO-DMT benzoate, characterised by peaks in an XRPD diffractogram at 17.5, 17.7 and 21.0°2θ±0.2°2θ.

In an embodiment, there is provided crystalline 5-MeO-DMT benzoate, characterised by peaks in an XRPD diffractogram at 17.5, 17.7 and 21.0°2θ±0.3°2θ.

In an embodiment, there is provided crystalline 5-MeO-DMT benzoate, characterised by peaks in an XRPD diffractogram at 17.5, 17.7 and 21.0°2θ±0.1°2θ as measured by x-ray powder diffraction using an x-ray wavelength of 1.5406 Å.

In an embodiment, there is provided crystalline 5-MeO-DMT benzoate, characterised by peaks in an XRPD diffractogram at 17.5, 17.7 and 21.0°2θ±0.2°2θ as measured by x-ray powder diffraction using an x-ray wavelength of 1.5406 Å.

In an embodiment, there is provided crystalline 5-MeO-DMT benzoate, characterised by peaks in an XRPD diffractogram at 17.5, 17.7 and 21.0°2θ±0.3°2θ as measured by x-ray powder diffraction using an x-ray wavelength of 1.5406 Å.

In an embodiment, there is provided crystalline 5-MeO-DMT benzoate, characterised by peaks in an XRPD diffractogram at 17.5, 17.7, 21.0 and 25.3°2θ±0.1°2θ.

In an embodiment, there is provided crystalline 5-MeO-DMT benzoate, characterised by peaks in an XRPD diffractogram at 17.5, 17.7, 21.0 and 25.3°2θ±0.2°2θ.

In an embodiment, there is provided crystalline 5-MeO-DMT benzoate, characterised by peaks in an XRPD diffractogram at 17.5, 17.7, 21.0 and 25.3°2θ±0.3°2θ.

In an embodiment, there is provided crystalline 5-MeO-DMT benzoate, characterised by peaks in an XRPD diffractogram at 17.5, 17.7, 21.0 and 25.3°2θ±0.1°2θ as measured by x-ray powder diffraction using an x-ray wavelength of 1.5406 Å.

In an embodiment, there is provided crystalline 5-MeO-DMT benzoate, characterised by peaks in an XRPD diffractogram at 17.5, 17.7, 21.0 and 25.3°2θ±0.2°2θ as measured by x-ray powder diffraction using an x-ray wavelength of 1.5406 Å.

In an embodiment, there is provided crystalline 5-MeO-DMT benzoate, characterised by peaks in an XRPD diffractogram at 17.5, 17.7, 21.0 and 25.3°2θ±0.3°2θ as measured by x-ray powder diffraction using an x-ray wavelength of 1.5406 Å.

In an embodiment, there is provided crystalline 5-MeO-DMT benzoate, characterised by peaks in an XRPD diffractogram at 9.0, 11.5, 14.5, 16.5, 17.5, 17.7, 18.5, 21.0, 22.7, 24.7 and 25.3°2θ±0.1°2θ.

In an embodiment, there is provided crystalline 5-MeO-DMT benzoate, characterised by peaks in an XRPD diffractogram at 9.0, 11.5, 14.5, 16.5, 17.5, 17.7, 18.5, 21.0, 22.7, 24.7 and 25.3°2θ±0.2°2θ.

In an embodiment, there is provided crystalline 5-MeO-DMT benzoate, characterised by peaks in an XRPD diffractogram at 9.0, 11.5, 14.5, 16.5, 17.5, 17.7, 18.5, 21.0, 22.7, 24.7 and 25.3°2θ±0.3°2θ.

In an embodiment, there is provided crystalline 5-MeO-DMT benzoate, characterised by peaks in an XRPD diffractogram at 9.0, 11.5, 14.5, 16.5, 17.5, 17.7, 18.5, 21.0, 22.7, 24.7 and 25.3°2θ±0.1°2θ as measured by x-ray powder diffraction using an x-ray wavelength of 1.5406 Å.

In an embodiment, there is provided crystalline 5-MeO-DMT benzoate, characterised by peaks in an XRPD diffractogram at 9.0, 11.5, 14.5, 16.5, 17.5, 17.7, 18.5, 21.0, 22.7, 24.7 and 25.3°2θ±0.2°2θ as measured by x-ray powder diffraction using an x-ray wavelength of 1.5406 Å.

In an embodiment, there is provided crystalline 5-MeO-DMT benzoate, characterised by peaks in an XRPD diffractogram at 9.0, 11.5, 14.5, 16.5, 17.5, 17.7, 18.5, 21.0, 22.7, 24.7 and 25.3°2θ±0.3°2θ as measured by x-ray powder diffraction using an x-ray wavelength of 1.5406 Å.

In an embodiment, there is provided crystalline 5-MeO-DMT benzoate, characterised by peaks in an XRPD diffractogram at 9.0, 11.5, 14.5, 16.3, 16.5, 17.5, 17.7, 18.5, 21.0, 22.7, 24.7, 25.3 and 30.5°2θ±0.1°2θ.

In an embodiment, there is provided crystalline 5-MeO-DMT benzoate, characterised by peaks in an XRPD diffractogram at 9.0, 11.5, 14.5, 16.3, 16.5, 17.5, 17.7, 18.5, 21.0, 22.7, 24.7, 25.3 and 30.5°2θ±0.2°2θ.

In an embodiment, there is provided crystalline 5-MeO-DMT benzoate, characterised by peaks in an XRPD diffractogram at 9.0, 11.5, 14.5, 16.3, 16.5, 17.5, 17.7, 18.5, 21.0, 22.7, 24.7, 25.3 and 30.5°2θ±0.3°2θ.

In an embodiment, there is provided crystalline 5-MeO-DMT benzoate, characterised by peaks in an XRPD diffractogram at 9.0, 11.5, 14.5, 16.3, 16.5, 17.5, 17.7, 18.5, 21.0, 22.7, 24.7, 25.3 and 30.5°2θ±0.1°2θ as measured by x-ray powder diffraction using an x-ray wavelength of 1.5406 Å.

In an embodiment, there is provided crystalline 5-MeO-DMT benzoate, characterised by peaks in an XRPD diffractogram at 9.0, 11.5, 14.5, 16.3, 16.5, 17.5, 17.7, 18.5, 21.0, 22.7, 24.7, 25.3 and 30.5°2θ±0.2°2θ as measured by x-ray powder diffraction using an x-ray wavelength of 1.5406 Å.

In an embodiment, there is provided crystalline 5-MeO-DMT benzoate, characterised by peaks in an XRPD diffractogram at 9.0, 11.5, 14.5, 16.3, 16.5, 17.5, 17.7, 18.5, 21.0, 22.7, 24.7, 25.3 and 30.5°2θ±0.3°2θ as measured by x-ray powder diffraction using an x-ray wavelength of 1.5406 Å.

In an embodiment, there is provided crystalline 5-MeO-DMT benzoate, characterised by peaks in an XRPD diffractogram as substantially illustrated in FIG. 6, 7 or 8.

In an embodiment, there is provided crystalline 5-MeO-DMT benzoate, characterised by peaks in an XRPD diffractogram as substantially illustrated in FIG. 6.

In an embodiment, there is provided crystalline 5-MeO-DMT benzoate, characterised by peaks in an XRPD diffractogram as substantially illustrated in FIG. 7.

In an embodiment, there is provided crystalline 5-MeO-DMT benzoate, characterised by peaks in an XRPD diffractogram as substantially illustrated in FIG. 8.

In an embodiment, there is provided crystalline 5-MeO-DMT benzoate, characterised by one or more of:
Peaks in an XRPD diffractogram as previously or subsequently described;
An endothermic event in a DSC thermograph as previously or subsequently described;
An onset of decomposition in a TGA thermograph as previously or subsequently described;
A DVS isotherm profile as previously or subsequently described; and
A crystalline structure as previously or subsequently described.

### Example 18: Thermal Analysis of 5-MeO-DMT Benzoate

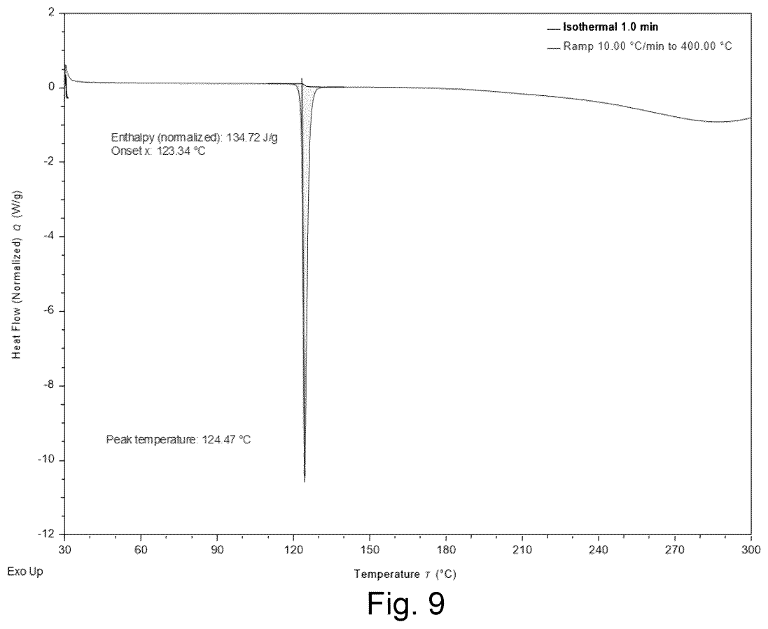

The differential scanning calorimetry (DSC) thermograph of 5-MeO-DMT benzoate salt, contained one endotherm with an onset of 123.34° C., peak of 124.47° C. and an enthalpy of 134.72 J/g. There were no other thermal events. The DSC thermograph, acquired at 10° C./min, can be seen in FIG. 9.

In an embodiment, there is provided crystalline 5-MeO-DMT benzoate, characterised by an endothermic event in a DSC thermograph having an onset temperature of between 12° and 130° C.

In an embodiment, there is provided crystalline 5-MeO-DMT benzoate, characterised by an endothermic event in a DSC thermograph having an onset temperature of between 12° and 130° C. as substantially illustrated in FIG. 9.

In an embodiment, there is provided crystalline 5-MeO-DMT benzoate, characterised by an endothermic event in a DSC thermograph having an onset temperature of between 12° and 130° C., between 121 and 129° C., between 122 and 128° C., between 123 and 127° C., between 124 and 126° C.

In an embodiment, there is provided crystalline 5-MeO-DMT benzoate, characterised by an endothermic event in a DSC thermograph having an onset temperature of between 12° and 130° C., between 121 and 129° C., between 122 and 128° C., between 123 and 127° C., between 124 and 126° C. as substantially illustrated in FIG. 9.

In an embodiment, there is provided crystalline 5-MeO-DMT benzoate, characterised by an endothermic event in a DSC thermograph having an onset temperature of 123° C.

In an embodiment, there is provided crystalline 5-MeO-DMT benzoate, characterised by an endothermic event in a DSC thermograph having an onset temperature of 123° C. a substantially illustrated in FIG. 9.

In an embodiment, there is provided crystalline 5-MeO-DMT benzoate, characterised by an endothermic event in a DSC thermograph having an onset temperature of 124° C.

In an embodiment, there is provided crystalline 5-MeO-DMT benzoate, characterised by an endothermic event in a DSC thermograph having an onset temperature of 124° C. as substantially illustrated in FIG. 9.

In an embodiment, there is provided crystalline 5-MeO-DMT benzoate, characterised by an endothermic event in a DSC thermograph having an onset temperature of between 12° and 130° C. and a peak of between 122 and 128° C.

In an embodiment, there is provided crystalline 5-MeO-DMT benzoate, characterised by an endothermic event in a DSC thermograph having an onset temperature of between 12° and 130° C. and a peak of between 122 and 128° C. as substantially illustrated in FIG. 9.

In an embodiment, there is provided crystalline 5-MeO-DMT benzoate, characterised by an endothermic event in a DSC thermograph having an onset temperature of between 12° and 130° C., between 121 and 129° C., between 122 and 128° C., between 123 and 127° C. and a peak of between 124 and 126° C.

In an embodiment, there is provided crystalline 5-MeO-DMT benzoate, characterised by an endothermic event in a DSC thermograph having an onset temperature of between 12° and 130° C., between 121 and 129° C., between 122 and 128° C., between 123 and 127° C. and a peak of between 124 and 126° C. as substantially illustrated in FIG. 9.

In an embodiment, there is provided crystalline 5-MeO-DMT benzoate, characterised by an endothermic event in a DSC thermograph having an onset temperature of between 12° and 130° C., between 121 and 129° C., between 122 and 128° C., between 123 and 127° C., and a peak of between 124 and 126° C. and an enthalpy of between −130 and −140 J/g.

In an embodiment, there is provided crystalline 5-MeO-DMT benzoate, characterised by an endothermic event in a DSC thermograph having an onset temperature of between 12° and 130° C., between 121 and 129° C., between 122 and 128° C., between 123 and 127° C., and a peak of between 124 and 126° C. and an enthalpy of between −130 and −140 J/g as substantially illustrated in FIG. 9.

In an embodiment, there is provided crystalline 5-MeO-DMT benzoate, characterised by an endothermic event in a DSC thermograph having an onset temperature of between 12° and 130° C., between 121 and 129° C., between 122 and 128° C., between 123 and 127° C., and a peak of between 124 and 126° C. and an enthalpy of between −130 and −135 J/g.

In an embodiment, there is provided crystalline 5-MeO-DMT benzoate, characterised by an endothermic event in a DSC thermograph having an onset temperature of between 12° and 130° C., between 121 and 129° C., between 122 and 128° C., between 123 and 127° C., and a peak of between 124 and 126° C. and an enthalpy of between −130 and −135 J/g as substantially illustrated in FIG. 9.

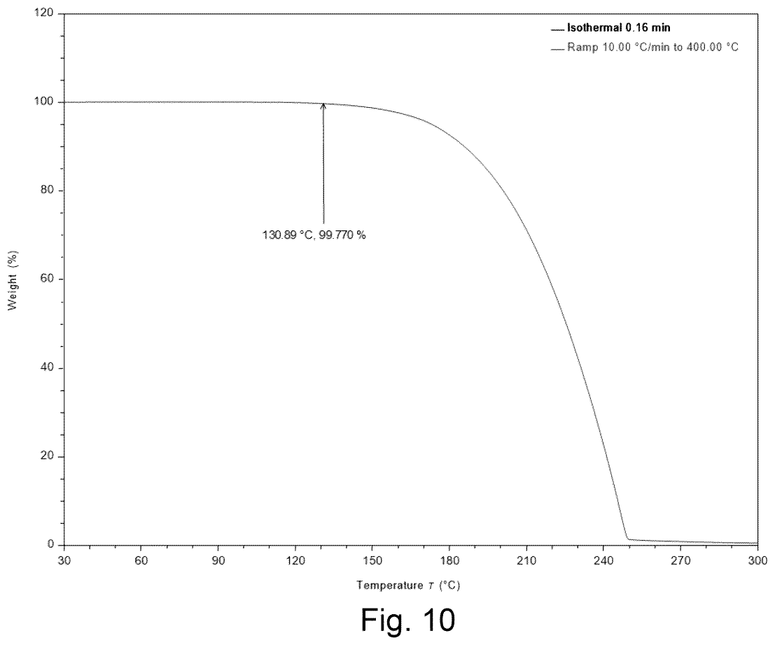

The thermogravimetric analysis (TGA) thermograph of 5-MeO-DMT benzoate salt, revealed that the onset of decomposition was ca 131° C., which is past the melt at ca 125° C. The TGA thermograph, acquired at 10° C./min, can be seen in FIG. 10.

In an embodiment, there is provided crystalline 5-MeO-DMT benzoate, characterised by an onset of decomposition in a TGA thermograph of between 128 and 135° C., between 129 and 134° C., between 13° and 133° C. or between 13° and 132° C.

In an embodiment, there is provided crystalline 5-MeO-DMT benzoate, characterised by an onset of decomposition in a TGA thermograph of between 128 and 135° C., between 129 and 134° C., between 13° and 133° C. or between 13° and 132° C. as substantially illustrated in FIG. 10.

In an embodiment, there is provided crystalline 5-MeO-DMT benzoate, characterised by an onset of decomposition in a TGA thermograph of 131° C.

In an embodiment, there is provided crystalline 5-MeO-DMT benzoate, characterised by an onset of decomposition in a TGA thermograph of 131° C. as substantially illustrated in FIG. 10.

In an embodiment, there is provided crystalline 5-MeO-DMT benzoate, characterised by one or more of:
an endothermic event in a DSC thermograph having an onset temperature of between 12° and 130° C., between 121 and 129° C., between 122 and 128° C., between 123 and 127° C., between 124 and 126° C.; and
an onset of decomposition in a TGA thermograph of between 128 and 135° C., between 129 and 134° C., between 13° and 133° C. or between 13° and 132° C.

In an embodiment, there is provided crystalline 5-MeO-DMT benzoate, characterised by one or more of:
an endothermic event in a DSC thermograph having an onset temperature of between 12° and 130° C., between 121 and 129° C., between 122 and 128° C., between 123 and 127° C., between 124 and 126° C. as substantially illustrated in FIG. 9; and
an onset of decomposition in a TGA thermograph of between 128 and 135° C., between 129 and 134° C., between 13° and 133° C. or between 13° and 132° C. as substantially illustrated in FIG. 10.

In an embodiment, there is provided crystalline 5-MeO-DMT benzoate, characterised by one or more of:
an endothermic event in a DSC thermograph having an onset temperature of 123° C.; and
an onset of decomposition in a TGA thermograph of 131° C.

In an embodiment, there is provided crystalline 5-MeO-DMT benzoate, characterised by one or more of:
an endothermic event in a DSC thermograph having an onset temperature of between 12° and 130° C., between 121 and 129° C., between 122 and 128° C., between 123 and 127° C., between 124 and 126° C. and a peak of between 124 and 126° C.; and
an onset of decomposition in a TGA thermograph of between 128 and 135° C., between 129 and 134° C., between 13° and 133° C. or between 13° and 132° C.

In an embodiment, there is provided crystalline 5-MeO-DMT benzoate, characterised by one or more of:
an endothermic event in a DSC thermograph having an onset temperature of between 12° and 130° C., between 121 and 129° C., between 122 and 128° C., between 123 and 127° C., between 124 and 126° C. and a peak of between 124 and 126° C. as substantially illustrated in FIG. 9; and
an onset of decomposition in a TGA thermograph of between 128 and 135° C., between 129 and 134° C., between 13° and 133° C. or between 13° and 132° C. as substantially illustrated in FIG. 10.

In an embodiment, there is provided crystalline 5-MeO-DMT benzoate, characterised by one or more of:
an endothermic event in a DSC thermograph having an onset temperature of 123° C., a peak of 124° C.; and
an onset of decomposition in a TGA thermograph of 131° C.

In an embodiment, there is provided crystalline 5-MeO-DMT benzoate, characterised by one or more of:
an endothermic event in a DSC thermograph having an onset temperature of 123° C., a peak of 124° C. as substantially illustrated in FIG. 9; and
an onset of decomposition in a TGA thermograph of 131° C. as substantially illustrated in FIG. 10.

In an embodiment, there is provided crystalline 5-MeO-DMT benzoate, characterised by one or more of:
an endothermic event in a DSC thermograph having an onset temperature of between 12° and 130° C., between 121 and 129° C., between 122 and 128° C., between 123 and 127° C., between 124 and 126° C., a peak of between 124 and 126° C. and an enthalpy of between −130 and −140 J/g; and
an onset of decomposition in a TGA thermograph of between 128 and 135° C., between 129 and 134° C., between 13° and 133° C. or between 13° and 132° C.

In an embodiment, there is provided crystalline 5-MeO-DMT benzoate, characterised by one or more of:
an endothermic event in a DSC thermograph having an onset temperature of between 12° and 130° C., between 121 and 129° C., between 122 and 128° C., between 123 and 127° C., between 124 and 126° C., a peak of between 124 and 126° C. and an enthalpy of between −130 and −140 J/g as substantially illustrated in FIG. 9; and
an onset of decomposition in a TGA thermograph of between 128 and 135° C., between 129 and 134° C., between 13° and 133° C. or between 13° and 132° C. as substantially illustrated in FIG. 10.

In an embodiment, there is provided crystalline 5-MeO-DMT benzoate, characterised by one or more of:
an endothermic event in a DSC thermograph having an onset temperature of 123° C., a peak of 124° C. and an enthalpy of −135° C.; and
an onset of decomposition in a TGA thermograph of 131° C.

In an embodiment, there is provided crystalline 5-MeO-DMT benzoate, characterised by one or more of:
an endothermic event in a DSC thermograph having an onset temperature of 123° C., a peak of 124° C. and an enthalpy of −135° C. as substantially illustrated in FIG. 9; and
an onset of decomposition in a TGA thermograph of 131° C. as substantially illustrated in FIG. 10.

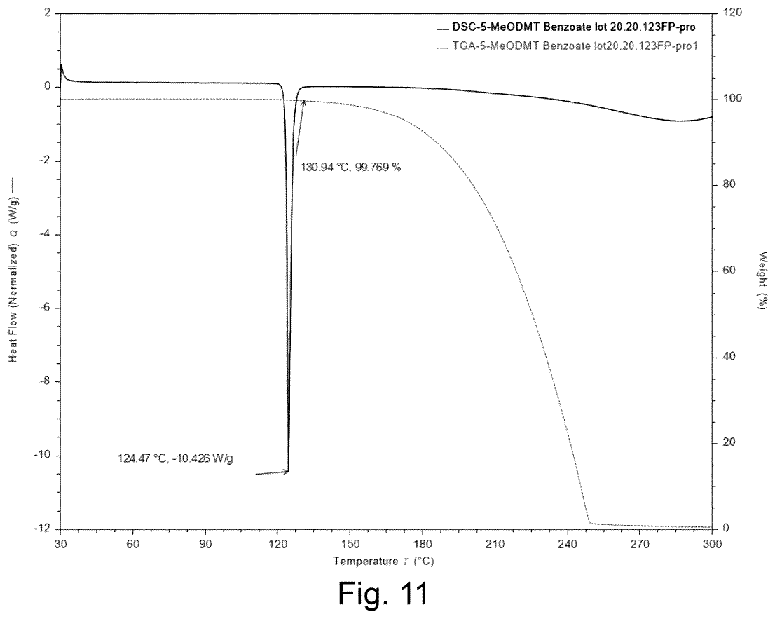

A combined TGA/DSC thermograph, acquired at 10° C./min, can be seen in FIG. 11.

### Example 19: Dynamic Vapour Sorption (DVS) of 5-MeO-DMT Benzoate

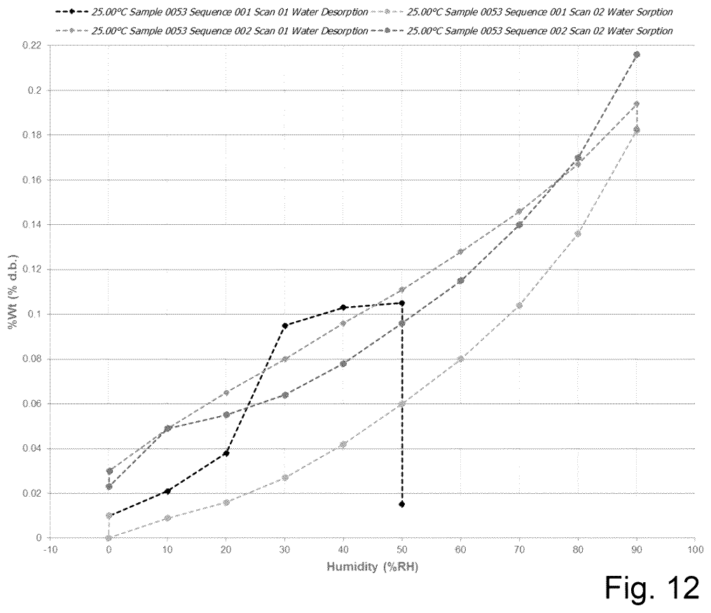

The DVS profile for 5-MeO-DMT benzoate salt, revealed reversible water uptake/loss over the humidity range and no hysteresis. The water uptake/loss from 0 to 90% was gradual and amounted to a maximum of ca 0.20% and was a consequence of wetting of the solid. There was no evidence of form/version modification as a consequence of exposure of 5-MeO-DMT benzoate salt to variable humidity. The DVS isotherm can be seen in FIG. 12.

The DVS isotherm of a 5-MeO-DMT Hydrochloride, lot 20/20/126-FP (FIG. 17) was found to undergo significant moisture uptake upon the first sorption cycle from 70% RH. Approximately 23%^{w}/w uptake is observed between 70−80% RH, whereas less than 0.3%^{w}/w moisture uptake from 0-70% RH was observed. A further 20%^{w}/w moisture uptake is observed up to and when held at 90% RH before commencement of the second desorption cycle. Subsequent sorption and desorption cycles follow a similar profile with some observed hysteresis between operations that do not match the original desorption step. These return to ca. 6-9%^{w}/w above the minimum mass recorded at 0% RH, which indicates significant retention of moisture. Upon completion of the DVS cycle, the input material was noted to have completed deliquesced.

A modified DVS isotherm of lot 20/45/006-FP (the same crystalline version) was undertaken to examine material behaviour from 60% RH and above. A 2 cycle DVS with desorption beginning from 40-0% RH with sorption from 0−60% RH in 10% RH intervals, followed by incremental 5% RH increases to 65, 70, 75, 80 and finally 85% RH. This is to obtain in-depth profiling of the material towards humidity at these elevated levels.

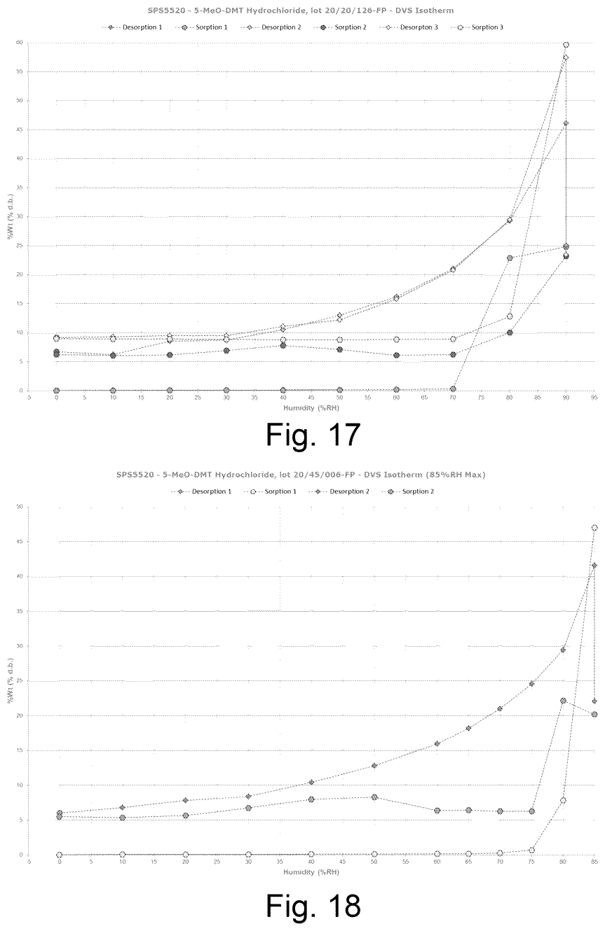

No significant moisture uptake/loss in first desorption-sorption profile between 0-70% RH was noted (FIG. 18) followed by a ca. 0.46% w/w increase from 70-75% RH. A further ca. 7% uptake is observed from 75-80% RH, then ca. 40% from 80-85% w/w. Complete deliquescence of the solids was observed upon isolation of the material post DVS analysis, which has likely occurred above 80% RH.

Temperature and humidity are important factors in the processing and storage of pharmaceuticals. DVS provides a versatile and sensitive technique for evaluating the stability of pharmaceutical formulations.

The DVS profiles show that the stability of the benzoate salt of 5-MeO-DMT is significantly higher than that of the hydrochloride salt and is therefore a more promising salt for development as a pharmaceutical composition.

There is thus provided in an embodiment of the invention an increased stability composition of 5-MeO-DMT wherein the composition comprises the benzoate salt. There is further provided a composition of 5-MeO-DMT having an increased stability wherein the composition comprises the benzoate salt.

In an embodiment there is thus provided a pharmaceutical composition of 5-MeO-DMT benzoate having an increased shelf-life compared to a pharmaceutical composition of 5-MeO-DMT hydrochloride.

In an embodiment, there pharmaceutical composition may be a nasal inhalation composition.

It is advantageous that the 5-MeO-DMT benzoate salt retains a low/consistent moisture content over its shelf-life preserving its ability to be consistently formulated, and preserving its ability to be inhaled in a free flowing powder form.

In an embodiment, there is provided crystalline 5-MeO-DMT benzoate, characterised by a DVS isotherm profile as substantially illustrated in FIG. 12.

In an embodiment, there is provided crystalline 5-MeO-DMT benzoate, characterised by one or more of:
an endothermic event in a DSC thermograph having an onset temperature of between 12° and 130° C., between 121 and 129° C., between 122 and 128° C., between 123 and 127° C., between 124 and 126° C., optionally a peak of between 124 and 126° C. and optionally an enthalpy of between −130 and −140 J/g as substantially illustrated in FIG. 9;
an onset of decomposition in a TGA thermograph of between 128 and 135° C., between 129 and 134° C., between 13° and 133° C. or between 13° and 132° C. as substantially illustrated in FIG. 10; and
a DVS isotherm profile as substantially illustrated in FIG. 12.

In an embodiment, there is provided crystalline 5-MeO-DMT benzoate, characterised by one or more of:
an endothermic event in a DSC thermograph having an onset temperature of 123° C., optionally a peak of 124° C. and optionally an enthalpy of −135° C. as substantially illustrated in FIG. 9;
an onset of decomposition in a TGA thermograph of 131° C. as substantially illustrated in FIG. 10; and
a DVS isotherm profile as substantially illustrated in FIG. 12.

The person skilled in the art will appreciate the defining characteristics of one of more of the previously or subsequently described embodiments may be interchanged with those of one or more other embodiments.

### Example 20: Microscopy, Optical of 5-MeO-DMT Benzoate

Optical microscopy examination was undertaken using an Olympus BX53M polarised light microscope and an Olympus SC50 digital video camera for image capture using imaging software Olympus Stream Basic, V2.4. The image scale bar was verified against an external graticule, 1.5/0.6/0.01 mm DIV, on a monthly basis.

A small amount of each sample was placed onto a glass slide and dispersed using mineral dispersion oil if required. The samples were viewed with appropriate magnification and various images recorded.

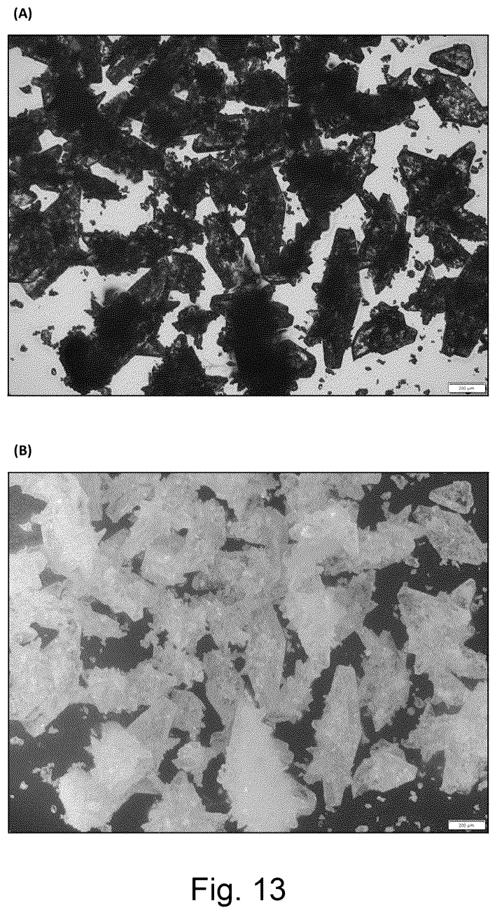

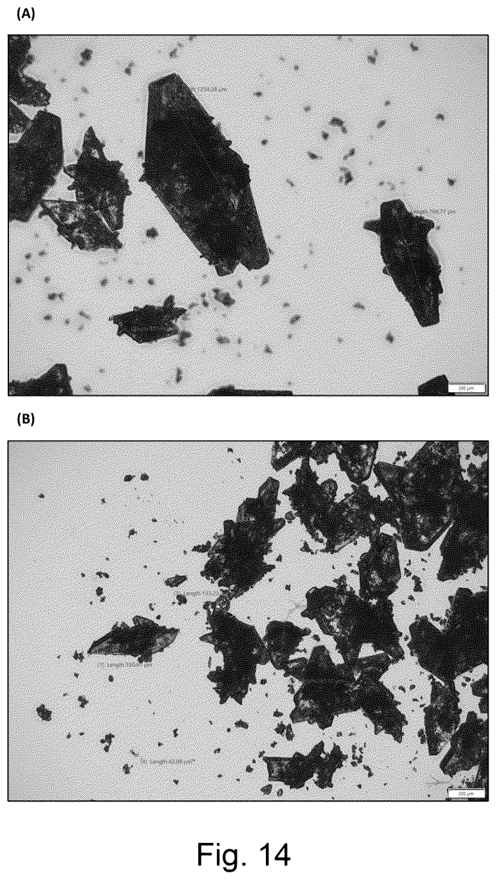

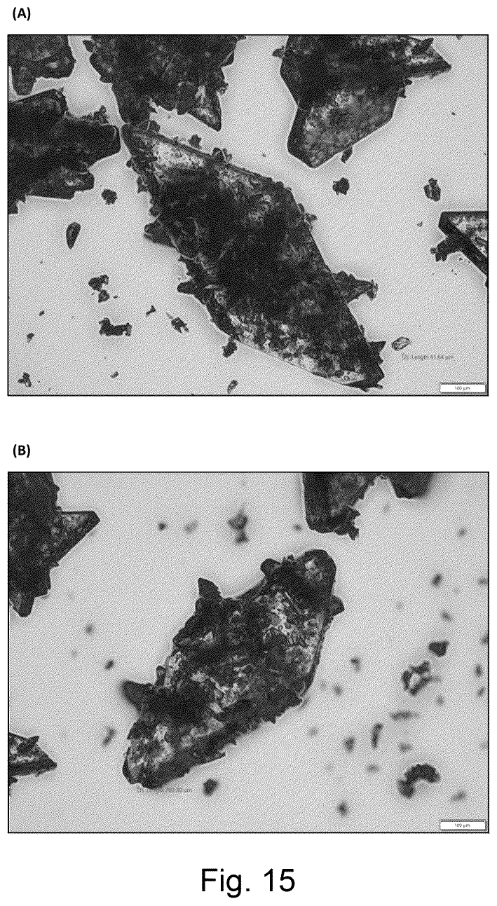

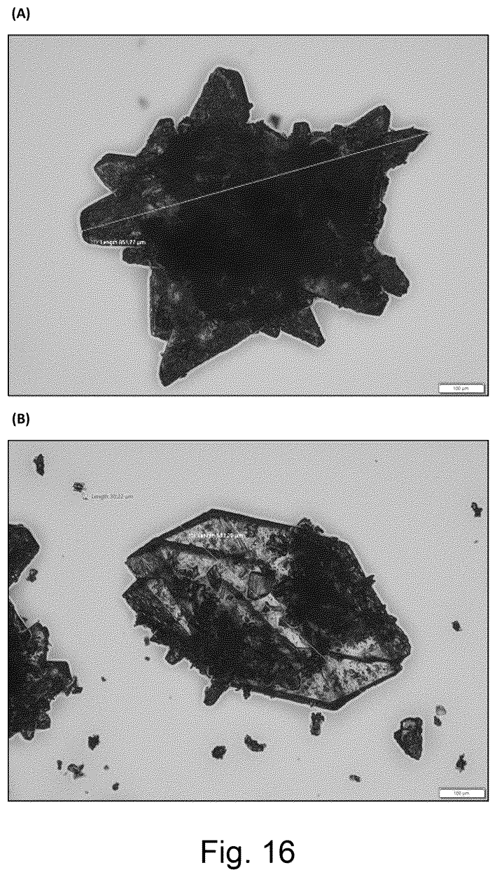

Optical micrographs of 5-MeO-DMT benzoate salt, were acquired. The material is composed of large rhombohedral/trigonal crystals, ranging from 400 to 1000 microns. There are also small crystals adhering to the large crystals. Some of the small crystals, from 10 microns, are a consequence of mechanical attrition, but others have formed by crystallisation. There are also large aggregates composed of various habits. FIGS. 13 to 16 show various optical micrographs of 5-MeO-DMT benzoate at various magnifications.

### Example 21: Further Characterisation of 5-MeO-DMT Benzoate

The propensity of 5-MeO-DMT benzoate to polymorphism was investigated and is considered low with solids isolated with two different XRPD patterns.

The equilibration of 5-MeO-DMT benzoate in solvents with thermal modulation induced a form or version change which are not considered to be solvates.

The anti-solvent mediated crystallisation investigation of 5-MeO-DMT benzoate did not afford any solids indicating form or version change.

The controlled cooling crystallisation investigation of 5-MeO-DMT benzoate did not afford any solids indicating form or version change.

The reverse anti-solvent mediated crystallisation investigation of 5-MeO-DMT benzoate did induce a form or version change.

Two versions of 5-MeO-DMT benzoate have been identified, the Pattern A form (see Example 17, hereafter this form is referred to as Pattern A) version and a second, Pattern B form, believed to be meta-stable.

The equilibration investigation of 5-MeO-DMT benzoate in a range of solvents with thermal modulation returned Pattern A by XRPD from most solvents. The equilibration solvents toluene, chlorobenzene, and anisole induced a form or version change in the 5-MeO-DMT benzoate and is defined as Pattern B by XRPD. Solvate formation can be excluded based upon TGA.

The anti-solvent mediated crystallisation investigation of 5-MeO-DMT benzoate afforded solids which were concordant Pattern A by XRPD indicating no form or version change.

The controlled cooling crystallisation investigation of 5-MeO-DMT benzoate afforded solids which were concordant Pattern A by XRPD indicating no form or version change.

The reverse anti-solvent mediated crystallisation investigation of 5-MeO-DMT benzoate returned Pattern A form from most mixtures. The methanol:toluene and IPA:toluene mixtures produced material which is considered to be Pattern B form with improved characteristics compared to the Pattern B form solids isolated via solvent equilibration.

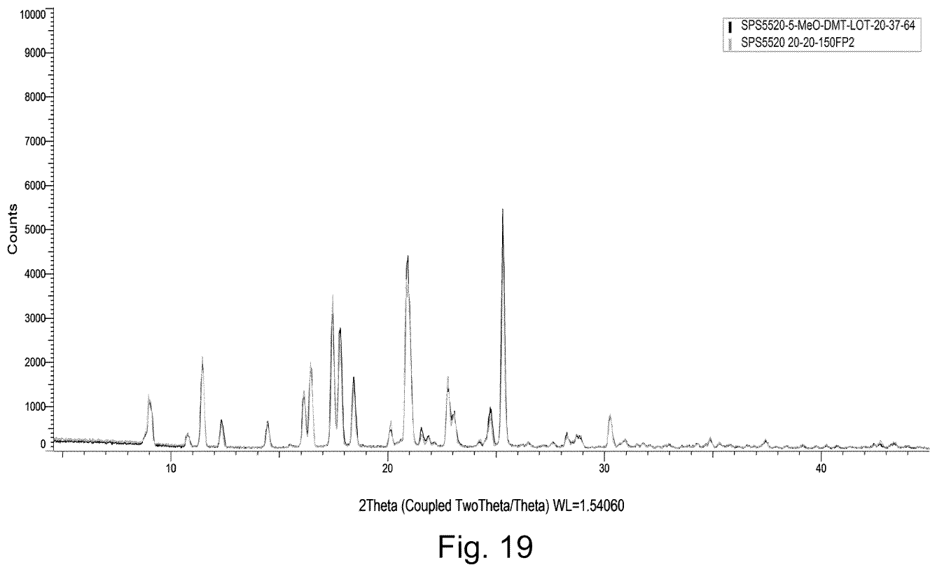

XRPD examination (FIG. 19) revealed a powder pattern of 5-MeO-DMT benzoate that was concordant with that found in previous XRPD examinations (see Example 17, Pattern A form).

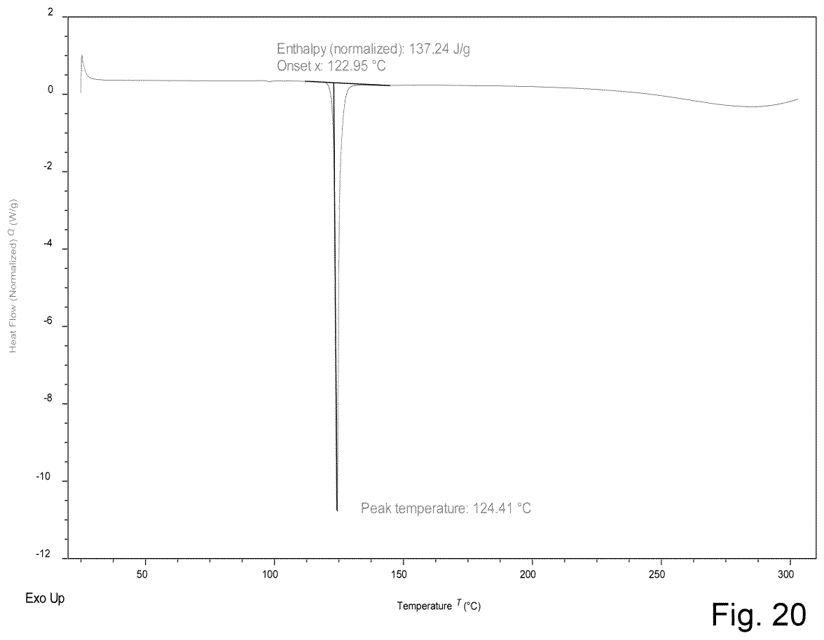

DSC examination (FIG. 20) revealed one sharp endotherm with an onset of 122.95° C. and a peak at 124.41° C. which was a match with Pattern A form (see Example 18 wherein the onset is 123.34° C. and the peak at 124.47° C.).

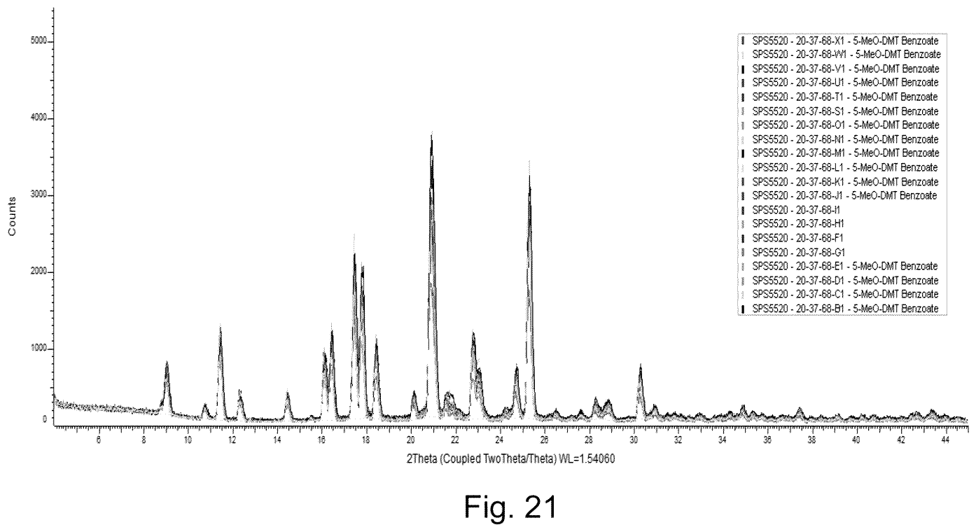

Additional XRPD examination of multiple lots of 5-MeO-DMT benzoate can be seen in FIG. 21, matching Pattern A.

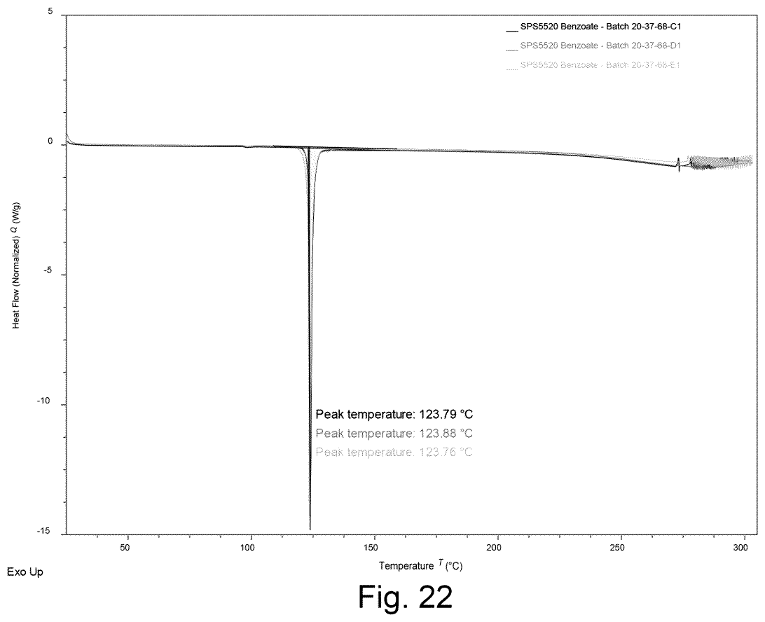

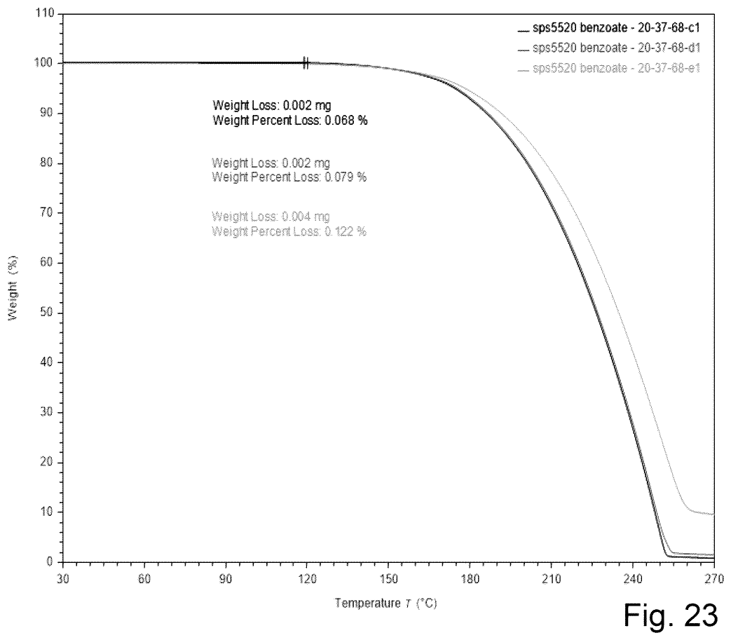

DSC examination of 5-MeO-DMT benzoate lots C1, D1 and E1 revealed a common endothermic event with a peak temperature of 123.76° C. to 123.88° C. (FIG. 22). TGA analysis of C1, D1 and E1 revealed a negligible weight loss before major decomposition (FIG. 23).

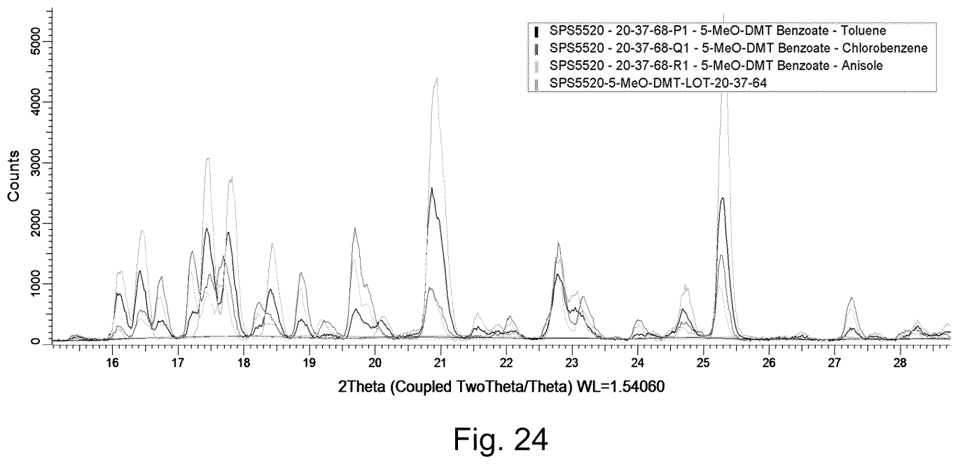

The XRPD patterns of P1 (Toluene), Q1 (Chlorobenzene), and R1 (Anisole) revealed a new diffraction pattern referred to as ‘Pattern B’. These samples contained 3 common diffractions between 18.5 and 20°2θ (FIG. 24).

A selection of samples of Pattern A form: C1 (IPA:Heptane [1:1]), D1 (3-Methyl-1-butanol:Heptane [1:1], and E1 (TBME) were thermally characterised.

DSC examination of samples P1, Q1, and R1 revealed a major common endothermic event with a peak temperature of 123.73° C. to 124.40° C. and a minor common endothermic-exothermic event between 113.01 and 115.27° C.

Sample R1 contained a unique endothermic event between the minor endothermic-exothermic event and the major endotherm with a peak temperature of 117.24° C.

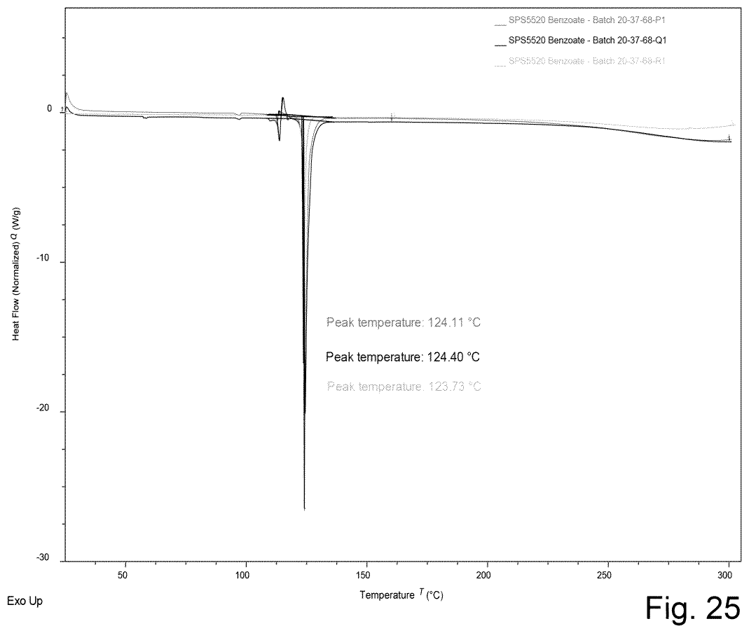

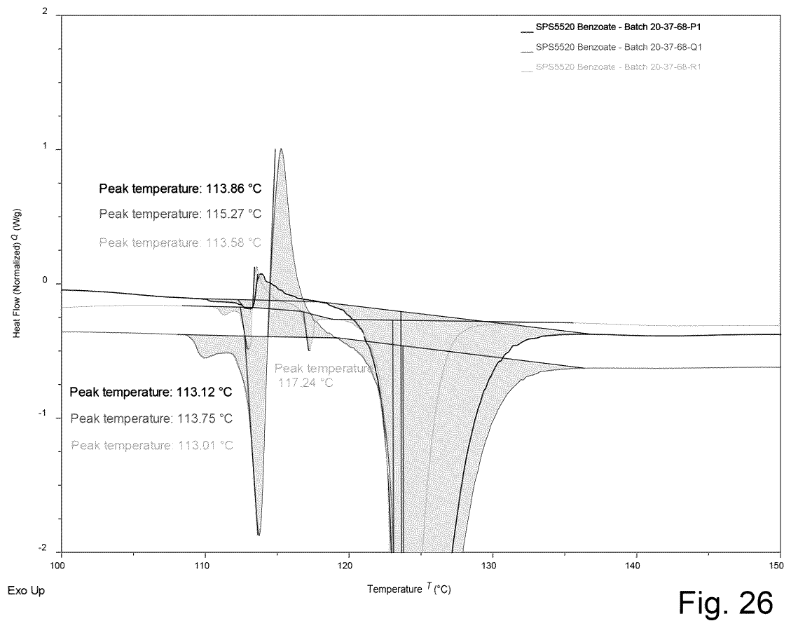

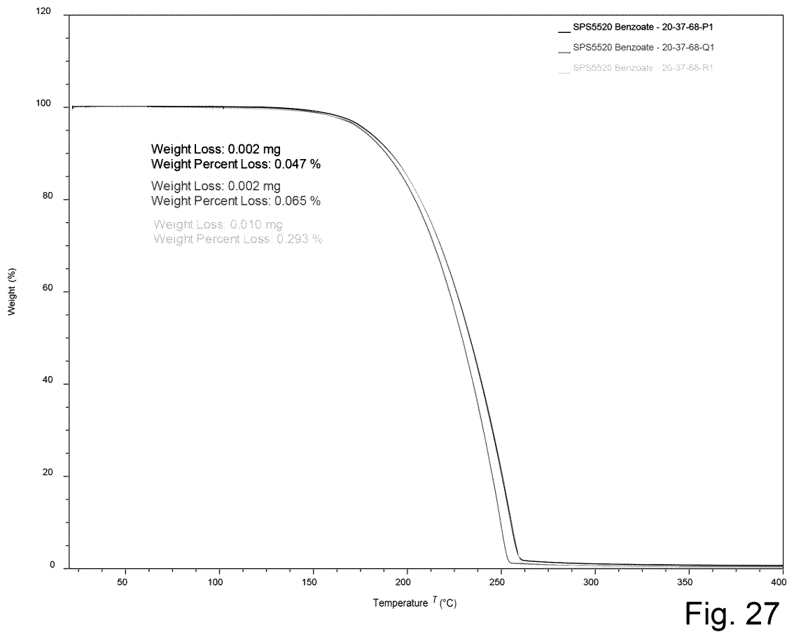

TGA examination revealed a negligible weight loss for samples P1 and Q1. For sample R1 there was a weight reduction of 0.293% weight before decomposition. DSC thermographs of P1, Q1 and R1 at 10° C.min⁻¹ can be seen in FIG. 25. DSC thermograph expansions of 5-MeO-DMT benzoate lots P1, Q1 and R1 at 10° C.min⁻¹ can be seen in FIG. 26. TGA thermographs of 5-MeO-DMT benzoate lots P1, Q1 and R1 at 10° C.min⁻¹ can be seen in FIG. 27.

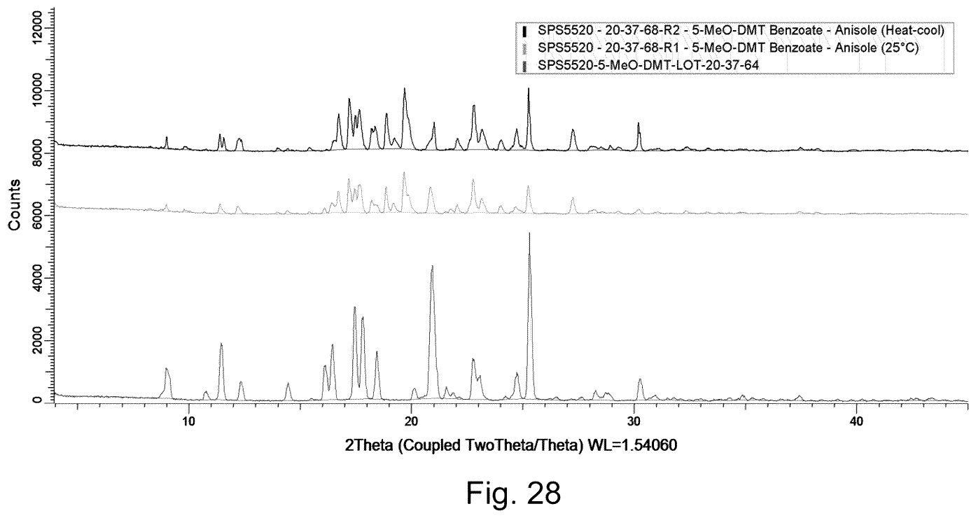

XRPD examination of samples P2, Q2, and R2 (thermally cycled suspensions) revealed P2 and Q2 had converted to Pattern A form. However, R2 remained as Pattern B form but with larger diffractions concordant with Pattern B. The XRPD diffractogram of lots R1 and R2 (thermally cycled suspensions) compared with a reference Pattern A XRPD diffractogram can be seen in FIG. 28.

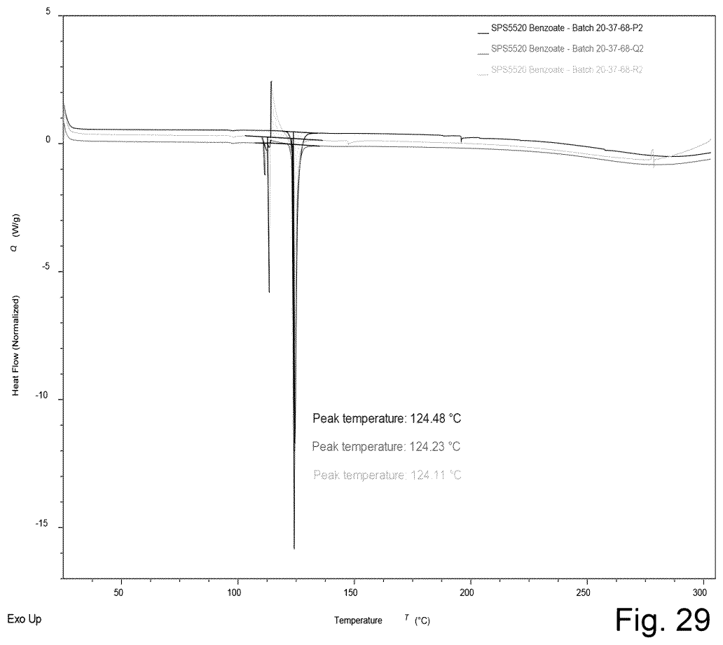

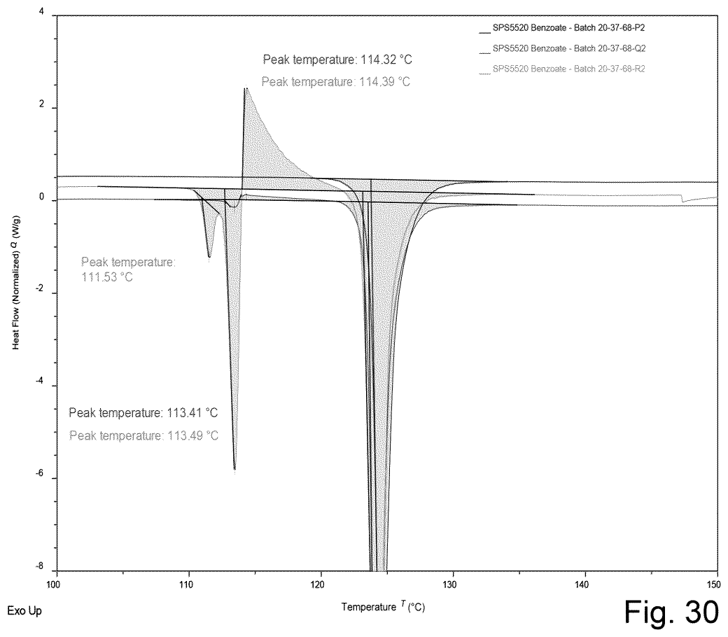

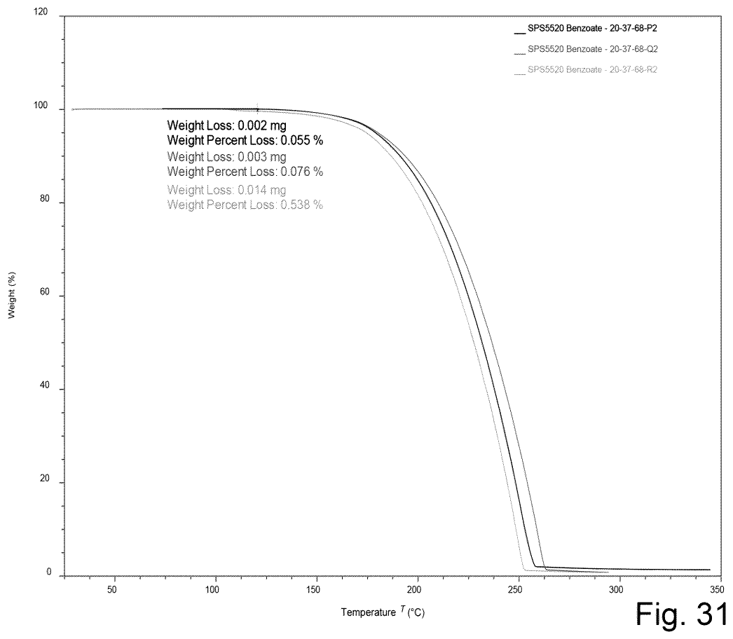

DSC examination of P2 revealed only the major endothermic event characteristic of the Pattern A form was present with a peak temperature of 124.48° C. (FIGS. 29-31).

DSC revealed the minor endo-exotherm was smaller for sample Q2 with peak temperatures of 113.41 and 114.32° C. but the major endotherm was unaffected with a peak temperature of 124.23° C. (FIGS. 29-31).

DSC examination of sample R2 revealed the endothermic event in the minor endo-exotherm had two peaks of 111.53 and 113.49° C. followed by the exotherm with a peak temperature of 114.39° C., the minor events were much larger compared to R1 and the second minor endothermic event was not present (FIGS. 29-31).

TGA examination revealed a negligible weight loss for samples P2 and Q2. For sample R2 there was a weight reduction of 0.583% before decomposition. The increase in weight loss corresponds to the increase in the magnitude of the minor events revealed by DSC (FIGS. 29-31).

The solvent mediated equilibration of 5-MeO-DMT benzoate with temperature modulation revealed the salt to be stable to version or form change except for the solvents toluene, chlorobenzene, and anisole. Solids isolated from these solvents had different XRPD patterns and thermal events indicating a version of form change of the salt. Solvate formation can be excluded based upon TGA.

In an embodiment, there is provided crystalline 5-MeO-DMT benzoate as described above.

### Anti-Solvent Addition Driven Crystallisation of 5-MeO-DMT Benzoate

Equilibration of Pattern A form in a variety of solvents and solvent mixtures with thermal modulation identified a range of potentially suitable solvents and anti-solvents. An investigation of the anti-solvent driven crystallisation of 5-MeO-DMT benzoate from solution was conducted.

5-MeO-DMT benzoate, 6×220 mg, was dissolved in six solvents at 50° C. (detailed in the Table below) and the stock solutions clarified through 0.45 μm syringe filters. Aliquots of each solution containing 50 mg of 5-MeO-DMT benzoate were charged to 4 crystallisation tubes.

The THF and Acetonitrile solutions of 5-MeO-DMT benzoate crystallised post-clarification. All crystallisation tubes were heated to 55° C. to afford solutions and cooled to 50° C. Samples were agitated via stirrer bead at 400 rpm for the duration of the experiment.

Various anti-solvents (detailed in the Table below), 2.5 vol., were charged to the solutions and the mixtures, then equilibrated at 50° C. for 30 minutes and the anti-solvent addition repeated.

The mixtures were cooled to 25° C. over ca. 1.5 hours and equilibrated for 17 hours.

Suspensions were isolated via isolutes and vacuum dried for 1 minute to remove excess solvent. The isolutes were transferred to a vacuum oven at 50° C. for 24 hours.

The remaining solutions were heated to 50° C. and anti-solvent, 5 vol. charged. The mixtures were equilibrated for 30 minutes and then repeated. Additional anti-solvent, 10 vol., was charged, equilibrated for 30 minutes, cooled to 25° C. over 1.5 hours and equilibrated for 30 minutes.

Suspensions were isolated via isolutes and vacuum dried to remove excess solvent and then dried in a vacuum oven at 50° C. for 24 hours.

The remaining solutions were reduced to ca. 0.25 mL volume under N₂ flow at 25° C. Anti-solvent, 20 vol., was charged and the mixtures equilibrated for 30 minutes.

In an embodiment, there is provided crystalline 5-MeO-DMT benzoate as described above.

Observations with anti-solvent addition and temperature equilibration

| | | | 2.5 vol.; | 5 vol.; | | 10 vol.; | 20 vol.; | 20 vol.; | Reduced; |
|---|---|---|---|---|---|---|---|---|---|
| | | | 50° C.; | 50° C.; | 25° C.; | 50° C.; | 50° C.; | 25° C.; | 20 vol.; |
| ID | Solvent | Anti-solvent | 30 mins | 30 mins | 18 hours | 30 mins | 30 mins | 30 mins | 30 mins |
| A1 | MeOH | Toluene | Solution | Solution | Solution | Solution | Solution | Solution | Suspension |
| A2 | 200.07 mg/mL | Heptane | Solution | Solution | Solution | Solution | Solution | Solution | Suspension |
| A3 | | TBME | Solution | Solution | Solution | Solution | Solution | Solution | Suspension |
| A4 | | DI Water | Solution | Solution | Solution | Solution | Solution | Solution | Solution |
| B1 | IPA | Toluene | Solution | Solution | Solution | Solution | Solution | Solution | Suspension |
| B2 | 50.08 mg/mL | Heptane | Solution | Solution | Suspension | N/a | N/a | N/a | N/a |
| B3 | | TBME | Solution | Solution | Solution | Solution | Solution | Solution | Suspension |
| B4 | | DI Water | Solution | Solution | Solution | Solution | Solution | Solution | Solution |
| C1 | THF | Toluene | Suspension | Suspension | Suspension | N/a | N/a | N/a | N/a |
| C2 | 200.35 mg/mL | Heptane | Suspension | Suspension | Suspension | N/a | N/a | N/a | N/a |
| C3 | | TBME | Suspension | Suspension | Suspension | N/a | N/a | N/a | N/a |
| C4 | | DI Water | Solution | Solution | Solution | Solution | Solution | Solution | Solution |
| D1 | 2-MeTHF | Toluene | Solution | Solution | Solution | Solution | Solution | Solution | Suspension |
| D2 | 50.02 mg/mL | Heptane | Solution | Solution | Solution | Solution | Suspension | Suspension | N/a |
| D3 | | TBME | Solution | Solution | Solution | Solution | Solution | Suspension | N/a |
| D4 | | DI Water | Solution | Solution | Solution | Solution | Solution | Solution | Solution |
| E1 | Acetone | Toluene | Solution | Solution | Suspension | N/a | N/a | N/a | N/a |
| E2 | 100.22 mg/mL | Heptane | Suspension | Suspension | Suspension | N/a | N/a | N/a | N/a |
| E3 | | TBME | Solution | Solution | Suspension | N/a | N/a | N/a | N/a |
| E4 | | DI Water | Solution | Solution | Solution | Solution | Solution | Solution | Solution |
| F1 | MeCN | Toluene | Solution | Solution | Suspension | N/a | N/a | N/a | N/a |
| F2 | 100.25 mg/mL | Heptane | Solution | Solution | Suspension | N/a | N/a | N/a | N/a |
| F3 | | TBME | Solution | Solution | Suspension | N/a | N/a | N/a | N/a |
| F4 | | DI Water | Solution | Solution | Solution | Solution | Solution | Solution | Solution |

Despite the initial suggestion that water was a potentially suitable anti-solvent, the utilisation of water as an anti-solvent failed to afford suspensions.

All THF, Acetone and MeON containing mixtures (excluding water) afforded suspensions by cooling to 25° C. with 10 volumes of anti-solvent. All other mixtures (excluding water) either required an increased anti-solvent charge or significant solution volume reduction and anti-solvent addition to afford suspensions.

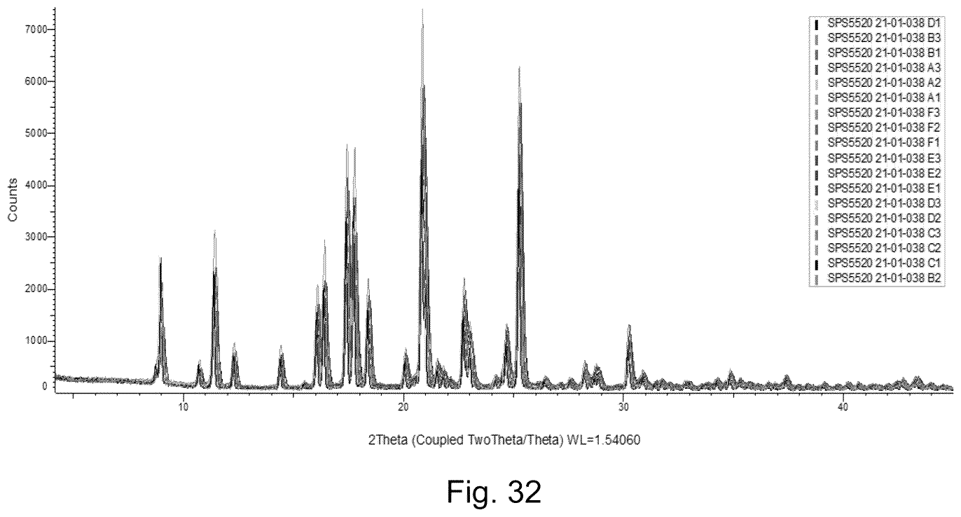

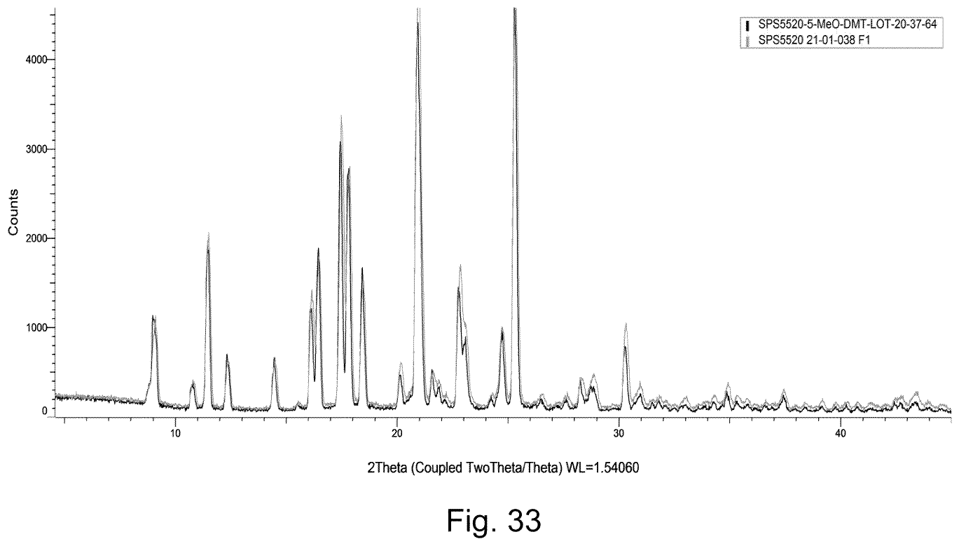

The XRPD examination of all isolated and dried solid samples were Pattern A as shown in FIGS. 32 and 33. The XRPD characterisation of the 5-MeO-DMT benzoate solids isolated from anti-solvent mediated crystallisation are concordant with Pattern A. This implies that there is no form/version modification of 5-MeO-DMT benzoate under the conditions investigated.

### Controlled Cooling Crystallisation Investigation of 5-MeO-DMT Benzoate

Observations from both the initial equilibration investigation and the first anti-solvent based investigations of 5-MeO-DMT benzoate identified potentially suitable solvents for the dissolution of 5-MeO-DMT benzoate at temperature to afford saturated solutions that could then be subject to a controlled gradual cooling operation.

5-MeO-DMT benzoate, 25±0.5 mg, was dissolved in the minimal volume of solvent at 50° C. (detailed in the Table below). The solutions were clarified through a 0.45 μm Teflon syringe filter into pre-heated crystallisation tubes and cooled from 50° C. to −10° C. over 60 hours (1° C. Hr−1 cooling rate) and held at −10° C. for 50 hours (no agitation).

Several crystallizations contained large off-white crystals on the base of the crystallisation tube (detailed in the Table below). The crystals were directly transferred from the crystallisation tube to the XRPD sample holder and were left open to the atmosphere for ca. 1 hour prior to analysis.

The remaining mixtures were agitated at 400 rpm at ambient temperature, open to the atmosphere to allow partial solvent evaporation, over 18 hours.

| | | | Observations with cooling and reduction | | |
|---|---|---|---|---|---|
| | | Solubility | −10° C.; | Volume reduced; | |
| ID | Solvent | (mg · mL⁻¹) | 50 hours | 25° C.; 18 hours | XRPD |
| A | MeOH | 250 | Solution | Solution | N/a |
| B | IPA | 42 | Crystallites | N/a | Pattern A |
| C | THF | 83 | Solution | Suspension | TBD |
| D | 2-MeTHF | 31.25 | Crystallites | N/a | Pattern A |
| E | Acetone | 62.5 | Crystallites | N/a | Pattern A |
| F | MeCN | 50 | Crystallites | N/a | Pattern A |
| G | MEK | 62.5 | Crystallites | N/a | Pattern A |
| H | Nitromethane | 125 | Crystallites | N/a | Pattern A |
| I | 3-methyl-1-butanol | 31.25 | Crystallites | N/a | Pattern A |
| J | Chlorobenzene | 12.5 | Solution | Suspension | — |
| K | iPrOAc | 12.5 | Solution | Suspension | — |
| L | MeOH:TBME (1:1) | 125 | Solution | Solid | — |

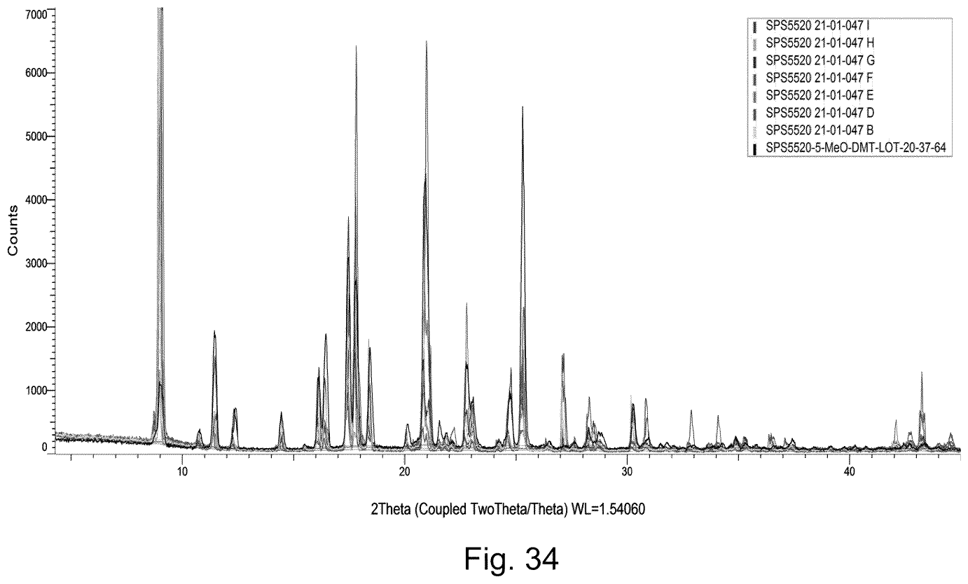

XRPD examination of the solid samples isolated following cooling of the solutions (observed as relatively large particles) revealed evidence of preferred orientation (FIG. 34).

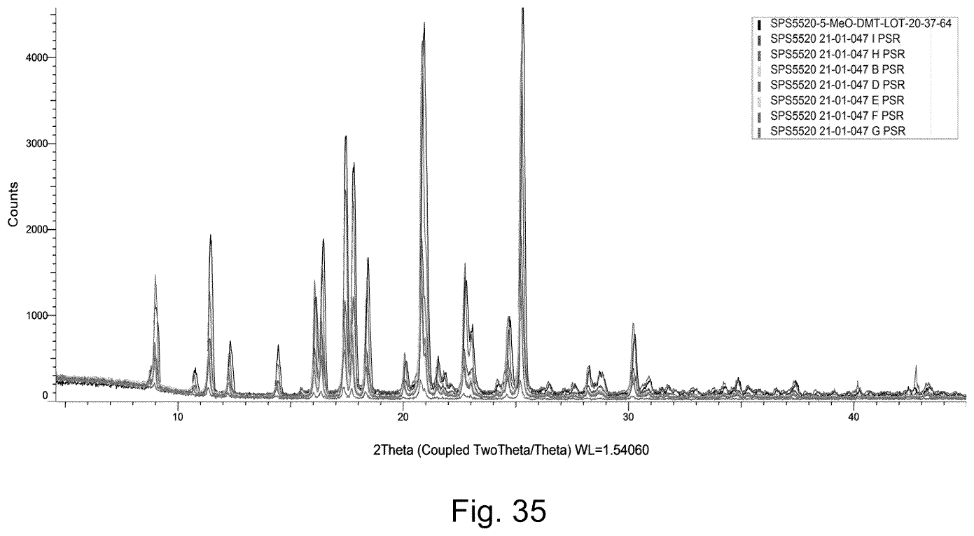

The particle size of the samples was reduced via particle size reduction with a mortar and pestle. Subsequent re-examination by XRPD revealed all solids to be Pattern A (FIG. 35).

The XRPD characterisation of the 5-MeO-DMT benzoate solids isolated to date from the single solvent mediated crystallisation of 5-MeO-DMT benzoate are concordant with Pattern A. This implies that there is no form or version modification 5-MeO-DMT benzoate under the conditions investigated.

In an embodiment, there is provided crystalline 5-MeO-DMT benzoate as described above.

### Reverse Addition Anti-Solvent Driven Crystallisation of 1-MeO-DMT Benzoate

The first anti-solvent-driven crystallisation of 5-MeO-DMT benzoate, revealed a selection of suitable solvent/anti-solvent mixtures. Utilising relatively gradual anti-solvent addition and cooling from elevated temperature afforded only solids classed as Pattern A by XRPD. The suitable solvent/anti-solvent mixtures were re-examined with reverse addition of hot stock solution to cold anti-solvent to potentially rapidly precipitate a new and/or meta-stable solid form version of 5-MeO-DMT benzoate.

5-MeO-DMT benzoate 1650.5 mg, was charged to vials A to F and dissolved in the minimal amount of solvent at 50HC as detailed in the Table below.

Anti-solvent, 1 ml, was charged to crystallisation tubes then cooled to −10° C. and agitated at 400 rpm.

Aliquots of the stock solutions of 5-MeO-DMT benzoate, ca. 50 mg, were charged directly to the anti-solvents.

All crystallisation tubes afforded suspensions within 5 minutes of addition of the 5-MeO-DMT benzoate solution.

Suspensions were isolated immediately in vacuo via solute then transferred to vacuum oven and dried at 50° C. for 18 hours.

TABLE
Summary of solvents, anti-solvents and observations

| | | | Observations upon charging | |
|---|---|---|---|---|
| | | Anti- | warm saturated solutions to | |
| ID | Solvent | solvent | cold anti-solvent | XRPD |
| A1 | MeOH | Toluene | suspension within 1 minute. | Pattern B |
| A2 | | Heptane | suspension within 1 minute. | N/a |
| A3 | | TBME | suspension within 1 minute. | Pattern A |
| B1 | IPA | Toluene | suspension within 5 minutes. | Pattern B |
| B2 | | Heptane | suspension within 1 minute. | Pattern A |
| B3 | | TBME | suspension within 5 minutes. | Pattern A |
| C1 | THF | Toluene | suspension within 1 minute. | Pattern A |
| C2 | | Heptane | Suspension upon addition | Pattern A |
| C3 | | TBME | suspension within 1 minute. | Pattern A |
| D1 | 2-MeTHF | Toluene | suspension within 1 minute. | Pattern A |
| D2 | | Heptane | Suspension upon addition | Pattern A |
| D3 | | TBME | suspension within 1 minute. | Pattern A |
| E1 | Acetone | Toluene | suspension within 1 minute. | Pattern A |
| E2 | | Heptane | suspension within 1 minute. | Pattern A |
| E3 | | TBME | suspension within 1 minute. | Pattern A |
| F1 | MeCN | Toluene | suspension within 1 minute. | Pattern A |
| F2 | | Heptane | Precipitate upon addition | Pattern A |
| F3 | | TBME | suspension within 1 minute. | Pattern A |

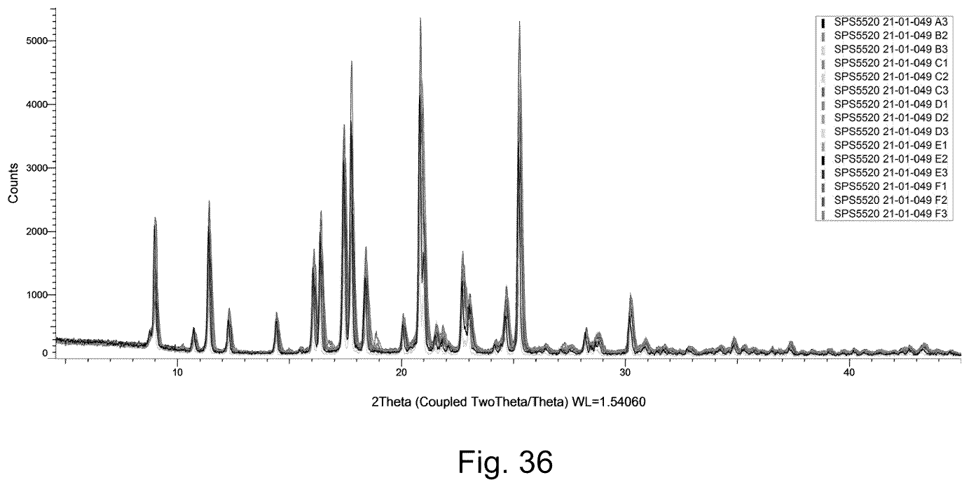

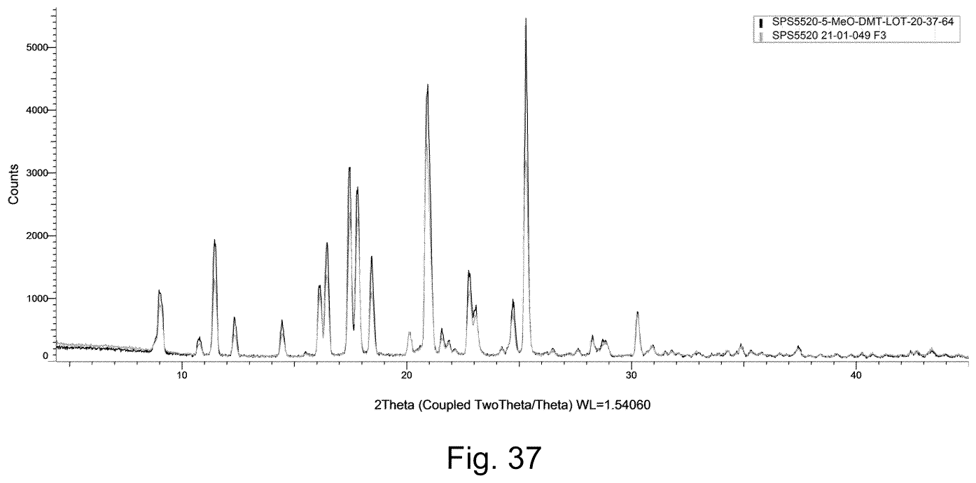

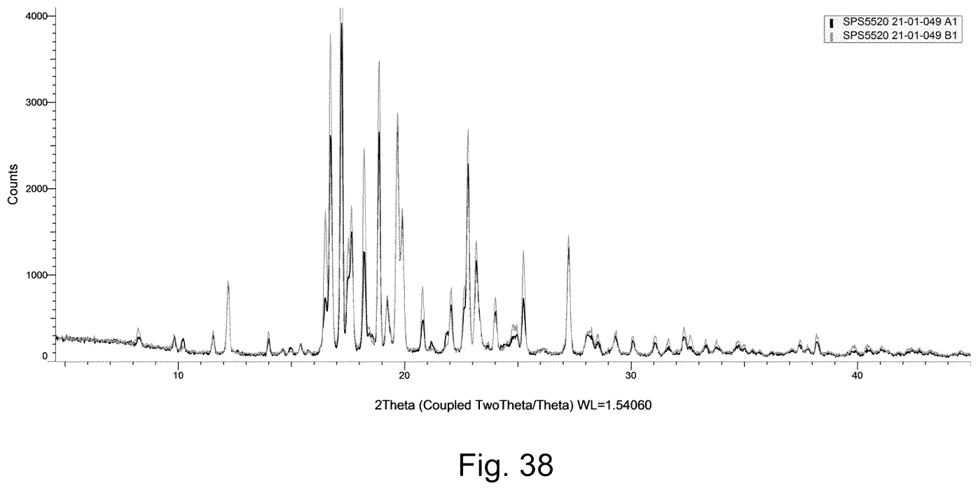

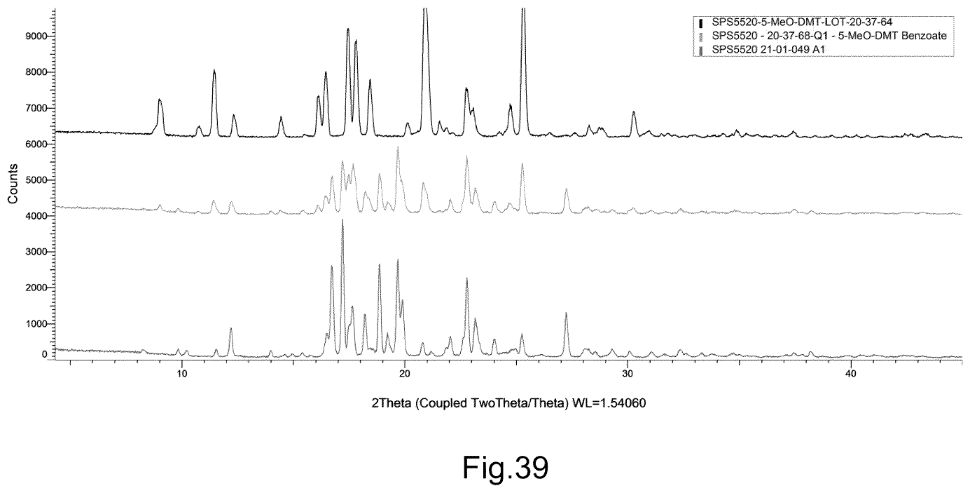

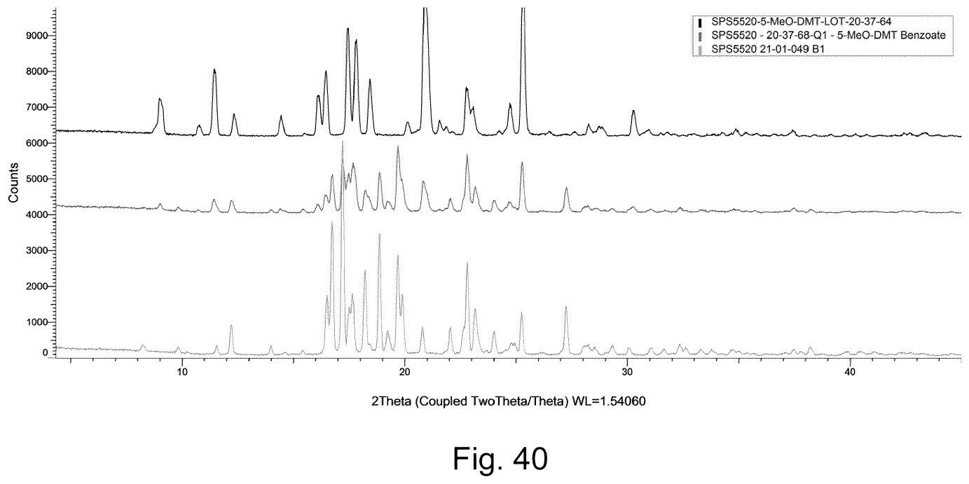

XRPD examination of most isolated solids (except for A1 and B1) were concordant with Pattern A (see FIGS. 36 and 37).
XRPD examination of solids A1 and B1 were concordant with one another but not Pattern A (FIGS. 38, 39)

Lots A1 and B1 shared diffractions with 5-MeO-DMT benzoate lot 01 (a pattern previously identified as Form B). However, on closer inspection, 01 was observed to share diffractions with Pattern A. As lot 01 shared diffractions with both lots A1 and B1 and Pattern A.

The diffraction patterns for lots A1 and B1 were considered to be characteristic of Pattern B.

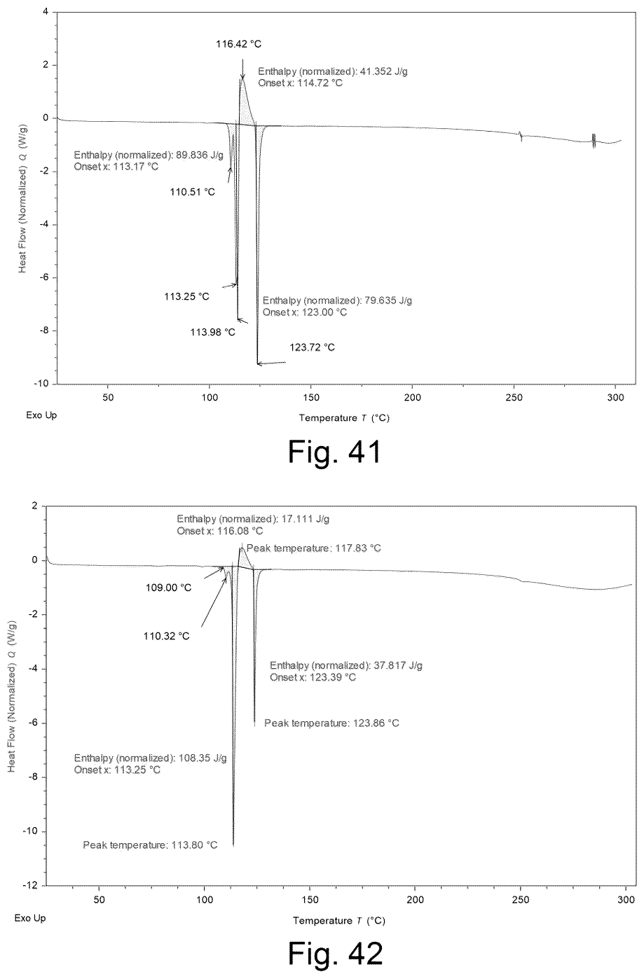

The DSC thermograph of sample A1 (FIG. 41) revealed an endothermic event with onset ca. 110° C. and major peak at 113.98° C., followed by an exotherm with onset 114.72° C. and peak at 116.42° C., followed by a second endotherm with an onset of 123.00° C. and peak at 123.72° C.

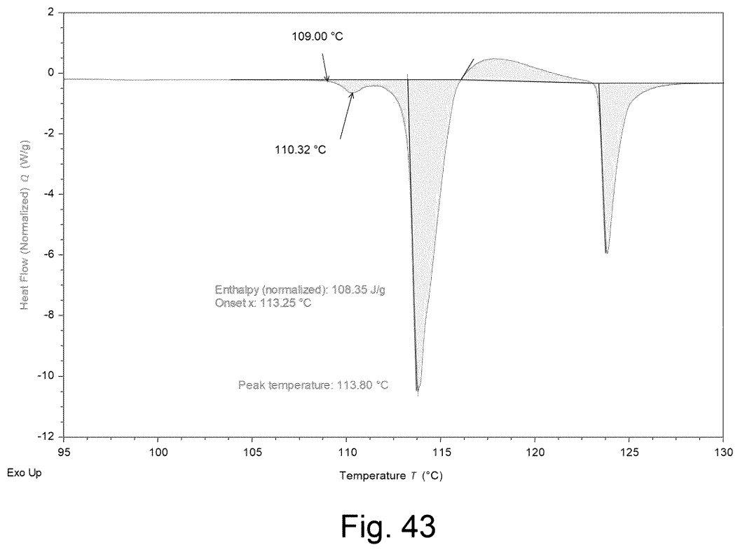

DSC examination of sample B1 (FIG. 42 and FIG. 43) revealed a similar DSC thermograph to A1 but the first endothermic event was larger, 108 J·g⁻¹ compared 90 J·g⁻¹ and only contained 2 peak temperatures of 109.00 and 110.32° C. instead of the 3 present in A1. The exothermic event that immediately followed was smaller, 17 J·g⁻¹ compared to 41 J·g⁻¹. The second main endotherm was also smaller for B1 at 38 J·g⁻¹ compared to 80 J·g⁻¹ for A1.

In an embodiment, there is provided crystalline 5-MeO-DMT benzoate as described above.

In an embodiment, there is provided crystalline 5-MeO-DMT salt, characterised by an endothermic or exothermic event in a DSC thermograph as substantially illustrated in any one of the Figures.

In an embodiment, there is provided a composition comprising 5-MeO-DMT benzoate Pattern A form.

In an embodiment, there is provided a composition comprising 5-MeO-DMT benzoate Pattern B form.

In an embodiment, there is provided a composition comprising a mixture of 5-MeO-DMT benzoate Pattern A form and Pattern B form.

### Example 22: Generation of the Amorphous 5-MeO-DMT Benzoate

### Rapid in Vacuo Concentration

5-MeO-DMT benzoate, 101.55 mg, was dissolved in THF, 4 mL and clarified into a 100 mL round bottom flask. The solution was concentrated in vacuo 40° C. at 200 rpm. The liquid evaporated from the flask, yielding a concentrated clear colourless liquid residue around the flask.

The residue was dissolved in acetone, 4 ml, concentrated in vacuo at 40° C. at 200 rpm. The liquid evaporated from the flask, yielding a concentrated clear colourless liquid residue around the flask. Small crystals were visible on the inside of the flask, these were isolated after 18 hours affording 21-01-051 Å.

### Quench of Melt

5-MeO-DMT benzoate was held at 125° C. for 5 minutes by TGA then cooled to ambient over 3 minutes affording 21−01-051 B. The sample was analysed immediately and after 20 hours held in a sealed container.

### Lyophilisation

5-MeO-DMT benzoate, 200 mg, was dissolved in deionised water, 10 ml, and clarified through a 0.45 μm nylon filter into a 500 mL round bottom flask, then frozen into a thin layer. The flask was transferred to a vacuum and equilibrated to ambient temperature affording a fluffy white solid, 21-01-051 C.

The solid transformed into gum over ca. 1 hour. The sample was analysed immediately and after 20 hours held in a sealed container.

### Lyophilisation for Amorphous Solid Equilibration

Lyophilisation was repeated as described above with 5-MeO-DMT benzoate, 800 mg, dissolved in 25 ml, affording 21−01-051 D. The solid was heated to 60° C. for 10 minutes then cooled yielding 21-01-051 E. The sample was analysed immediately.

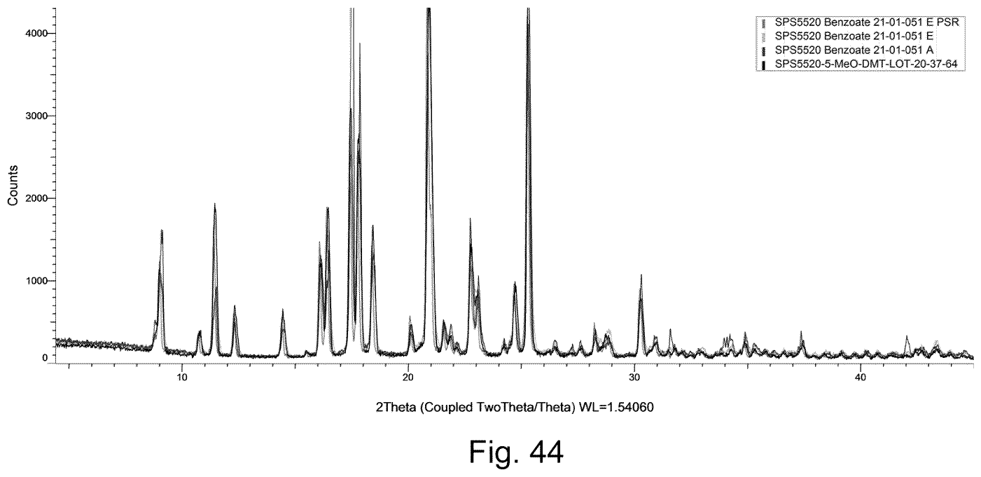

FIG. 44 shows XRPD comparison of 5-MeO-DMT benzoate lot 21-01-051 A, E, E Particle size reduced and Pattern A reference.

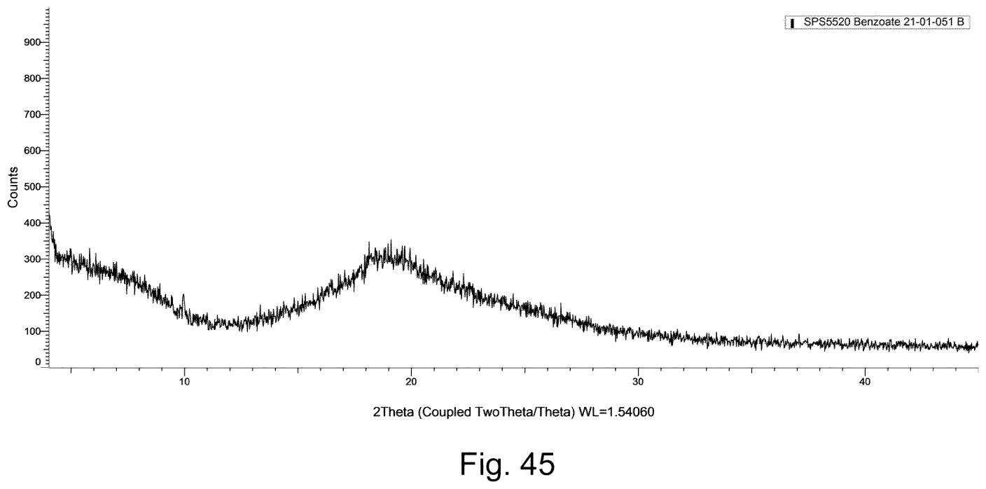

FIG. 45 shows XRPD of 5-MeO-DMT benzoate lot 21-01-051 B, obtained from quenching the melt.

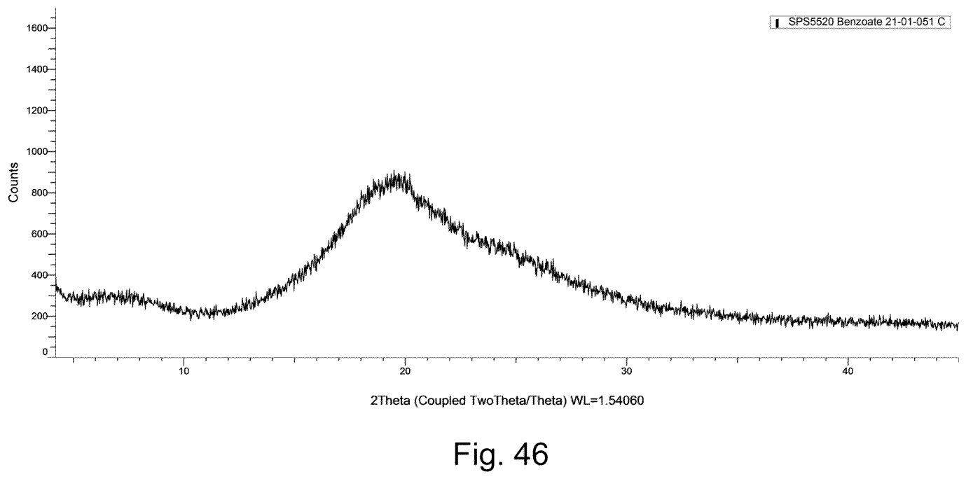

FIG. 46 shows XRPD of 5-MeO-DMT benzoate lot 21-01-051 C, obtained by lyophilisation.

The XRPD patterns of 5-MeO-DMT benzoate 21-01-051 B and C were concordant with Pattern A, indicating that the amorphous form converts to Pattern A form in a sealed container at ambient temperature and pressure.

The XRPD pattern of 5-MeO-DMT benzoate 21-01-051 A, the solid isolated by acetone concentration, was concordant with Pattern A form. Rapid in vacuo concentration did not produce the amorphous version.

The XRPD patterns revealed 5-MeO-DMT benzoate 21-01-051 B and C to have an amorphous ‘halo’, indicating quenching molten material and lyophilisation produced amorphous 5-MeO-DMT benzoate.

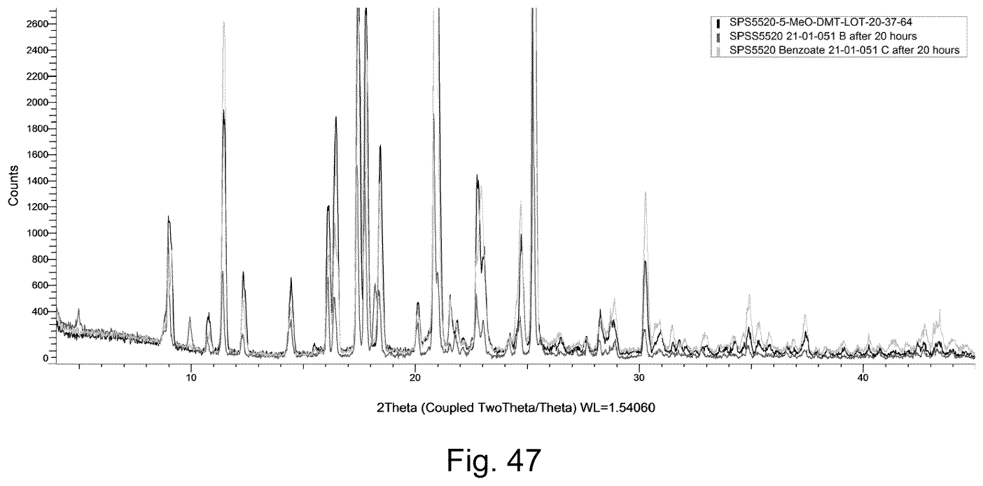

FIG. 47 shows XRPD comparison of 5-MeO-DMT benzoate lot 21-01-051 B after 20 hours, C after 20 hours, and Pattern A reference.

The XRPD pattern of 5-MeO-DMT benzoate 21-01-051 E were concordant with Pattern A, indicating that the amorphous form converts to Pattern A form at 60° C. for 10 minutes.

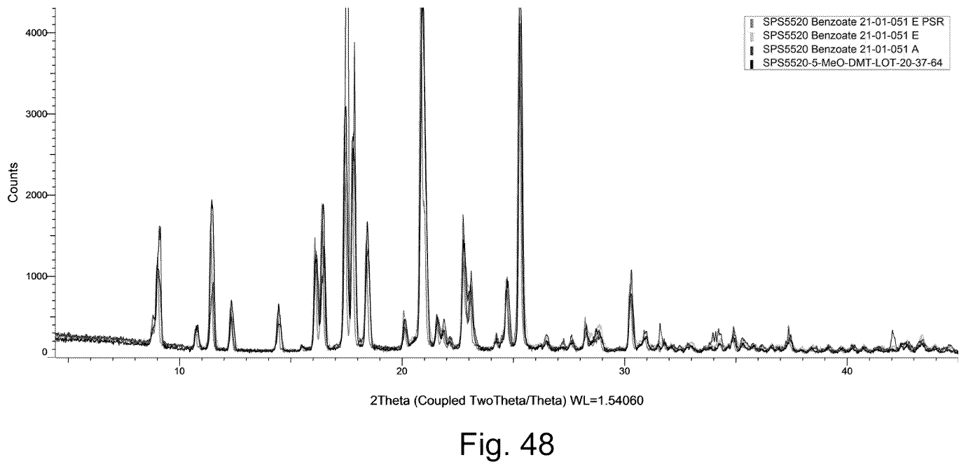

FIG. 48 shows XRPD comparison of 5-MeO-DMT benzoate lot 21-01-051 A, E, E particle size reduced, and Pattern A reference.

DSC examination revealed amorphous 5-MeO-DMT benzoate 21-01-051 C and D obtained by lyophilisation, contained an exothermic event with a peak temperature between 65.63 and 70.84° C., followed by a broad endothermic shoulder leading into a endothermic event with a peak temperature between 120.2° and 121.22° C. The major endothermic event is ca. 3° C. lower compared to Pattern A form material.

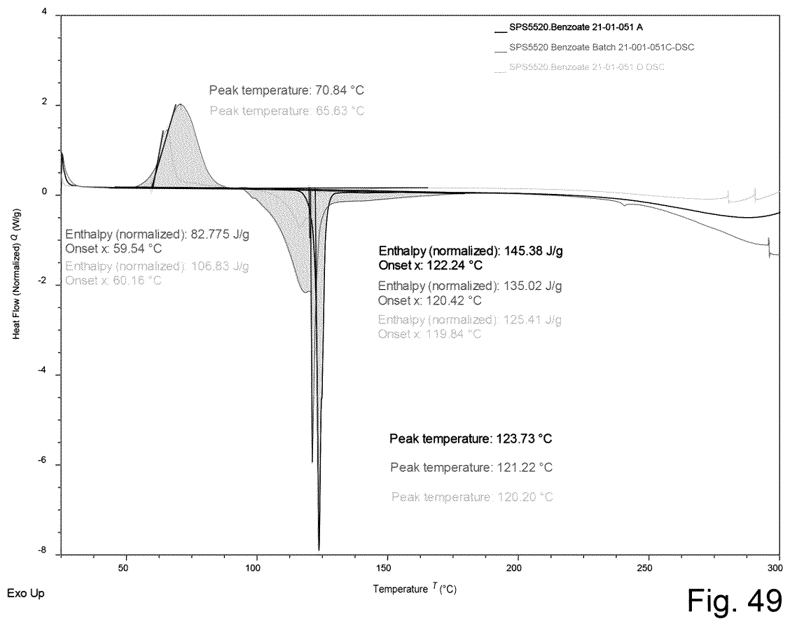

FIG. 49 shows DSC thermograph comparison of 5-MeO-DMT benzoate lot 21-01-051 A, C, and D at 10° C.min⁻¹, isolated from acetone concentrate, 051 A, and lyophilisation, 051 C and 051 D.

DSC examination revealed 5-MeO-DMT benzoate 21-01-051 C post 20 hours no longer contained an exothermic event and the endothermic event at ca. 123° C. was sharper and concordant with Pattern A form.

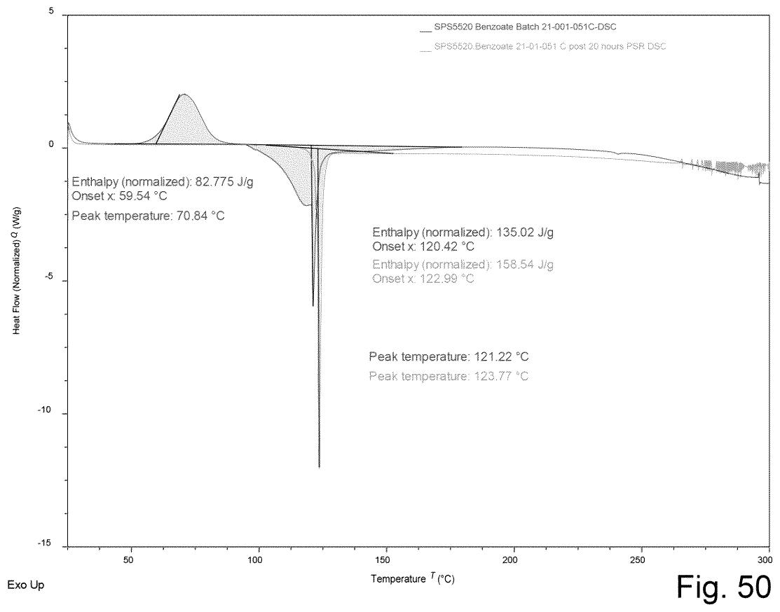

FIG. 50 shows DSC thermograph comparison of 5-MeO-DMT benzoate lot 21-01-051 C and C post 20 hours at 10° C.min⁻¹.

Amorphous 5-MeO-DMT benzoate can be generated by lyophilisation of an aqueous solution and the quenched melt.

The amorphous 5-MeO-DMT benzoate will convert to Pattern A form material on standing.

In one embodiment, there is provided an amorphous 5-MeO-DMT benzoate. In one embodiment, there is provided a composition comprising an amorphous 5-MeO-DMT benzoate.

In one embodiment, there is provided a composition comprising an amorphous 5-MeO-DMT benzoate salt produced as detailed above or below.

### Example 23: Further Characterisation of Amorphous 5-MeO-DMT Benzoate

The thermal examination of amorphous 5-MeO-DMT benzoate by DSC and hot stage microscopy revealed a crystallisation event and endothermic melt. The endothermic melt is not consistent with the DSC thermograph of Pattern A form.

The solvent mediated equilibration of amorphous 5-MeO-DMT benzoate with thermal modulation afforded Pattern A by XRPD and DSC from all solvents except anisole. New variations were generated.

Amorphous 5-MeO-DMT benzoate generated by lyophilisation, 21-01-051 D (21-01-051) was examined by hot-stage microscopy at a heating rate of 5° C.min⁻¹ for corroboration with the DSC thermograph of the amorphous solid.

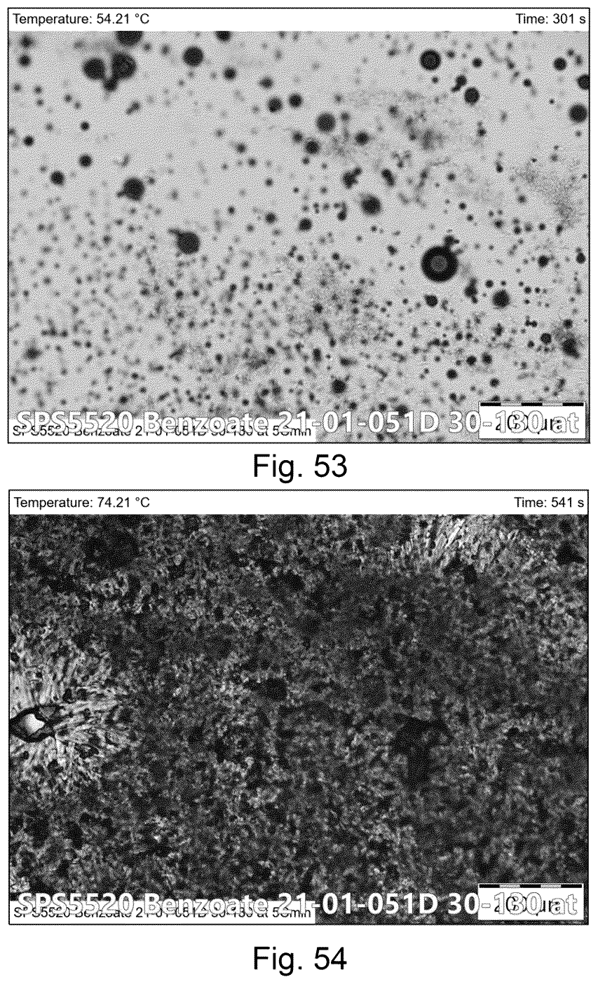

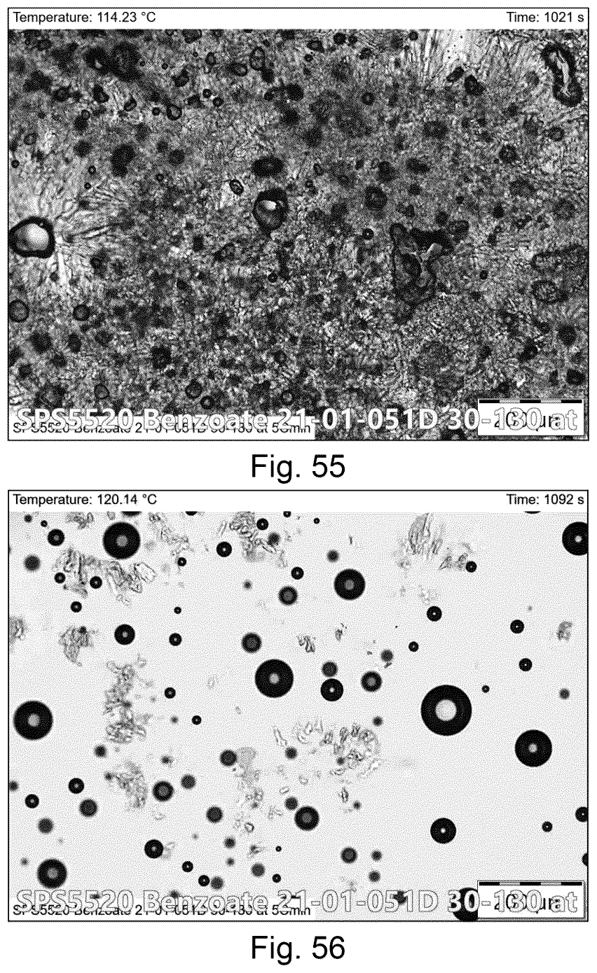

Initially, 5-MeO-DMT benzoate was a sticky translucent gum (FIG. 52) that upon heating to 54.21° C. reduced in viscosity and spread out into a thinner uniform layer (FIG. 53). At 54.21° C. the liquid began to crystallise (FIG. 53) which neared completion by 74.21° C. (FIG. 54). The newly formed crystals began to melt at 114.24° C. (FIG. 55) which neared completion by 120.14° C. (FIG. 56).

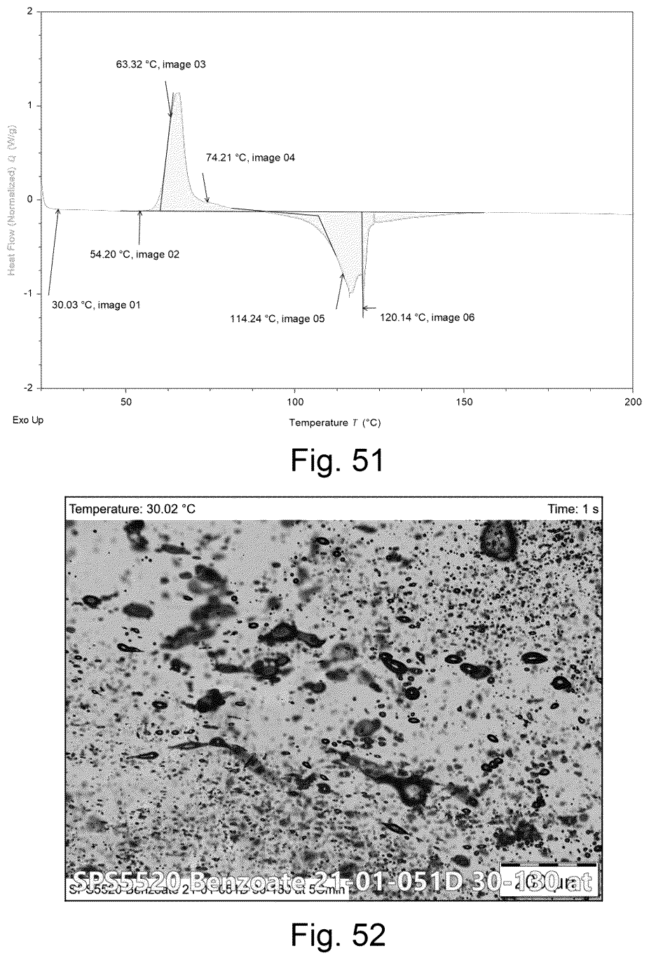

The hot stage microscopy examination corroborated with events in the DSC thermograph (FIG. 51); the crystallisation exotherm at ca. 65° C. and the melt endotherm at ca. 115° C.

FIG. 51 shows DSC thermograph of 5-MeO-DMT benzoate lot 21-01-051 D, large scale lyophilised material, with temperature stamps corresponding to hot-stage microscopy images.

FIG. 52 shows Micrograph image of 5-MeO-DMT benzoate lot 21-01-051 D at 30.02° C.

FIG. 53 shows Micrograph image of 5-MeO-DMT benzoate lot 21-01-051 D at 54.21° C.

FIG. 54 shows Micrograph image of 5-MeO-DMT benzoate lot 21-01-051 D at 74.21° C.

FIG. 55 shows Micrograph image of 5-MeO-DMT benzoate lot 21-01-051 D at 114.23° C.

FIG. 56 shows Micrograph image of 5-MeO-DMT benzoate lot 21-01-051 D at 120.14° C.

Solvent Mediated Equilibration of Amorphous 5-MeO-DMT Benzoate with Thermal Manipulation

The action of agitating the amorphous version of a solid in a series of solvents can lead to dissolution and crystallisation to more ordered and energetically stable solids. In this manner, alternate crystal forms of a solid can be potentially generated for comparison and evaluation.

Amorphous 5-MeO-DMT benzoate 21-01-51 D, 24×25±2 mg was transferred to crystallisation tubes and solvent, 0.125 mL charged as detailed in the Error! Reference source not found. The mixtures were agitated at 300 rpm at 25° C. for 30 minutes. Solvent, 0.125 mL, was charged to relevant mixtures and equilibrated for 18 hours.

Mixtures were heated to 55° C. for 8 hours then cooled to 25° C. over 1 hour then equilibrated for 18 hours at 300 rpm, observations following each manipulation is detailed in the Error! Reference source not found.

Suspensions were transferred to Isolute tubes for isolation and dried under vacuum for 2 mins then dried in vacuo at 50° C. for 24 hours.

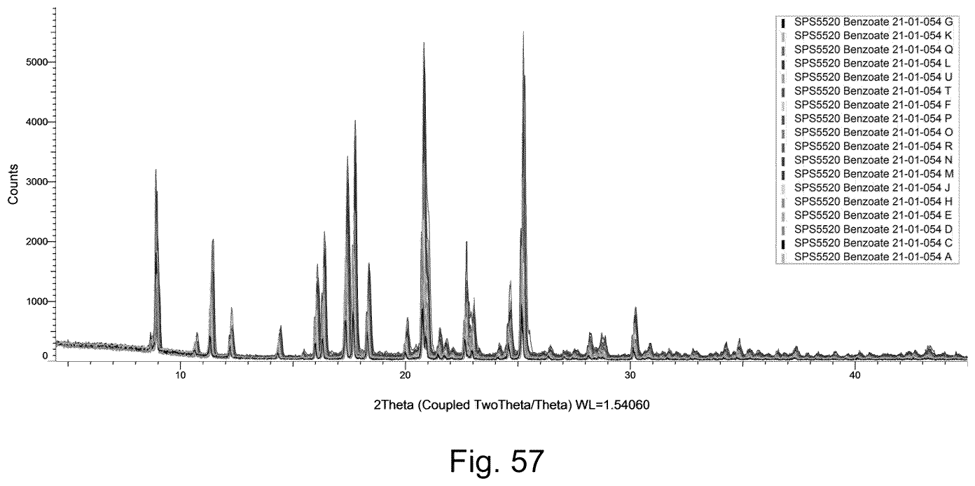

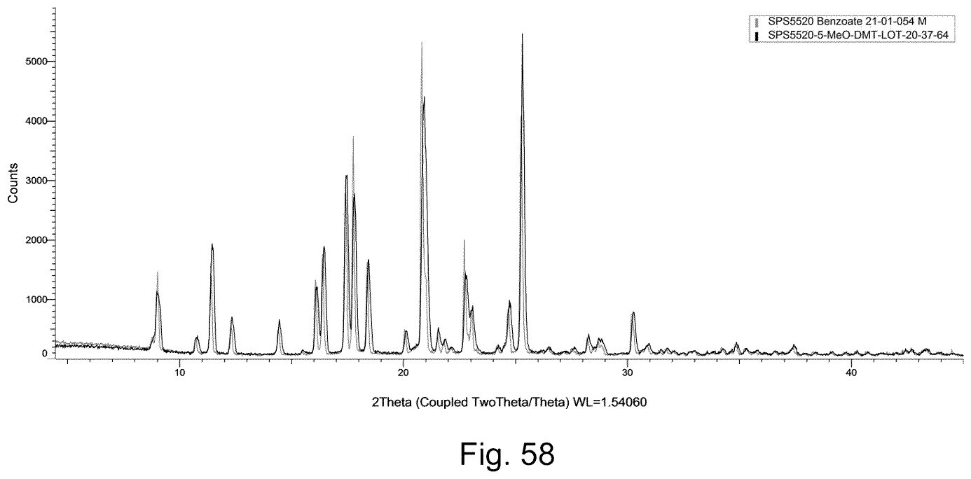

XRPD examination of the solids isolated from the equilibration of amorphous 5-MeO-DMT benzoate with thermal modulation revealed all powder patterns to be concordant with Pattern A (FIG. 57 and FIG. 58).

FIG. 57 shows XRPD pattern comparison of 5-MeO-DMT benzoate lot 21-01-054 solids isolated from the equilibration of amorphous 5-MeO-DMT benzoate with thermal modulation.

FIG. 58 shows XRPD pattern comparison of 5-MeO-DMT benzoate lot 21-01-054 M isolated from the equilibration of amorphous 5-MeO-DMT benzoate in α,α,α-trifluorotoluene with thermal modulation with lot 20-37-64 (Pattern A).

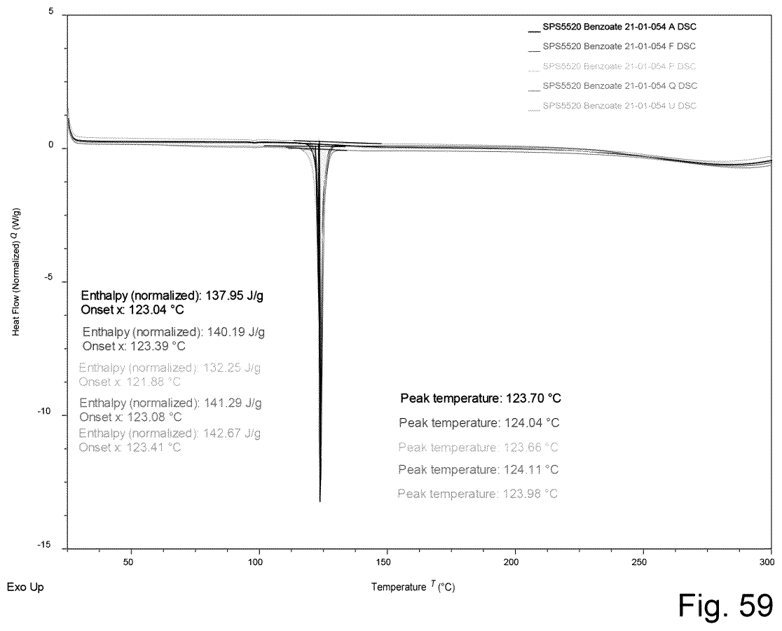

The DSC examination of a selection of 5-MeO-DMT benzoate solids classified as Pattern A revealed a major endothermic event with onset temperatures between 121.88 and 123.39° C. and peak temperatures between 123.66 and 124.11° C. This endotherm is characteristic of Pattern A form (FIG. 59).

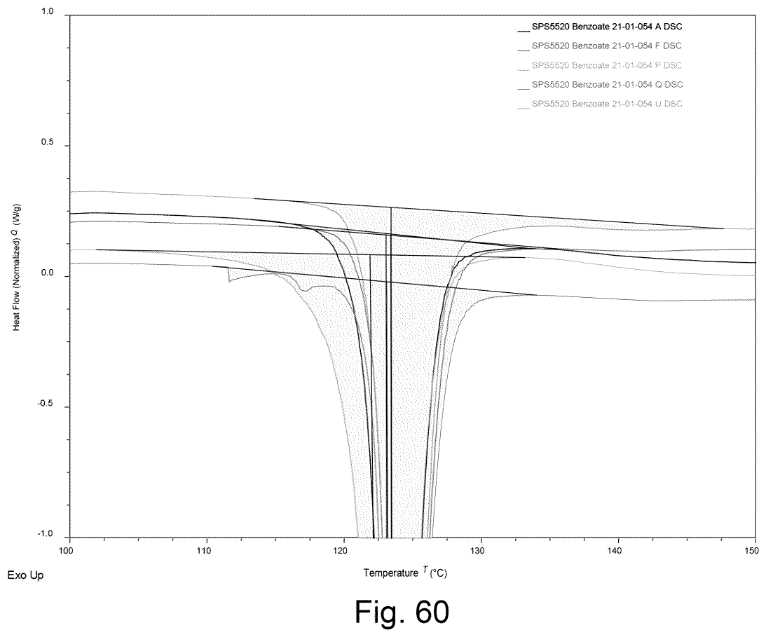

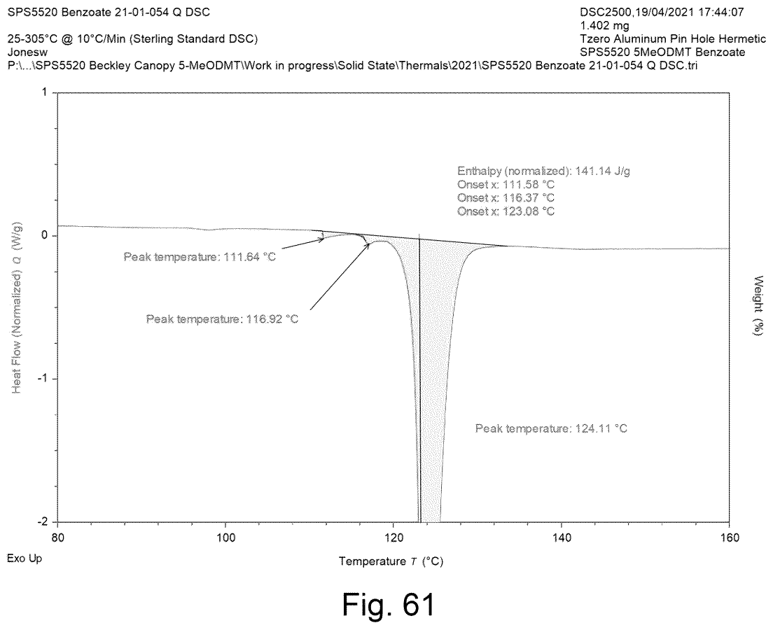

5-MeO-DMT benzoate 21-01-054 Q, solid isolated from anisole, contained events within the major endothermic event with peak temperatures of 111.64° C. and 116.92° C. (FIG. 60, FIG. 61). This is in line with the DSC thermograph of 5-MeO-DMT benzoate isolated following equilibration in anisole, 20-37-64-R1, although less pronounced.

FIG. 59 shows DSC thermograph comparison of a selection of 5-MeO-DMT benzoate lot 21-01-054 solids isolated from the equilibration of amorphous 5-MeO-DMT benzoate with thermal modulation classified as Pattern A form.

FIG. 60 shows DSC thermograph expansion comparison of a selection of 5-MeO-DMT benzoate lot 21-01-054 solids isolated from the equilibration of amorphous 5-MeO-DMT benzoate with thermal modulation classified as Pattern A form, highlighting an event in lot 21-01-054 Q, solid isolated from anisole.

FIG. 61 shows Expanded DSC thermograph expansion highlighting an event in lot 21-01-054 Q, isolated from anisole.

### Example 24: Pattern C

Additional 5-MeO-DMT benzoate Pattern B form material was required for further characterisation. The procedure of charging 5-MeO-DMT benzoate/IPA solution to cold toluene was employed.

5-MeO-DMT benzoate 20/20/150FP2, 250 mg, was dissolved in IPA, 5 ml, and heated to 50° C. and clarified. The clarified solution, 2×2 ml, 100 mg of 5-MeO-DMT benzoate, was charged to toluene, 4 ml, at −10° C. and agitated at 750 rpm.

Upon addition, both mixtures remained as clear colourless solutions.

After 30 minutes a solid had formed in tube A. The solid, 21-01-060 A, was isolated immediately via isolute and dried in vacuo for 2 minutes. A portion, 21-01-060 A1 was removed for XRPD analysis, a portion was dried in vacuo at 50° C. for 20 hours, 21-01-060 Å2.

After 50 minutes a solid had formed in tube B and was allowed to equilibrate at −10° C. and agitated at 750 rpm for 3 hours. The solid, 21-01-060 B, was isolated immediately via isolute and dried in vacuo for 2 minutes. A portion 21−01-060 B1 was removed for XRPD analysis, the remainder was dried in vacuo at 50° C. for 20 hours, 21-01-060 B2.

| | 5-MeO-DMT benzoate | 5-MeO-DMT benzoate |
|---|---|---|
| Sample | 21-01-060 A1 and A2 | 21-01-060 B1 and B2 |
| Tube | 21-01-060 A | 21-01-060 B |
| Origin | Reverse anti-solvent addition of salt/ | |
| | IPA solution to toluene at −10° C. | |
| Time to form | 30 minutes | 50 minutes |
| suspension | | |
| Time left as | ca. 0 minutes | 3 hours |
| suspension | | |
| Analysis | XRPD pattern collected taken | |
| | after 0 hours air dried | |
| | XRPD pattern and DSC | None |
| | thermograph collected after | |
| | 1 hour air dried | |

XRPD pattern and DSC thermograph
collected after 20 hours air drying
XRPD pattern and DSC thermograph collected
after 20 hours drying in vacuo at 50° C.

Samples 21-01-060 A1 and 21-01-060 B1 were air dried under ambient conditions for 20 hours and assessed by XRPD and DSC.

Immediately following isolation, 21-01-060 A1 was analysed by XRPD. This revealed a new diffraction pattern that was not concordant with Pattern A or Pattern B. This is referred to as Pattern C.

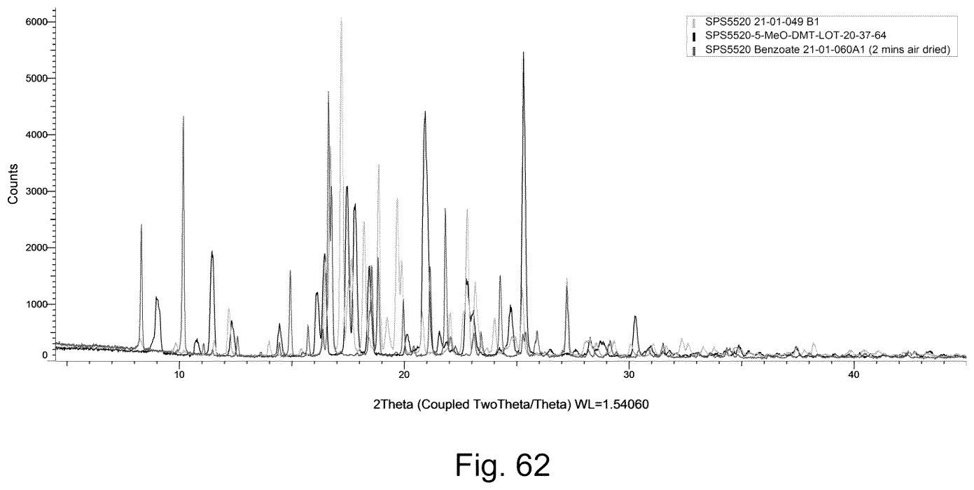

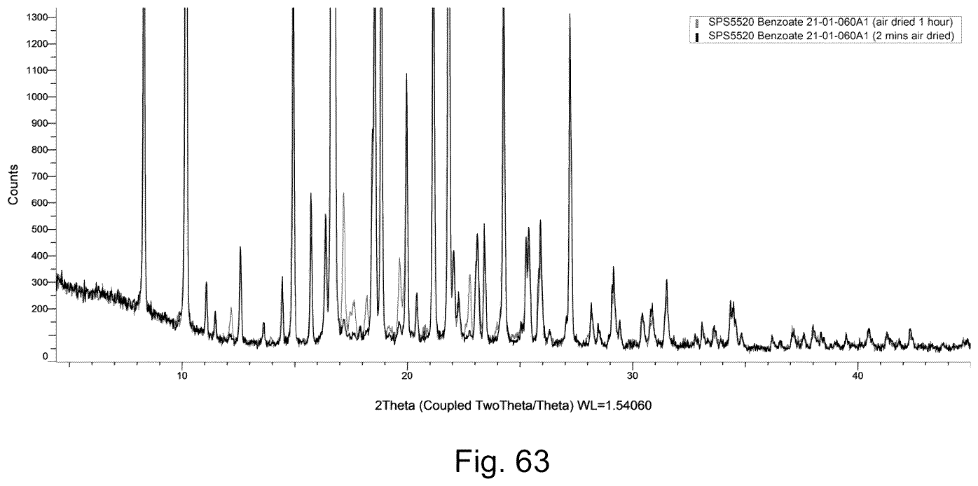

The XRPD pattern of 21-01-060 A1 (2 mins air dried) was reacquired following a further 1 hour of air drying under ambient conditions (FIG. 62). Additional diffractions were present in the XRPD of 21-01-060 A1 (air dried 1 hour) compared to 21-01-060 A1 (2 mins air dried), which suggests conversion to Pattern B form (FIG. 63).

FIG. 62 shows XRPD pattern comparison of 5-MeO-DMT benzoate lot 21-01-060 A1 air dried 2 minutes, lot 21-01−049 B1, Pattern B, and lot 20-37-64, Pattern A.

FIG. 63 shows XRPD pattern comparison of 5-MeO-DMT benzoate lot 21-01-060 Å1-air dried 1 hour and lot 21-01−060 Å1-air dried 2 minutes.

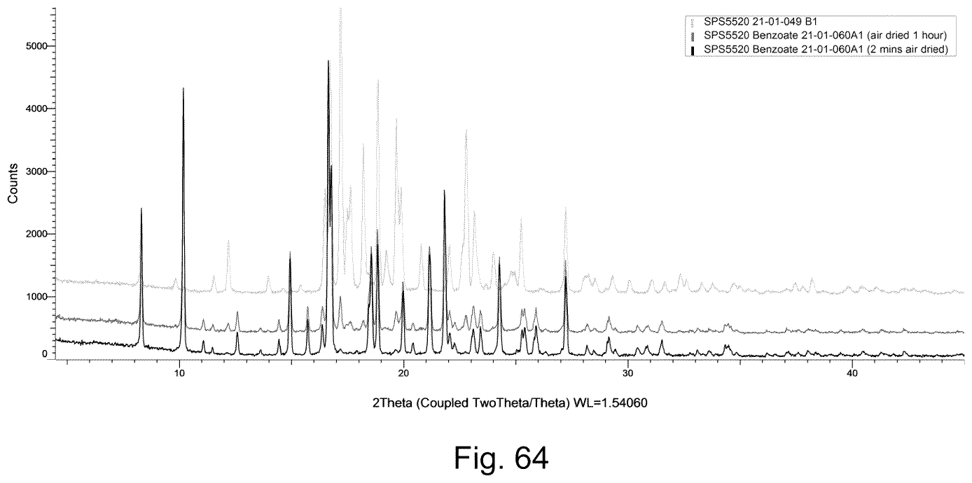

FIG. 64 shows XRPD pattern comparison of 5-MeO-DMT benzoate lot 21-01-060 Å1-air dried 2 minutes, lot 21-01−060 Å1-air dried 1 hour, and lot 21-01-049 B1, Pattern B.

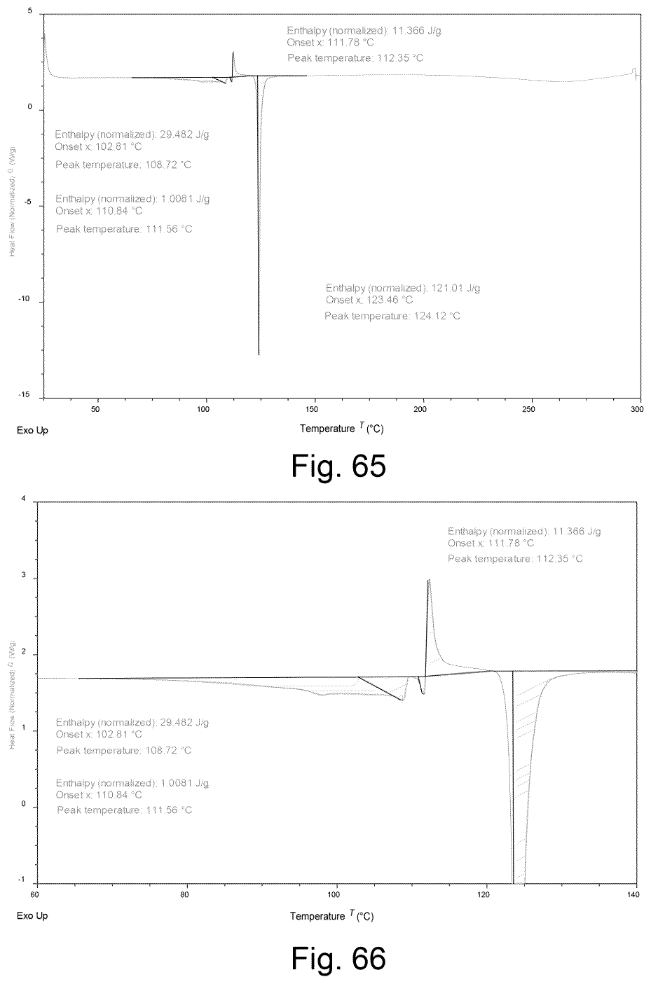

The DSC thermograph of 5-MeO-DMT benzoate 21-01-060 A1 (air dried 1 hour) (FIG. 65 and FIG. 66) revealed a minor broad endotherm with a peak temperature of 108° C. which is considered characteristic of Pattern C form solid.

This is followed by an exotherm with a peak temperature of 112.35° C. which is considered to be the conversion of Pattern C form to Pattern A form, since the main endotherm has a peak temperature of 124.12° C., which is characteristic of Pattern A form.

FIG. 65 shows DSC thermograph of 5-MeO-DMT benzoate lot 21-01-060 Å1, isolated immediately from IPA/toluene and air dried for 1 hour.

FIG. 66 shows DSC thermograph expansion of 5-MeO-DMT benzoate lot 21-01-060 Å1, isolated immediately from IPA/toluene and air dried for 1 hour.

An XRPD pattern of 5-MeO-DMT benzoate lot 21-01-060 A1 was acquired following a total of 20 hours air drying.

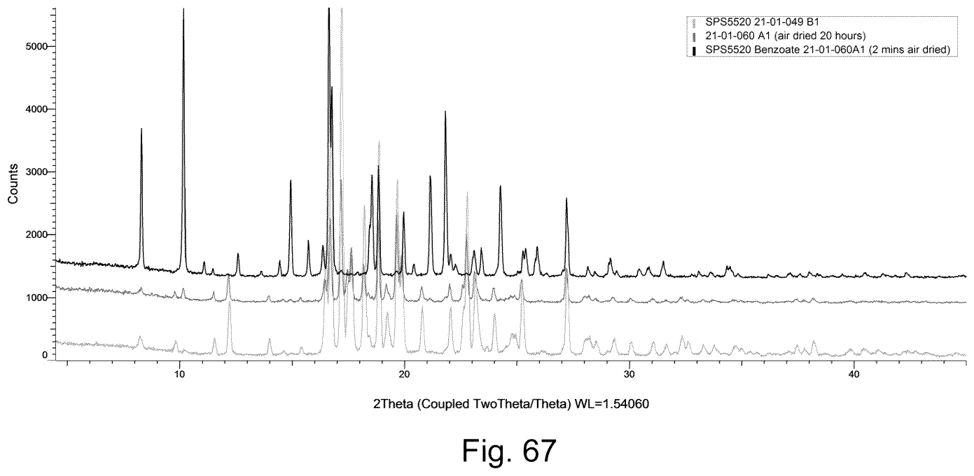

This revealed the pattern (FIG. 67) to be concordant with SPS5520 21-01-049 B1, Pattern B, but contained diffractions indicative of Pattern C such as 10.3°2θ (FIG. 67).

FIG. 67 shows XRPD pattern comparison of 5-MeO-DMT benzoate lot 21-01-060 A1 air dried 20 hours, lot 21-01−

060 A1 air dried 2 minutes, and lot 21-01-049 B1, Pattern B ref.

5-MeO-DMT benzoate 21-01-060 B1 produced from reverse anti-solvent addition, equilibrated for 3 hours, then isolated and air drying at ambient temperature

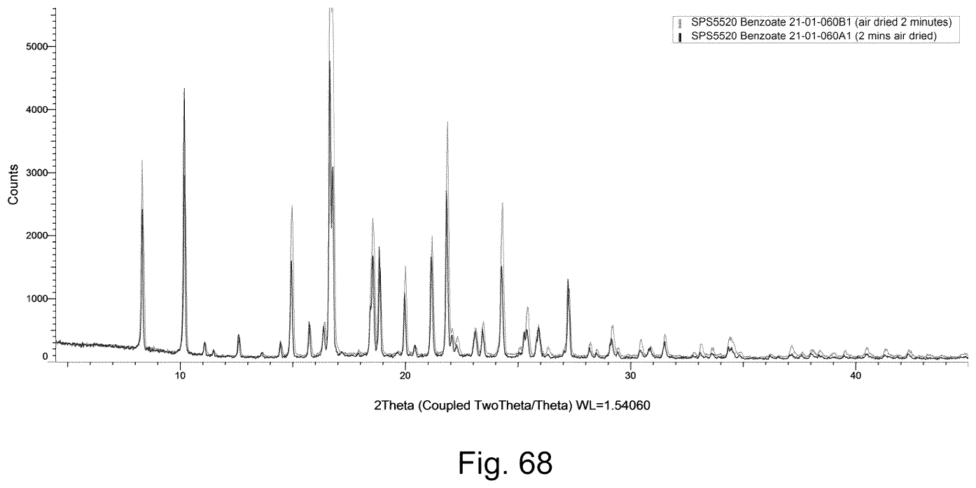

Immediately following isolation, the solid was analysed by XRPD. This revealed a diffraction pattern concordant with 21-01-060 Å1, Pattern C (FIG. 68).

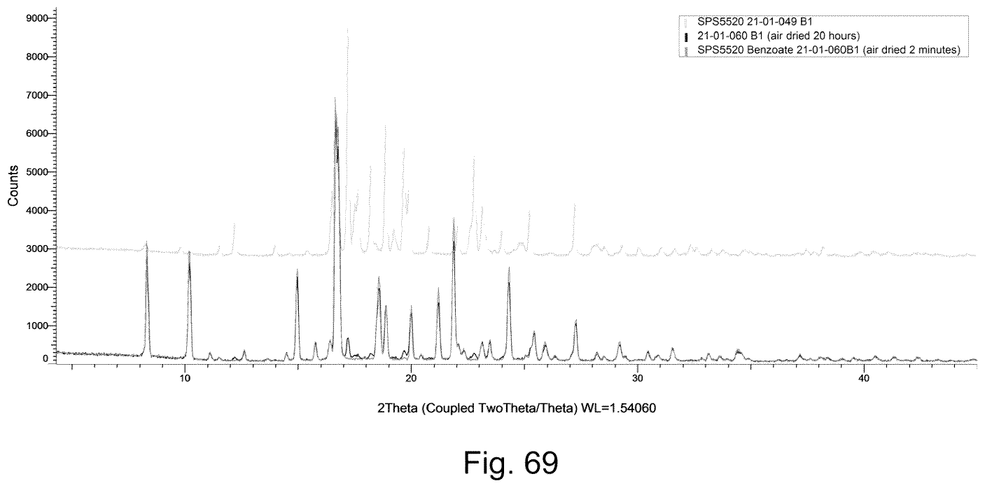

The XRPD pattern (FIG. 69) was reacquired following 20 hours air drying and revealed the solid was still Pattern C but contained diffractions at 17.2° and 19.5 2θ indicative of Pattern B.

FIG. 68 shows XRPD pattern comparison of 5-MeO-DMT benzoate lot 21-01-060 B1, isolated after 3 hours equilibration then air dried for 2 mins and A1 isolated immediately then air dried for 2 minutes.

FIG. 69 shows XRPD pattern comparison of 5-MeO-DMT benzoate lot 21-01-060 B1, isolated after 3 hours equilibration then air dried for 20 hours and B1 isolated after 3 hours equilibration then air dried for 2 minutes, and lot 21-01-049 B1, Pattern B.

### Example 25: Investigation of the Impact of Solvent Vapour Diffusion Upon Amorphous 5-MeO-DMT Benzoate

Subjecting an amorphous solid to solvent vapour is considered to be a low energy process for inducing form or version change of the solid in order to generate meta stable versions and/or solvates from the amorphous solid for comparison and evaluation.

5-MeO-DMT benzoate, 497.44 mg, was dissolved in deionised water, 10 mL, and clarified into a 500 mL round bottom flask and lyophilised as detailed previously. The fluffy white solid produced, 12×25 mg, was charged to HPLC vials and placed in a sealed container with ca. 2 mL of solvent. The solvents employed and observations are detailed in the Table below.

Following equilibration for 7 days, solids were transferred to XRPD sample holder directly and analysed by XRPD. DSC was collected for all notable samples by XRPD and a selection of Pattern A form solids.

| | | Observations | | |
|---|---|---|---|---|
| ID | Solvent | Upon charge | Post 1 day | Post 7 days |
| A | Methanol | Off-white gum | White Opaque solid | Yellow solution |
| B | Ethyl acetate | Off-white gum | Off-white gum | Off-white agglomerate |
| C | Acetone | Off-white gum | White Opaque solid | Solids adhered to glass above a |
| | | | | clear solution |
| D | Anisole | Off-white gum | Off-white gum | Off-white agglomerate |
| E | TBME | Off-white gum | Off-white gum | Off-white agglomerate |
| F | THF | Off-white gum | Off-white gum | Off-white agglomerate |
| G | Toluene | Off-white gum | Off-white gum | Off-white agglomerate |
| H | 1,4-Dioxane | Off-white gum | Off-white gum | Off-white agglomerate |
| I | DCM | Off-white gum | Off-white gum | Solids adhered to glass above a |
| | | | | clear solution |
| J | Heptane | Off-white gum | Off-white gum | Off-white agglomerate |
| K | Acetonitrile | Off-white gum | Off-white gum | Off-white agglomerate |
| L | Water | Off-white gum | Off-white gum | Off-white agglomerate |

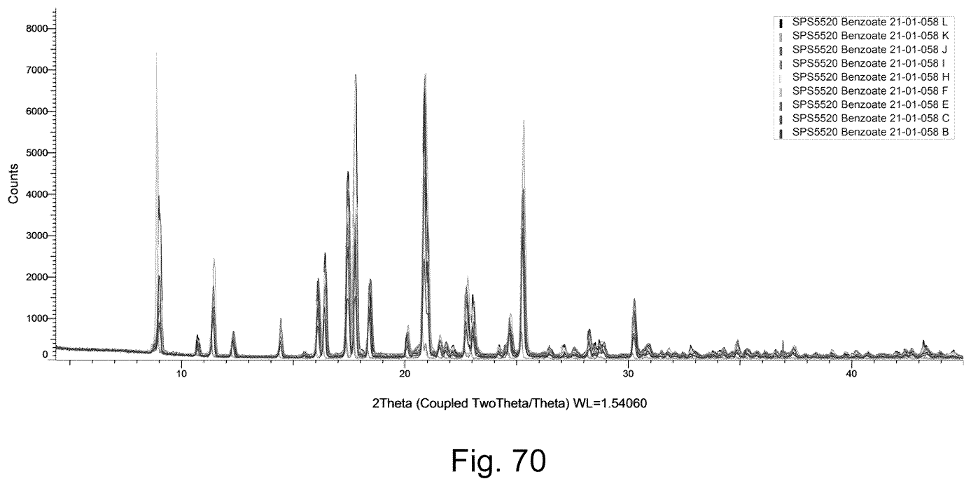

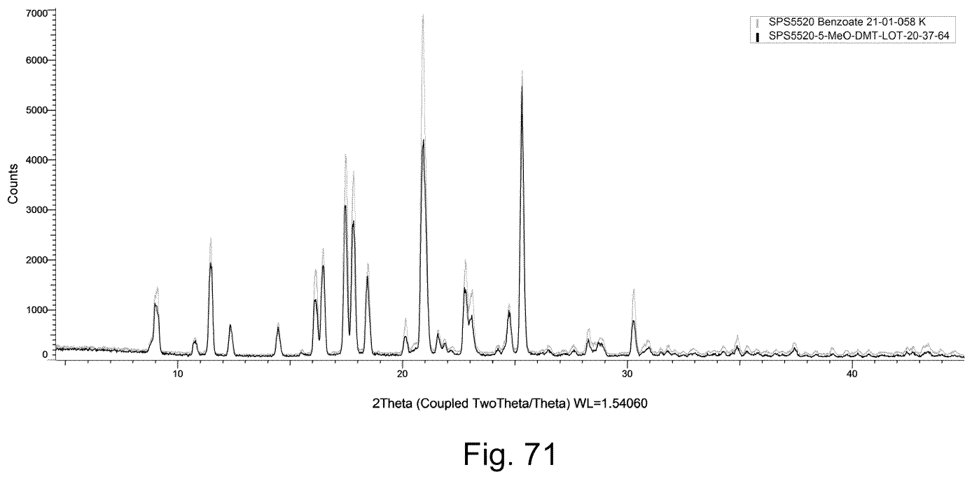

XRPD pattern for all samples (FIG. 70) except for 21-01-058 D and 21-01-058 G, isolated from anisole and toluene respectively, were concordant with Pattern A form material (FIG. 71).

FIG. 70 shows XRPD pattern comparison of 5-MeO-DMT benzoate lot 21-01-058 solids isolated from amorphous 5-MeO-DMT benzoate exposed to solvent vapour.

FIG. 71 shows XRPD pattern comparison of 5-MeO-DMT benzoate lot 21-01-058 K, isolated from amorphous 5-MeO-DMT benzoate exposed to solvent vapour, with lot 20-37-64, Pattern A.

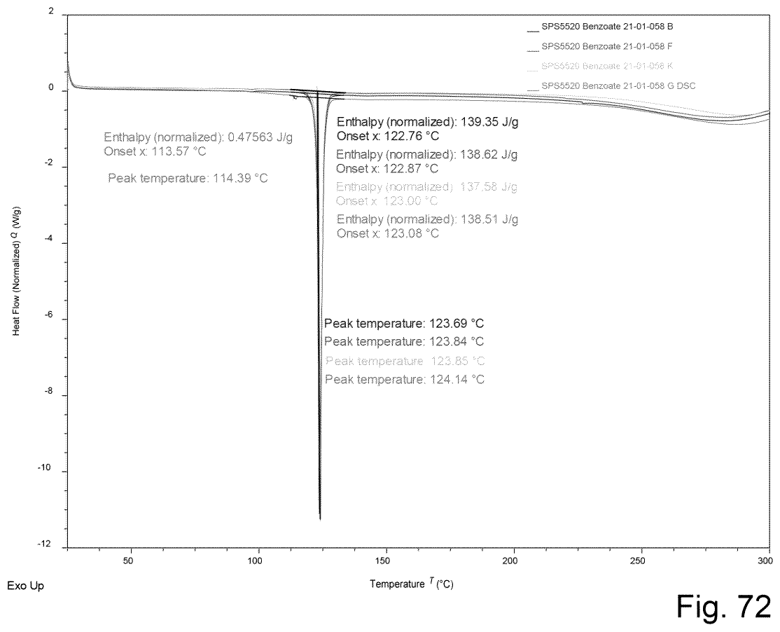

The DSC thermograph comparison of a selection of Pattern A form solids (FIG. 72) revealed an endothermic event with peak temperatures between 123.69° C. and 124.14° C. which is indicative of Pattern A form and corroborates the XRPD data.

The DSC thermograph of lot 21-01-058 G (not Pattern A form, by XRPD) demonstrates a minor endothermic event prior to the main endotherm and is elaborated on below.

FIG. 72 shows DSC thermograph comparison of 5-MeO-DMT benzoate lot 21-01-058 B, lot 21-01-058 F, lot 21-01−058 K, and lot 21-01-062 G.

### Example 26: Pattern D

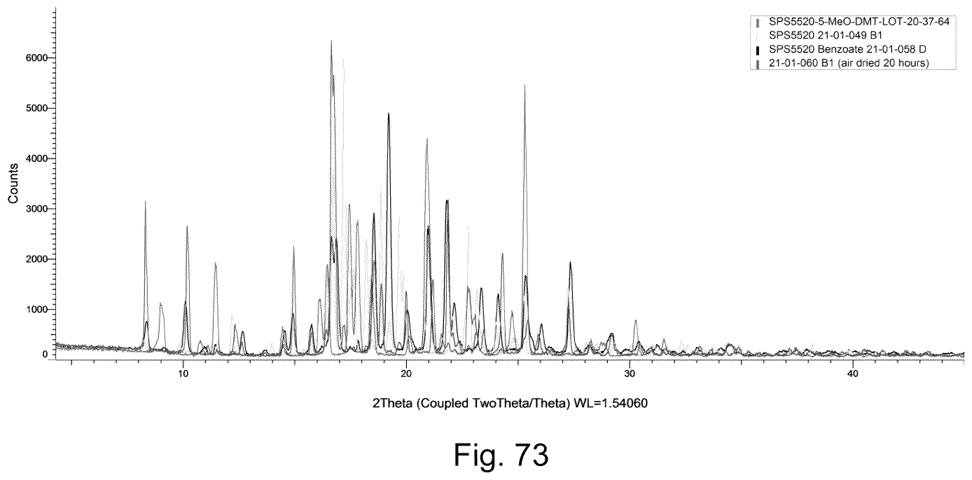

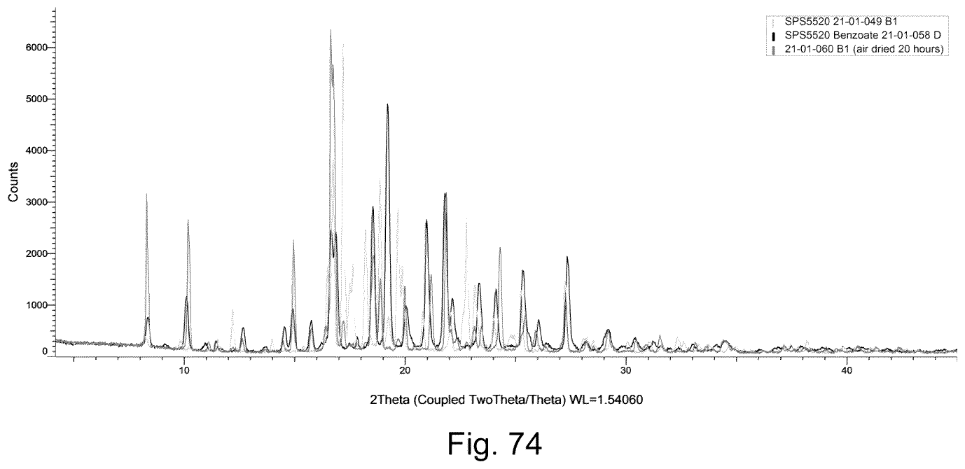

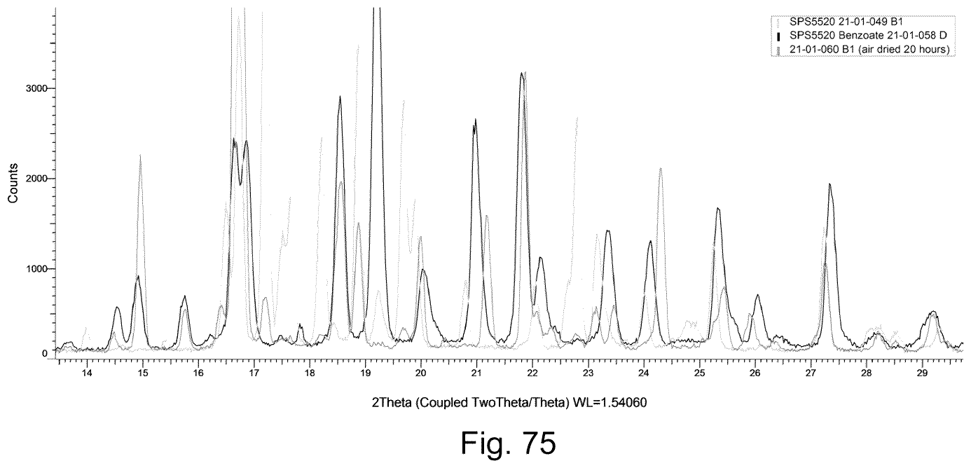

5-MeO-DMT benzoate 21-01-058 D, solid isolated from exposure of amorphous 5-MeO-DMT benzoate to anisole vapour for 7 days XRPD of 5-MeO-DMT benzoate lot 21-01-058 D, isolated from amorphous 5-MeO-DMT benzoate exposed to anisole vapour, revealed a unique powder pattern (FIG. 73 and FIG. 74). The diffractions of 21-01-058 D are similar to Pattern C but vary in intensity and position (FIG. 75).

FIG. 73 shows XRPD pattern comparison of 5-MeO-DMT benzoate lot 21-01-058 D, lot 20-37-64, Pattern A, lot 21−01-049 B1, Pattern B, and lot 21-01-060 B1, Pattern C (air dried 20 hours).

FIG. 74 shows XRPD pattern comparison of 5-MeO-DMT benzoate lot 21-01-058 D, lot 21-01-049 B1, Pattern B, and lot 21-01-060 B1, Pattern C (air dried 20 hours).

FIG. 75 shows XRPD pattern expansion comparison of 5-MeO-DMT benzoate lot 21-01-058 D, lot 21-01-049 B1, Pattern B, and lot 21-01-060 B1, Pattern C (air dried 20 hours).

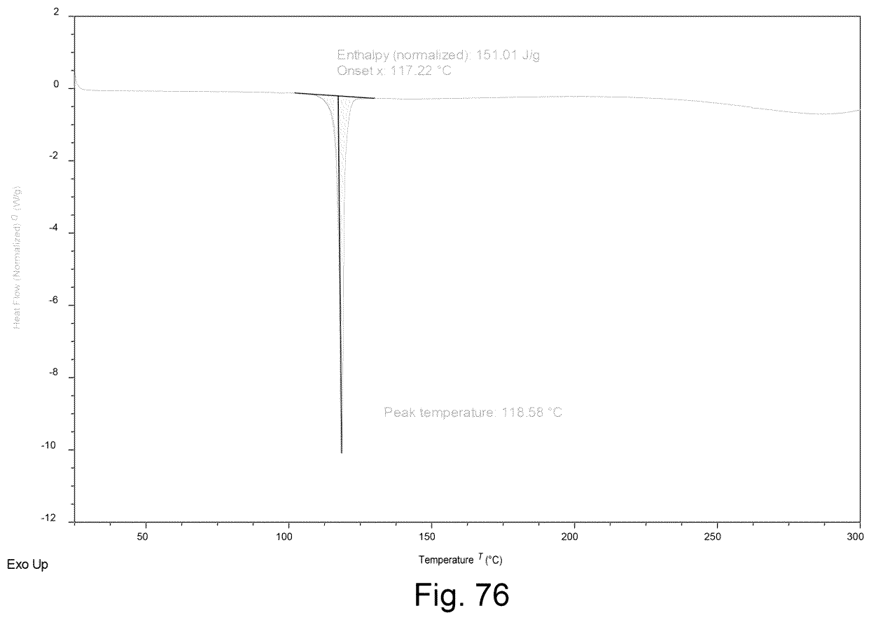

The DSC thermograph of 5-MeO-DMT benzoate lot 21-01-058 D (FIG. 76), isolated from amorphous 5-MeO-DMT benzoate exposed to anisole vapour revealed an endothermic event with a peak temperature of 118.58° C. This corroborates the XRPD data, confirming a new version has been isolated.

FIG. 76 shows DSC thermograph of 5-MeO-DMT benzoate lot 21-01-058 D, isolated from exposure of anisole vapour to amorphous form.

Amorphous 5-MeO-DMT benzoate exposed to anisole vapour afforded an anisole hemi-solvate, nominated herein as Pattern D form. The XRPD pattern of Pattern D form is similar to Pattern C, the toluene hemi-solvate, but with variance in peak position.

Amorphous 5-MeO-DMT benzoate exposed to toluene vapour afforded a mixed form version that was predominantly Pattern A form with some evidence of Pattern C form, the toluene hemi-solvate, observed by XRPD and DSC.

Amorphous 5-MeO-DMT benzoate exposed to all other solvent vapours returned exclusively Pattern A by XRPD and DSC.

| Sample | Solvent | XRPD | DSC | 1H NMR |
|---|---|---|---|---|
| A | Methanol | N/A - solution by day 7 | | |
| B | Ethyl acetate | Pattern A | Endo at 123.69° C. | NC |
| C | Acetone | Pattern A | NC | NC |
| D | Anisole | Pattern D | Endo at 118.58° C. | Salt to anisole ratio of 1:0.47 |
| E | TBME | Pattern A | NC | NC |
| F | THF | Pattern A | Endo at 123.84° C. | NC |
| G | Toluene | Predominantly | Endo at 114.39° C. | Salt to toluene ratio of 1:0.04 |
| | | Pattern A and | Endo at 124.14° C. | |
| | | some Pattern C | | |
| H | 1,4-Dioxane | Pattern A | NC | NC |
| I | DCM | Pattern A | NC | NC |
| J | Heptane | Pattern A | NC | NC |
| K | Acetonitrile | Pattern A | Endo at 123.85° C. | NC |
| L | Water | Pattern A | NC | NC |

### Example 27: Pattern E

5-MeO-DMT benzoate Pattern C form was isolated via reverse anti-solvent addition of isopropanol solution of 5-MeO-DMT benzoate to toluene, this solid is believed to be a hemi-solvate which when desolvated afforded Pattern B form. Pattern B form has been accessed by equilibration of 5-MeO-DMT benzoate in anisole and chlorobenzene.

Pattern B form may be accessed from anisole and chlorobenzene hemi-solvates, consequently reverse anti-solvent addition to chlorobenzene and anisole is believed to afford a hemi-solvate as with toluene.

5-MeO-DMT benzoate 20/20/150FP2, 650 mg, was charged to sample vial with IPA, 13 ml, and heated to 50° C. The clear solution was clarified through a 0.45 μm nylon syringe filter.

Anti-solvent, 4 ml, was charged to crystallisation tubes and cooled to −10° C. with agitation via stirrer bead at 750 rpm as detailed in the Table below.

IPA stock solution at 50° C., 2 ml, was charged to cold anti-solvent, 4 ml, at −10° C.

Observations are detailed in the Table below, with B, D, and F isolated immediately.

Tubes A, C, and E were equilibrated for 3 hours then isolated.

Suspensions were transferred to isolute cartridge and dried in vacuo for NMT 60 seconds and analysed immediately, following 4 hours, and 44 hours open to atmosphere.

5-MeO-DMT benzoate 21-01-064 E was damp after air drying for 60 seconds.

| | | Time to form a | | Equilibration period after | |
|---|---|---|---|---|---|
| Tube | Anti-solvent | suspension | | suspension formed | |
| A | Toluene | 3.5 | hours | 3 | hours |
| B | Toluene | 3 | hours | 0 | hours |
| C | Chlorobenzene | 3.5 | hours | 3 | hours |
| D | Chlorobenzene | 3.5 | hours | 0 | hours |
| E | Anisole | 3.5 | hours | 3 | hours |
| F | Anisole | 3 | hours | 0 | hours |

5-MeO-DMT benzoate 21-01-064 D was isolated immediately following the formation of the suspension afforded by the addition of concentrated IPA solution to chlorobenzene at −10° C.

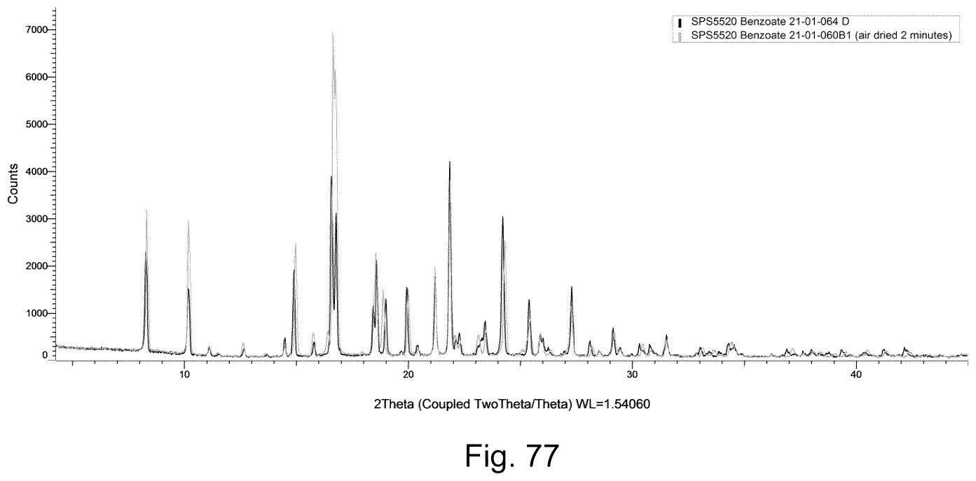

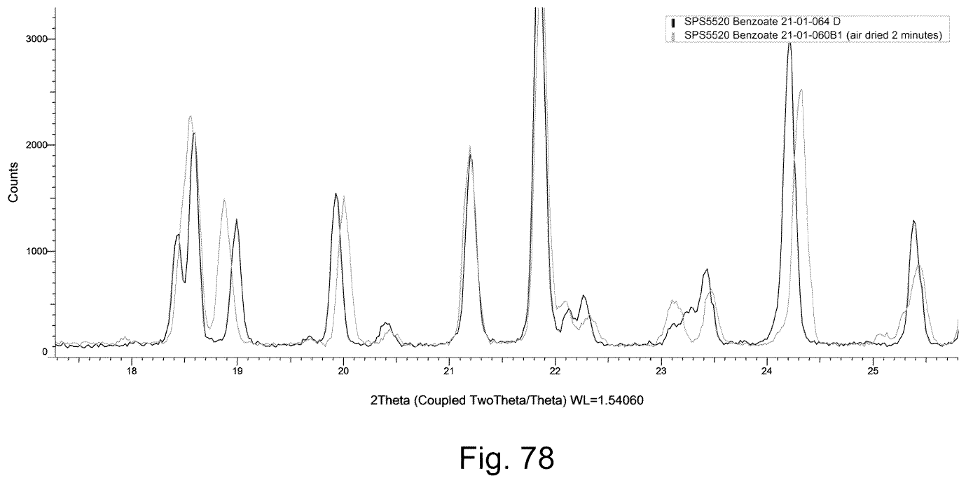

The XRPD revealed the diffraction pattern of 5-MeO-DMT benzoate lot 21-01-064 D was similar to 21-01-060 B1 (air dried 2 minutes), Pattern C (FIG. 77). Several diffractions including 19 and 20°2θ are slightly higher and lower compared to Pattern C which are not consequences of the sample presentation (FIG. 78).

5-MeO-DMT benzoate lot 21-01-064 D is a new diffraction pattern, and defined herein as Pattern E.

FIG. 77 shows XRPD pattern comparison of 5-MeO-DMT benzoate lot 21-01-064 D, and 21-01-060 B1 (air dried 2 minutes).

FIG. 78 shows XRPD pattern expansion comparison of 5-MeO-DMT benzoate lot 21-01-064 D, and 21-01-060 B1 (air dried 2 minutes).

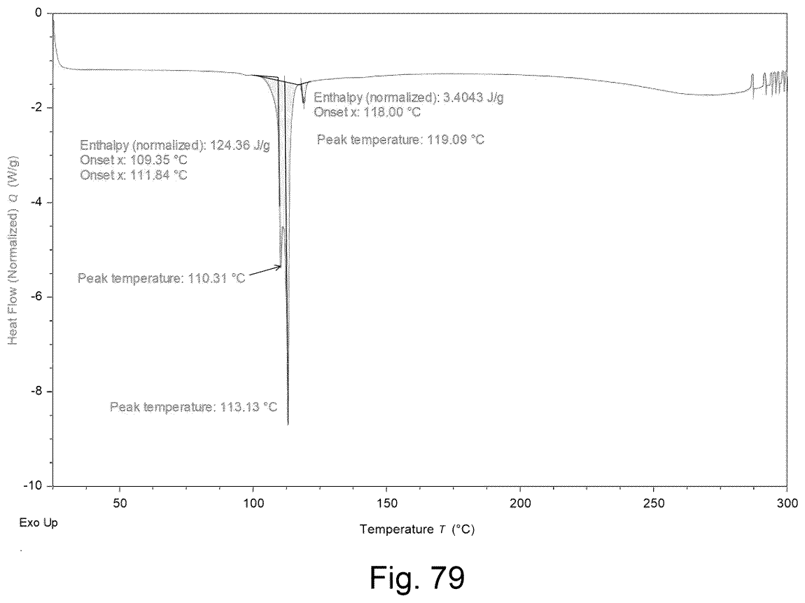

The DSC thermograph of 5-MeO-DMT benzoate lot 21-01-064 D revealed a major bimodal endothermic event with peak temperatures of 110.31° C. and 113.13° C. (FIG. 79), followed by a minor endothermic event with a peak temperature of 119.09° C.

FIG. 79 shows DSC thermograph of 5-MeO-DMT benzoate lot 21-01-064 D at 10° C.min⁻¹.

The 1H NMR spectrum of 5-MeO-DMT benzoate lot 21-01-064 D isolated immediately following equilibration revealed the stoichiometry of the salt to be 1:1 and also revealed a salt to solvent ratio for chlorobenzene of 1:0.512 and a salt to solvent ratio for IPA of 1:0.013.

The isolated salt is a chlorobenzene hemi-solvate.

There is no evidence of a Pattern A form endothermic at ca. 123° C. in the DSC thermograph, 21-01-064 D (FIG. 79) since it is considered that the residual chlorobenzene is inhibiting crystallisation of 5-MeO-DMT benzoate.

5-MeO-DMT benzoate 21-01-064 C was isolated following a 3 hour equilibration of the suspension afforded by the addition of concentrated IPA solution to chlorobenzene at −10° C.

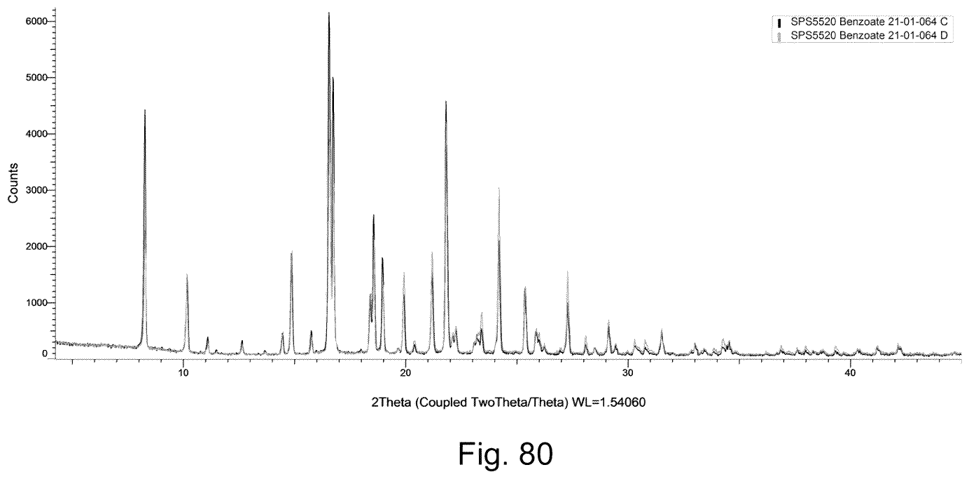

The XRPD revealed the diffraction pattern of 5-MeO-DMT benzoate lot 21-01-064 C was concordant with 21-01-064 D, Pattern E (FIG. 80).

FIG. 80 shows XRPD pattern comparison of 5-MeO-DMT benzoate lot 21-01-064 C, and 21-01-064 D.

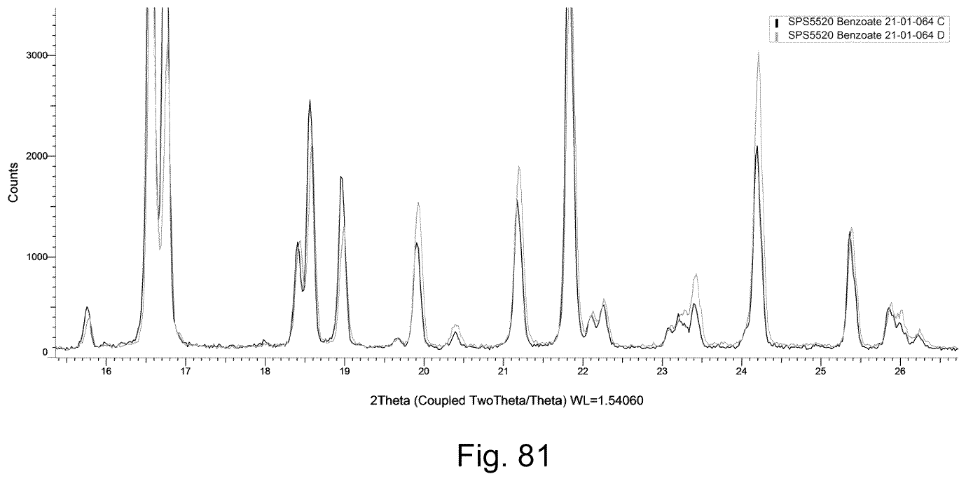

FIG. 81 shows XRPD pattern expansion comparison of 5-MeO-DMT benzoate lot 21-01-064 C, and 21-01-064 D.

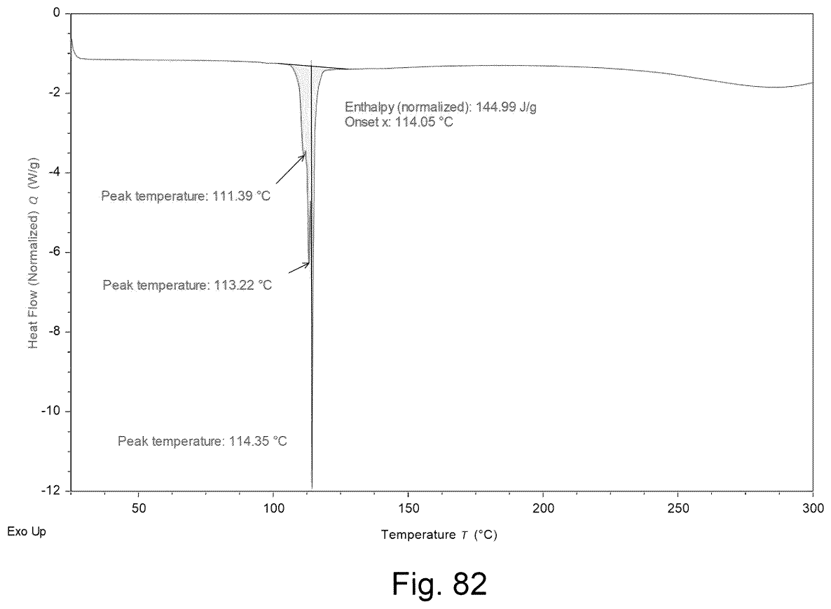

The DSC thermograph of 5-MeO-DMT benzoate lot 21-01-064 C revealed a major endothermic event with peak temperatures of 111.39° C., 113.22° C., and 114.35° C. (FIG. 82).

The DSC thermograph of 21-01-064 C is similar to that of the thermograph of 21-01-064 D.

FIG. 82 shows DSC thermograph of 5-MeO-DMT benzoate lot 21-01-064 C at 10° C.min⁻¹.

The 1H NMR spectrum of 5-MeO-DMT benzoate lot 21-01-064 C isolated following a 3 hour equilibration revealed the stoichiometry of the salt to be 1:1 and also revealed a salt to solvent ratio for chlorobenzene of 1:0.506 and a salt to solvent ratio for IPA of 1:0.004.

The isolated salt is a chlorobenzene hemi-solvate.

The XRPD of 5-MeO-DMT benzoate lot 21-01-064 C (4 hours air dried) revealed a diffraction pattern concordant with 21-01-064 C, Pattern E.

The XRPD of 5-MeO-DMT benzoate lot 21-01-064 C (44 hours air dried) revealed a diffraction pattern concordant with 21-01-064 C and 21-01-064 C (4 hours air dried), Pattern E.

The XRPD of 5-MeO-DMT benzoate lot 21-01-064 F revealed a diffraction pattern concordant with 21-01-058 D, Pattern D from the vapour diffusion investigation of amorphous 5-MeO-DMT benzoate in anisole, but more crystalline and does not contain minor diffractions characteristic of Pattern A.

The XRPD of 5-MeO-DMT benzoate 21-01-064 E revealed a diffraction pattern concordant with 21-01-064 F, Pattern D.

The XRPD of 5-MeO-DMT benzoate 21-01-064 E (air dried 4 hours) revealed a diffraction pattern concordant with 21-01-064 A, Pattern D.

The XRPD of 5-MeO-DMT benzoate 21-01-064 F (air dried 44 hours) revealed a diffraction pattern concordant with 21-01-064 F, Pattern D but with an additional diffraction at 18.30 2θ, which is believed to be an indication of Pattern B.

### Example 28: Further Discussion of Patterns b to d

### Pattern B

Below is a Table which summarises lots of 5-MeO-DMT benzoate with predominantly Pattern B form compositional and crystallographic characteristics.

| | | Crystalline | Composition by |
|---|---|---|---|
| Sample name | Comments | character | 1H NMR |
| 5-MeO-DMT benzoate | Addition of methanol solution to cold | Pattern B | 1:0.03 toluene |
| 21-01-049 A1 | toluene then isolated and dried in vacuo | and C | 0 MeOH |
| | at 50° C. | | |
| 5-MeO-DMT benzoate | Addition of IPA solution to cold toluene | Pattern B | 1:0.01 toluene |
| 21-01-049 B1 | then isolated and dried in vacuo at 50° C. | | 0 IPA |
| 5-MeO-DMT benzoate | Crystallised from cooling a saturated | Pattern B | |
| 21-01-047 J | solution of chlorobenzene and dried in | and A | |
| | vacuo at 50° C. | | |
| 5-MeO-DMT benzoate | Addition of IPA solution to cold toluene | Pattern B | 1:0.04 toluene |
| 21-01-060 A1 (air | then isolated immediately and air dried | and C | 1:0.20 IPA |
| dried 20 hours) | for 20 hours | | |
| 5-MeO-DMT benzoate | Addition of IPA solution to cold toluene | Pattern B | 1:0.007 toluene |
| 21-01-060 A2 | then isolated immediately and dried in | | 1:0.09 IPA |
| | vacuo at 50° C. | | |
| 5-MeO-DMT benzoate | Addition of IPA solution to cold toluene, | Pattern B | 1:0.05 toluene |
| 21-01-060 B2 | equilibrated for 3 hours, then isolated | and C | 1:0.07 IPA |
| | and dried in vacuo at 50° C. | | |

Below is a Table which summarises predominantly Pattern B thermal characteristics.

| | Broad | | | | | | | | |
|---|---|---|---|---|---|---|---|---|---|
| Sample | exo at | Endo at | Endo at | Endo at | Exo at | Endo at | Exo at | Exo at | Endo at |
| name | 101° C. | 109.5° C. | 110.5° C. | 113° C. | 113.4° C. | 114° C. | 114.1° C. | 117.8° C. | 124° C. |
| 5-MeO- | | | Y | Y | | Y | | Y | Y |
| DMT | | | | | | | | | |
| benzoate | | | | | | | | | |
| 21-01-049 | | | | | | | | | |
| A1 | | | | | | | | | |
| 5-MeO- | | | Y | | | Y | | Y | Y |
| DMT | | | | | | | | | |
| benzoate | | | | | | | | | |
| 21-01-049 | | | | | | | | | |
| B1 | | | | | | | | | |
| 5-MeO- | | Y | | Y | | | Y | | Y |
| DMT | | | | | | | | | |
| benzoate | | | | | | | | | |
| 21-01-047 | | | | | | | | | |
| J | | | | | | | | | |
| 5-MeO- | Y | | | | Y | | | | Y |
| DMT | | | | | | | | | |
| benzoate | | | | | | | | | |
| 21-01-060 | | | | | | | | | |
| A1 (air | | | | | | | | | |
| dried 20 | | | | | | | | | |
| hours) | | | | | | | | | |
| 5-MeO- | | | Y | | | Y | | Y | Y |
| DMT | | | | | | | | | |
| benzoate | | | | | | | | | |
| 21-01-060 | | | | | | | | | |
| A2 | | | | | | | | | |
| 5-MeO- | | Y | | | | | | Y | Y |
| DMT | | | | | | | | | |
| benzoate | | | | | | | | | |
| 21-01-060 | | | | | | | | | |
| B2 | | | | | | | | | |

| | |
|---|---|
| Characteristic of Pattern B | Characteristic |
| | of Pattern A |

5-MeO-DMT benzoate lot 21-01-049 B1 was produced via reverse anti-solvent addition of an IPA solution to toluene, isolated immediately, then dried in vacuo at 50° C. XRPD revealed a diffraction pattern that was defined as Pattern B. DSC examination identified an endothermic event at 110° C. which coincides with the boiling point of toluene, this is followed by an endothermic event immediately followed by an exothermic event indicating the melt-crystallisation of Pattern B form to Pattern A form then the endothermic event indicating the melt of Pattern A form material. 1H NMR revealed low amounts of residual toluene and no IPA.

5-MeO-DMT benzoate lot 21-01-060 Å2 was produced by the same methodology as 049 B1 except on a larger scale and afforded an identical product by XRPD and DSC but contained residual IPA by 1H NMR.

5-MeO-DMT benzoate lot 21-01-049 A1 was produced by the same methodology as 049 B1 except it was initially dissolved in methanol, XRPD revealed a powder pattern concordant with Pattern B with some Pattern C. 1H NMR revealed a salt to toluene ratio of 1:0.03. DSC examination revealed a similar thermograph to 049 B1 but the first endothermic event at 110° C. was larger and the subsequent endothermic melt of Pattern B form is bimodal and peaks at a lower temperature. Following the melt of Pattern B form, Pattern A form crystallises, and melts as expected.

5-MeO-DMT benzoate lot 21-01-060 B2 was produced by the same methodology as 060 Å2 but equilibrated for 3 hours before isolation and drying in vacuo. XRPD revealed a mixture of Pattern B with some Pattern C. 1H NMR revealed a salt to toluene ratio of 1:0.05. DSC examination revealed a similar thermograph to 049 A1 (a mixture of Pattern B and C forms) but the Pattern B form melt endothermic event is not bimodal. The endothermic event at 110° C. is considered to be a consequence of a slightly increased amount of toluene in the sample in the form of the toluene hemi-solvate.

5-MeO-DMT benzoate lot 21-01-060 A1 (air dried 20 hours) was produced by the same methodology as 060 Å2 but was air dried instead of at 50° C. in vacuo. XRPD revealed a mixture of Pattern B and C. 1H NMR revealed a salt to toluene ratio of 1:0.04. However, 060 A1 contained a significant amount more IPA than other samples (1:0.2 instead of 1:0.05). This may have modified the endothermic events during the DSC examination of the sample, but the Pattern A form melt endothermic event is present.

5-MeO-DMT benzoate lot 21-01-047J was produced by crystallisation from chlorobenzene at 50° C. and dried in vacuo at 50° C. XRPD revealed the sample to be a mixture of Pattern B and some Pattern A. DSC examination revealed an endothermic event similar to the endothermic event considered to be loss of toluene, which is believed to indicate the loss of chlorobenzene. The melting endotherm of Pattern B form occurs earlier than for 049 B1 but the crystallisation of Pattern A form is very exothermic and is accompanied by a melt of Pattern A form.

5-MeO-DMT benzoate Pattern B form material contains a characteristic endo-exothermic event as it melts then crystallises as Pattern A form, Pattern B form is produced by the desolvation of hemi-solvates, therefore an endothermic event characteristic of the residual hemi-solvate is present in all samples isolated.

For those solids that contain toluene at low levels, which is believed to be the hemi-solvate version of the salt, the thermal characteristics will be modified by the loss of toluene.

### Pattern C

Below is a Table which summarises lots of 5-MeO-DMT benzoate with predominantly Pattern C compositional and crystallographic characteristics.

| | | Crystalline | Composition |
|---|---|---|---|
| Sample name | Comments | character | by 1H NMR |
| 5-MeO-DMT benzoate | Addition of IPA solution to cold toluene | Pattern C | |
| 21-01-060 A1 (air | then isolated and air dried for 1 hour | and B | |
| dried 1 hour) | | | |
| 5-MeO-DMT benzoate | Addition of IPA solution to cold toluene, | Pattern C | 1:0.43 toluene |
| 21-01-060 B1 (air | equilibrated for 3 hours, then isolated | and B | 1:0.12 IPA |
| dried 20 hours) | and air dried for 20 hours | | |
| 5-MeO-DMT benzoate | Addition of IPA solution to cold toluene, | Pattern C | 1:0.49 toluene |
| 21-01-064 A | equilibrated for 3 hours, then isolated | | 1:0.004 IPA |
| 5-MeO-DMT benzoate | Addition of IPA solution to cold toluene, | Pattern C | |
| 21-01-064 A (air | equilibrated for 3 hours, then isolated | and B | |
| dried 4 hours) (DSC | and air dried for 4 hours | | |
| at 2.5° C. min⁻¹) | | | |
| 5-MeO-DMT benzoate | Addition of IPA solution to cold toluene, | Pattern C | |
| 21-01-064 A (air | equilibrated for 3 hours, then isolated | and B | |
| dried 44 hours) | and air dried for 44 hours | | |
| 5-MeO-DMT benzoate | Addition of IPA solution to cold toluene | Pattern C | 1:0.5 toluene |
| 21-01-064 B | then isolated | | 1:0.006 IPA |

Below is a Table which summarises predominantly Pattern C form thermal characteristics.

| | Exo between | | | | | | |
|---|---|---|---|---|---|---|---|
| | 105 and | Endo at | Endo at | Endo at | Exo at | Endo at | Exo at |
| Sample name | 113° C. | 111.0° C. | 111.3° C. | 112.1° C. | 112.4° C. | 113.3° C. | 113.6° C. |
| 5-MeO- | P | | | | Y | | |
| DMT | | | | | | | |
| benzoate | | | | | | | |
| 21-01-060 | | | | | | | |
| A1 (air | | | | | | | |
| dried 1 | | | | | | | |
| hour) | | | | | | | |
| 5-MeO- | | | Y | | | | |
| DMT | | | | | | | |
| benzoate | | | | | | | |
| 21-01-060 | | | | | | | |
| B1 (air | | | | | | | |
| dried 20 | | | | | | | |
| hours) | | | | | | | |
| 5-MeO- | | | Y | | | Y | |
| DMT | | | | | | | |
| benzoate | | | | | | | |
| 21-01-064 | | | | | | | |
| A | | | | | | | |
| 5-MeO- | Y | | | | | Y | Y |
| DMT | | | | | | | |
| benzoate | | | | | | | |
| 21-01-064 | | | | | | | |
| A (air | | | | | | | |
| dried 4 | | | | | | | |
| hours) | | | | | | | |
| (DSC at | | | | | | | |
| 2.5° C. min⁻¹) | | | | | | | |
| 5-MeO- | | Y | Y | | | | |
| DMT | | | | | | | |
| benzoate | | | | | | | |
| 21-01-064 | | | | | | | |
| A (air | | | | | | | |
| dried 44 | | | | | | | |
| hours) | | | | | | | |
| 5-MeO- | | | Y | Y | | | Y |
| DMT | | | | | | | |
| benzoate | | | | | | | |
| 21-01-064 | | | | | | | |

Characteristic of Pattern B

| | Endo at | Endo at | Endo at | Endo at | Endo at | Endo at |
|---|---|---|---|---|---|---|
| Sample name | 115.0° C. | 115.5° C. | 117.8° C. | 120.2° C. | 122.0° C. | 124° C. |
| 5-MeO- | | | | | | Y |
| DMT | | | | | | |
| benzoate | | | | | | |
| 21-01-060 | | | | | | |
| A1 (air | | | | | | |
| dried 1 | | | | | | |
| hour) | | | | | | |
| 5-MeO- | Y | Y | Y | | | |
| DMT | | | | | | |
| benzoate | | | | | | |
| 21-01-060 | | | | | | |
| B1 (air | | | | | | |
| dried 20 | | | | | | |
| hours) | | | | | | |
| 5-MeO- | Y | Y | | Y | | |
| DMT | | | | | | |
| benzoate | | | | | | |
| 21-01-064 | | | | | | |
| A | | | | | | |
| 5-MeO- | | | | | | Y |
| DMT | | | | | | |
| benzoate | | | | | | |
| 21-01-064 | | | | | | |
| A (air | | | | | | |
| dried 4 | | | | | | |
| hours) | | | | | | |
| (DSC at | | | | | | |
| 2.5° C. min⁻¹) | | | | | | |
| 5-MeO- | Y | | | | Y | |
| DMT | | | | | | |
| benzoate | | | | | | |
| 21-01-064 | | | | | | |
| A (air | | | | | | |
| dried 44 | | | | | | |
| hours) | | | | | | |
| 5-MeO- | Y | | | | Y | |
| DMT | | | | | | |
| benzoate | | | | | | |
| 21-01-064 | | | | | | |
| | | | | | | Characteristic |
| | | | | | | of Pattern A |

5-MeO-DMT benzoate lot 21-01-064 B was produced by reverse anti-solvent addition of an IPA solution to toluene. XRPD revealed Pattern C which was supported by a ratio of 1:0.5 of salt to toluene by 1H NMR indicating a toluene hemi-solvate. DSC examination revealed a bimodal endothermic event with peak temperatures of 111.3° C. and 112.1° C., this indicates the endothermic event at 111° C. in the Pattern B mixtures was a result of residual Pattern C. There were endothermic events indicative of Pattern B form, which suggested transformation to Pattern B form then Pattern A form.

5-MeO-DMT benzoate lot 21-01-064 A was produced by the same methodology as 064 B but was equilibrated for 3 hours before isolation. XRPD and 1H NMR revealed identical characteristics as 064 B. However, DSC examination revealed a different major multi-modal endothermic event with a peak temperature of 115.0° C.

5-MeO-DMT benzoate lot 21-01-064 A (air dried 44 hours) and 21-01-060 B1 air dried (20 hours) were produced similarly to 064 A but air dried for longer. XRPD revealed a mixture of Pattern C and Pattern B for both, 1H NMR revealed less toluene in 060 B1 than for 064 A, which is believed to be a result of air drying which supports the presence of Pattern B form in the sample by XRPD. DSC examination revealed an endothermic event with a peak temperature of 111.3° C. for both, followed by multiple unique endothermic events.

5-MeO-DMT benzoate lot 21-01-064 A (air dried 4 hours) was produced by air drying 064 Å. XRPD revealed a mixture of Pattern C with some Pattern B. DSC examination revealed a broad exothermic event between 105 and 113° C. followed by a weak endothermic event indicative of Pattern C form and endothermic events indicative of Pattern B form. The change to the heating rate is the cause of the change to thermal behaviour, as the DSC thermograph of 21-01-064 A (44 hour air dried) sample is similar to 21-01-064 A the transformation of Pattern C form occurred in situ during the examination.

5-MeO-DMT benzoate 21-01-060 A1 (air dried 1 hour) was produced by the same methodology as 064 A but isolated immediately. XRPD revealed a mixture of Pattern C and some Pattern B. DSC examination revealed a thermograph indicative of Pattern B form with a minor exothermic event at ca 109° C.

5-MeO-DMT benzoate Pattern C form is a toluene hemi-solvate it has no characteristic endothermic event except for a melt between 110° C. and 115° C. The XRPD pattern of the toluene hemi-solvate of 5-MeO-DMT benzoate is distinct to 5-MeO-DMT benzoate. Desolvation may occur under ambient conditions and it is considered that Pattern B form is produced.

The thermal characteristics will be influenced by the loss of toluene during DSC examination.

### Pattern D

The Table below is a summary of predominantly Pattern D form compositional and crystallographic characteristics.

| | | Crystalline | Composition |
|---|---|---|---|
| Sample name | Comments | character | by 1H NMR |
| 5-MeO-DMT | Exposure of amorphous form to | Pattern | 1:0.47 anisole |
| benzoate 21- | anisole vapours | D and A | |
| 01-058 D | | | |
| 5-MeO-DMT | Addition of IPA solution to cold | Pattern D | 1:1.04 anisole |
| benzoate 21- | anisole, equilibrated for 3 hours, | | 1:0.11 IPA |
| 01-064 E | then isolated | | |
| 5-MeO-DMT | Addition of IPA solution to cold | Pattern D | |
| benzoate 21- | anisole, equilibrated for 3 hours, | | |
| 01-064 E (air | then isolated and air dried for 4 | | |
| dried 4 hours) | hours | | |
| 5-MeO-DMT | Addition of IPA solution to cold | Pattern | |
| benzoate 21- | anisole, equilibrated for 3 hours, | D and B | |
| 01-064 E (air | then isolated and air dried for 44 | | |
| dried 44 hours) | hours | | |
| 5-MeO-DMT | Addition of IPA solution to cold | Pattern D | 1:0.503 anisole |
| benzoate 21- | anisole then isolated | | 1:0.01 IPA |
| 01-064 F | | | |

The table below shows a summary of predominantly Pattern D form thermal characteristics.

| | Endo at | Endo at | Endo at | Endo at |
|---|---|---|---|---|
| Sample name | 111.2° C. | 117.8° C. | 118.6° C. | 119.2° C. |
| 5-MeO-DMT benzoate | | | Y | |
| 21-01-058 D | | | | |
| 5-MeO-DMT benzoate | Y | | | |
| 21-01-064 E | | | | |
| 5-MeO-DMT benzoate | Y | Y | | |
| 21-01-064 E (air | | | | |
| dried 4 hours) | | | | |
| 5-MeO-DMT benzoate | Y | Y | Y | |
| 21-01-064 E (air | | | | |
| dried 44 hours) | | | | |
| 5-MeO-DMT benzoate | | | Y | Y |
| 21-01-064 F | | | | |

5-MeO-DMT benzoate lot 21-01-064 F was produced by reverse anti-solvent addition of an IPA solution to anisole and isolated immediately. XRPD revealed a diffraction pattern concordant with Pattern D, which was supported by a ratio of 1:0.503 for anisole by 1H NMR indicating a hemi-solvate. DSC examination revealed a bimodal endothermic event with peak temperatures of 118.61° C. and 119.21° C.

5-MeO-DMT benzoate lot 21-01-064 E was produced by reverse anti-solvent addition of an IPA solution to anisole, then equilibrated for 3 hours before isolation. XRPD revealed Pattern D but this was not supported by 1H NMR which revealed a ratio of salt to anisole of 1:1.04, the isolated solid was damp after isolation. DSC examination revealed very poorly defined broad endothermic events with peak temperatures of 113.51° C. and 161.93° C., the endothermic event at 113.51° C. is believed to be a result of the melting of the hemi-solvate present by XRPD followed by evaporation of anisole. The DSC thermograph is not considered representative of Pattern D form due to the solvent content.

5-MeO-DMT benzoate lot 21-01-058 D was produced by exposure of the amorphous form to anisole vapour. XRPD revealed a mixture of Pattern D and some Pattern A diffractions which was supported by 1H NMR which revealed a ratio of salt to anisole of 1:0.47 indicating an anisole hemi-solvate. DSC examination revealed an endothermic event with a peak temperature of 118.6° C., which is concordant with the data collected from 064 F. However, the melt of Pattern A form is not revealed in the DSC thermograph, this could be modified by the liberated anisole solvent present in the sample.

5-MeO-DMT benzoate lot 21-01-064 E (air dried 4 hours) was produced by air drying 064 E for 4 hours. XRPD revealed Pattern D. DSC examination was performed at 2.5° C.min⁻¹ with the aim to resolve the bimodal endothermic event observed in the thermograph of 064 E. DSC examination revealed a minor endothermic event with a peak temperature of 111.24° C., this endothermic event is concordant with the broad endothermic event observed in 064 E. The better resolution of this endothermic is believed to be a result of the slower heating rate, or due to removal of residual anisole by air drying. This was followed by a major endothermic event with a peak temperature of 117.90° C. which is concordant with 058 D and 064 F.

5-MeO-DMT benzoate lot 21-01-064 E (air dried 44 hours) was produced by air drying 064 E (air dried 4 hours) for a further 40 hours. XRPD revealed a mixture of Pattern D with some Pattern B diffractions. DSC examination revealed a thermograph concordant with 064 E (4 hours air dried). The Pattern B form content was not evident in the DSC thermograph this is believed to be caused by the liberated anisole solvent present in the sample, similar to 058 D.

5-MeO-DMT benzoate Pattern D form is an anisole hemi-solvate and has been produced directly from exposure of the amorphous form to anisole vapour as well as reverse anti-solvent addition from an IPA solution to cold anisole. No characteristic thermal behaviour has been identified although, endothermic events near 118° C. are common and the lack of recrystallisation to Pattern B or A forms is believed to be due to the presence of residual anisole.

### Pattern E

The Table below is a summary of predominantly Pattern E form compositional and crystallographic characteristics.

| | | Crystalline | Composition |
|---|---|---|---|
| Sample name | Comments | character | by 1H NMR |
| 5-MeO-DMT | Addition of IPA solution to cold chlorobenzene, | Pattern E | 1:0.506 |
| benzoate 21-01- | equilibrated for 3 hours, then isolated | | chlorobenzene |
| 064 C | | | 1:0.04 IPA |
| 5-MeO-DMT | Addition of IPA solution to cold chlorobenzene, | Pattern E | |
| benzoate 21-01- | equilibrated for 3 hours, then isolated and air dried | | |
| 064 C (air dried 4 | for 4 hours | | |
| hours) | | | |
| 5-MeO-DMT | Addition of IPA solution to cold chlorobenzene, | Pattern E | |
| benzoate 21-01- | equilibrated for 3 hours, then isolated and air dried | | |
| 064 C (air dried 44 | for 44 hours | | |
| hours) | | | |
| 5-MeO-DMT | Addition of IPA solution to cold chlorobenzene then | Pattern E | 1:0.512 |
| benzoate 21-01- | isolated | | chlorobenzene |
| 064 D | | | 1:0.01 IPA |

The table below is a summary of predominantly Pattern E form thermal characteristics, the endothermic event at 123.7° C. is characteristic of Pattern A.

| Sample | Exo between | Endo at | Endo at | Endo at | Endo at | Endo at | Endo at | Endo at | Endo at |
|---|---|---|---|---|---|---|---|---|---|
| name | 105 and 115° C. | 110.3° C. | 111.3° C. | 113.1° C. | 114.3° C. | 115.1° C. | 115.8° C. | 119.1° C. | 123.7° C. |
| 5-MeO-DMT benzoate | | | Y | Y | Y | | | | |
| 21-01-064 C | | | | | | | | | |
| 5-MeO-DMT benzoate | Y | | | | Y | | | | Y |
| 21-01-064 C (air dried 4 | | | | | | | | | |
| hours) | | | | | | | | | |
| 5-MeO-DMT benzoate | | | | | | Y | Y | | |
| 21-01-064 C (air dried 44 | | | | | | | | | |
| hours) | | | | | | | | | |
| 5-MeO-DMT benzoate | | Y | | Y | | | | Y | |
| 21-01-064 D | | | | | | | | | |

5-MeO-DMT benzoate lot 21-01-064 D was produced by reverse anti-solvent addition of an IPA solution to chlorobenzene. XRPD revealed Pattern E, this was supported by 1H NMR which revealed a ratio of salt to chlorobenzene of 1:0.506 indicating a chlorobenzene hemi-solvate. DSC examination revealed a bimodal endothermic event with peak temperatures of 111.3° C. and 113.1° C., followed by a minor endothermic event with a peak temperature of 119.1° C.

5-MeO-DMT benzoate lot 21-01-064 C was produced by reverse anti-solvent addition of an IPA solution to cold chlorobenzene, then equilibrated for 3 hours before isolation. XRPD revealed Pattern E, this was supported by 1H NMR which revealed a ratio of salt to chlorobenzene of 1:0.512 indicating a hemi-solvate. DSC examination revealed a trimodal endothermic event with peak temperatures of 111.3° C., 113.1° C., and 114.3° C. There are similarities between DSC thermographs of 064 D and C but the endothermic event at 119.1° C. is not present in 064 C and 064 D did not reveal a trimodal endothermic event. The differences in the DSC thermograph are of note since the XRPD patterns were identical and 1H NMR revealed hemi-solvates.

5-MeO-DMT benzoate lot 21-01-064 C (air dried 4 hours) was produced by air drying 064 C for 4 hours. XRPD revealed Pattern F. DSC examination was performed at 2.5° C.min⁻¹ and revealed a broad exothermic event followed by a minor endothermic event at 114.3° C. but much weaker in comparison to the same endothermic event in 064 C. This was followed by the major endothermic event at 123.7° C. which is indicative of Pattern A form. The DSC thermograph is similar to the previous 2.5° C.min⁻¹ DSC examination and is generating Pattern A form during the DSC examination.

5-MeO-DMT benzoate lot 21-01-064 C (air dried 44 hours) was produced by air drying 064 C (air dried 4 hours) for a further 40 hours. XPRD revealed Pattern F. DSC examination revealed a bimodal endothermic event with peak temperatures of 115.1° C. and 115.8° C. The endothermic event of 064 C (air dried 44 hours) is similar to 064 C but peaks at a slightly higher temperature.

5-MeO-DMT benzoate Pattern F form is a chlorobenzene hemi-solvate with no defined thermal characteristics except for a multi-modal endothermic event between 11° and 117° C. Similarly, to the anisole hemi-solvate, Pattern A and B forms do not recrystallise from the melt. Chlorobenzene hemi-solvate appears to not desolvate when open to ambient conditions and did not desolvate over 44 hours.

### Example 29: Hemi-Solvates

Equilibration of suspensions in anti-solvent (toluene, anisole, and chlorobenzene) at −10° C. afforded the expected hemi-solvate by XRPD and 1H NMR spectroscopy and TGA.

The partial desolvation of hemi-solvates is considered to afford multi-modal endothermic events observed in the DSC thermographs, a consequence of changing composition and the applied heating rate.

Desolvation of hemi-solvates in vacuo at 50° C. for 22 hours afforded Pattern B form material by XRPD, DSC, however, some residual hemi-solvate remained in all samples.

The DSC thermograph of the hemi-solvates were similar to those isolated from IPA/antisolvent but with minor differences which are considered to be a consequence of how they were prepared.

Drying 5-MeO-DMT benzoate toluene hemi-solvate and chlorobenzene hemi-solvate in vacuo at 50° C. for 67 hours afforded Pattern A form, but the anisole hemi-solvate afforded predominantly Pattern B form.

Addition of 5-MeO-DMT benzoate/IPA solution to toluene at −10° C. then air dried for 5 minutes afforded the toluene hemi-solvate when performed on a 1 g input.

Drying 5-MeO-DMT benzoate toluene hemi-solvate at 50° C. for 24 hours afforded Pattern B form.

5-MeO-DMT benzoate batches 20/53/057-FP and 20/20/123FP demonstrated similar particle habits of large hexagonal/rhombus plates (ca. 500 μm to 1 mm in length) and some smaller plates that demonstrated accretion on the plate surfaces and significant evidence of broken fine particles and plates, potentially due to attrition.

This was different to batches 20/20/150FP2 T=0 and 20/20/154FP which demonstrated similar particle habits of accreted, jagged clusters of irregular plates, (ca. 250 to 600 μm in length) and broken, irregular plates and crystallites (some <20 μm in length) that were indicative of particle attrition.

The significant difference in particle size and habit between the batches is believed to have an impact on isolation, flowability and kinetic dissolution rate of the solids, highlighting the importance of a controlled crystallisation.

### Example 30: Patterns F and G

5-MeO-DMT benzoate methyl benzoate hemi-solvate (Pattern F form) has been isolated from controlled cooling of a clarified 5-MeO-DMT benzoate methyl benzoate solution from 50° C. to −10° C.

5-MeO-DMT benzoate 2-chlorotoluene hemi-solvate (Pattern G form) has been isolated from controlled cooling of a clarified 5-MeO-DMT benzoate 2-chlorotoluene solution from 80° C. to −10° C.

Equilibration in α,α,α-trifluorotoluene did not afford a hemi-solvate as anticipated from a monosubstituted aromatic solvent. Equilibration in cumene afforded Pattern B form, which indicated a cumene hemi-solvate.

DVS examination of amorphous 5-MeO-DMT benzoate revealed a weight loss of ca. 2% indicating the elimination of a component and confirming that a stable hydrate of 5-MeO-DMT benzoate was not isolated.

Pattern A form is the most stable version of 5-MeO-DMT benzoate and is the thermodynamically favoured product except when isolated from a small selection of solvents, which afforded the respective hemi-solvate.

Stability studies revealed conversion of all patterns to Pattern A form when dried in vacuo at 50° C. However, Pattern B form has been shown to be stable when open to atmosphere at ca. 20° C. for up to 12 days. Pattern C form underwent partial conversion to Pattern B form within 24 hours when open to atmosphere at ca. 20° C., but failed to convert any further from a Pattern B/C mixed version over an additional 11 days.

FTIR spectra for Patterns A, B and C were overall similar though there were some unique bands in Pattern A form and absent bands that were otherwise present and shared by Patterns B and C forms.

Controlled Cooling Crystallisation Investigation with an Expanded Solvent Selection

Initial cooling crystallisation investigation of 5-MeO-DMT benzoate revealed Pattern A form was isolated from most solvents except chlorobenzene which was consistent with Pattern B form. The range of solvents was expanded, with an emphasis on esters and aromatics.

5-MeO-DMT benzoate lot 20/20/150FP2, 50 mg±1 mg, was charged to crystallisation tubes A-L. Minimal solvent at 50° C. was charged to afford a clear solution as detailed in the Table below. Crystallisation tubes I, J, K, and L remained as suspensions at 12.5 mg·ml−1 at 50° C. and so were heated to 80° C. to afford clear solutions.

Solutions were clarified into crystallisation tubes at 50° C. and were cooled to −10° C. at a rate of 10° chr-1, then equilibrated at −10° C. for 12 hours, then agitated at −10° C. at 400 rpm for 30 minutes which afforded a mobile suspension for all samples except Sample I which remained a solution. Further equilibration with agitation at −10° C. at 400 rpm for 3 hours afforded a thin suspension. All samples were isolated via isolute cartridge and air dried for 5 minutes before characterisation.

Sample F isolated from methyl benzoate was a thick white paste after air drying for 5 minutes and was left to air dry on the XRPD sample holder for a further 30 minutes which then afforded a dry powder.

| Cryst. | | Solubilitymg · | |
|---|---|---|---|
| tube | Solvent | ml⁻¹ at° C. | Observations |
| A | Methyl acetate | 33.3 at 50 | Crystals grew during controlled cooling, then agitated to form |
| | | | a mobile suspension |
| B | n-Propyl acetate | 20 at 50 | Clear solution post equilibration that afforded a mobile |
| | | | suspension following brief agitation |
| C | Iso-Propyl acetate | 16.7 at 50 | Crystals grew during controlled cooling, then agitated to form |
| | | | a mobile suspension |
| D | Iso-Butyl acetate | 12.5 at 50 | Clear solution post equilibration that afforded a mobile |
| | | | suspension following brief agitation |
| E | Ethyl formate | 40 at 50 | Crystals grew during controlled cooling, then agitated to form |
| | | | a mobile suspension |
| F | Methyl benzoate | 50 at 50 | Clear solution post equilibration that afforded a mobile |
| | | | suspension following brief agitation |
| G | Methyl propionate | 40 at 50 | Crystals grew during controlled cooling, then agitated to form |
| | | | a mobile suspension |
| H | 4-Methyl-2-pentanone | 25 at 50 | Clear solution post equilibration that afforded a mobile |
| | | | suspension following brief agitation |
| I | Cumene | 12.5 at 80 | Clear solution post equilibration that afforded a mobile |
| | | | suspension following agitation for 3 hours |
| J | Toluene | 12.5 at 80 | Crystals grew during controlled cooling, then agitated to form |
| | | | a mobile suspension |
| K | 2-Chlorotoluene | 12.5 at 80 | Crystals grew during controlled cooling, then agitated to form |
| | | | a mobile suspension |
| L | α,α,α-Trifluorotoluene | 12.5 at 80 | Crystals grew during controlled cooling, then agitated to form |
| | | | a mobile suspension |

5-MeO-DMT benzoate lots 21-01-073 B, C, D, E, G, H, and L were isolated from n-propyl acetate, isopropyl acetate, iso-butyl acetate, ethyl formate, methyl propionate, 4-methyl-2-pentanone, and α,α,α-trifluorotoluene respectively.

The XRPD of these samples revealed powder patterns concordant with 5-MeO-DMT benzoate lot 20-37-64, Pattern A.

The DSC thermograph of a selection of pattern A material revealed a common endothermic event with a peak temperature ranging from 123.07° C. to 124.17° C. with an enthalpy of ca. 140 J·g⁻¹, which is characteristic of Pattern A form. The ¹H NMR spectra of 5-MeO-DMT benzoate lots 21-01-073 B, E, H, and L isolated following controlled cooling, then air dried for 5 minutes revealed the stoichiometry of the salts to be 1:1 and also revealed a salt to solvent ratio ranging from 1:0.0155 to 1:0.027.

5-MeO-DMT benzoate lot 21-01-073 A was isolated from controlled cooling of a methyl acetate solution from 50° C. to −10° C., then air dried for 5 minutes.

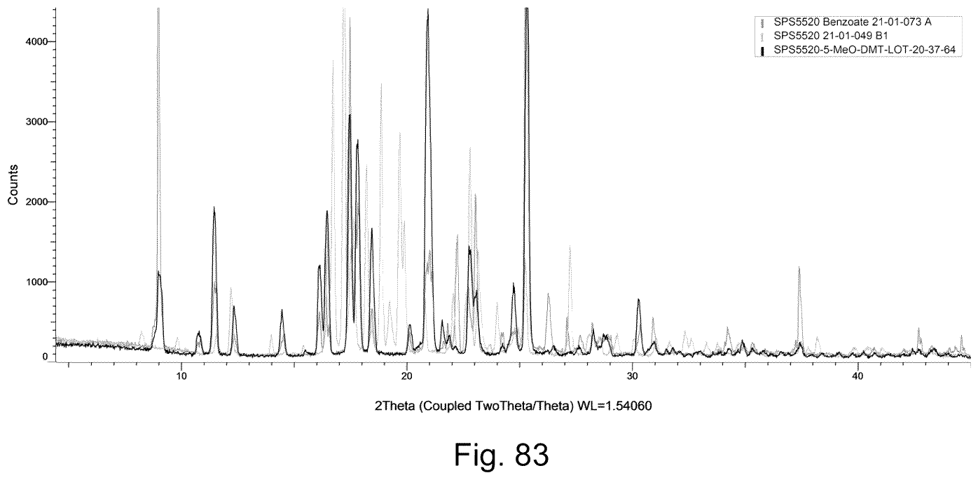

The XRPD of 5-MeO-DMT benzoate lot 21-01-073 A revealed the diffraction pattern was concordant with 5-MeO-DMT benzoate lot 20-37-64, Pattern A (FIG. 83), but featured diffractions at 21 and 24.6°2θ that were more intense. The difference in intensity was likely a result of preferred orientation.

FIG. 83 shows XRPD pattern comparison of 5-MeO-DMT benzoate lot 21-01-073 A, 21-01-049 B1, Pattern B, and 20-37-64, Pattern A.

The DSC thermograph of 5-MeO-DMT benzoate lot 21-01-073 A revealed an endothermic event with a peak temperature of 123.58° C., this is characteristic of Pattern A form.

The 1H NMR spectrum of 5-MeO-DMT benzoate lots 21-01-073 A isolated following controlled cooling, then air dried for 5 minutes revealed the stoichiometry of the salts to be 1:1 and also revealed a salt to solvent ratio of methyl acetate of 1:0.033. 5-MeO-DMT benzoate lot 21-01-073 F was isolated from controlled cooling of a methyl benzoate solution from 50° C. to −10° C., then air dried for 5 minutes. After air drying for 5 minutes the sample was a paste, air drying further for 30 minutes afforded a damp powder.

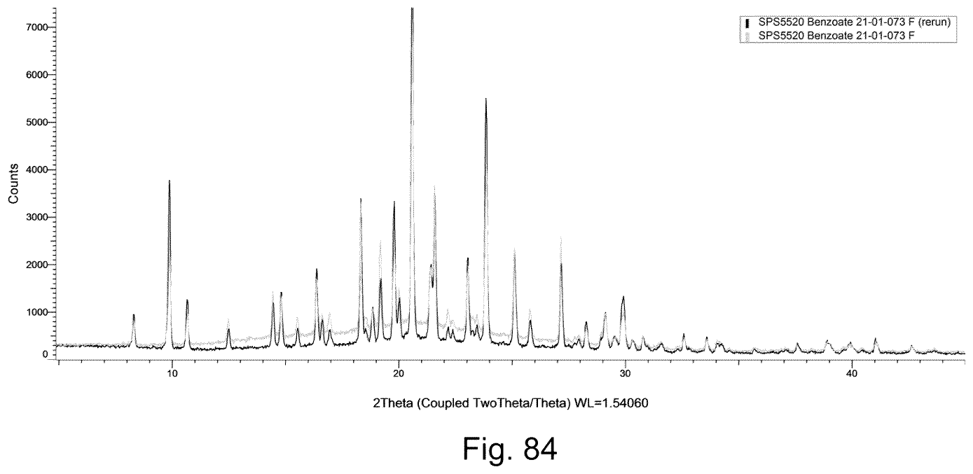

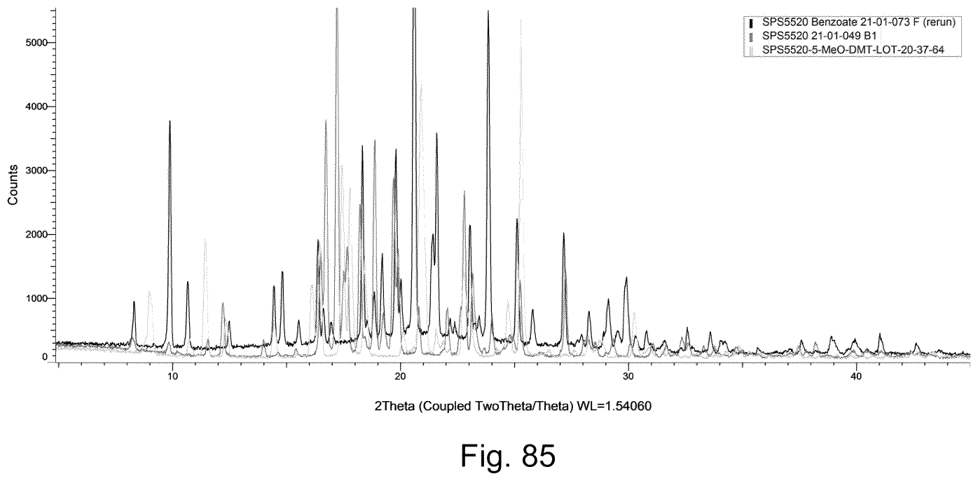

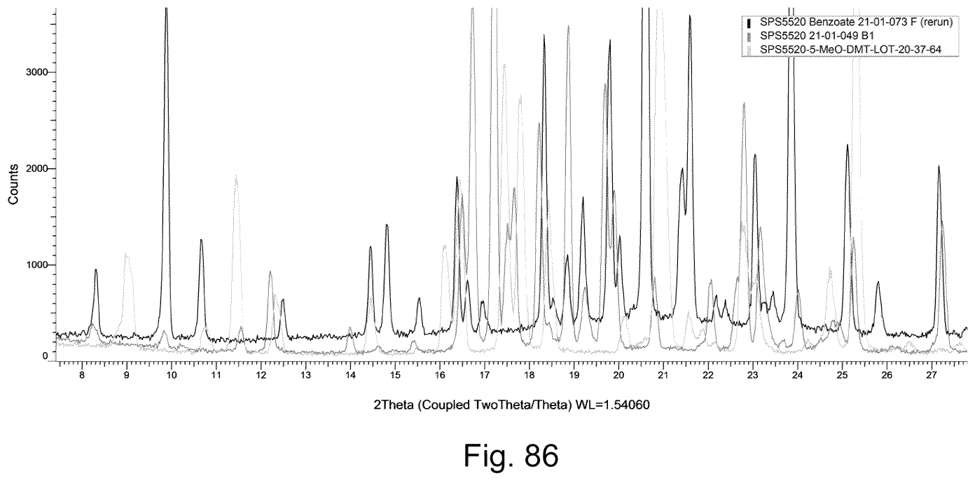

The XRPD of 5-MeO-DMT benzoate lot 21-01-073 F revealed an XRPD pattern with an amorphous halo (FIG. 84). The sample was re-run after further air drying. The XRPD of 5-MeO-DMT benzoate 21-01-073 F (re-run) revealed a diffraction pattern concordant with the initial measurement but with a reduced amorphous halo (FIG. 85). The diffraction pattern demonstrated some similarities with both Pattern A and B (FIG. 86) but the presence of unique diffractions and absence of characteristic Pattern A and Pattern B diffractions indicate this material to be a unique solid form version, identified herein as Pattern F form.

FIG. 84 shows XRPD pattern comparison of 5-MeO-DMT benzoate lot 21-01-073 F and 21-01-073 F rerun.

FIG. 85 shows XRPD pattern comparison of 5-MeO-DMT benzoate lot 21-01-073 F rerun, 21-01-049 B1, Pattern B, and 20-37-64, Pattern A.

FIG. 86 shows XRPD pattern expansion comparison of 5-MeO-DMT benzoate lot 21-01-073 F rerun, 21-01-049 B1, Pattern B, and 20-37-64, Pattern A.

The DSC thermograph of 5-MeO-DMT benzoate lot 21-01-073 F (re-run) revealed a broad endothermic event with a peak temperature of 90.50° C., this was followed by a small endothermic event with a peak temperature of 106.65° C. This was followed by a broad and shallow endothermic event with a peak temperature of 180.35° C.

DSC examination was repeated after the sample was stored in a sealed container for 24 hours. The DSC thermograph revealed a major endothermic event with a peak temperature of 95.33° C., followed by an exothermic event with a peak temperature of 102.70° C. This was followed by an endothermic event with a peak temperature of 113.77° C.

The 1H NMR spectrum of 5-MeO-DMT benzoate lots 21-01-073 F isolated following controlled cooling, then air dried for 5 minutes, revealed the stoichiometry of the salts to be 1:1 and also revealed a salt to solvent ratio of 1:0.59. After air drying, the paste-like consistency indicated the presence of methyl benzoate, the visually damp powder following 30 minutes of air drying, indicates that residual methyl benzoate was still present. However, due to the unique diffraction pattern and DSC thermograph, combined with the stoichiometry close to 1:0.5 and the propensity of the 5-MeO-DMT benzoate salt to form hemi-solvates with aromatic solvents, this sample is believed to be a methyl benzoate hemi-solvate.

5-MeO-DMT benzoate lot 21-01-073 I was isolated from controlled cooling of a 5-MeO-DMT benzoate cumene solution from 50° C. to −10° C., then air dried for 5 minutes.

The XRPD of 5-MeO-DMT benzoate lot 21-01-073 I revealed the diffraction pattern was concordant with SPS5520 21-01-049 B1, Pattern B.

The DSC thermograph of 5-MeO-DMT benzoate lot 21-01-073 I revealed an endothermic event with a peak temperature of 109.24° C. with a broad shoulder at ca. 100° C. This was followed by an exothermic event with a peak temperature of 111.35° C., then an endothermic event with a peak temperature of 120.31° C. This was followed by a broad exothermic event with a peak temperature of 146.19° C. This thermal profile resemble historic Pattern B samples, although the post-final melt exotherm was known.

The 1H NMR spectrum of 5-MeO-DMT benzoate lots 21-01-073 I isolated following controlled cooling, then air dried for 5 minutes revealed the stoichiometry of the salts to be 1:1 and also revealed a salt to solvent ratio of 1:0.035.

5-MeO-DMT benzoate lot 21-01-073 J was isolated from controlled cooling of an 5-MeO-DMT benzoate toluene solution from 50° C. to −10° C., then air dried for 5 minutes.

The XRPD of 5-MeO-DMT benzoate lot 21-01-073 J revealed the diffraction pattern was concordant with 5-MeO-DMT benzoate lot 21-01-064 A, Pattern C.

The DSC thermograph of 5-MeO-DMT benzoate lot 21-01-073 J revealed an endothermic event with peak temperatures of 110.00° C., 115.03° C., and 120.60° C. The DSC thermograph is similar to 5-MeO-DMT benzoate lot 21−01-071 C1, previously isolated Pattern C form material, although the minor peaks are different which is believed to be a consequence of sample preparation.

The 1H NMR spectrum of 5-MeO-DMT benzoate lots 21-01-073 J isolated following controlled cooling, then air dried for 5 minutes revealed the stoichiometry of the salts to be 1:1 and also revealed a salt to solvent ratio of 1:0.473, confirming the isolation of the Pattern C form toluene hemi-solvate.

5-MeO-DMT benzoate lot 21-01-073 K was isolated from controlled cooling of an 5-MeO-DMT benzoate 2-chlorotoluene solution from 50° C. to −10° C., then air dried for 5 minutes.

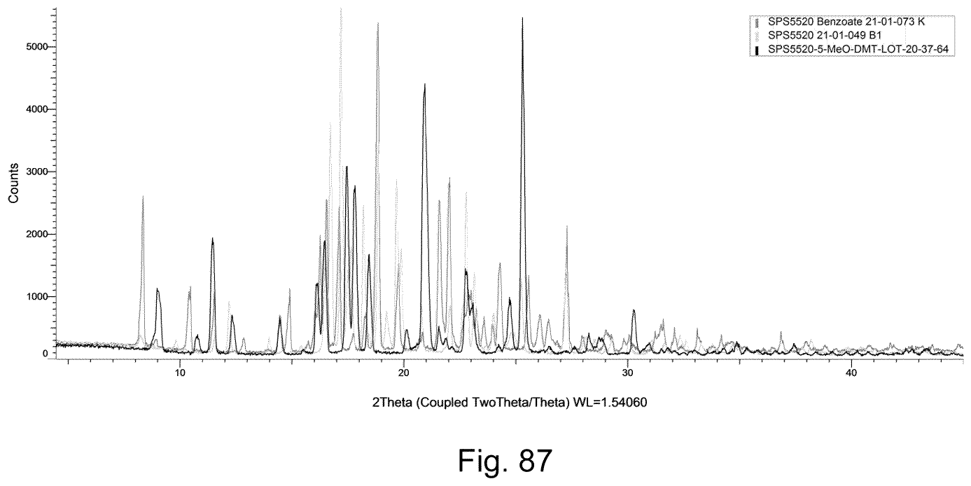

The XRPD of 5-MeO-DMT benzoate lot 21-01-073 K revealed a diffraction pattern that was unique (FIG. 87) and is herein identified as Pattern G.

FIG. 87 shows XRPD pattern comparison of 5-MeO-DMT benzoate lot 21-01-073 K, 21-01-049 B1, Pattern B, and 20-37-64.

The DSC thermograph of 5-MeO-DMT benzoate lot 21-01-073 K revealed an endothermic event with peak temperatures of 111.28° C. and 119.61° C.

The 1H NMR spectrum of 5-MeO-DMT benzoate lots 21-01-073 K isolated following controlled cooling, then air dried for 5 minutes revealed the stoichiometry of the salts to be 1:1 and also revealed a salt to solvent ratio of 1:0.516, thus Pattern G form is believed to correspond to a 2-Chlorotoluene hemi-solvate.

The Table below is a summary of samples isolated from this controlled cooling experiment and the XRPD patterns afforded.

| | | XRPD | | Composition |
|---|---|---|---|---|
| Sample | Solvent | pattern | DSC | by ¹H NMR |
| A | Methyl acetate | A | N/C | 1:0.033 solvent |
| B | n-Propyl acetate | A | A | 1:0.027 solvent |
| C | Iso-Propyl acetate | A | N/C | N/C |
| D | Iso-Butyl acetate | A | N/C | N/C |
| E | Ethyl formate | A | A | 1:0.016 solvent |
| F | Methyl benzoate | F | 95.33° C. | 1:0.59 solvent |
| G | Methyl propionate | A | N/C | N/C |
| H | 4-Methyl-2-pentanone | A | A | 1:0.016 solvent |
| I | Cumene | B | 109.24° C. + | 1:0.035 solvent |
| | | | 120.31° C. | |
| J | Toluene | C | 120.60° C. | 1:0.473 solvent |
| K | 2-Chlorotoluene | G | 119.61° C. | 1:0.516 solvent |
| L | α,α,α- | A | A | Obscured |
| | Trifluorotoluene | | | |

### Example 31: DVS Examination of Amorphous 5-MeO-DMT Benzoate Produced Via Lyophilisation

5-MeO-DMT benzoate 20/20/150FP2, 150 mg, was dissolved in deionised (DI) water, 5 ml affording a clear solution.

The solution was clarified into a 500 ml round bottom flask, the round bottom flask was rotated in an acetone/dry ice bath to freeze the solution in a thin layer around the flask. The ice was sublimed in vacuo at ambient temperature affording a fluffy white solid. The solid was removed from the round bottom flask and transferred to the DVS instrument. During this transfer, the solid collapsed to a sticky gum.

The sample was examined by DVS from 40% RH and cycled between 0% RH and 90% RH twice.

XRPD was collected on a portion of the sample post-lyophoilisation and post-DVS examination.

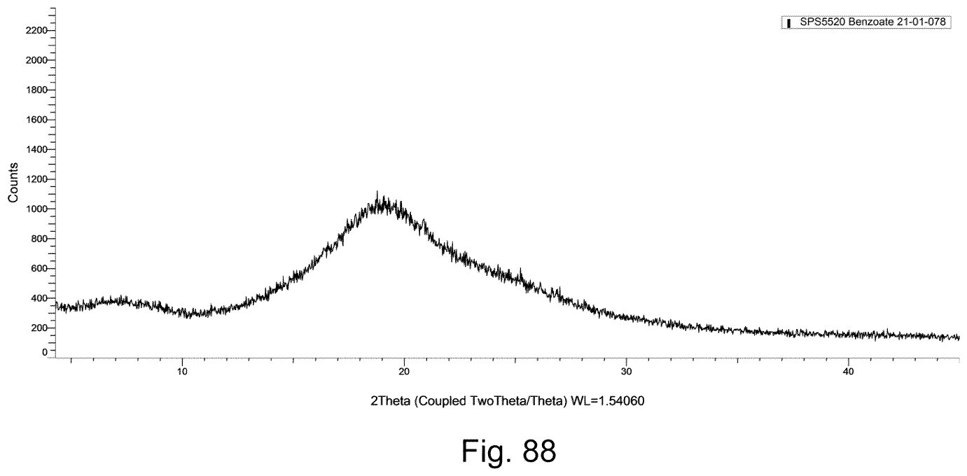

The XRPD of 5-MeO-DMT benzoate before DVS analysis revealed an amorphous diffraction pattern which was expected (FIG. 88). FIG. 88 shows XRPD of 5-MeO-DMT benzoate lot 21-01-078.

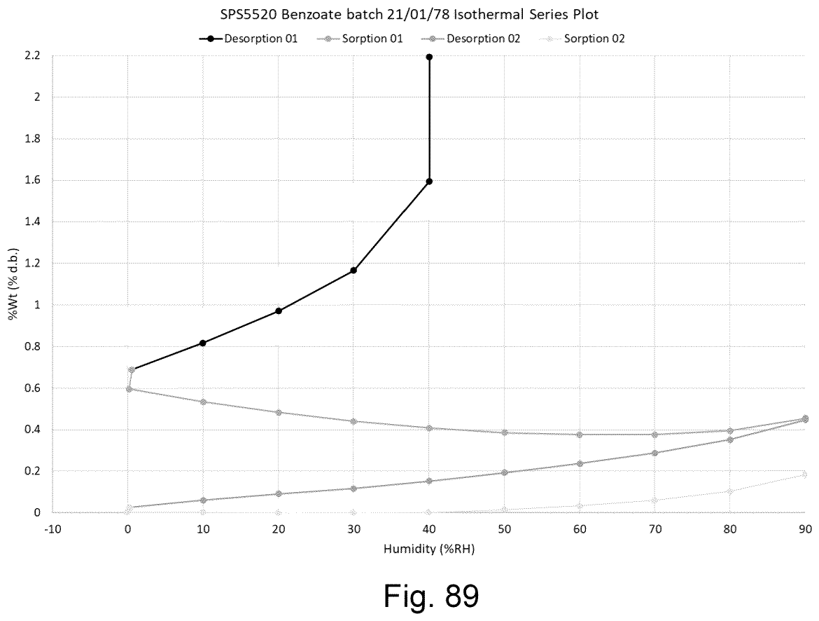

The DVS examination demonstrates an initial weight reduction of ca. 1.4% from the start of the investigation during the first desorption cycle (FIG. 89) which was much lower than the 5 wt % required for a 5-MeO-DMT benzoate monohydrate. Weight reduction continues despite the RH increasing to 70% RH during the first sorption. At 80 and 90% RH on the first sorption cycle, there is a small increase in weight. Following this there is a weight reduction to the minimum on the second desorption cycle, on the subsequent sorption cycle there is no change in weight until 50% RH, between 50% RH and 90% RH there is a weight increase of 0.2%.

FIG. 89 shows DVS isothermal plot of 5-MeO-DMT benzoate lot 21-01-078.

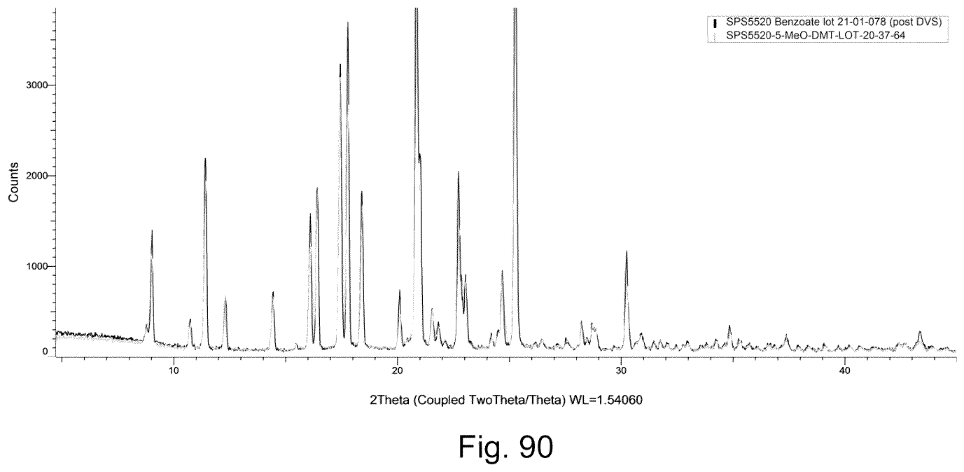

The XRPD of 5-MeO-DMT benzoate lot 21-01-078 after DVS examination at 90% RH revealed a diffraction pattern concordant with Pattern A (FIG. 90).

FIG. 90 shows XRPD pattern comparison of 5-MeO-DMT benzoate lot 21-01-078 (post-DVS) and 20-37-64.

Amorphous 5-MeO-DMT benzoate is unstable and undergoes transformation to Pattern A form under all conditions studied. Under ambient conditions it is believed that the amorphous version uptakes moisture from the atmosphere which is eliminated from the sample following conversion to Pattern A form. Such a conversion is not considered to be via a hydrate as there has been no observed evidence of a 5-MeO-DMT benzoate hydrate. Alternatively, the process of lyophilisation could seem complete when in fact some moisture remains bound to the solid. Upon evacuation of the lyophilisation vessel to atmospheric pressure, the low density, voluminous solid contracts, entrapping the moisture to afford the gum that is then ejected as the amorphous gum and converts to the more stable, ordered Pattern A form version.

### Example 32: FTIR Spectroscopy of 5-MeO-DMT Benzoate Patterns a, B and C

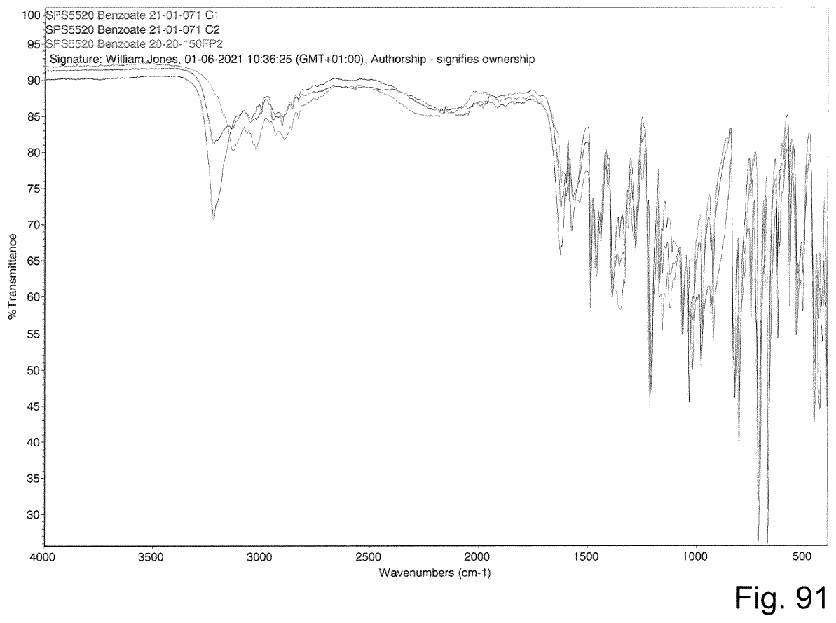

FIG. 91 shows FTIR overlay of 5-MeO-DMT benzoate Pattern A form (20-20-150FP2), Pattern B form (21-01-071 C2) and Pattern C form (21-010071 C1).

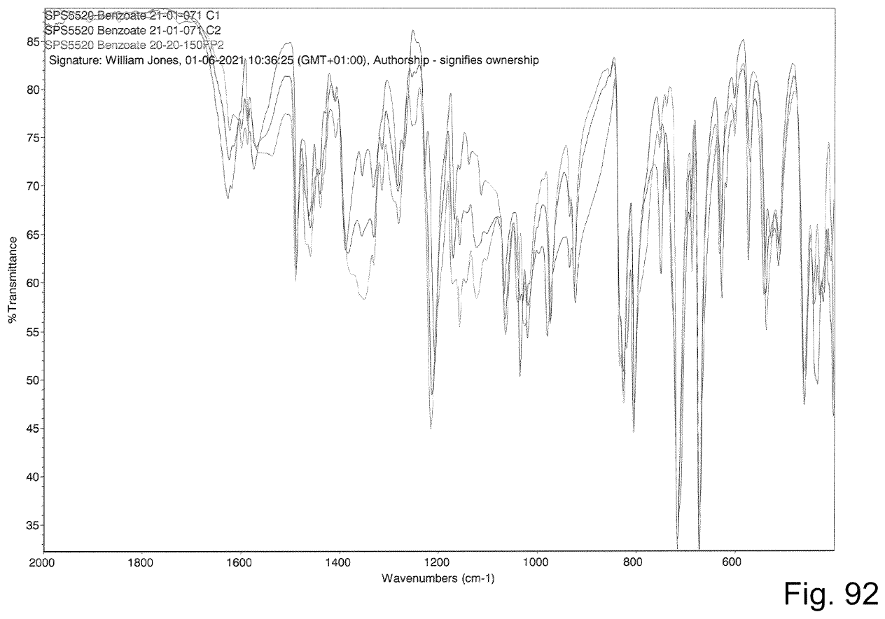

FIG. 92 shows FTIR overlay of 5-MeO-DMT benzoate Pattern A form (20-20-150FP2), Pattern B form (21-01-071 C2) and Pattern C form (21-010071 C1) at 450 to 2000 cm−1.

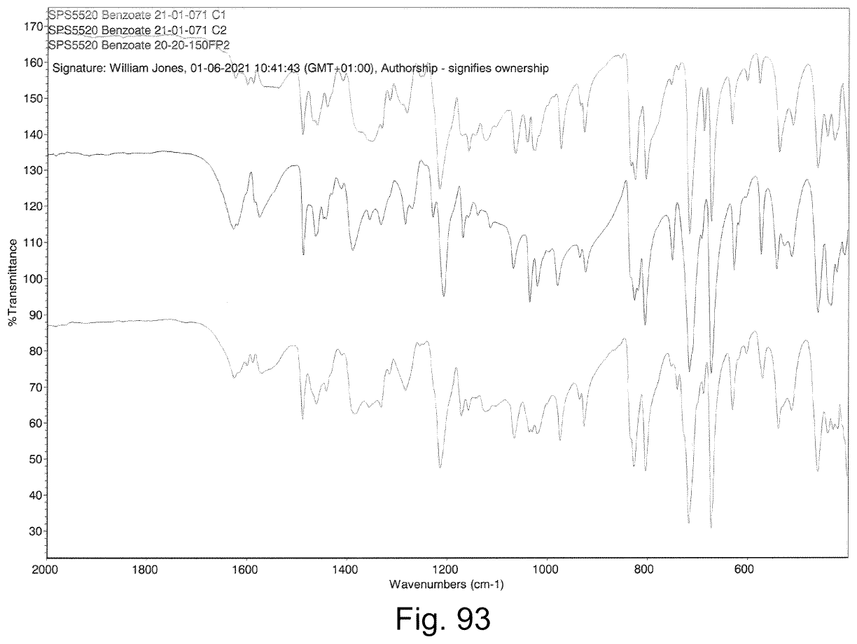

FIG. 93 shows FTIR overlay of 5-MeO-DMT benzoate Pattern A form (20-20-150FP2), Pattern B form (21-01-071 C2) and Pattern C form (21-010071 C1) at 450 to 2000 cm−1; spectra separated.

Inspection of FTIRs reveals the Pattern A form demonstrates a number of bands of significantly different intensity compared to Patterns B form and C form. Such notable bands were observed at ca. 3130, 1540, 1460, 1160 and 690 cm−1, whilst key absent (or significantly reduced intensity) bands present in Patterns B and C included those observed at ca. 3230 and 1640 cm−1.

Patterns B and C forms demonstrated far fewer differences in their FTIRs to one another, as when compared to the FTIR of the Pattern A form.

This was anticipated when it is considered that the Pattern C form hemi-solvate desolvates somewhat readily to afford the Pattern B form, resulting in a relatively small change to the crystal lattice compared to the energy required (i.e.; drying in vacuo at elevated temperature) to induce conversion of Pattern B form to Pattern A form, restructuring the crystal lattice to a greater extent than facile desolvation.

### Example 33: Stability of Patterns B and C

Drying 5-MeO-DMT benzoate Pattern C form in vacuo at 50° C. for 24 hours historically often afforded Pattern B form and Pattern B form is known to transform to Pattern A form at 90° C. as observed by hot stage microscopy. The stability of Pattern A form and Pattern B form under both atmospheric conditions and in vacuo at 50° C. was investigated to determine the relationship between the forms.

5-MeO-DMT benzoate lot 21-01-071 C1, Pattern C form, and lot 21-01-071 C2, Pattern B form, were charged to XRPD sample holders and sample vials and left open to the atmosphere for 12 days.

5-MeO-DMT benzoate lot 21-01-071 C1, Pattern C form, was dried in vacuo at 50° C. for 5 days.

XRPD was performed regularly. DSC and 1H NMR spectroscopy were performed on samples where significant differences to the diffraction patterns were observed.

The Table below shows a summary of solid form conversion by XRPD during the stability tests.

| | Drying | XRPD pattern throughout drying | | | | | | | | |
|---|---|---|---|---|---|---|---|---|---|---|
| Sample | method | Day 0 | Day 1 | Day 2 | Day 3 | Day 4 | Day 5 | Day 6 | Day 8 | Day 12 |
| 21-01- | Open to | C | C + B | n/c | n/c | C + B | n/c | C + B | C + B | C + B |
| 071 C1, | atmosphere at | | | | | | | | | |
| Pattern C | 20 ± 2° C. | | | | | | | | | |
| 21-01- | Open to | B | B | n/c | n/c | B | n/c | B | B | B |
| 071 C2, | atmosphere at | | | | | | | | | |
| Pattern B | 20 ± 2° C. | | | | | | | | | |
| 21-01- | In vacuo at | C | B | B + A | A + B | n/c | A | n/c | n/c | n/c |
| 071 C1, | 50° C. | | | | | | | | | |
| Pattern C | | | | | | | | | | |

### Example 34: Competitive Equilibration of 5-MeO-DMT Benzoate Pattern a, B, and C Forms in Solvents

The relationship between 5-MeO-DMT benzoate Pattern A, B, and C forms was investigated to determine the thermodynamically stable version and hierarchy. Competitive equilibration was conducted between Pattern A and B forms, and Pattern A and C forms in a variety of solvents including IPA and toluene. Pattern A form was expected to be the most stable form given its melting point of 124° C. and prevalence during most investigations performed.

5-MeO-DMT benzoate 20/20/150FP2, Pattern A form, 15 mg, was charged to all crystallisation tubes. 5-MeO-DMT benzoate lot 21-01-071 C2, Pattern B form, 30 mg, was charged to AB crystallisation tubes. 5-MeO-DMT benzoate toluene hemi-solvate lot 21-01-071 C1, Pattern C form, 30 mg, was charged to AC crystallisation tubes. Solvent, 0.5 ml, was charged to crystallisation tubes as detailed in the Table below. Suspensions were agitated at 100 rpm at 2θ±2° C. for 24 hours. Suspensions were isolated via isolute cartridge and air dried for 5 minutes and characterised by XRPD and DSC.

| Solid | | | Summary of solid form characterisation | |
|---|---|---|---|---|
| mixture | Solvent | ID | XRPD | DSC |
| Pattern A | IPA | AB1 | Pattern A | Endotherm at 124° C. |
| (15 mg) + | Toluene | AB2 | Pattern C | Endotherm at 122° C. |
| Pattern B | iPrOAc | AB3 | Pattern A | Endotherm at ca. |
| (30 mg) | | | | 124° C. + minor events |
| | MeCN | AB4 | Pattern A | Endotherm at 124° C. |
| | MEK | AB5 | Pattern A | Endotherm at 124° C. |
| | 2-MeTHF | AB6 | Pattern A | Endotherm at 124° C. |
| Pattern A | IPA | AC1 | Pattern A | Endotherm at 124° C. |
| (15 mg) + | Toluene | AC2 | Pattern C | Endotherm at 123° C. + |
| Pattern B | | | | minor events |
| (30 mg) | iPrOAc | AC3 | Pattern A | Endotherm at 124° C. |
| | MeCN | AC4 | Pattern A | Endotherm at 124° C. |
| | MEK | AC5 | Defined | Endotherm at 124° C. |
| | | | Pattern A | |
| | 2-MeTHF | AC6 | Pattern A | Endotherm at 124° C. |

The XRPD of all samples revealed the majority gave Pattern A.

Sample AC5 isolated from MEK revealed an additional diffraction at 8.8°2θ however this was considered to be caused by the splitting of the diffraction at 9°2θ due to better resolution between diffractions of this sample.

The DSC thermograph of most Pattern A form samples revealed an endothermic event with peak temperatures ranging from 123.74° C. to 124.22° C. which is indicative of Pattern A form.

The DSC thermograph of 5-MeO-DMT benzoate lot 21-01-079 AB3, isolated from isopropyl acetate, revealed a series of events between 109° C. and 115° C., then a minor endothermic event with a peak temperature of 115.69° C. This was followed by a major endothermic event with a peak temperature of 123.85° C. indicative of the Pattern A form.

The minor endothermic events are believed to be due to the incomplete conversion of Pattern B form to Pattern A form via equilibration.

The XRPD of 5-MeO-DMT benzoate lot 21-01-079 AB2 and AC2, both equilibrated in toluene, revealed a diffraction pattern concordant with 5-MeO-DMT benzoate lot 21-01-064 A toluene hemi-solvate, Pattern C form.

The DSC thermograph of 5-MeO-DMT benzoate lot 21-01-079 AB2 revealed a bimodal endothermic event with peak temperatures of 114.96° C. and 121.92° C. The thermal characteristics are similar to previously isolated pattern C samples, including 5-MeO-DMT benzoate lot 21-01-073 J.

The DSC thermograph of 5-MeO-DMT benzoate lot 21-01-079 AC2 revealed a minor endothermic event with a peak temperature of 110.11° C., followed by overlapping endothermic and exothermic events between 110.73° C. and 113.23° C. This was followed by an endothermic event with a peak temperature of 122.82° C., this endothermic event is comparable to the melt of Pattern A form when recrystallised from Pattern B form.

Competitive equilibration of both Pattern A/B form mixtures and Pattern A/C form mixtures in solvents that were not previously observed to produce hemi-solvates demonstrated conversion to the Pattern A form. It is anticipated that all other hemi-solvates will convert to the Pattern A form in these solvents.

Competitive equilibration of both Pattern A/B forms and Pattern A/C forms in toluene demonstrated conversion to the Pattern C form. It is anticipated that equilibration of 5-MeO-DMT benzoate in a solvent (typically an aromatic solvent) that has the propensity to form a hemi-solvate will afford that particular 5-MeO-DMT benzoate hemi-solvate over the otherwise thermodynamically stable Pattern A form solid form version.

### Example 35: Administration of a 5-MeO-DMT Salt

The physical surroundings of the participant/patient/subject are of high importance in the character of many psychedelic experiences. The space should be private, meaning that there should be no chance of intrusion by others. Ideally, sound from outside (e.g. the hallway, the street, etc.) will be minimal. The dosing sessions should take place in rooms that feel like a living room or den rather than a clinical setting. Artwork, plants, flowers, soft furniture, soft lighting, and related décor should be employed in creating a cozy and relaxing aesthetic. Artwork with any specific religious iconography, ideological connotation, or tendency to evoke negative emotions should be avoided. The dosing room may also provide comfortable furniture for the participant and the therapists, who may sit on either side of the participant. Participants under the effect of 5-MeO-DMT may exhibit spontaneous movement or slide off of the bed or couch in their prone position. It is therefore important to make sure no sharp or hard objects are nearby that the participant may fall on. Additionally, pillows may be useful to physically support participants who are mobile during the experience. A therapist can provide physical support to the participant by placing a pillow between their hands and the participant's body.

Music may accompany the experience, so the dosing room should be equipped with a stereo. The room should shield the participant from sights and sounds of the world beyond the room, and the participant should not have any cause for concern of observation or interruption by anyone other than the therapists.

The space may also contain:
The tools for safety procedures and medical devices necessary to respond in the unlikely event of a medical complication. The participant should be made aware of these procedures and the equipment, but as much as possible they should be hidden from view.
A secured and locked space for study materials and documentation in the session room or nearby.
An approved safe for storing the 5-MeO-DMT in the session room or nearby.
Audio and video-recording equipment: If allowed in the study protocol the participant will have already consented to being recorded, and should be made aware of the equipment, but it should be placed to be as unobtrusive as possible. Participants may request the cessation of recording at any time.

### Physical Space

The space may be large enough to accommodate chairs for two therapists, the stereo equipment and cabinet for storage of the participant's belongings and any extra supplies the therapists may need during the day. The space may accommodate a bed or couch on which the participant can either sit up or lie down with a comfortable surroundings of pillows. The space may be at least 100² feet or 10² meters so that participants do not feel cramped or too physically close to therapists. Participants should have room to explore a variety of positions including sitting on the floor or stretch their bodies without restriction. A bathroom should be either accessible directly from the session room or nearby.

### Music

5-MeO-DMT sessions may use a pre-set playlist of nature sounds for creating a calm atmosphere. These nature sounds are considered to be a background element, helping drown out any noise from outside the room, and keep the participant focused on their experience. Participants are not instructed to listen to the sounds in any particular way, but may be asked to focus on it as a way of grounding their senses and relaxing before or after session.

### Medication Discontinuation

Medication discontinuation can be challenging for participants. Participants are to have discontinued all contraindicated medications and completed washout periods prior to Prep-1 with the therapist. The study team members, including the therapist, may provide supportive check-in calls with the participant prior to this, as-needed during the washout period, but should not start Prep-1 until washout is complete and the participant confirms intention to continue with the therapy.

### Preparatory Sessions

This treatment model includes three, 60-90 minute preparatory sessions with the therapist. These take place 7 days, 4 days, and 1 day before the 5-MeO-DMT session. Preparatory sessions are designed to take place via telemedicine, but can be in-person if possible.

### Preparatory Session 1

The following topics may be covered in the first preparatory session.

### Getting to Know the Participant

The therapist will spend some of the preparation session time getting to know the participant. The therapist may ask open-ended questions about:
How they found out about the treatment and what their expectations are;
Current life situation with regards to living situation, work, school, and important relationships;
Understanding of their own depression;
Key life events that the participant feels might be of relevance

The therapist should be listening for how the participant talks about themselves and their relationship to their depression, how they relate to the therapist and study environment, and stay attuned to establishing a sense of trust and rapport with the participant. Clinical impressions of difficulty forming a trusting relationship with the therapist or any other clinical factors that could interfere with the participants' ability to engage in the treatment should be noted and discussed with the study team. Although in the preparatory session stage, the therapist may learn more of the participant that could be reasons for study exclusion.

### Establishing the Role of the Therapist

Therapists in the 5-MeO-DMT-assisted therapy treatment model form a relationship with study participants which becomes part of the container in which the 5MDE (subjective experience of 5-MeO-DMT) takes place. This formation of this relationship is deliberate on the therapists part and characterized by the therapist establishing transparency and trust, taking clinical responsibility for the patient's wellbeing, and relational and emotional safety for the patient. The therapeutic relationship is understood as a critical component of the set and setting for the therapeutic use of the 5MDE. The communication and establishment of this relationship is both explicit (overt) and implicit (covert) in the therapists behaviors and mannerisms throughout the treatment.

Explaining the Therapeutic Model with Participant as Active Participant in their Process

The therapist should explain the therapeutic model used in this research study to the participant in the first preparation session. The explanation should include:
Practical aspects:
   How many meetings with the therapist will occur, and for how long.
   That the therapy is thought to work by:
   Creating a safe container for the experience so that the participant knows what to expect and can fully let go into their experience,
   Helping the participant focus on and explore their own responses to the experience,
   Facilitating a process of the participant determining for themselves how they will put their insights into practice in their life.
That the therapists role is:
   Supporting the participant through the session, engaging in a series of activities to elicit the participant's unique experience and insights, fostering the participant's process of implementing the resulting changes in their life.
   That the therapy is:
   Not a full deep dive into participant's personal history, not a place to do specific problem solving or engage in CBT, Psychodynamic interpretations, get general advice, or receive other interventions the participant may be familiar with.

### Establishing Physical, Emotional, and Psychological/Relational Safety

Beginning in the first preparatory session the therapist establishes the environment of physical, emotional, and psychological safety. The therapist explains the safety of 5-MeO-DMT and the safety procedures relevant to the participants physical health for the session. With regards to emotional safety the therapist states that all emotional experiences are welcomed, that there is no area of experience that the participant is not welcome to share. Safety can also be established through the calm reassuring presence of the therapist, which does not always require the use of language.

The use of self-disclosure is not prohibited, but should be used very sparingly. A participant may be seeking safety by asking personal questions of the therapist. If the therapist chooses to disclose, it should be brief and under the condition the participant share why this personal information is important to them.

Psychological/relational safety is established by assuring the participant that their wishes will be respected with regards to the use of touch. Also, the participant is to be reassured that if they choose not to participate in the 5MDE experience they may do so at any point up until drug administration and that this will be respected, and that the therapy sessions will still be available to them if they make that choice.

The therapist can use the following techniques to establish safety with the participant:
Ask open-ended questions that invite the expression of doubts, hesitancies, or concerns:
   What questions do you have for me?
   What more would you like to know about 5-MeO-DMT?
   What would you find helpful in the event . . . ?
   How could I be of assistance to you if you feel . . . ?
Encourage and engage with the full range of participant's emotions and experiences without trying to fix or resolve them:
   Participant expresses skepticism about the 5-MeO-DMT Experience: I appreciate you sharing that doubt with me. What do you make of that in light of your presence here at this time?
   Participant expresses fear about the 5MDE Experience: What more can you tell me about your fear and how it manifests for you?How could I be helpful to you as you experience this?
   Use affirmations to establish an environment of valuing the participant's time and effort:
   I really appreciate the time you are putting into this treatment and your willingness to participate in research.
   Your experience is unique to you and I appreciate the opportunity to see you through this process.

### Expected Potential Subjective Drug Effects (Unity, “Feeling Like Dying”, “the Void”,)

It may be helpful to discuss the concept of “non-ordinary state of consciousness” with participants. In the past, “altered state of consciousness” was often associated with experienced engendered by psychedelic compounds. However, alterations of consciousness are experienced on a daily basis, as moods or feelings shift, or when people shift from awake alertness to feeling tired and drowsy. “Non-ordinary state of consciousness” emphasizes the quality of an experience that is not ordinarily had on a daily occurrence, but can still be within human experience.

The therapist may begin this conversation by asking the participant about their existing knowledge of 5-MeO-DMT effects, and listen for specific expectation or ideas about it. The therapist is to encourage an attitude of openness toward the experience, encouraging participants to explore what kinds/ideas they may have and be open to the possibility that it will not be possible to imagine what this will be like. Participants may have specific expectations based on the media, prior experience with 5-MeO-DMT or other psychedelics, or other kinds of non-ordinary states of consciousness. It is important for therapists to provide a balanced description of what the participant may experience.

Different people have different levels of comfort with “not knowing” what something will be like, or what to expect. The therapist may explore the participant's level of comfort with the unknown, their relationship to the idea the future not being fully knowable in any situation, and how they generally relate to this. Among participants with depression there may be deep fear of the unknown, anticipation of what is expected in the future (more negative experiences), resulting in a feedback loop of feeling fearful and depressed. Therapists should elicit and explore this area during preparation.

Common 5-MeO-DMT Experiences: The therapist should also introduce a few key terms and commonly reported experiences known to occur under 5-MeO-DMT. These include a feeling of unity, a feeling of dying, and a feeling of entering or experiencing a “void” (absence of material reality). Some participants may have an existing spiritual, philosophical, or religious belief system through which they will interpret or make meaning of these experiences. Therapists should enquire about this and work with the participant's own explanation and terms, without taking a stance as to whether these are correct or erroneous.

### Social Support and Social Media

Participant's social support may be assessed during preparation sessions and be determined by the therapist to be adequate to support the patient through the process of change, especially in the event of either disappointment or dramatic symptom reduction. In the event the participant has a psychotherapist outside of the study the study therapist may, with the participant's permission, have a phone call with the participants therapist to describe the nature of the study and therapeutic approach and answer any questions the therapist may have. The study therapist may also educate any friends or family members who are close to the participant and have questions regarding the nature of the study, the 5-MeO-DMT experience, and what to expect. The therapist should discuss social support with the participant including preparing the participant for the variety of reactions their friends and family may have.

Therapists may advise participants to take caution around posting about their experience on social media so as not to elicit excessive public commentary. Inadequate social support or use of social media in a way that may be disruptive to the therapeutic process may be discussed and resolved prior to 5-MeO-DMT administration.

### Preparatory Session 2

The following topics may be covered in the second preparatory session.

Drug experience preparation: trust, surrender (let go), embrace, transcendence.

There are several key attitudes towards psychedelic experiences that are considered to be conducive to a positive and clinically helpful experience. The more participants can embody a relaxed stance toward their experience the less likely they are to struggle, inadvertently creating a loop of stress and distress that heightens attention to negative aspects and interpretations. The therapist may educate the participant on the purpose of deliberately generating an attitude of trust, surrendering to the experience, and letting go of attempts to control the experience. Therapists may encourage participants to develop an attitude of welcoming and embracing all experiences they may have as part of their 5-MeO-DMT experience. The therapist may suggest to a participant that all aspects of the experience (feelings, sensations, and thoughts) can be welcomed. Previous research with psychedelics has demonstrated that a capacity to be absorbed by the experience can contribute to the potency of a mystical experience.

### The Drug Administration

The therapist should explain that on the day of the session that a member of the research team will enter the room briefly to administer the study drug. The therapist should explain the participant positioning, e.g. they will be in a seated position on the bed or couch, that the research team member will insert the nasal spray device in one nostril, and that they will be asked to allow the therapist to assist them in lying down on the bed or couch immediately afterward.

### Session Procedures Including Boundaries, Use of Touch, Safety, Etc.

The therapist will explain the process of the session. The session is contained by the timing of the dosing and the physical environment of the dosing room. It begins when the participant enters the room and engages with the therapist in the Session Opening. Session Opening is a formal moment in which the participant and therapist sit together in the room, all preparations having been made, and playlist started. The therapist may lead a breathing exercise of the participant's choice, if the participant is open to engaging in one, and ask the participant to reflect on the values they choose in the preparation session, or any other value or intention that is important to them. Once the participant signals that they are ready, a member of the research team will administer the nasal spray to the participant. Trust and safety are not only communicated verbally, but also this may be nonverbally through how a therapist holds themselves in the presence of the participant. If a therapist is overly anxious, or fearful, this may be felt by the participant. It is important that the therapist is centered throughout the dosing session, particularly at times when a participant is expressing intense affect, unusual somatic expressions, or is asking for support.

Somatic Changes and Shifts in One's Sense of their Body

Some participants may experience an intensified awareness of their body such as feeling their heart rate more strongly or physical sensations in their temple. Other participants may be aware of a tingling in their body, changes or perceived difficulty breathing, or other unusual physiological experiences. It is important for the therapist to communicate that these changes in perception are normal and should not be a focus of preoccupation or fear. If these sensations arise, the participant should be encouraged to communicate these to the therapist, if they so desire. The therapist should reassure the participant that these sensations are expected and are normal to have. The therapist can inform and remind the participant that naturally occurring 5-MeO-DMT has been consumed in other settings for hundreds of years with no indication that it is physically harmful, and that these changes are expected and will resolve shortly.

### Discussing Expectations and Intentions

Expectations can be defined as mental representations and beliefs of how something in the future will be. Sometimes expectations can be explicitly identified, and sometimes they are subperceptual, taken for granted. Both kinds of expectations may be important to treatment. The therapist should ask about explicit expectations and encourage the participant to acknowledge and set these aside such that they do not engage in comparing their experience to expectations. The therapist is also listening for subperceptual expectations that may come into awareness through the therapy. Intentions are ways of relating to a behavior or experience. In the 5-MeO-DMT treatment, it can be important for the therapist to elicit and understand the participant's intentions as these can vary greatly and may be taken for granted. Therapists are to engage participants in a process of identifying and setting their intentions such that these are explicit and can be referenced later in integration. The purpose of the intention is for it to be identified and then let go of, with the knowledge that it can be part of the 5MED.

### Recurrence of Acute Effects

Some individuals who used 5-MeO-DMT in non-clinical contexts have reported re-experiencing 5-MeO-DMT's subjective effects in the days after. The dose used, purity, and other factors were not monitored in these cases. The likelihood of these reactivations occurring in a controlled clinical study context is not known, but estimated to be less likely. Nonetheless, it is important for participants to be made aware of this phenomenon. The experience of reactivations are often reported as pleasant, brief (lasting a few moments to minutes), and do not occur with enough frequency to interfere with a person's life. These reactivations are thought by some as part of the integration process. If a participant notices certain activities trigger reactivations, such as certain meditative states, stimulants, or other drugs, and the participant finds these reactivations unpleasant, it should be suggested to the participant that they avoid such triggers. Processing the 5-MeO-DMT experience in therapy, as part of integration, may also be helpful.

### Discussing the Use of Touch

Therapists in this modality may engage in two types of touch: therapeutic touch, and touch for safety reasons. During preparation the therapist should explain and define each. Therapeutic touch is touch that is intended to connect with, sooth, or otherwise communicate with the participant for therapeutic aims. It is always fully consensual, non-sexual, and the participant is encouraged to decline or cease therapeutic touch at any time. Touch for safety reasons can include supporting a participant who is having trouble walking by offering an arm to hold, or blocking a patient back from leaving the room while under acute drug effects. This touch is agreed to in advance, is always non-sexual, and limited to specific safety concerns. Therapists should discuss both of these and establish boundaries with participants ahead of session.

Preparing for after the Session (What to Expect, What to do, Setting Aside Time for Integration)

Participants should be encouraged to take some time to rest and integrate their experience after their session day. Study therapists should ask participants to plan for time off after their session, at least the full day of the session and the day after the session. Therapists should explain that after the acute effects of the 5-MeO-DMT have worn off they will stay together in the room for a while. This period of time will be for the participant to readjust to their experience after the acute effects. They will be asked to share what they can recall about their experience and any reactions they have. They will not be asked to share anything they don't want to share, and are welcome to keep their experience private. They may choose to write or draw about their experience, art supplies and writing supplies will be available. They may be encouraged to spend some time continue to stay with their experience, with the therapist's support, for around an hour. They will then meet with the study team for a safety assessment before going home. Once at home they are encouraged to rest and continue to stay with the experience and the insights, ideas, or new understanding they may have from it. Participants should be reminded that they do not need to share their experience with others unless they want to, and are encouraged to continue to focus on it in whatever way they find most helpful. Participants should refrain from returning to work, from driving, drinking alcohol, drug use, or being a sole caregiver for a child or dependent for the rest of the day.

### Therapist Teaches Breathing Exercise for Dosing Session

When stressed, breaths become shorter and shallower, and when relaxed, the breath becomes longer and slower. Working with the breath is a way of modulating and regulating one's mental state. The therapist may teach and practice two breathing techniques with the participant. These are designed to help the participant relax their body and mind, tolerate stressful or uncomfortable experiences, and develop autonomy through practice on their own. These are not for use during the acute effects of 5-MeO-DMT, but can be used prior to dosing and afterward.

When teaching the practices, the therapist elicits the participant's individual response to each practice to assess suitability of using it. Breathing practices include: Balancing Breath, Diaphragmatic Breath and Counted Breath.

### Preparatory Session 3

Values Card Sort with Prompts

The therapeutic protocol may use a customized Personal Values Card Sort to assist with the therapeutic focus on shift in sense of self. This is done by asking about how people relate to their chosen values before the session, and how they relate to them afterward, drawing attention to shifts, changes, and using these as a guide for the kind of changes the participant may desire to make. It is used as a way to elicit conversation about the participant's sense of self, beliefs about self, and changes in those senses/beliefs throughout the therapy. Therapists may engage participants in the card sort exercise in the third preparation session such that it occurs 1-2 days before the dosing session.

### The Values Card Sort Instructions are:

1. Place five anchor cards in order from 1-5 in front of the participant from left to right in order of least to most important.
2. Shuffle the 100 value cards; keep the 2 blank cards separate.
3. Instruct the participant to sort the cards using the following script: “I placed five title cards in front of you—not important to me, somewhat important to me, important to me, very important to me, most important to me. I'm going to give you a stack of 100 personal value cards. I would like you to look at each card and place it under one of the five title cards. There are also two blank cards. If there is a value you would like to include, write it on the card and put it in whichever pile you would like. I would like you to sort all 100 cards, but whether you use the two additional cards is optional. Do you have any questions?”
4. When the participant is finished sorting, thank them and invite them to look at the “most important” category, removing the other cards from the table.
5. Read the following: “For the second task, I'd like you to focus on the top values you put in the “most important” category and choose the top five.”
6. When the participant has chosen their top five cards, thank them read the following: “For the third task, I'd like you to focus on the top five values you chose and rank them in order from most to least important.”
7. When the participant indicates they are finished ordering, check to make sure you understand how the cards were sorted (ascending or descending). Point to the #1 spot and say, “I want to make sure I have this right—is this your number one value?”
8. Record values on a scoring sheet, journal or by taking a picture of the cards. Participants should keep a record of their card selections as well.

### Debriefing and Discussion:

Next, invite the participant to engage in a structured discussion of each value using a few of the following open-ended prompts, or similar prompts depending on the context of your work:
You selected ______ as your #______ value?;
Please tell me more about what ______ means to you?
What are some ways ______ has been represented in your life?
What are some ways you'd like to see more of ______ in your life?
How does your decision to ______ or not relate to this value?
How much ______ would you like to have in your life?
How would you know if ______ was increasing or decreasing in your life?
How does ______ relate to the change you are trying to make (or considering making)?

Invite the participant to journal about their answers to the same questions with the remaining cards afterwards. In later sessions it can be helpful to check in on the values and revisit these questions, see how answers have changed, and how participants are currently relating to their values.

### Assistant Therapist

The session may be conducted by the therapist with an assistant therapist such that a second person is available to assist in case of any adverse event or physical complication in the participants safety. The assistant who will be present for the session should be introduced in Prep Session 3 and included in a conversation such that they get to know the participant.

### Session-Specific Therapeutic Tasks

Therapists should aim to complete the therapeutic tasks outlined above according to the chart below, while acknowledging that some variation will occur based on individual participant needs.

| | |
|---|---|
| Prep | Getting to know the participant |
| Session 1 | Establishing the role of the therapist |
| | Explaining the therapeutic approach/model with |
| | participant as active participant in their process |
| | Establishing physical, emotional, and |
| | psychological/relational safety. |
| | Expected potential subjective drug effects |
| | (unity, “feeling like dying”, “the void”,) |
| | Social Support and Social Media |
| Prep | Drug specific preparation: trust, surrender |
| Session 2 | (let go), embrace, transcendence, |
| | Drug Administration |
| | Session procedures including boundaries, |
| | use of touch, safety, etc. |
| | Discussing expectations and intentions |
| | Discussing the use of touch |
| | Preparing for after the session (what to expect, |
| | what to do, setting aside time for integration) |
| | Teach and practice Breathing Exercises |
| Prep | Values card sort with prompts |
| Session 3 | Instruction to continue values card sort inquiry |
| | for homework after session if needed. |
| | Confirm plans for session and review any |
| | questions participant has. |
| | Assistant Therapist joins the session |
| | for an introduction if needed |

### 5-MeO-DMT Experience Session

The therapist is present with the participant during the session—including pre-experience and post-experience times. This is the only session that must be conducted in-person. The site and therapist should schedule about 3 hours for the session, including pre-experience and post-experience time. This does not include the time allotted to engage in baseline measures and enrolment confirmation prior to the session. Local regulatory approvals will determine the minimum length of time a participant must be under observation following 5-MeO-DMT administration.

### Pre-Experience (Around 30 Minutes)

After the participant has completed all enrolment confirmation and randomization procedures and is cleared to participate, the Therapist, Assistant Therapist, and participant together in the room review all aspects of the room and safety procedures. The therapist should introduce the participant to the team member administering the 5-MeO-DMT, to create a sense of familiarity. Therapist introduces any Assistant Therapist and reviews safety features of the room and the equipment present. Participant has time to ask any questions. The therapist will ask about any responses to the situation and how the participant is feeling about their session. The participant should not be rushed into the dosing by the therapists. The therapist will ask the participant to engage in a period of relaxation prior to dosing. Participant will be asked to lie down, close their eyes, listen to the music, and, if willing, engage in at least one of the breathing exercises with the therapist's guidance. When the participant is settled and comfortable, the therapist will initiate the Session Opening. This practice helps contain and emphasize the specialness of the experience. Therapists will contact the member of the research team to come to the room and administer the 5-MeO-DMT. The team member should be aware not to disrupt the peaceful atmosphere of the room. The participant should be in a seated position when insufflating the 5-MeO-DMT, as the effects may be felt quickly, the participant should be transitioned to a prone position and remain prone for the duration of the effect of the 5-MeO-DMT.

### Experience (Around 60 Minutes)

It is expected that the onset of acute effects will occur very rapidly after administration. Therapists should be aware of the time of administration so they can be aware of the participant's response in relation to the expected course of duration. Some participants may want to know how long they experienced the effects of the 5-MeO-DMT and it is appropriate to share this information if asked. A significant portion of the time the participant may be nonverbal, focused inward, and engaging in their experience. It is important for the therapist to be mindfully aware of the participant, but not interfering with the participant's experience, unless it is clear that participant is seeking the therapist's support. Therapists are encouraged to engage in self-regulation techniques while the participant is undergoing their experience. This may be in the form of slow intentional inhaling and exhaling, or any other activity that helps the therapist ground and self-regulate. This is both for the therapist's benefit, as well as the participants', because a participant in a heightened non-ordinary state may be particularly attune to or pick up on their therapist's anxiety. It is optimal for the therapist to follow the participant's lead when choosing to verbally engage as the 5-MeO-DMT experience appears to be subsiding. Therapists may be eager to ask the participant about their experience, but it is preferable to wait until the participant is ready to share on their own. A participant may wish to remain in a period of silence, even after the apparent acute 5-MeO-DMT effect is gone. It is appropriate for therapists to greet participants with a friendly smile and welcoming nonverbal behavior, and allow participants to take the lead on sharing when they feel ready.

### Post-Experience (Around 90 Minutes)

Therapist will encourage the participant to stay with their experience for a period of time of at least one hour after the acute effects of the 5-MeO-DMT have worn off and the participant is once again aware of their surroundings and situation in the treatment room. To stay with the experience means to continue directing attention toward it in whatever way feels most appropriate to the participant, without turning to engagement in distractions, entertainment, or the concerns of daily life. During this time the therapist will invite the participant to describe their experience, if they choose to, and respect the choice not to if the participant is unready. If the participant does describe their experience the therapist is to listen and encourage the participant to express whatever they would like to share without interpretation or attempts to make meaning. The therapist practices simply listening, encouraging the participant to describe what they can about the experience. The therapist also offers the participant the option of resting and listening to the music, or to write about or draw any aspects of the experience they desire. At the end of this time period, the therapist will verify with the participant that they feel ready to close the session, will engage in the Session Closing, and contact the study team for exit assessment.

### Integration Sessions

The key principle of integration sessions is to help the participant focus on shifts in their perception of themselves and the implications of these as they relate to their depression. Self, for the purpose of this study, is broadly defined as the narrative or historical self, the sense of a coherent “I” that moves through experiences, and the self-identities one may use. It is key to remember that the sense of self, or the “I,” is reflected in both the experiencer's self-experience and experience of the object of experience, therefore descriptions may, on the surface, be of changes in the perception of the external world, but reflect shifts in the internal processes. To this end, the following therapeutic tasks will guide the integration sessions.

These sessions are less structured than preparatory sessions to accommodate variations in participant responses. There are three tasks: The first should occur at all sessions, the second and third may be introduced and engaged in if and when the participant is ready and willing. The tasks are:

Listening and Hearing about the Participant's Experience

Therapists ask open-ended questions about the participant's experience and listen with non-judgmental curiosity to the participant's descriptions. Therapists ask only that participants focus on the 5MDE and related material, such that their time together is focused on the treatment. Therapists should focus inquiry on the participant's experience, asking them to tune into any aspect of the three types of sense of self they can identify.

### Reintroducing the Values and Discussing Relationship to Each

The therapist will reintroduce the values identified in the Values Card Sort from preparation and bring discussion back to them if and when appropriate in the integration sessions. There is by no means a requirement to engage in the structured discussion of the values, but it serves as a framework where needed to direct the focus of sessions toward participants' shift in sense of self.

### The Therapist May Ask for Example, to Reintroduce the Values:

Therapist: Before your 5MDE we discussed a list of Values you hold and how you were relating to each of those. I'd like to draw our attention back to that and ask for a little detail about how those ways of relating might have shifted. For instance you named “Family” as one thing that was important to you, but you were concerned that you weren't feeling well enough to be present for family relationships. You said you were isolating from your family a lot by working on your computer from your makeshift office in the garage every evening. How do you relate to the value of “Family” now?

In the dialogue, the therapist can for example continue to focus on shifts in how the participant is relating to his value of “Family” by enquiring about what he is noticing in this area.

Create ways the participant can act to enhance their relationship to their chosen values; identify value-oriented action in their life as an integration practice. Integration can be understood as a process of embodying or living out the insights one has. In at least one of the integration sessions, the earliest the therapist feels the participant can engage in this stage, the therapist should introduce the idea of identifying value-oriented actions they can take in their lives as integration practices. Explaining the concept as above, the therapist can invite the participant to recall the values they identified (or any other that is important to them), recall the insights or experiences of their 5-MeO-DMT session, and think creatively about things they might try intentionally doing differently in order to implement positive change in their relationship to the values based on those insights and experiences

### Items:

1. A method of administering 5-MeO-DMT or a pharmaceutically acceptable salt thereof to a patient who is diagnosed with depression, the method comprising:
   the discontinuation of the use by the patient of any mood-altering substance or any other substance, medications or preparation which may affect serotonergic function;
   the relaxation of the patient, such as the patient is instructed to lay down, close their eyes, and listen to music and/or engage in one or more breathing exercises guided by a therapist;
   optionally, the clearing of their nasal passages, by blowing their nose, by the patient e.g. whilst sat down;
   the administration of 5-MeO-DMT, optionally by via insufflation, and optionally wherein the patient is in a prone position for the duration of the effects of 5-MeO-DMT.
2. The method of item 1, wherein the patient has discontinued the use of monoamine oxidase (MAO) inhibitors, CYP2D6 inhibitors, selective serotonin reuptake inhibitors (SSRIs), serotonin-norepinephrine reuptake inhibitors (SNRIs), tricyclic antidepressants (TCAs), lithium, antipsychotics, triptans, tramadol, 5-hydroxytryptophan, herbal preparations which may contain 5-HTP, St John's Wort and any benzodiazepines prior to administration of 5-MeO-DMT.
3. The method of item 1 or item 2, wherein the 5-MeO-DMT is administered via the Aptar Unidose (UDS) liquid delivery system.
4. The method of item 1, item 2 or item 3, wherein the 5-MeO-DMT is the benzoate salt, optionally a polymorph of the benzoate salt.
5. The method of any one of items 1 to 4, wherein the patient participates in at least one psychological support session before administration of the 5-MeO-DMT.
6. The method of item 5, wherein the patient participates in at least three psychological support sessions before administration of the 5-MeO-DMT.
7. The method of item 6, wherein the patient participates in three psychological support sessions, wherein these sessions take place 7 days, 4 days and 1 day before the administration of the 5-MeO-DMT.
8. The method of any one of items 5 to 7, wherein the psychological support sessions are 60-90 minutes in length.
9. The method of any one of items 5 to 8, wherein at least one therapeutic intention is discussed during the psychological support session.
10. The method of any one of items 5 to 9, wherein self-directed inquiry and experiential processing are practiced during the psychological support session.
11. The method of any one of items 1 to 10, wherein the patient participates in at least one psychological support session after administration of the 5-MeO-DMT.
12. The method of item 11, wherein the patient participates in at least three psychological support sessions after administration of the 5-MeO-DMT.
13. The method of item 11 or item 12, wherein the patient participates in three psychological support sessions, wherein these sessions take place 1 day, 4 days and 7 days after the administration of the 5-MeO-DMT.
14. The method of any one of items 11 to 13, wherein the psychological support sessions are 60-90 minutes in length.
15. The method of any one of items 1 to 14, wherein the 5-MeO-DMT is administered to the patient in a room with a substantially non-clinical appearance.
16. The method of item 15, wherein the room comprises soft furniture.
17. The method of item 15 or 16, wherein the room is decorated using muted colours.
18. The method of any one of items 15 to 17, wherein the room comprises a high-resolution sound system.
19. The method of any one of items 15 to 18 wherein the room comprises food and drink for the patient and therapist.
20. The method of any one of items 15 to 19 wherein the room comprises an approved safe for storing 5-MeO-DMT.
21. The method of any one of items 15 to 20 wherein the room is insulated such that the patient is shielded from sights and sounds of the world beyond the room.
22. The method of any one of items 15 to 21 wherein the room does not contain any artwork or decoration with any specific religious iconography, ideological connotation, or other such artwork or decoration which may evoke negative emotions in a patient.
23. The method of any one of items 15 to 22, wherein the room comprises a bed or a couch.
24. The method of item 23, wherein the patient lies in the bed or on the couch for approximately 0.5-8 hours, or a substantial fraction thereof, after administration of the 5-MeO-DMT.
25. The method of any one of items 1 to 24, wherein the patient listens to music for approximately 0.5-8 hours, or a substantial fraction thereof, after administration of the 5-MeO-DMT.
26. The method of any one of items 1 to 25, wherein the patient wears an eye mask for approximately 0.5-8 hours, or a substantial fraction thereof, after administration of the 5-MeO-DMT.
27. The method of any one of items 1 to 26, wherein a therapist provides psychological support to the patient for approximately 0.5-8 hours after administration of the 5-MeO-DMT
28. The method of any one of items 1 to 27, wherein the therapist uses guided imagery and/or breathing exercises to calm the patient and/or focus the patient's attention.
29. The method of any one of items 1 to 28, wherein the therapist provides reassuring physical contact with the patient.
30. The method of item 29, wherein the therapist holds the hand, arm, or shoulder of the patient.
31. The method of any one of items 1 to 30, wherein the therapist encourages the patient to perform self-directed inquiry and experiential processing.
32. The method of item 31, wherein the therapist reminds the patient of at least one therapeutic intention.
33. The method of any one of items 1 to 32, wherein the therapist counsels the patient to do one or more of the following:
   (1) to accept feelings of anxiety,
   (2) to allow the experience to unfold naturally,
   (3) to avoid psychologically resisting the experience,
   (4) to relax, and/or
   (5) to explore the patient's own mental space.
34. The method of any one of items 1 to 33, wherein the therapist does not initiate conversation with the patient.
35. The method of item 34, wherein the therapist responds to the patient if the patient initiates conversation.
36. The method of any one of items 5 to 35, wherein psychological support is provided remotely to the patient.
37. The method of item 36, wherein the psychological support is provided via a digital or electronic system.
38. The method of item 37, wherein the digital or electronic system is a mobile phone app.
39. The method of item 38, wherein the digital or electronic system is a website.

### Example 36: Mouse Forced Swim Test

This study aimed to assess the effect of 5-MeO-DMT Benzoate at three doses in the mouse Forced Swim Test (FST). The forced swim test is a model of behavioural despair and is sensitive to detection of various classes of antidepressant drugs.

### Husbandry

### Housing and Acclimation

Animals received a 72-hour period of acclimation to the test facility prior to the commencement of testing. Animals were housed four per cage in polycarbonate cages bedded with ¼″ bed-o′cob. Cages were changed, and enrichment provided according to standard operating procedures. Animals were maintained on a 12-hour light/12-hour dark cycle with all experimental activity occurring during the animals' light cycle. All animal use procedures were performed in accordance with the principles of the Canadian Council on Animal Care (CCAC).

### Food and Water

Certified Rodent Diet (LabDiet® 5001) was offered ad libitum. Animals were not fasted prior to, or after the experiment was initiated. Water was provided ad libitum in glass bottles with stainless steel sippers.

### Study Design

### Test Subjects

Male CD-1 mice from Charles River Laboratories (St. Constant, Quebec, Canada) served as test subjects in this study. Animals generally weighed 25-30 g at the time of testing.

### Schedule of Events

| Study Day | Key Event | Procedure |
|---|---|---|
| −8 | Animal arrival | Acclimation to the animal facility |
| −7, to −1 | Daily obs. | Daily health observations |
| 0 | Forced Swim Test | Body weights and observations |
| | | Dosing with 5-MeO DMT Benzoate, |
| | | Imipramine, and vehicle |
| | | Pre-FST behavioural test |
| | | Forced swim test |

### Treatment Groups

Animals were randomly allocated into the following treatment groups:

| | | | Pre- | |
|---|---|---|---|---|
| | | | treatment | Group |
| Group | Treatment | Route | time | Size |
| A | Vehicle | SC | 3 hr | N = 8 |
| B | 5-MeO DMT Benzoate | SC | 3 hr | N = 8 |
| | (0.5 mg/kg) | | | |
| C | 5-MeO DMT Benzoate | SC | 3 hr | N = 8 |
| | (1.5 mg/kg) | | | |
| D | 5-MeO DMT Benzoate (5 mg/kg) | SC | 3 hr | N = 8 |
| E | Imipramine (30 mg/kg) | IP | 3 hr | N = 8 |

### Pre-FST Behavioural Test

On day 0, in addition to the forced swim test animals were evaluated for signs of 5-HT (serotonin) syndrome. Animals were exposed to activity chambers for 10 minutes at two timepoints post dose: (1) 5-15 minutes post dose, and (2) 2.5 hours post dose.

### Forced Swim Test

Male CD-1 mice received the appropriate dose of vehicle, test article, or positive control (treatments summarized above). Following the appropriate pre-treatment time, animals were gently placed into tall glass cylinders filled with water (20-25° C.). After a period of vigorous activity, each mouse adopted a characteristic immobile posture which is readily identifiable. The swim test involves scoring the duration of immobility. Over a 6-minute test session, the latency to first immobility is recorded (in seconds). The duration of immobility (in seconds) during the last 4 minutes of the test is also measured. Activity or inactivity from 0-2 minutes is not recorded.

### Test Articles

### 5-MeODMT Benzoate

BEW: 1.59 (Benzoate salt form)
MW: 340.40 g/mol
Doses: 0.5, 1.5, 5 mg/kg (doses corrected to base)
Route of administration, dose volume: SC., 10 mL/kg
Pre-treatment time: 3 hr
Vehicle: 0.9% Saline

### Imipramine

BEW: 1.13
MW: 280.415 g/mol
Doses: 30 mg/kg (doses corrected to base)
Route of administration, dose volume: IP., 10 mL/kg
Pre-treatment time: 3 hr
Vehicle: 0.9% Saline

### Results

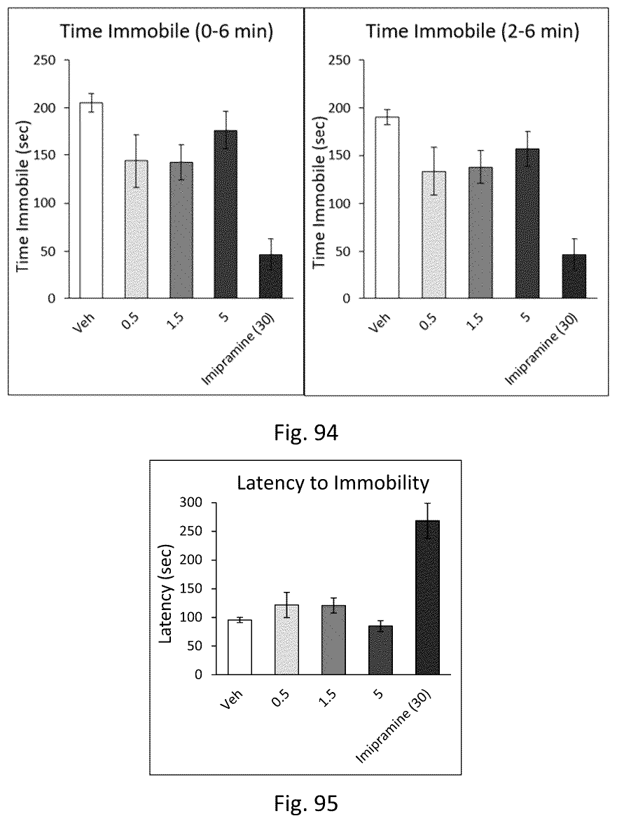

At 3-hour post-dose, over the 6-minute test session, there is a positive trend in reducing the duration of immobility and increasing latency to immobility by the low doses of 5-MeO-DMT benzoate (0.5 and 1.5 mg/kg), compared to vehicle-treated mice (time immobile 2-6 minutes, vehicle: 190.4±7.7 seconds—5-MeO-DMT benzoate: 133.2±24.9 seconds (0.5 mg/kg), 137.6±17.0 seconds (1.5 mg/kg), 156.8±18.7 seconds (5 mg/kg)—Imipramine 46.8±16.6 seconds, FIG. 94. Latency to immobility, vehicle: 95.5±4.6 seconds—5-MeO-DMT benzoate 121.8±22.0 seconds (0.5 mg/kg), 120.9±13.3 seconds (1.5 mg/kg), 85.0±9.5 seconds (5 mg/kg), imipramine 268.6±30.3 second, FIG. 95).

### Example 37: Study 5MEO-TOX-PK-DOG

The objective of this toxicokinetic study was to assess and compare the toxicokinetic profile of the test items, 5-MeO-DMT-HCl (in a vehicle of 0.1% metolose, Group 2) and 5-MeO-DMT-benzoate (in a vehicle of 0.2% metolose+0.01% BZK, Group 4).

On day 1, the vehicle or active test item formulations were administered to male Beagle dogs intranasally, at a dose level of 0.4 mg/kg in the active groups (corresponding to freebase). Following administration, a series of blood samples was collected from each dog at the following time points: pre-dose (0), 2, 5, 8, 10, 15, 30 and 60 minutes, and 2- and 8-hours post-dose. Plasma samples were analysed for quantification of concentration of 5-MeO-DMT in each sample using a validated method.

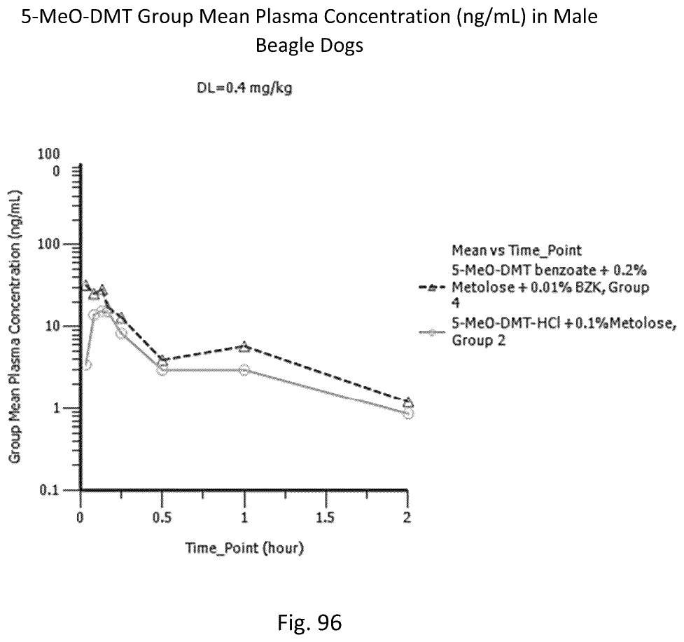

5-MeO-DMT was not detected in any of the samples collected from the control animals on Day 1 (not shown). Peak plasma exposure levels (Cₘₐₓ) were reported at 16.4 ng/mL and 35.4 ng/mL, for Groups 2 and 4, respectively (see table below). FIG. 96 presents the time-course plot of mean plasma concentrations, which shows a broadly comparable TK profile between the HCl and benzoate salt formulations.

### Mean Cₘₐₓ Values for 5-MeO-DMT in Groups 2 and 4 on Day 1

| | | Dose | Cₘₐₓ | | |
|---|---|---|---|---|---|
| Group | | Level | (ng/mL) | | |
| Designation | Day | (mg/kg) | Mean | SE | N |
| Group 2 | | | | | |
| 5-MeO-DMT- HCl + | 1 | 0.4 | 16.4 | 1.37 | 3 |
| 0.1% Metolose | | | | | |
| Group 4 | | | | | |
| 5-MeO-DMT benzoate + | 1 | 0.4 | 35.4 | 16.6 | 3 |
| 0.2% Metolose + | | | | | |
| 0.01% BZK | | | | | |

See also FIG. 96 which shows 5-MeO-DMT Group Mean Plasma Concentration (ng/mL) in Male Beagle Dogs—Group 2 (the 5-MeO-DMT HCl salt formulation) and Group 4 (the 5-MeO-DMT benzoate salt formulation)—Dose Level (0.4 mg/kg); wherein the Mean Plasma Concentration of Groups 2 and 4 are substantially the same with dose time.

### Example 38: Further Embodiments

In one embodiment, there is provided a polymorph of 5-MeO-DMT benzoate as characterised by an XRPD pattern as substantially illustrated in any one of the Figures or as previously or subsequently described.

In one embodiment, there is provided a polymorph of 5-MeO-DMT benzoate as characterised by one or more peaks in an XRPD diffractogram as substantially illustrated in any one of the Figures or as previously or subsequently described.

In one embodiment, there is provided a polymorph of 5-MeO-DMT benzoate as characterised by one or more endothermic events in a DSC thermograph as substantially illustrated in any one of the Figures or as previously or subsequently described.

In one embodiment, there is provided a polymorph of 5-MeO-DMT benzoate as characterised by TGA thermograph as substantially illustrated in any one of the Figures or as previously or subsequently described.

In one embodiment, there is provided a polymorph of 5-MeO-DMT benzoate as characterised by a DVS isotherm profile as substantially illustrated in any one of the Figures or as previously or subsequently described.

In one embodiment, there is provided a polymorph of 5-MeO-DMT benzoate as characterised by a crystalline appearance as substantially illustrated in any one of the Figures or as previously or subsequently described.

In one embodiment, there is provided a polymorph of 5-MeO-DMT benzoate as characterised by a particle size distribution as substantially illustrated in any one of the Figures or as previously or subsequently described.

In one embodiment, there is provided a polymorph of 5-MeO-DMT benzoate as characterised by a FITR spectra as substantially illustrated in any one of the Figures or as previously or subsequently described.

In one embodiment, there is provided a polymorph of 5-MeO-DMT benzoate produced as previously or subsequently described. In one embodiment, there is provided a method of producing a polymorph of 5-MeO-DMT benzoate as previously or subsequently described.

In one embodiment, there is provided a composition comprising a polymorph of 5-MeO-DMT benzoate as previously or subsequently described.

In one embodiment, there is provided a 5-MeO-DMT benzoate solvate as characterised as substantially illustrated in any one of the Figures or as previously or subsequently described.

In one embodiment, there is provided a 5-MeO-DMT benzoate hemi-solvate as characterised as substantially illustrated in any one of the Figures or as previously or subsequently described.

In one embodiment, there is provided the use of any previously or subsequently described form of 5-MeO-DMT benzoate in any previously or subsequently described method of treatment.

Herein disclosed is the use of a composition as herein described for the manufacture of a medicament for the treatment of any one of: conditions caused by dysfunctions of the central nervous system, conditions caused by dysfunctions of the peripheral nervous system, conditions benefiting from sleep regulation (such as insomnia), conditions benefiting from analgesics (such as chronic pain), migraines, trigeminal autonomic cephalgias (such as short-lasting unilateral neuralgiform headache with conjunctival injection and tearing (SUNCT), and short-lasting neuralgiform headaches with cranial autonomic symptoms (SUNA)), conditions benefiting from neurogenesis (such as stroke, traumatic brain injury, Parkinson's dementia), conditions benefiting from anti-inflammatory treatment, depression, treatment resistant depression, anxiety, substance use disorder, addictive disorder, gambling disorder, eating disorders, obsessive-compulsive disorders, or body dysmorphic disorders.

Herein disclosed is a method of treating any one of: conditions caused by dysfunctions of the central nervous system, conditions caused by dysfunctions of the peripheral nervous system, conditions benefiting from sleep regulation (such as insomnia), conditions benefiting from analgesics (such as chronic pain), migraines, trigeminal autonomic cephalgias (such as short-lasting unilateral neuralgiform headache with conjunctival injection and tearing (SUNCT), and short-lasting neuralgiform headaches with cranial autonomic symptoms (SUNA)), conditions benefiting from neurogenesis (such as stroke, traumatic brain injury, Parkinson's dementia), depression, treatment resistant depression, anxiety, substance use disorder, addictive disorder, gambling disorder, eating disorders, obsessive-compulsive disorders, or body dysmorphic in a patient by the administration of a composition as described herein.

### Example 39: Pattern H

Further characterisation work has revealed the existence of an additional Pattern H. This Pattern is demonstrated to be metastable and to undergo conversion to Pattern A via solvent equilibration.

The equilibration of multiple polymorphic variants of a solid in a range of favourable solvent systems with temperature modulation is an accepted approach to investigate the relationship between polymorphs and deduce thermodynamically preferred polymorphs or preferential solvent systems for metastable, solvated/hydrated or kinetically favoured versions.

5-MeO-DMT Benzoate versions Form I (Pattern A), Pattern B and Pattern H (ca. 20 mg of each) were charged to crystallisation tubes fitted with stirrer bead agitation, and subjected to equilibration in selected favourable solvent systems for each version of varying chemotypes (600 μl, 10 volumes).

A set of Pattern B and Pattern H samples (ca. 20 mg of each) were also amassed in the same set of solvents (400 μl, 10 volumes) to assess the proposition that there might be an enantiotropic relationship between Pattern B and Pattern H.

The samples were then agitated at 25° C. before sampling the solids via filtration for XRPD analysis for form fate after ca. 16 hours of agitation. The solid charges, solvents employed and form classification of the isolated solids following competitive equilibration is described in the Table below.

| | | Form I | | | XRPD |
|---|---|---|---|---|---|
| | | (Pattern A) | Pattern B | Pattern H | T = 16 Hrs |
| Sample ID | Solvent | Mass (mg) | Mass (mg) | Mass (mg) | 25° C. |
| 21/32/10/A | Isopropyl acetate | 21.44 | 20.64 | 20.48 | Form I |
| 21/32/10/B | 2-Propanol | 20.59 | 20.95 | 21.15 | Form I |
| 21/32/10/C | 2-Methyltetrahydorfuran | 20.66 | 20.88 | 20.90 | Form I |
| 21/32/10/D | Toluene | 20.74 | 20.28 | 20.32 | Pattern C - Toluene |
| | | | | | Hemi-solvate |
| 21/32/10/E | 2-Butanone | 20.92 | 20.75 | 20.48 | Form I |
| 21/32/10/F | Acetonitrile | 20.81 | 21.50 | 22.22 | Form I |
| 21/32/10/G | Isopropyl acetate | N/A | 23.05 | 21.21 | Form I |
| 21/32/10/H | 2-Propanol | N/A | 20.74 | 20.71 | Form I |
| 21/32/10/I | 2-Methyltetrahydorfuran | N/A | 20.34 | 22.11 | Form I |
| 21/32/10/J | Toluene | N/A | 21.25 | 28.15 | Pattern C - Toluene |
| | | | | | Hemi-solvate |
| 21/32/10/K | 2-Butanone | N/A | 21.99 | 21.38 | Form I |
| 21/32/10/L | Acetonitrile | N/A | 20.94 | 21.22 | Form I |

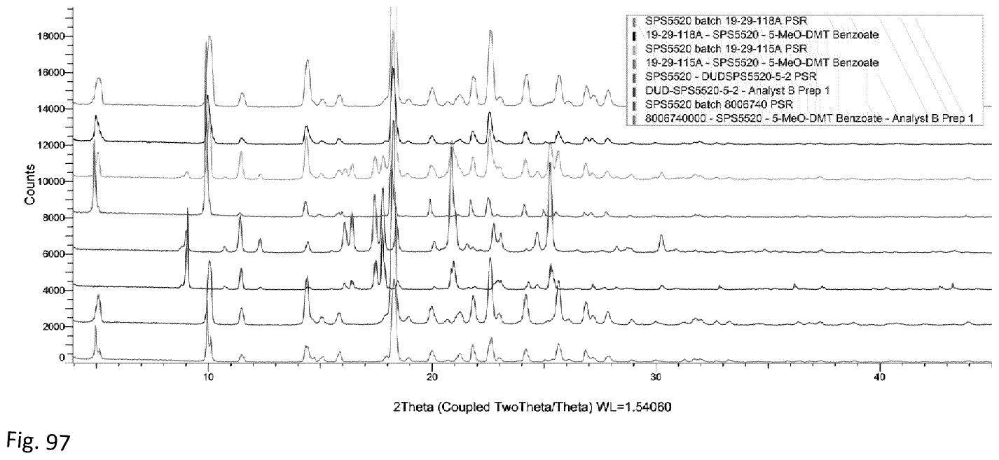

In an embodiment, Pattern H is characterised by an XRPD as substantially illustrated in FIG. 97. In FIG. 97, lots 8006740000 and 8006740000 PSR (particle size reduced) are Pattern H, 5520-5-2 and 5520-5-2 PSR are Pattern A, 19-29-115 A is Pattern H but 19-29-115 A PSR is a mixture of Pattern H and Pattern A and 19-29-118A and 19-29−118A PSR are Pattern H.

In an embodiment, Pattern H is characterised by a succinct melt-endo-exo crystallisation event from Pattern H to Pattern A at a 1° C./Min heating rate.

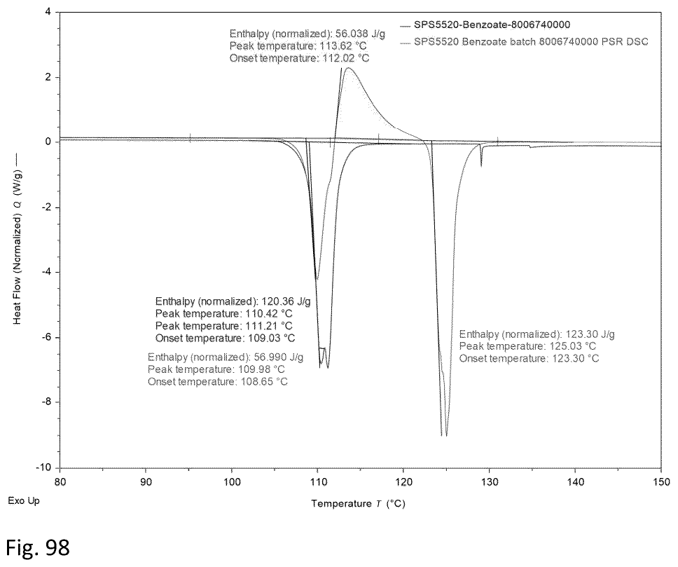

In an embodiment, Pattern H is characterised by a DSC thermograph as substantially illustrated in FIG. 98.

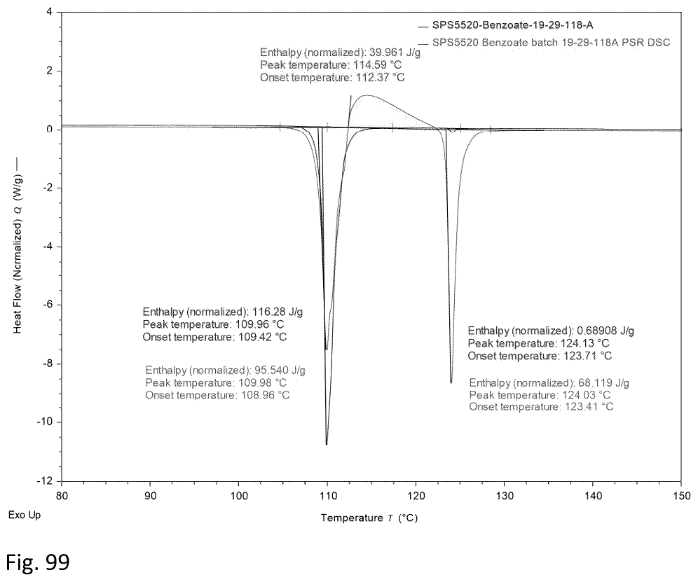

In an embodiment, Pattern H is characterised by a DSC thermograph as substantially illustrated in FIG. 99.

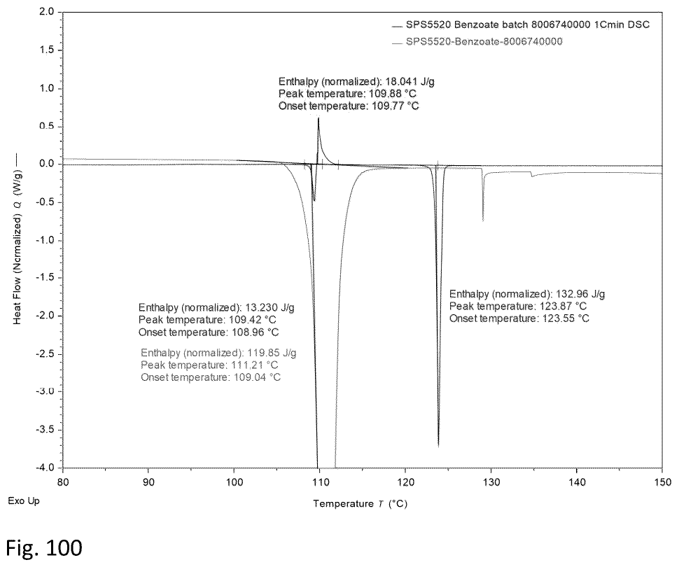

In an embodiment, Pattern H is characterised by a DSC thermograph as substantially illustrated in FIG. 100.

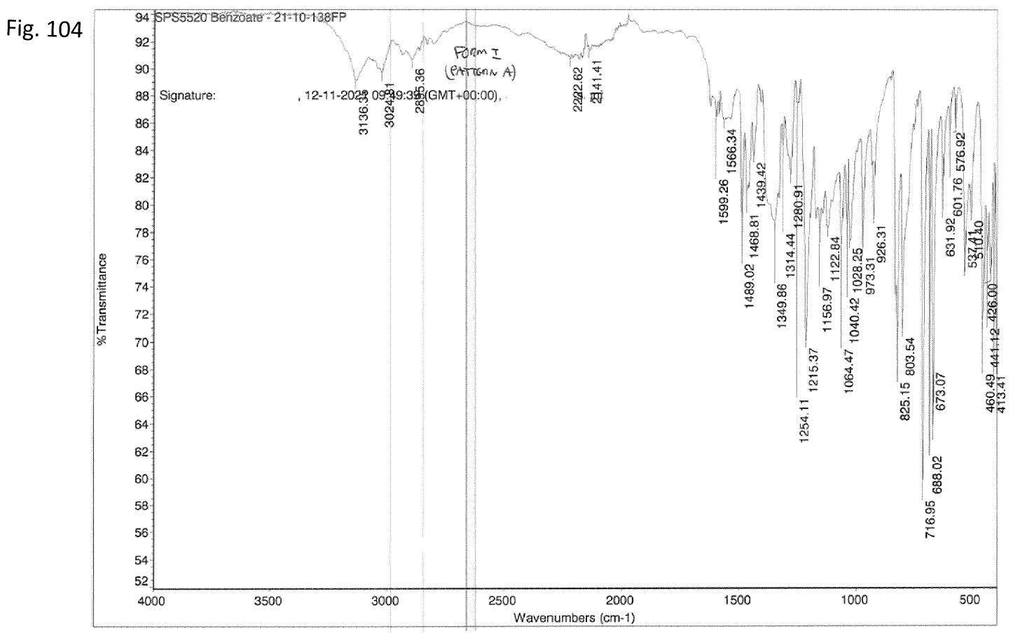

In an embodiment, Pattern A is characterised by FTIR spectra as substantially illustrated in FIG. 104.

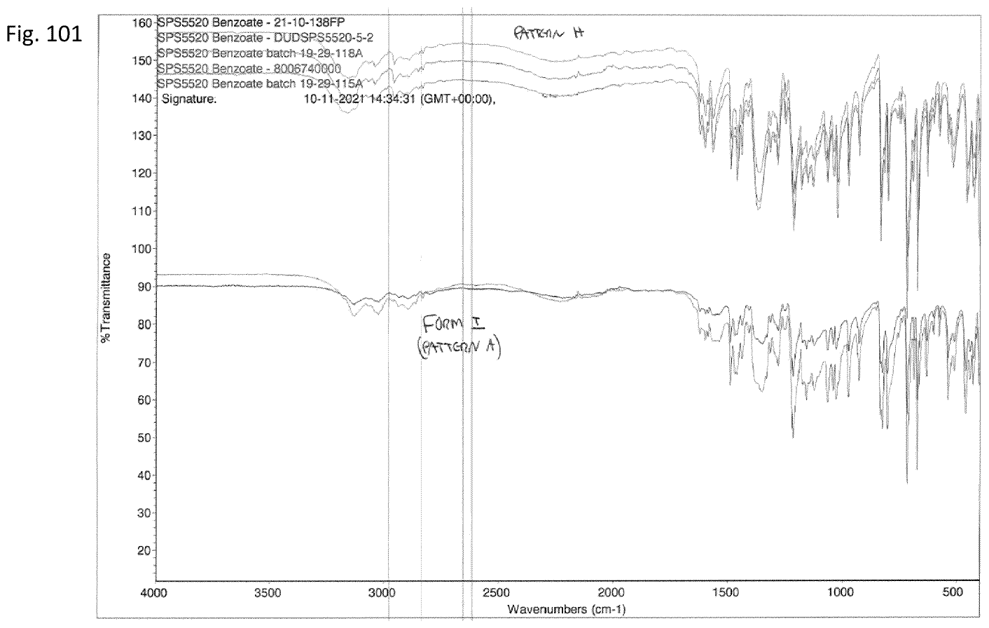

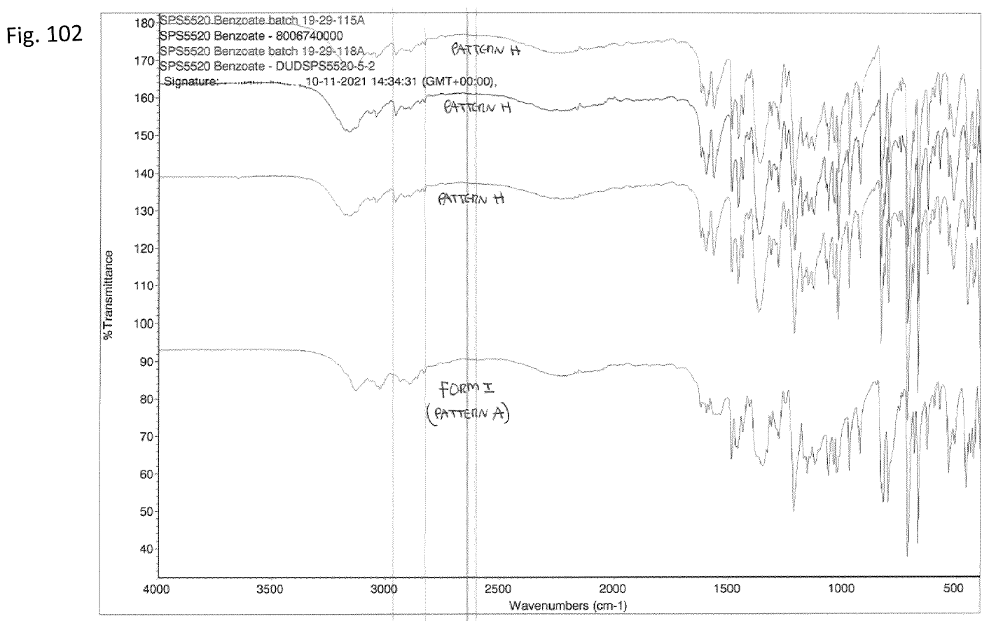

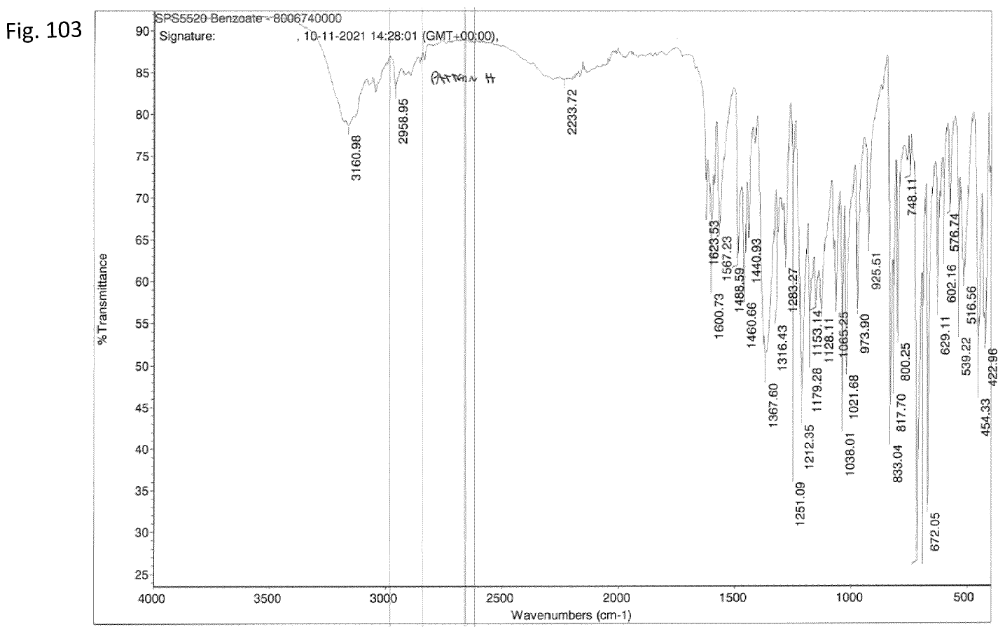

In an embodiment, Pattern H is characterised by FTIR spectra as substantially illustrated in any one of FIGS. 101,102 and 103.

In an embodiment, Pattern H is characterised by highly coloured large crystals >200 microns.

In an embodiment, Pattern H is characterised by irregularly shaped blue coloured small crystals ca.20-100 microns.

In an embodiment, Pattern A is characterised by rhombic shaped non birefringent large crystals ca. 400 microns.

In an embodiment, Pattern H is obtained following manufacture of 5-MeO-DMT benzoate in isopropyl acetate.

### Example 40: Formulations

### Sub-Lingual

In an embodiment, there is provided a sub-lingual formulation comprising 5-MeO-DMT benzoate.

In an embodiment, the sub-lingual formulation is a fast-dissolve sub-lingual formulation.

In an embodiment, the sub-lingual formulation is produced by freeze-drying/lyophilisation.

In an embodiment, the sub-lingual formulation is produced by:
Formulation of 5-MeO-DMT benzoate into a liquid solution or suspension;
Filling pre-formed blisters with said liquid;
Passing said blisters through a cryogenic freezing process; and
Transfer of said blisters to a lyophilizer followed by lyophilisation.

In an embodiment, passing said blisters through a cryogenic freezing process controls the size of ice crystals.

In an embodiment, the sub-lingual formulation disintegrates in less than 30 seconds from coming into contact with saliva.

In an embodiment, the sub-lingual formulation disintegrates in 3-10 seconds.

In an embodiment, there is provided the use of a sub-lingual formulation of 5-MeO-DMT benzoate in a method of treatment.

In an embodiment, there is provided the use of a sub-lingual formulation of 5-MeO-DMT benzoate T in the method of manufacture of a medicament for a therapeutic application.

In an embodiment, there is provided an orally disintegrating tablet (ODT) comprising 5-MeO-DMT benzoate.

In an embodiment, the ODT is a fast-dissolve sub-lingual formulation.

In an embodiment, the ODT is produced by freeze-drying/lyophilisation.

In an embodiment, the ODT is produced by:
Formulation of 5-MeO-DMT benzoate into a liquid solution or suspension;
Filling pre-formed blisters with said liquid;
Passing said blisters through a cryogenic freezing process; and
Transfer of said blisters to a lyophilizer followed by lyophilisation.

In an embodiment, passing said blisters through a cryogenic freezing process controls the size of ice crystals.

In an embodiment, the ODT disintegrates in less than 30 seconds from coming into contact with saliva.

In an embodiment, the ODT disintegrates in 3-10 seconds.

In an embodiment, there is provided the use of a 5-MeO-DMT benzoate ODT in a method of treatment.

In an embodiment, there is provided the use of a 5-MeO-DMT benzoate ODT in the method of manufacture of a medicament for a therapeutic application.

### Nasal

In an embodiment, there is provided a nasal formulation of 5-MeO-DMT benzoate.

In an embodiment, there is provided a spray-dried nasal formulation of 5-MeO-DMT benzoate.

In an embodiment, there is provided a spray-dried amorphous particulate powder formulation of 5-MeO-DMT benzoate.

In an embodiment, there is provided a spray-dried amorphous particulate powder formulation of 5-MeO-DMT benzoate, wherein the formulation has been co-sprayed with hydroxypropyl methylcellulose (HPMC).

In an embodiment, the nasal formulation has a median particle size of 10 to 100 micron, 20 to 90 micron, 30 to 80 micron, 40 to 70 micron, 30 to 60 micron or 40 to 50 micron. In an embodiment, the nasal formulation has a median particle size of 20 to 40 micron.

In an embodiment, there is provided the use of a nasal formulation of 5-MeO-DMT benzoate in a method of treatment.

In an embodiment, there is provided the use of a nasal formulation of 5-MeO-DMT benzoate in the method of manufacture of a medicament for a therapeutic application.

### Example 41: HPLC Method

An isocratic RP-HPLC method was also developed for assay of delivered-dose and the method parameters are listed in the Table below.

| Column | XSelect CSH C18, | | |
|---|---|---|---|
| | 3.5 μm, 4.6 × 150 mm | | |
| | Guard: XSelect CSH, c18, | | |
| | 3.5 μm, 4.6 × 20 mm | | |
| Mobile Phase | A | 10 mM Ammonium Acetate | |
| | B | Acetonitrile | |
| Autosampler Temperature | Ambient (20° C.) | | |
| Column Temperature | 30° C. | | |
| Injection volume | 10 μL | | |
| Wavelength | 224 nm | | |
| Wash Vial | 50:50 Water:MeOH | | |
| Flow Rate | 0.7 mL/min | | |
| | Time | % MPA | % MPB |
| Gradient | 0.00 | 80 | 20 |
| | 10.0 | 80 | 20 |

| | |
|---|---|
| Run Time | 10 minutes |
| Standard and Sample | 0.05 mg/mL |
| Concentration | |
| Typical 5-MeO-DMT RT | 6.3 minutes |

The reversed phase High-Performance Liquid Chromatography (RP-HPLC) method for the quantitative determination of assay and chemical purity of 5-MeO-DMT has been verified using the development of the 5-MeO-DMT benzoate drug substance. The method has been verified in terms of system suitability, specificity, limit of quantitation, linearity, accuracy and precision over a quantification range of 0.00005 to 0.13 mg/mi 5-MeO-DMT benzoate. A summary of the verification results can be found in the Table below.

| Qualification Parameters | | | Result |
|---|---|---|---|
| System Suitability | Blanks | | Free from interfering peaks |
| | % RSD | % RSD <2% | 0.06% |
| | n = 6 standard A injections | | |
| | Standard Concordance | 98.0-102.0% | 100.1% |
| | Bracketing standard | 98.0-102.0% | 99.4-100.6% |
| | injections | | |
| Specificity | Diluent containing HPMC at working sample | | No interfering peaks > LOQ |
| | concentration | | (0.05%) |
| Limit of | — | | 0.05% nominal concentration |
| Quantification | | | |
| Linearity | 0.05-130% nominal | R² >0.995 | R² = 1.000 |
| | concentration | | |
| | (0.1 mg/mL 5-MeO-DMT. | | |
| | Benzoate) | | |
| Accuracy | n = 3 at 70% nominal | % RSD <3.0% | 0.2% |
| % RSD | n = 3 at 100% nominal | % RSD <3.0% | 0.2% |
| | n = 3 at 130% nominal | % RSD <3.0% | 1.2% |
| Precision | % recovery at 70% nominal | 97.0-103.0% | 99.8-100.1% |
| | (n = 3) | | Average: 100.0% |
| | % recovery at 100% nominal | 97.0-103.0% | 99.2-99.6% |
| | (n = 3) | | Average: 99.4% |
| | % recovery at 130% nominal | 97.0-103.0% | 97.9-100.2% |
| | (n = 3) | | Average: 99.3% |
| Solution Stability | % recovery of T = 0 | 97.0-103.0% | Standard B - 100.8% |
| | 2-8° C./48 hrs | of T = 0 | 100% Rep 1 - 101.1% |
| | % recovery of T = 0 | 97.0-103.0% | Standard B - 101.0% |
| | Ambient (15-25° C.)/48 hrs | of T = 0 | 100% Rep 1 - 100.9% |

The isocratic RP-HPLC method was also verified, and a summary of the results are listed in the Table below.

| Qualification Parameters | | | Result |
|---|---|---|---|
| System | Blanks | | Free from interfering peaks |
| Suitability | % RSD | % RSD <2% | 0.16% |
| | n = 6 standard A injections | | |
| | Standard Concordance | 98.0-102.0% | 99.0% |
| | Bracketing standard | 98.0-102.0% | 99.9% |
| | injections | | |
| Linearity | 10-130% nominal | R² >0.995 | R² = 1.000 |
| | concentration | | |
| | (0.05 mg/mL 5-MeO-DMT. | | |
| | Benzoate) | | |
| Accuracy | n = 3 at 70% nominal | <3.0% | 0.2% |
| % RSD | n = 3 at 100% nominal | <3.0% | 0.2% |
| | n = 3 at 130% nominal | <3.0% | 0.3% |
| Precision | % recovery at 70% nominal | 97.0-103.0% | 100.3-100.6% |
| | (n = 3) | | Average: 100.4% |
| | % recovery at 100% nominal | 97.0-103.0% | 100.2-100.6% |
| | (n = 3) | | Average: 100.5% |
| | % recovery at 130% nominal | 97.0-103.0% | 100.2-100.9% |
| | (n = 3) | | Average: 100.6% |

### Example 42: An Improved Method of 5-MeO-DMT Benzoate Synthesis

A method for synthesising 5-MeO-DMT benzoate comprises the reduction of compound (1), this reaction requires refluxing in a large excess of lithium aluminium hydride and proceeds via a partially-reduced hydroxy impurity (2), see the reaction scheme below:

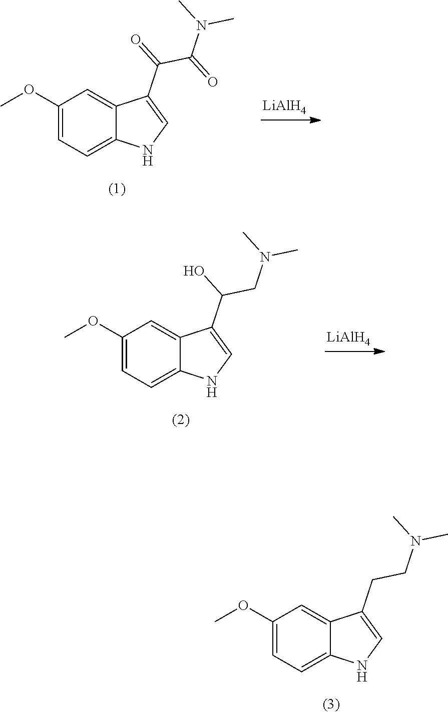

The initial reduction proceeds quite quickly. However, to reduce the hydroxy impurity all the way to (3) is more difficult to achieve and requires extended stir-out times at reflux. Even under such conditions it has proven difficult to reduce the hydroxy impurity level down to less than 1.5%. Under such aggressive conditions there runs the risk of degrading (3) in trying to achieve reaction completion either by charging additional lithium aluminium hydride or extending the time at reflux.

Surprisingly, the inventors have discovered that isolating (3) as the HCl salt followed by a salt break process and benzoate salt formation greatly improves the purging (2). Under the more acidic conditions of the HCl salt formation, dehydration of the impurity can occur to give two more readily purged species (4) and (5), see the reaction scheme below:

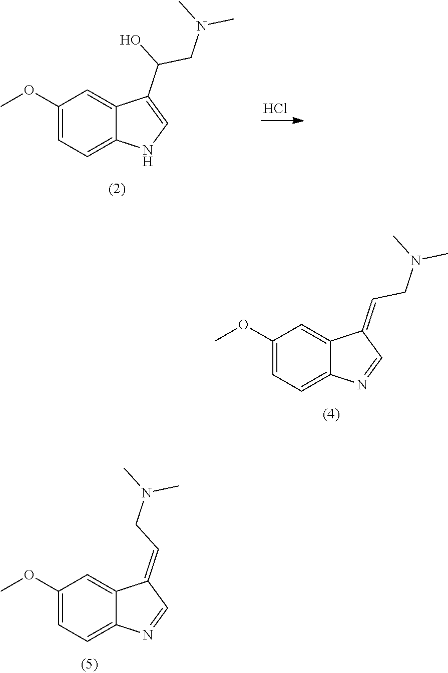

The above disclosed improved method allows for the provision of batches of 5-MeO-DMT benzoate in which the levels of the hydroxyl impurity are very low, 0.1% or lower, compared with 1.5% for the aforementioned method.

### Example 43: X-Ray Powder Diffractogram (XRPD) Analysis of 5-MeO-DMT Hydrochloride

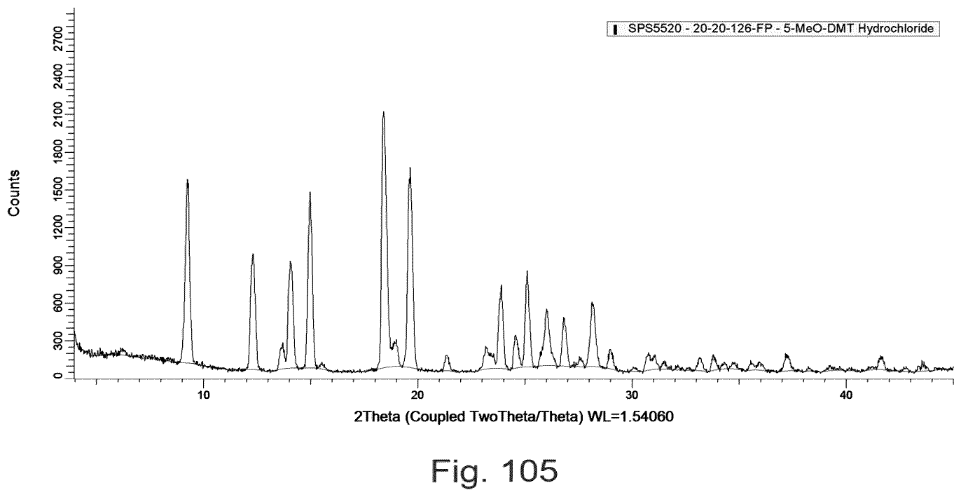

XRPD analysis of two lots of 5-MeO-DMT hydrochloride (lot 20/20/126-FP and lot 20/45/006-FP) was performed, both were of well-ordered material with moderate relative crystallinity and exhibited the same crystalline pattern, which can be seen in FIGS. 105-107. In the absence of any other known sample label, the pattern was assigned as 5-MeO-DMT Hydrochloride Pattern A. In one embodiment, there is provided crystalline 5-MeO-DMT hydrochloride, characterised by peaks in an XRPD diffractogram at 9.2, 12.2, 14.1, 15.0, 18.5 and 19.5°2θ±0.1°2θ.

In one embodiment, there is provided crystalline 5-MeO-DMT hydrochloride, characterised by peaks in an XRPD diffractogram at 9.2, 12.2, 14.1, 15.0, 18.5 and 19.5°2θ±0.2°2θ.

In one embodiment, there is provided crystalline 5-MeO-DMT hydrochloride, characterised by peaks in an XRPD diffractogram at 9.2, 12.2, 14.1, 15.0, 18.5 and 19.5°2θ±0.3°2θ.

In one embodiment, there is provided crystalline 5-MeO-DMT hydrochloride, characterised by peaks in an XRPD diffractogram at 9.2, 12.2, 14.1, 15.0, 18.5 and 19.5°2θ±0.1°2θ as measured by x-ray powder diffraction using an x-ray wavelength of 1.5406 Å.

In one embodiment, there is provided crystalline 5-MeO-DMT hydrochloride, characterised by peaks in an XRPD diffractogram at 9.2, 12.2, 14.1, 15.0, 18.5 and 19.5°2θ±0.2°2θ as measured by x-ray powder diffraction using an x-ray wavelength of 1.5406 Å.

In one embodiment, there is provided crystalline 5-MeO-DMT hydrochloride, characterised by peaks in an XRPD diffractogram at 9.2, 12.2, 14.1, 15.0, 18.5 and 19.5°2θ±0.3°2θ as measured by x-ray powder diffraction using an x-ray wavelength of 1.5406 Å.

In one embodiment, there is provided crystalline 5-MeO-DMT hydrochloride, characterised by peaks in an XRPD diffractogram at 9.2, 12.2, 14.1, 15.0, 18.5, 19.5, 23.9, 24.5, 25.1, 26.0, 26.9 and 28.3°2θ±0.1°2θ.

In one embodiment, there is provided crystalline 5-MeO-DMT hydrochloride, characterised by peaks in an XRPD diffractogram at 9.2, 12.2, 14.1, 15.0, 18.5, 19.5, 23.9, 24.5, 25.1, 26.0, 26.9 and 28.3°2θ±0.2°2θ.

In one embodiment, there is provided crystalline 5-MeO-DMT hydrochloride, characterised by peaks in an XRPD diffractogram at 9.2, 12.2, 14.1, 15.0, 18.5, 19.5, 23.9, 24.5, 25.1, 26.0, 26.9 and 28.3°2θ±0.3°2θ.

In one embodiment, there is provided crystalline 5-MeO-DMT hydrochloride, characterised by peaks in an XRPD diffractogram at 9.2, 12.2, 14.1, 15.0, 18.5, 19.5, 23.9, 24.5, 25.1, 26.0, 26.9 and 28.3°2θ±0.1°2θ as measured by x-ray powder diffraction using an x-ray wavelength of 1.5406 Å.

In one embodiment, there is provided crystalline 5-MeO-DMT hydrochloride, characterised by peaks in an XRPD diffractogram at 9.2, 12.2, 14.1, 15.0, 18.5, 19.5, 23.9, 24.5, 25.1, 26.0, 26.9 and 28.3°2θ±0.2°2θ as measured by x-ray powder diffraction using an x-ray wavelength of 1.5406 Å.

In one embodiment, there is provided crystalline 5-MeO-DMT hydrochloride, characterised by peaks in an XRPD diffractogram at 9.2, 12.2, 14.1, 15.0, 18.5, 19.5, 23.9, 24.5, 25.1, 26.0, 26.9 and 28.3°2θ±0.3°2θ as measured by x-ray powder diffraction using an x-ray wavelength of 1.5406 Å.

In one embodiment, there is provided crystalline 5-MeO-DMT hydrochloride, characterised by peaks in an XRPD diffractogram at 9.2, 12.2, 13.7, 14.1, 15.0, 18.5, 19.0, 19.5, 21.2, 23.3, 23.9, 24.5, 25.1, 26.0, 26.9, 27.5, 28.3, 29.0, 30.9 and 31.1°2θ±0.1°2θ.

In one embodiment, there is provided crystalline 5-MeO-DMT hydrochloride, characterised by peaks in an XRPD diffractogram at 9.2, 12.2, 13.7, 14.1, 15.0, 18.5, 19.0, 19.5, 21.2, 23.3, 23.9, 24.5, 25.1, 26.0, 26.9, 27.5, 28.3, 29.0, 30.9 and 31.1°2θ±0.1°2θ.

In one embodiment, there is provided crystalline 5-MeO-DMT hydrochloride, characterised by peaks in an XRPD diffractogram at 9.2, 12.2, 13.7, 14.1, 15.0, 18.5, 19.0, 19.5, 21.2, 23.3, 23.9, 24.5, 25.1, 26.0, 26.9, 27.5, 28.3, 29.0, 30.9 and 31.1°2θ±0.2°2θ.

In one embodiment, there is provided crystalline 5-MeO-DMT hydrochloride, characterised by peaks in an XRPD diffractogram at 9.2, 12.2, 13.7, 14.1, 15.0, 18.5, 19.0, 19.5, 21.2, 23.3, 23.9, 24.5, 25.1, 26.0, 26.9, 27.5, 28.3, 29.0, 30.9 and 31.1°2θ±03°2θ.

In one embodiment, there is provided crystalline 5-MeO-DMT hydrochloride, characterised by peaks in an XRPD diffractogram at 9.2, 12.2, 13.7, 14.1, 15.0, 18.5, 19.0, 19.5, 21.2, 23.3, 23.9, 24.5, 25.1, 26.0, 26.9, 27.5, 28.3, 29.0, 30.9 and 31.1°2θ±0.1°2θ as measured by x-ray powder diffraction using an x-ray wavelength of 1.5406 Å.

In one embodiment, there is provided crystalline 5-MeO-DMT hydrochloride, characterised by peaks in an XRPD diffractogram at 9.2, 12.2, 13.7, 14.1, 15.0, 18.5, 19.0, 19.5, 21.2, 23.3, 23.9, 24.5, 25.1, 26.0, 26.9, 27.5, 28.3, 29.0, 30.9 and 31.1°2θ±0.2°2θ as measured by x-ray powder diffraction using an x-ray wavelength of 1.5406 Å.

In one embodiment, there is provided crystalline 5-MeO-DMT hydrochloride, characterised by peaks in an XRPD diffractogram at 9.2, 12.2, 13.7, 14.1, 15.0, 18.5, 19.0, 19.5, 21.2, 23.3, 23.9, 24.5, 25.1, 26.0, 26.9, 27.5, 28.3, 29.0, 30.9 and 31.1°2θ±0.3°2θ as measured by x-ray powder diffraction using an x-ray wavelength of 1.5406 Å.

In one embodiment, there is provided crystalline 5-MeO-DMT hydrochloride, characterised by peaks in an XRPD diffractogram as substantially illustrated in FIG. 105, 106 or 107.

In one embodiment, there is provided crystalline 5-MeO-DMT hydrochloride, characterised by peaks in an XRPD diffractogram as substantially illustrated in FIG. 105.

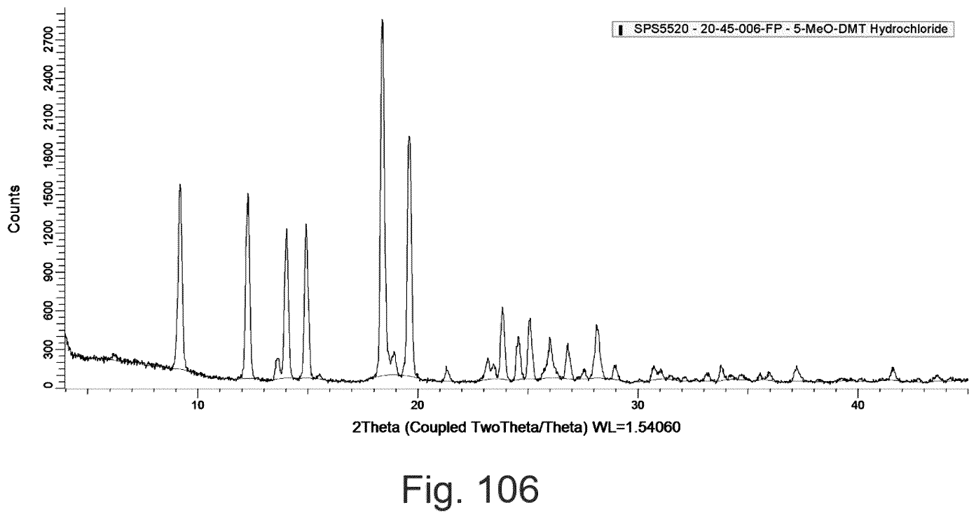

In one embodiment, there is provided crystalline 5-MeO-DMT hydrochloride, characterised by peaks in an XRPD diffractogram as substantially illustrated in FIG. 106.

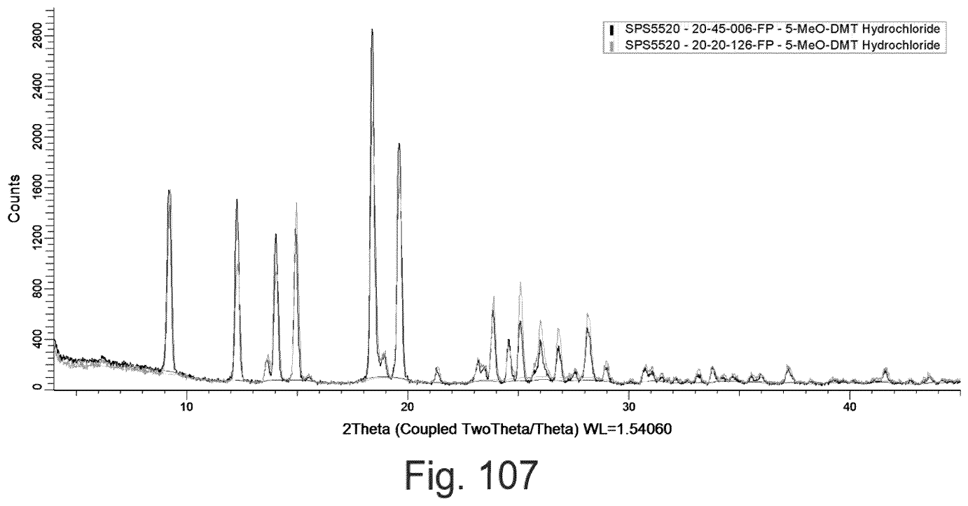

In one embodiment, there is provided crystalline 5-MeO-DMT hydrochloride, characterised by peaks in an XRPD diffractogram as substantially illustrated in FIG. 107. In one embodiment, there is provided crystalline 5-MeO-DMT hydrochloride, characterised by one or more of:
Peaks in an XRPD diffractogram as previously or subsequently described;
An endothermic event in a DSC thermograph as previously or subsequently described;
An onset of decomposition in a TGA thermograph as previously or subsequently described;
A DVS isotherm profile as previously or subsequently described; and
A crystalline structure as previously or subsequently described.

### Example 44: Thermal Analysis of 5-MeO-DMT Hydrochloride

Differential scanning calorimetry (DSC) and thermogravimetric analysis (TGA) was performed on both lots at a standard heating rate of 10° C./Min from 30-400° C. In addition, DSC assessments of the solids were also conducted at 5, 20 and 40° C./Min heating rates. No significant differences in profile were observed between samples via DSC or TGA or via the variable DSC heating rates. An unstable DSC baseline is observed from 290° C. onwards due to rapid decomposition of the material.

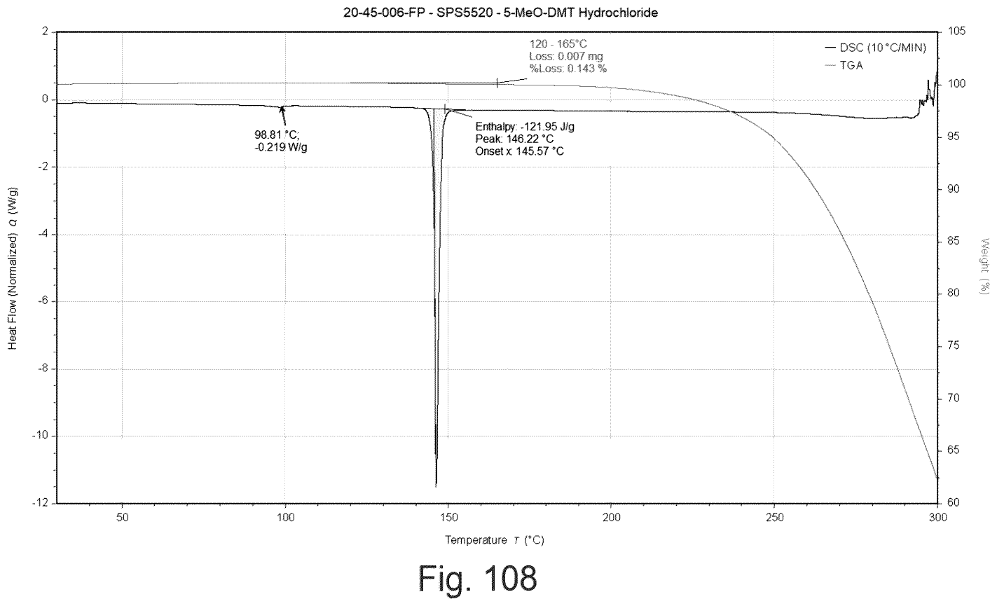

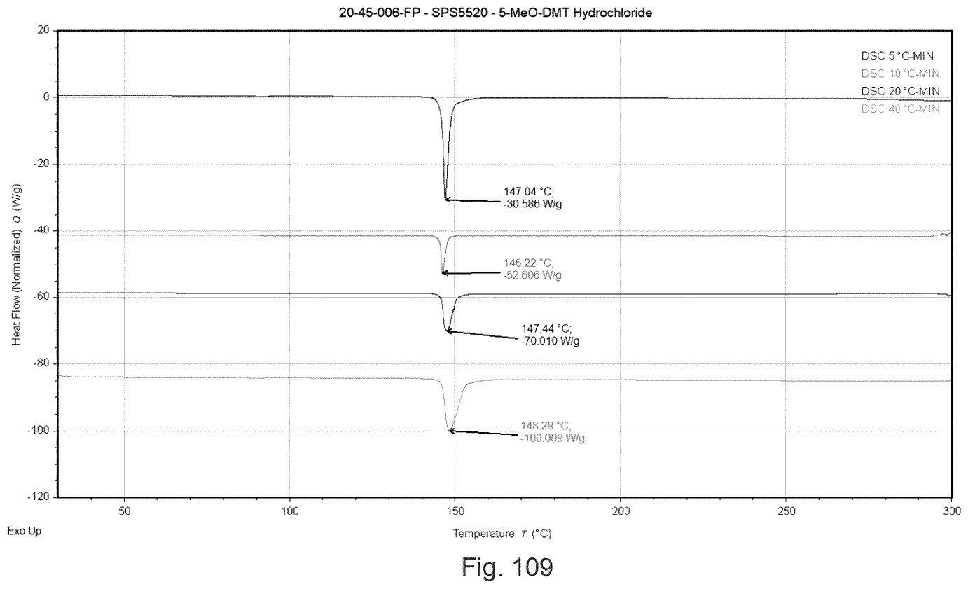

FIG. 108 shows a DSC and TGA thermograph of 5-MeO-DMT Hydrochloride, lot 20/20/126-FP at 10° C./Min heating rate. FIG. 109 shows DSC thermographs of 5-MeO-DMT hydrochloride, lot 20/20/126-FP at 5° C./Min (Black), 10° C./Min (Red), 20° C./Min (Blue) and 40° C./Min (Green) heating rates.

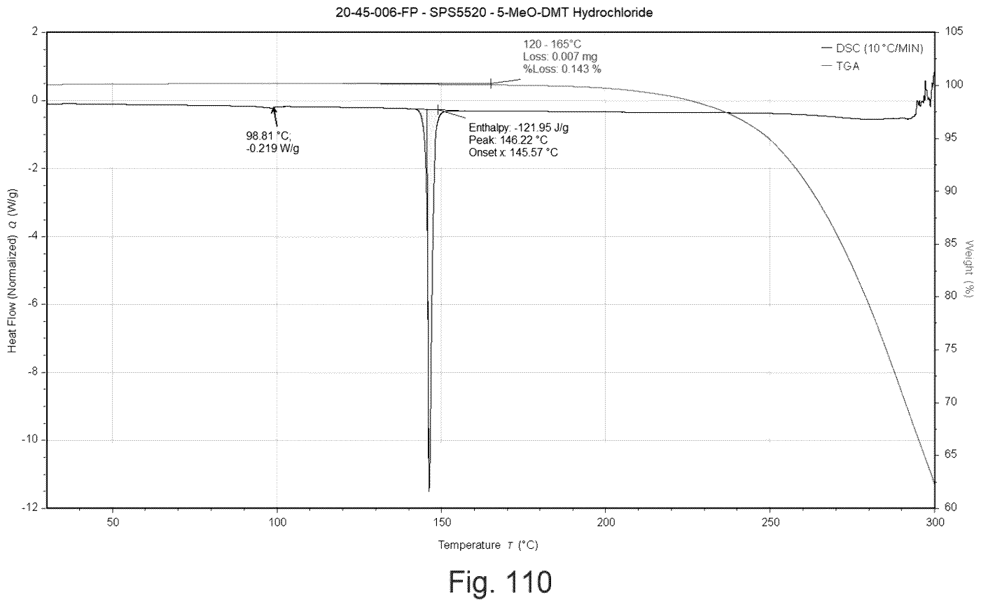

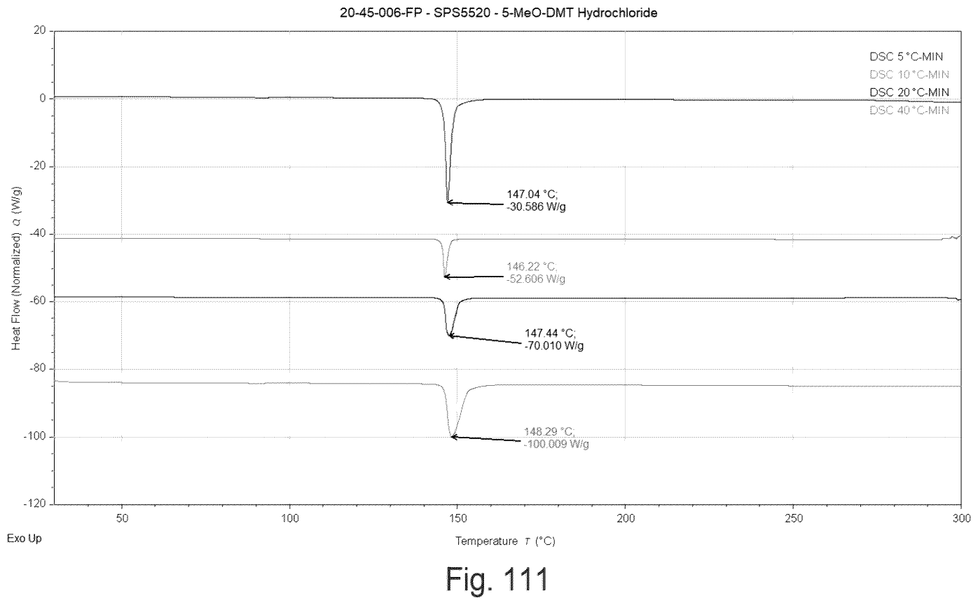

FIG. 110 shows a DSC and TGA thermograph of 5-MeO-DMT Hydrochloride, lot 20/45/006-FP at 10° C./Min heating rate. FIG. 111 shows DSC thermographs of 5-MeO-DMT hydrochloride, lot 20/45/006-FP at 5° C./Min (Black), 10° C./Min (Red), 20° C./Min (Blue) and 40° C./Min (Green) heating rates.

In one embodiment, there is provided a crystalline 5-MeO-DMT Hydrochloride, characterised by an endothermic event in a DSC thermograph having an onset temperature of between 14° and 150° C.

In one embodiment, there is provided a crystalline 5-MeO-DMT Hydrochloride, characterised by an endothermic event in a DSC thermograph having an onset temperature of between 14° and 150° C., as substantially illustrated in FIG. 108 or FIG. 109.

In one embodiment, there is provided a crystalline 5-MeO-DMT Hydrochloride, characterised by an endothermic event in a DSC thermograph having an onset temperature of 146° C.

In one embodiment, there is provided a crystalline 5-MeO-DMT Hydrochloride, characterised by an endothermic event in a DSC thermograph having an onset temperature of 146° C. as substantially illustrated in FIG. 108 or FIG. 109.

In one embodiment, there is provided a crystalline 5-MeO-DMT Hydrochloride, characterised by an endothermic event in a DSC thermograph having an onset temperature of between 14° and 150° C. and a peak of between 142 and 148° C.

In one embodiment, there is provided a crystalline 5-MeO-DMT Hydrochloride, characterised by an endothermic event in a DSC thermograph having an onset temperature of between 14° and 150° C. and a peak of between 142 and 148° C. as substantially illustrated in FIG. 108 or FIG. 109.

In one embodiment, there is provided a crystalline 5-MeO-DMT Hydrochloride, characterised by an endothermic event in a DSC thermograph having an onset temperature of 145.95° C. and a peak of 146.74° C.

In one embodiment, there is provided a crystalline 5-MeO-DMT Hydrochloride, characterised by an endothermic event in a DSC thermograph having an onset temperature of 145.95° C. and a peak of 146.74° C. as substantially illustrated in FIG. 108 or FIG. 109.

In one embodiment, there is provided a crystalline 5-MeO-DMT Hydrochloride, characterised by an endothermic event in a DSC thermograph having an onset temperature of between 14° and 150° C. and an enthalpy of between −113 J/g and −123 J/g.

In one embodiment, there is provided a crystalline 5-MeO-DMT Hydrochloride, characterised by an endothermic event in a DSC thermograph having an onset temperature of between 14° and 150° C. and an enthalpy of between −113 J/g and −123 J/g as substantially illustrated in FIG. 108 or FIG. 109.

In one embodiment, there is provided a crystalline 5-MeO-DMT Hydrochloride, characterised by an endothermic event in a DSC thermograph having an onset temperature of between 14° and 150° C., a peak of between 142 and 148° C. and an enthalpy of between −113 J/g and −123 J/g.

In one embodiment, there is provided a crystalline 5-MeO-DMT Hydrochloride, characterised by an endothermic event in a DSC thermograph having an onset temperature of between 14° and 150° C., a peak of between 142 and 148° C. and an enthalpy of between −113 J/g and −123 J/g as substantially illustrated in FIG. 108 or FIG. 109.

In one embodiment, there is provided a crystalline 5-MeO-DMT Hydrochloride, characterised by an endothermic event in a DSC thermograph having an onset temperature of 145.95° C., a peak of 146.74° C. and an enthalpy of −118.29/g.

In one embodiment, there is provided a crystalline 5-MeO-DMT Hydrochloride, characterised by an endothermic event in a DSC thermograph having an onset temperature of 145.95° C., a peak of 146.74° C. and an enthalpy of −118.29/g as substantially illustrated in FIG. 108 or FIG. 109.

In one embodiment, there is provided a crystalline 5-MeO-DMT Hydrochloride, characterised by an onset of decomposition in a TGA thermograph of between 12° and 165° C.

In one embodiment, there is provided a crystalline 5-MeO-DMT Hydrochloride, characterised by an onset of decomposition in a TGA thermograph of between 12° and 165° C. as substantially illustrated in FIG. 108.

In one embodiment, there is provided a crystalline 5-MeO-DMT Hydrochloride, characterised by one or more of:
an endothermic event in a DSC thermograph having an onset temperature of between 14° and 150° C.; and
an onset of decomposition in a TGA thermograph of between 12° and 165° C.

In one embodiment, there is provided a crystalline 5-MeO-DMT Hydrochloride, characterised by one or more of:
an endothermic event in a DSC thermograph having an onset temperature of between 14° and 150° C. as substantially illustrated in FIG. 108 or FIG. 109; and
an onset of decomposition in a TGA thermograph of between 12° and 165° C., as substantially illustrated in FIG. 7.

In one embodiment, there is provided a crystalline 5-MeO-DMT Hydrochloride, characterised by one or more of:
an endothermic event in a DSC thermograph having an onset temperature of between 14° and 150° C., a peak of between 142 and 148° C.; and
an onset of decomposition in a TGA thermograph of between 12° and 165° C.

In one embodiment, there is provided a crystalline 5-MeO-DMT Hydrochloride, characterised by one or more of:
an endothermic event in a DSC thermograph having an onset temperature of between 14° and 150° C., a peak of between 142 and 148° C. as substantially illustrated in FIG. 108 or FIG. 109; and
an onset of decomposition in a TGA thermograph of between 12° and 165° C. as substantially illustrated in FIG. 108.

In one embodiment, there is provided a crystalline 5-MeO-DMT Hydrochloride, characterised by one or more of:
an endothermic event in a DSC thermograph having an onset temperature of between 14° and 150° C., a peak of between 142 and 148° C. and an enthalpy of between −113 J/g and −123 J/g; and
an onset of decomposition in a TGA thermograph of between 12° and 165° C.

In one embodiment, there is provided a crystalline 5-MeO-DMT Hydrochloride, characterised by one or more of:
an endothermic event in a DSC thermograph having an onset temperature of between 14° and 150° C., a peak of between 142 and 148° C. and an enthalpy of between −113 J/g and −123J/gas substantially illustrated in FIG. 108 or FIG. 109; and
an onset of decomposition in a TGA thermograph of between 12° and 165° C. as substantially illustrated in FIG. 108.

In one embodiment, there is provided a crystalline 5-MeO-DMT Hydrochloride, characterised by one or more of:
an endothermic event in a DSC thermograph having an onset temperature of 145.95° C., a peak of 146.74° C. and an enthalpy of −118.29 J/g; and
an onset of decomposition in a TGA thermograph of between 12° and 165° C.

In one embodiment, there is provided a crystalline 5-MeO-DMT Hydrochloride, characterised by one or more of:
an endothermic event in a DSC thermograph having an onset temperature of 145.95° C., a peak of 146.74° C. and an enthalpy of −118.29 J/g as substantially illustrated in FIG. 108 or FIG. 109; and
an onset of decomposition in a TGA thermograph of between 12° and 165° C. as substantially illustrated in FIG. 108.

In one embodiment, there is provided a crystalline 5-MeO-DMT Hydrochloride, characterised by an endothermic event in a DSC thermograph having an onset temperature of between 14° and 150° C.

In one embodiment, there is provided a crystalline 5-MeO-DMT Hydrochloride, characterised by an endothermic event in a DSC thermograph having an onset temperature of between 14° and 150° C., as substantially illustrated in FIG. 110.

In one embodiment, there is provided a crystalline 5-MeO-DMT Hydrochloride, characterised by an endothermic event in a DSC thermograph having an onset temperature of 146° C.

In one embodiment, there is provided a crystalline 5-MeO-DMT Hydrochloride, characterised by an endothermic event in a DSC thermograph having an onset temperature of 146° C. as substantially illustrated in FIG. 110.

In one embodiment, there is provided a crystalline 5-MeO-DMT Hydrochloride, characterised by an endothermic event in a DSC thermograph having an onset temperature of 145° C.

In one embodiment, there is provided a crystalline 5-MeO-DMT Hydrochloride, characterised by an endothermic event in a DSC thermograph having an onset temperature of 145° C. as substantially illustrated in FIG. 110.

In one embodiment, there is provided a crystalline 5-MeO-DMT Hydrochloride, characterised by an endothermic event in a DSC thermograph having an onset temperature of between 14° and 150° C. and a peak of between 142 and 148° C.

In one embodiment, there is provided a crystalline 5-MeO-DMT Hydrochloride, characterised by an endothermic event in a DSC thermograph having an onset temperature of between 14° and 150° C. and a peak of between 142 and 148° C. as substantially illustrated in FIG. 110.

In one embodiment, there is provided a crystalline 5-MeO-DMT Hydrochloride, characterised by an endothermic event in a DSC thermograph having an onset temperature of 145.57° C. and a peak of 146.22° C.

In one embodiment, there is provided a crystalline 5-MeO-DMT Hydrochloride, characterised by an endothermic event in a DSC thermograph having an onset temperature of 145.57° C. and a peak of 146.22° C. as substantially illustrated in FIG. 110.

In one embodiment, there is provided a crystalline 5-MeO-DMT Hydrochloride, characterised by an endothermic event in a DSC thermograph having an onset temperature of between 14° and 150° C. and an enthalpy of between −115 J/g and −125 J/g.

In one embodiment, there is provided a crystalline 5-MeO-DMT Hydrochloride, characterised by an endothermic event in a DSC thermograph having an onset temperature of between 14° and 150° C. and an enthalpy of between −115 J/g and −125 J/g as substantially illustrated in FIG. 110.

In one embodiment, there is provided a crystalline 5-MeO-DMT Hydrochloride, characterised by an endothermic event in a DSC thermograph having an onset temperature of between 14° and 150° C., a peak of between 142 and 148° C. and an enthalpy of between −115 J/g and −125 J/g.

In one embodiment, there is provided a crystalline 5-MeO-DMT Hydrochloride, characterised by an endothermic event in a DSC thermograph having an onset temperature of between 14° and 150° C., a peak of between 142 and 148° C. and an enthalpy of between −115 J/g and −125 J/g as substantially illustrated in FIG. 110.

In one embodiment, there is provided a crystalline 5-MeO-DMT Hydrochloride, characterised by an endothermic event in a DSC thermograph having an onset temperature of 145.57° C., a peak of 146.22° C. and an enthalpy of −121.95 J/g.

In one embodiment, there is provided a crystalline 5-MeO-DMT Hydrochloride, characterised by an endothermic event in a DSC thermograph having an onset temperature of 145.57° C., a peak of 146.22° C. and an enthalpy of −121.95 J/g as substantially illustrated in FIG. 110.

In one embodiment, there is provided a crystalline 5-MeO-DMT Hydrochloride, characterised by an onset of decomposition in a TGA thermograph of between 12° and 165° C.

In one embodiment, there is provided a crystalline 5-MeO-DMT Hydrochloride, characterised by an onset of decomposition in a TGA thermograph of between 12° and 165° C. as substantially illustrated in FIG. 110.

In one embodiment, there is provided a crystalline 5-MeO-DMT Hydrochloride, characterised by one or more of:
an endothermic event in a DSC thermograph having an onset temperature of between 14° and 150° C.; and
an onset of decomposition in a TGA thermograph of between 12° and 165° C.

In one embodiment, there is provided a crystalline 5-MeO-DMT Hydrochloride, characterised by one or more of:
an endothermic event in a DSC thermograph having an onset temperature of between 14° and 150° C. as substantially illustrated in FIG. 110 or FIG. 111; and
an onset of decomposition in a TGA thermograph of between 12° and 165° C., as substantially illustrated in FIG. 110.

In one embodiment, there is provided a crystalline 5-MeO-DMT Hydrochloride, characterised by one or more of:
an endothermic event in a DSC thermograph having an onset temperature of between 14° and 150° C., a peak of between 142 and 148° C.; and
an onset of decomposition in a TGA thermograph of between 12° and 165° C.

In one embodiment, there is provided a crystalline 5-MeO-DMT Hydrochloride, characterised by one or more of:
an endothermic event in a DSC thermograph having an onset temperature of between 14° and 150° C., a peak of between 142 and 148° C. as substantially illustrated in FIG. 110 or FIG. 111; and
an onset of decomposition in a TGA thermograph of between 12° and 165° C. as substantially illustrated in FIG. 110.

In one embodiment, there is provided a crystalline 5-MeO-DMT Hydrochloride, characterised by one or more of:
an endothermic event in a DSC thermograph having an onset temperature of between 14° and 150° C., a peak of between 142 and 148° C. and an enthalpy of between −115 J/g and −125 J/g; and
an onset of decomposition in a TGA thermograph of between 12° and 165° C.

In one embodiment, there is provided a crystalline 5-MeO-DMT Hydrochloride, characterised by one or more of:
an endothermic event in a DSC thermograph having an onset temperature of between 14° and 150° C., a peak of between 142 and 148° C. and an enthalpy of between −115 J/g and −125J/gas substantially illustrated in FIG. 110 or FIG. 111; and
an onset of decomposition in a TGA thermograph of between 12° and 165° C. as substantially illustrated in FIG. 110.

In one embodiment, there is provided a crystalline 5-MeO-DMT Hydrochloride, characterised by one or more of:
an endothermic event in a DSC thermograph having an onset temperature of 145.57° C., a peak of 146.22° C. and an enthalpy of −121.95 J/g; and
an onset of decomposition in a TGA thermograph of between 12° and 165° C.

In one embodiment, there is provided a crystalline 5-MeO-DMT Hydrochloride, characterised by one or more of:
an endothermic event in a DSC thermograph having an onset temperature of 145.57° C., a peak of 146.22° C. and an enthalpy of −121.95 J/g as substantially illustrated in FIG. 110 or FIG. 111; and
an onset of decomposition in a TGA thermograph of between 12° and 165° C. as substantially illustrated in FIG. 110.

### Example 45: Dynamic Vapour Sorption (DVS) of 5-MeO-DMT Hydrochloride

Sorption isotherms were obtained using a Hiden Isochema moisture sorption analyser (IGAsorp Systems Firmware V20.19.005 COM3) and operated by Isochema HIsorp 2019 V4.02.0102. The sample was maintained at a constant temperature (25° C.) by the instrument controls. The humidity was controlled by mixing streams of dry and wet nitrogen, with a total flow of 250 ml·min-1. The weight change of the sample was monitored as a function of humidity by a microbalance (accuracy +/−0.005 mg). The instrument was verified for relative humidity content by measuring three calibrated Rotronic salt solutions (10-50-88%).

A defined amount of sample was placed in a tared mesh stainless steel basket under ambient conditions. A typical experimental run consisted of three cycles (desorption, sorption, desorption, sorption, desorption and sorption) at a constant temperature (25° C.) and 10% RH intervals over a 0-90% RH range (60 minutes for each humidity level). This type of experiment should demonstrate the ability of samples studied to absorb moisture (or not) over a set of well-determined humidity ranges.

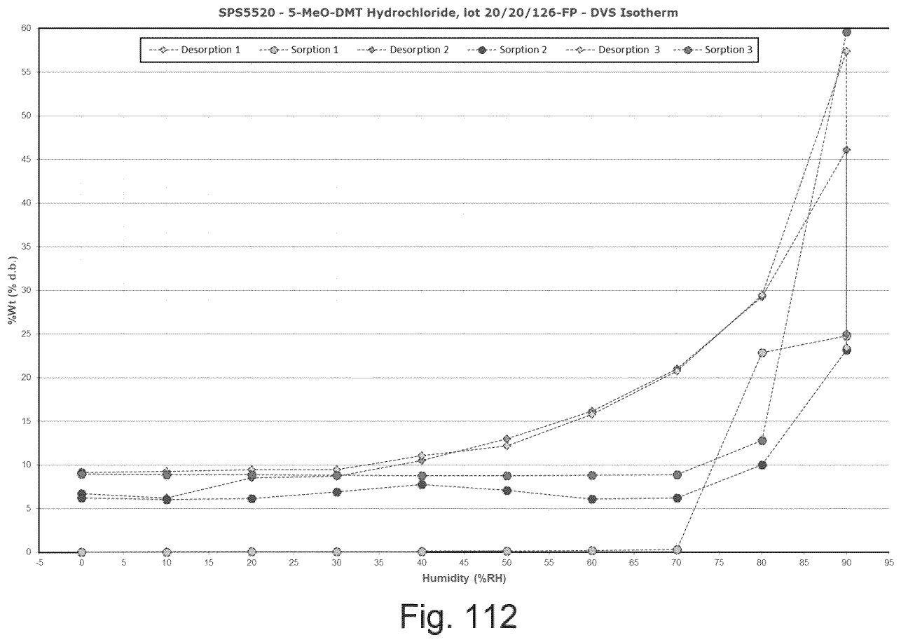

The DVS isotherm of 5-MeO-DMT Hydrochloride, lot 20/20/126-FP (FIG. 112) was found to undergo significant moisture uptake upon the first sorption cycle from 70% RH. Approximately 23%^{w}/w uptake is observed between 70−80% RH, whereas less than 0.3%^{w}/w moisture uptake from 0-70% RH was observed. A further 20%^{w}/w moisture uptake is observed up to and when held at 90% RH before commencement of the second desorption cycle. Subsequent sorption and desorption cycles follow a similar profile with some observed hysteresis between operations that do not match the original desorption step. These return to ca. 6-9%^{w}/w above the minimum mass recorded at 0% RH, which indicates significant retention of moisture. Upon completion of the DVS cycle, the input material was noted to have completed deliquesced.

Following the DVS observations of 5-MeO-DMT Hydrochloride, lot 20/20/126-FP, a modified DVS isotherm of lot 20/45/006-FP (the same crystalline version) was undertaken to examine material behaviour from 60% RH and above. A 2 cycle DVS with desorption beginning from 40-0% RH with sorption from 0-60% RH in 10% RH intervals, followed by incremental 5% RH increases to 65, 70, 75, 80 and finally 85% RH. This is to obtain in-depth profiling of the material towards humidity at these elevated levels.

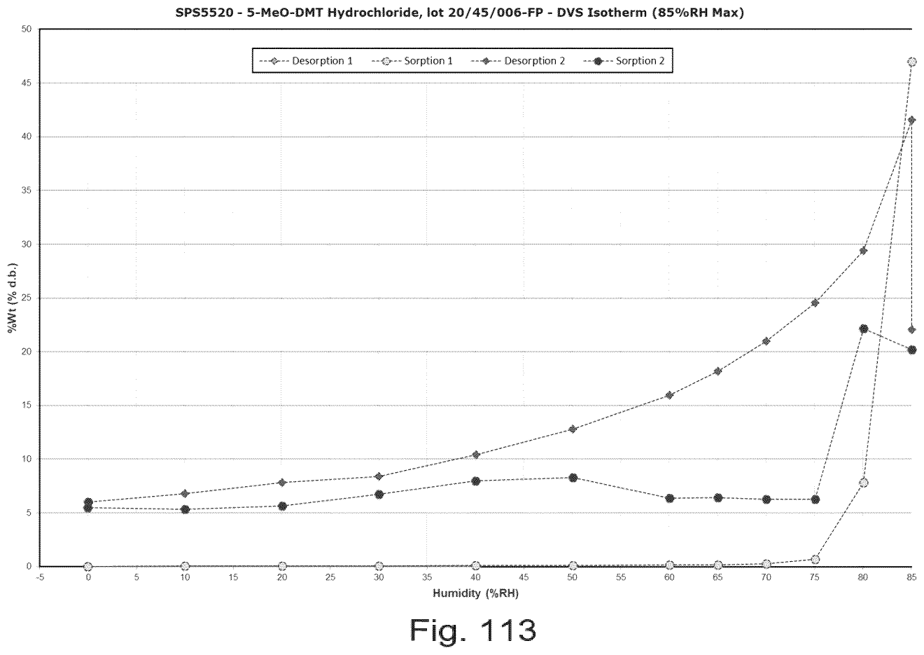

No significant moisture uptake/loss in first desorption-sorption profile between 0-70% RH was noted (FIG. 113), followed by a ca. 0.46% w/w increase from 70-75% RH. A further ca. 7% uptake is observed from 75-80% RH, then ca. 40% from 80-85% w/w. Complete deliquescence of the solids was observed upon isolation of the material post DVS analysis, which has likely occurred above 80% RH.

Despite the observed deliquescence above 80% RH, the solids demonstrate robustness between 0-75% RH and with adequate protection from moisture and conditional storage, this issue would likely be easily mitigated.

In one embodiment, there is provided crystalline 5-MeO-DMT Hydrochloride, characterised by a DVS isotherm profile as substantially illustrated in FIG. 112 or FIG. 113.

In one embodiment, there is provided a crystalline 5-MeO-DMT Hydrochloride, characterised by one or more of:
an endothermic event in a DSC thermograph having an onset temperature of between 14° and 150° C., optionally a peak of between 142 and 148° C. and optionally an enthalpy of between −115 J/g and −125 J/g;
an onset of decomposition in a TGA thermograph of between 12° and 165° C.; and
a DVS isotherm profile as substantially illustrated in FIG. 112 or FIG. 113.

In one embodiment, there is provided a crystalline 5-MeO-DMT Hydrochloride, characterised by one or more of:
an endothermic event in a DSC thermograph having an onset temperature of between 14° and 150° C., optionally a peak of between 142 and 148° C. and optionally an enthalpy of between −115 J/g and −125 J/g as substantially illustrated in FIG. 110 or FIG. 111;
an onset of decomposition in a TGA thermograph of between 12° and 165° C. as substantially illustrated in FIG. 110; and
a DVS isotherm profile as substantially illustrated in FIG. 112 or FIG. 113.

### Example 46: Optical Microscopy of 5-MeO-DMT Hydrochloride

Optical microscopy examination was undertaken using an Olympus BX53M polarised light microscope and an Olympus SC50 digital video camera for image capture using imaging software Olympus Stream Basic, V2.4. The image scale bar was verified against an external graticule, 1.5/0.6/0.01 mm DIV, on a monthly basis.

A small amount of each sample was placed onto a glass slide and dispersed as best as possible, using mineral dispersion oil if required. The samples were viewed with appropriate magnification and various images recorded.

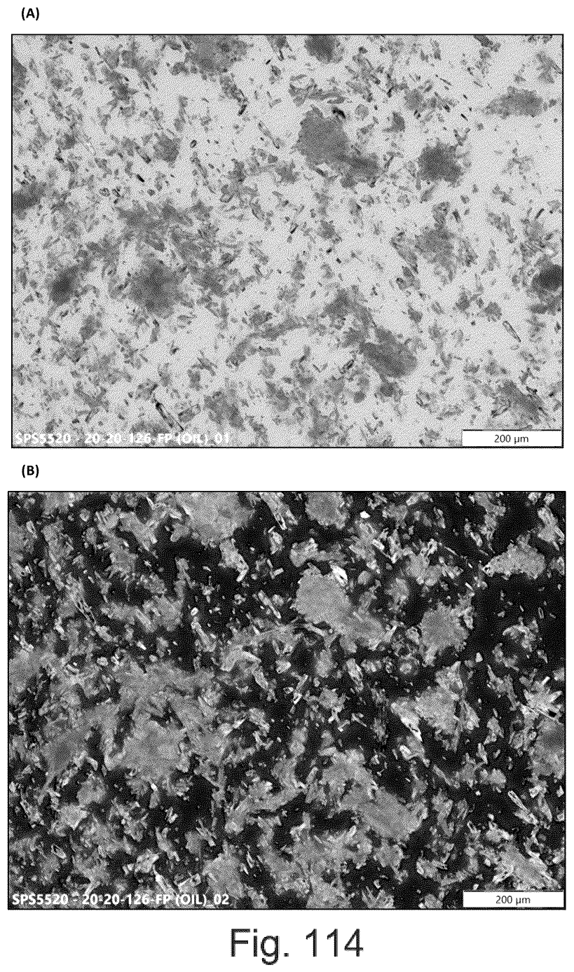

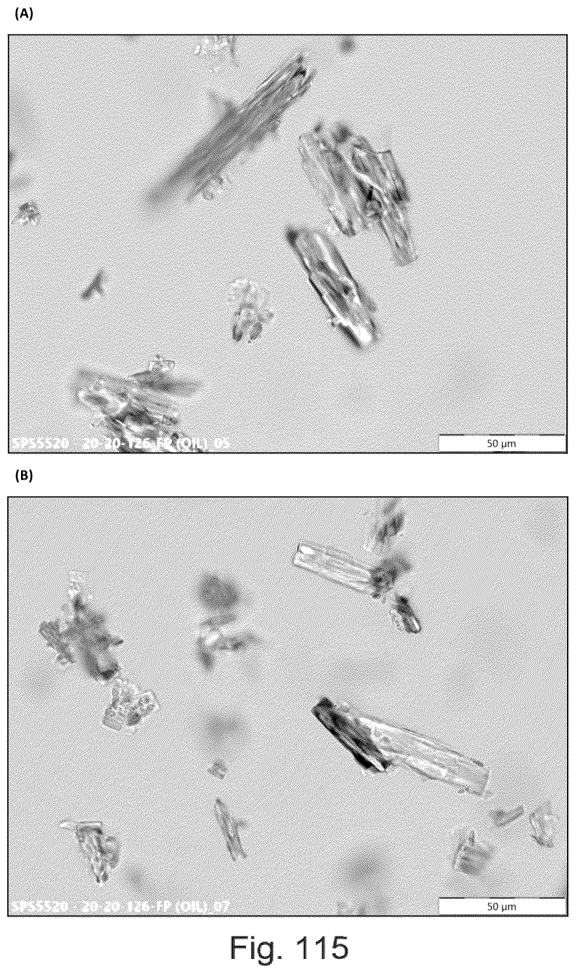

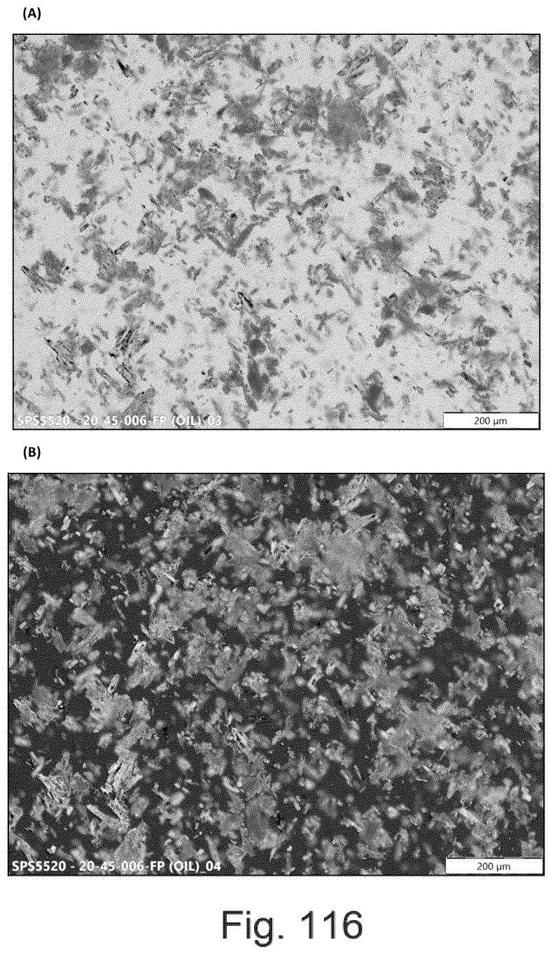

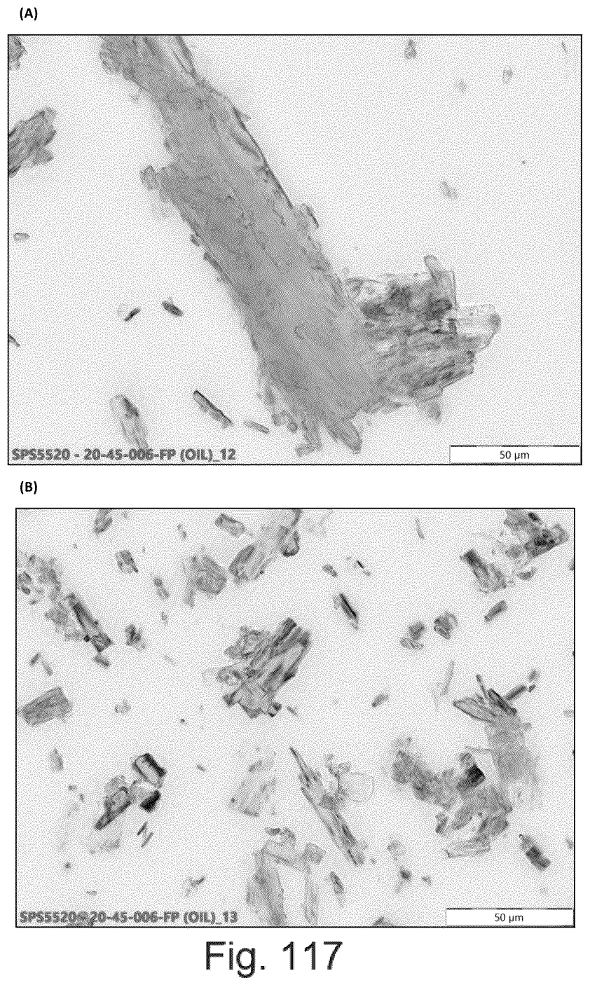

The microscopy of 5-MeO-DMT Hydrochloride, lots 20/20/126-FP (FIGS. 114-115) and 20/45/006-FP (FIGS. 116-117) do not differ significantly. Both consist of highly birefringent particulates with evidence of significant solid attrition of the particulates. A thin, columnar habit is, however, observed from individual crystallites that remain intact. There is also evidence of larger particulates that are made up of several individual crystallites that have accreted together during manufacture. Particle size of the main individual crystallites is estimated between ca. 10−50 μm, with some longer particulates up to 100 μm and a width of ca. 5-10 μm. A large quantity of smaller fragments of variable size and shape below 10 μm are noted, with several large solid accretions above 100 μm in diameter also present.

1. A method of synthesizing the benzoate salt of 5-MeO-DMT comprising the step of treating the hydrochloride salt of 5-MeO-DMT with a base, prior to the addition of benzoic acid.

2. The method of claim 1, wherein the benzoate salt is crystalline.

3. The method of claim 1, wherein the method comprises the step of suspending the hydrochloride salt in a suspending organic solvent; wherein optionally the suspending organic solvent is an alcohol, ester, an acetate and/or an acetate ester.

4. The method of claim 1, wherein the benzoic acid is in solution in an organic solvent; wherein optionally the organic solvent is an alcohol, ester, an acetate and/or an acetate ester.

5. The method of claim 1, wherein the benzoic acid and the hydrochloride salt are present in substantially equal molar amounts.

6. The method of claim 1, wherein the reaction with the benzoic acid takes place at an elevated temperature, optionally at a temperature between 40-65, 45-60, 50-55° C., or at/near the boiling point of the resultant reaction mixture.

7. The method of claim 1, wherein the reaction with the benzoic acid takes place at an elevated temperature, and the resultant reaction mixture is allowed to cool to room temperature or lower, is allowed to cool to below 10° C., is allowed to cool to below 5° C., or is allowed to cool to between 5 and 0° C.

8. The method of claim 1, wherein the benzoate salt is filtered from the resultant reaction mixture.

9. The method of claim 8, wherein the filtered benzoate salt is washed with a washing organic solvent; wherein optionally the washing organic solvent is an alcohol, ester, an acetate and/or an acetate ester.

10. The method of claim 8, wherein the benzoate salt is washed with cooled washing organic solvent, optionally the washing organic solvent is cooled to below room temperature, is cooled to below 10° C., is cooled to below 5° C., or cooled to between 5 and 0° C.

11. The method of claim 8, wherein the filtered benzoate salt is dried under vacuum.

12. The method of claim 1, wherein the hydrochloride salt is base-treated with an aqueous basic solution, prior to the addition of benzoic acid.

13. The method of claim 12, wherein the basic solution comprises an alkoxide, optionally the basic solution comprises sodium hydroxide, further optionally the basic solution is 5% aqueous sodium hydroxide.

14. The method of claim 12, wherein the hydrochloride salt is suspended in the suspending organic solvent prior to being base-treated, wherein optionally the suspending organic solvent is an alcohol, ester, an acetate and/or an acetate ester.

15. The method of claim 12, wherein the resultant based-treated reaction is partitioned with an extracting organic solvent to give an extract comprising the base-conditioned hydrochloride salt; wherein optionally the extracting organic solvent is an alcohol, ester, an acetate and/or an acetate ester.

16. The method of claim 3, wherein the suspending organic solvent is isopropyl acetate (IPAc).

17. The method of claim 4, wherein the organic solvent is IPAc.

18. The method of claim 9, wherein the washing organic solvent is IPAc.

19. The method of am claim 15, wherein the extracting organic solvent is IPAc.

20. The method of claim 15, wherein the organic phase is washed with water.

21-25. (canceled)
